(19) United States
(12) Patent Application Publication
(10) Pub. No.: US 2025/0340537 A1
(43) Pub. Date: Nov. 6, 2025

(54) PHARMACEUTICALLY ACTIVE COMPOUNDS, COMPOSITIONS, AND METHODS FOR MODULATING PENDRIN

(71) Applicants: ARBORMED Co., Ltd., Seoul (KR); UIF (University Industry Foundation), Yonsei University, Seoul (KR)

(72) Inventors: Jae Young CHOI, Seoul (KR); Gyoon Hee HAN, Hwaseong-si (KR); Min Goo LEE, Seoul (KR); Wan NAMKUNG, Incheon (KR); Jinsei JUNG, Seoul (KR); Sungwoo CHO, Seoul (KR); Shinhye NOH, Goyang-si (KR); Doona SONG, Pyeongtaek-si (KR); Dongsik PARK, Suwon-si (KR); Hong Jae LEE, Suwon-si (KR); Weonbin IM, Yongin-si (KR); So-Young EUN, Yongin-si (KR); Eok PARK, Seoul (KR); Mihwa SHIN, Seoul (KR)

(21) Appl. No.: 19/143,443

(22) PCT Filed: Dec. 29, 2023
(86) PCT No.: PCT/KR2023/022045

§ 371 Date: Jun. 26, 2025

(30) Foreign Application Priority Data
Dec. 30, 2022 (KR) 10-2022-0190973

(51) Int. Cl. C07D 403/04; A61K 31/337; A61K 31/36; A61K 31/4184; A61K 31/421; A61K 31/4439; A61K 31/496; A61K 31/497; A61K 31/506; C07D 401/04; C07D 401/14; C07D 403/14; C07D 405/14; C07D 413/14

(52) U.S. Cl. CPC C07D 403/04; A61K 31/337; A61K 31/36; A61K 31/4184; A61K 31/421; A61K 31/4439; A61K 31/496; A61K 31/497; A61K 31/506; C07D 401/04; C07D 401/14; C07D 403/14; C07D 405/14; C07D 413/14

(57) ABSTRACT

The present invention relates to a novel compound as pendrin corrector represented by the following Chemical formula 1 and a composition for preventing or treating pendred syndrome and the related disease thereof comprising the same as an active ingredient:

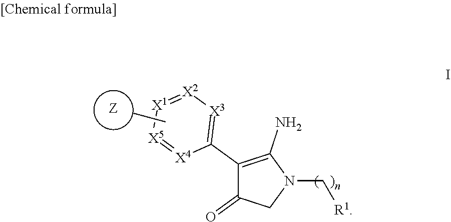

### TECHNICAL FIELD

The present invention relates to a novel compound as pendrin corrector and a composition for preventing or treating pendred syndrome and the related disease thereof comprising the same as an active ingredient.

### BACKGROUND ART

Pendrin is an anion exchange channel protein encoded by SLC26A4 (PDS) as a member of the SLC26A family. It is expressed on the apical cell membrane and mediates the transport of Cl⁻, HCO₃⁻, OH⁻, and I⁻ ions, as well as formate, nitrate and thiocyanate. It is prominently detected in the inner ear, thyroid and kidney, although other tissues also showed induced expression of pendrin under certain conditions. In the inner ear, pendrin was expressed in endolymphatic sac and hair cells. Pendrin defects caused by genetic mutations render endolymph acidification as well as reduced Ca²⁺ re-absorption, leading to auditory sensory transduction defects including Pendred Syndrome (Bassot, C., et al. 2017; Biochimie. 132: 109-120).

A few types of pharmacological modulators have been reported to rescue ion channel functions of ion transporters by directly interacting with targeted malfunctioning proteins such as Cystic Fibrosis Transmembrane Conductance Regulator (CFTR). Among those modulators, correctors, also known as pharmacological chaperons, and potentiators are the major types of such modulators that rescue the function of a particular membrane transporter. Correctors or pharmacological chaperones help mutated polypeptides properly fold into functional transporter proteins. Thus, correctors allow mutant proteins to form a functionally intact structure, thereby becoming targeted to the plasma membrane passing through trans-Golgi network with proper post-translational modifications. Potentiators, on the other hand, are the modulators augmenting the channel-gating function by stabilizing the protein and or by increasing the conductance function of the transporter proteins on the cell surface (Collawn, J. F., et al., 2014; Am. J. Physiol. Lung Cell. Mol. Physiol. 307: L431-L434). Multiple compounds with such strategies described above have been developed and marketed for certain diseases such as cystic fibrosis caused by genetic defects of CFTR (Lopes-Pacheco, M., 2020; Front. Pharmacol.).

### DISCLOSURE

### Technical Problem

The present invention is based on the discovery that a certain compound can act as a pendrin corrector with the potential to prevent, improve or treat pendred syndrome and the related disease thereof.

### Technical Solution

In one aspect, the present invention provides a compound represented by the following Chemical formula 1, optical isomer thereof, a mixture of two isomers thereof, precursor thereof, pharmaceutically acceptable salt thereof or solvate thereof:

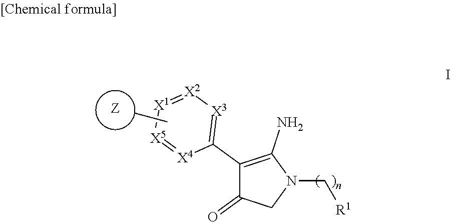

X¹ is, independently at each occurrence, selected from CH, C—Z and N;
X² is, independently at each occurrence, selected from CH, C—Z and N;
X³ is, independently at each occurrence, selected from CH, C—Z and N;
X⁴ is, independently at each occurrence, selected from CH, C—Z and N;
X⁵ is, independently at each occurrence, selected from CH, C—Z and N;
n is, independently at each occurrence, selected from 0, 1 and 2;
R¹ is, at each occurrence, independently selected from the group consisting of hydrogen, C1-C6 alkyl, C1-C6 alkyl substituted with one or several of halogen, C1-C6 alkyl, ═O, C3-C10 cycloalkyl, C3-C10 heterocycloalkyl, C1-C4 haloalkyl, OR⁶ and NR⁶R⁷; C3-C10 cycloalkyl, C3-C10 cycloalkyl substituted with one or several of halogen, C1-C6 alkyl, ═O, C3-C10 cycloalkyl, C3-C10 heterocycloalkyl, C1-C4 haloalkyl, OR⁶ and NR⁶R⁷; C3-C10 heterocycloalkyl, C3-C10 heterocycloalkyl substituted with one or several of halogen, C1-C6 alkyl, ═O, C3-C10 cycloalkyl, C3-C10 heterocycloalkyl, C1-C4 haloalkyl, OR⁶ and NR⁶R⁷; C6-C12 aryl, C6-C12 aryl substituted with one or several of halogen, C1-C6 alkyl, C3-C10 cycloalkyl, C3-C10 heterocycloalkyl, C1-C4 haloalkyl, OR⁶ and NR⁶R⁷; C3-C12 heteroaryl, C3-C12 heteroaryl substituted with one or several of halogen, C1-C6 alkyl, C3-C10 cycloalkyl, C3-C10 heterocycloalkyl, C1-C4 haloalkyl, OR⁶ and NR⁶R⁷;
Z is any structure of the following group A;

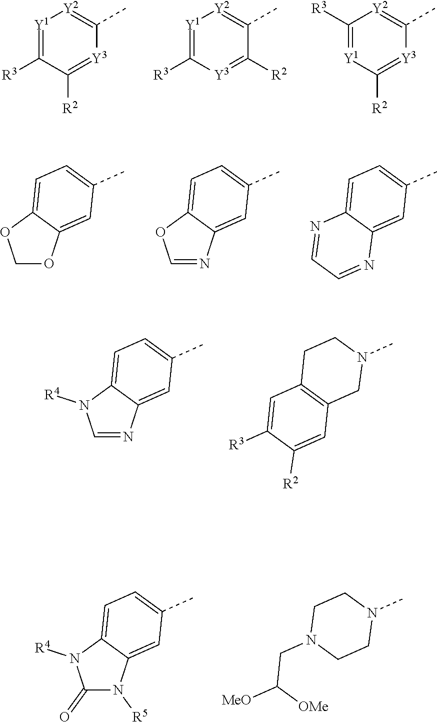

Group A
Y¹, Y² and Y³ are, independently at each occurrence, selected from CH and N;
R² and R³ are, at each occurrence, independently selected from the group consisting of hydrogen, halogen, C1-C6 alkyl, C3-C10 cycloalkyl, C3-C10 heterocycloalkyl, C1-C4 haloalkyl, OR⁶ and NR⁶R⁷, any of which is optionally substituted;
R⁴ and R⁵ are, are, at each occurrence, independently selected from the group consisting of hydrogen, C1-C6 alkyl, C3-C10 cycloalkyl, any of which is optionally substituted;
R⁶ and R⁷ are, at each occurrence, independently selected from the group consisting of hydrogen, C1-C4 haloalkyl, C1-C6 alkyl, C1-C6 alkyl substituted with one or several of halogen, C1-C6 alkyl, ═O, C3-C10 cycloalkyl, C3-C10 heterocycloalkyl, C1-C4 haloalkyl, OR⁸ and NR⁸R⁹; C3-C10 cycloalkyl, C3-C10 cycloalkyl substituted with one or several of halogen, C1-C6 alkyl, ═O, C1-C4 haloalkyl, OR⁸ and NR⁸R⁹; C3-C10 heterocycloalkyl, C3-C10 heterocycloalkyl substituted with one or several of halogen, C1-C6 alkyl, ═O, C1-C4 haloalkyl, OR⁸ and NR⁸R⁹;
R⁸ and R⁹ are, at each occurrence, independently selected from the group consisting of hydrogen, C1-C6 alkyl, C3-C10 cycloalkyl, C3-C10 heterocycloalkyl and C1-C4 haloalkyl, any of which is optionally substituted;
and pharmaceutically acceptable salts thereof.

In another aspect, the present invention provides a pharmaceutical composition for preventing, improving or treating of pendred syndrome or the related disease thereof, comprising at least one of the compounds, at least one optical isomer thereof, at least one mixture of two isomers thereof, at least one the precursor thereof, at least one pharmaceutically acceptable salt thereof or at least one solvate thereof as an active ingredient.

In an additional aspect, the present invention provides a composition comprising the compound represented by Chemical formula 1 or mixture thereof, and a composition comprising the compound represented by Chemical formula 1 or mixture thereof with a pharmaceutically acceptable carrier.

In an additional aspect, the present invention provides a use of the compound represented by Chemical formula 1 and pharmaceutical composition thereof, as a pendrin corrector.

In an additional aspect, the present invention provides a use of the compound represented by Chemical formula 1, mixture thereof and pharmaceutical composition thereof, for preventing or improving of pendred syndrome or the related disease thereof as an active ingredient in a health functional food.

In an additional aspect, the present invention provides a use of the compound and pharmaceutical composition thereof as a pendrin corrector, for preventing or improving of pendred syndrome or the related disease thereof as an active ingredient in a health functional food.

### Advantageous Effects

According to the present invention, the novel compound can act as a pendrin corrector, and thus, consequently, it can be usefully used as a composition for preventing, treating or improving of pendred syndrome or the related disease thereof.

### DESCRIPTION OF DRAWINGS

FIG. 1 shows western blot analysis results of compound 9 treated patient epithelial cells inferred from patient nasal epithelial cells. (A, B) B-form, which represents a specific non-glycosylated precursor form of pendrin, showed few noticeable changes in response to Compound 9 treatment; however, in C-form, which represents a fully-glycosylated functional pendrin, a notable dose-dependent increase is observed, especially at a high concentration of 10 μM. These observations, extrapolated to patient-derived nasal epithelial cells, suggest a concentration-dependent effect of Compound 9 treatment on C-form expression in cells harboring the H724R mutation.

FIG. 2 shows immunofluorescence staining results of compound 9, 18 and 105. (A) illustrates immunofluorescence staining outcomes for epithelial cells under different conditions. In the vehicle and IL-4 groups, pendrin expression is absent, indicated by no fluorescence. Conversely, treatment with Compound 9, Compound 18 and Compound 105 at 10 μM exhibits a clear red fluorescence, signifying enhanced pendrin expression localized on the epithelial cell membrane. Scale bars=20 μm. (B) is quantitative representation, comparing the mean fluorescence intensity levels between the vehicle, IL-4 and Compound 9, Compound 18 and Compound 105 (10 μM) groups. Statistical significance indicated a significant difference in pendrin expression intensity between experimental conditions. *p<0.05.

### BEST MODE

### 1. Definition

Unless defined otherwise, all technical and scientific terms used herein have the meaning commonly understood by those skilled in the art to which this disclosure belongs. As used herein, the following terms have the meanings mentioned below, unless specified otherwise.

Unless specifically stated or clear from the context, the term used herein “or” is understood as inclusive.

Unless specifically stated or clear from the context, the term used herein “about” is understood within the general acceptance range of the art, for example, within two standard deviations of the mean. About may be understood to be within 10%, 9%, 8%, 7%, 6%, 5%, 4%, 3%, 2%, 1%, 0.5%, 0.1%, 0.05% or 0.01% of the stated value. Unless otherwise clear from the context, all numerical values provided herein are modified by the term about.

The terms “activator, “drug” and “pharmaceutical formulation” are interchangeably used herein to refer to a chemical material or compound which induces a desired pharmacological effect (for example, such as reduction of inflammation) when administered to a subject by any means described herein (for example, any animal including a human or non-human animal).

“Additive” used herein may refer to any additional ingredient which can be added to the composition and chemical formula described herein. For example, providing that the additional ingredient is pharmaceutically acceptable for a particular condition being treated, the additive may include an excipient (for example, one or more excipients), an anti-oxidant (for example, one or more anti-oxidants), a stabilizer (for example, one or more stabilizers), a preservative (for example, one or more preservatives), a pH adjusting agent and/or buffers (for example, one or more pH adjusting agents and/or buffers), an isotonic adjusting agent (for example, one or more isotonic adjusting agents), a thickener (for example, one or more thickeners), a suspending agent (for example, one or more suspending agents), a binding agent (for example, one or more binding agents), a viscosity increasing agent (for example, one or more viscosity increasing agents), and the like. In addition, the additive may comprise a treatment agent and a drug delivery modifier, and an enhancer such as calcium phosphate, magnesium stearate, talc, monosaccharides, disaccharides, starch, gelatin, cellulose, methyl cellulose, sodium carboxymethyl cellulose, dextrose, hydroxypropyl-beta-cyclodextrin, polyvinylpyrrolidone, low melting point wax and ion exchange resin, and a combination of any two or more thereof.

The term used herein “administration” means oral administration, suppository, topical, intravenous, parenteral, trans-tympanic, intratympanic, intracochlear, intraperitoneal, intramuscular, intralesional, intrathecal, intranasal, intravitreal or subcutaneous administration, or a sustained release device such as implantation of a small-osmotic pump to a subject. The administration is administered by any route including parenteral and transmucosal (for example, oral, intranasal, intrapulmonary, intrarectal, intrabuccal, intravaginal, intraocular and dermal) routes. “Analogue” and “derivative” are interchangeably used herein and refer to a compound that has the same core as the parent compound, but differ from the parent compound in the order of bonding, in the absence or presence of one or more atoms and/or groups of atoms, and combinations thereof. The derivative may differ from the parent compound in one or more substituents present on the core which may comprise for example, one or more atoms, functional groups or substructures. In addition, the derivative may differ from the parent compound in the order of boning between atoms in the core. In general, the derivative may be at least theoretically, predicted to be formed from the parent compound through a chemical and/or physical process.

“Anti-oxidant” used herein may refer to an artificial or natural substance capable of preventing or delaying a certain type of damage and/or oxidation. The anti-oxidants are found in many foods including fruits and vegetables. In addition, they can be used as a dietary supplement. Exemplary anti-oxidants may include β-carotene, lutein, lycopene, selenium, vitamin A, vitamin C and vitamin E. Furthermore, other anti-oxidants known to those skilled in the art may be used. The anti-oxidant described herein may be used in an any appropriate amount.

“Co-administration” means that a compound or composition described herein is administered simultaneously immediately prior to or immediately following administration of an additional treatment or activator or additive described herein. The compound or composition of the present disclosure may be administered alone or co-administered to a patient. Co-administration is construed to include administration of the compounds individually or in combination (one or more compounds or agents) simultaneously or sequentially. If desired, agents may also be combined with other active substances.

In the present disclosure, “comprise”, “comprising”, “containing” and “having” and the like may have the meaning belonging to them and may mean “include”, “including” and the like; and “consisting essentially of” or “consist essentially of” may likewise have the meaning pertaining to them, and the terms are open-ended and permits the existence of more than the recited, unless the basic or novel features of the recited are altered by the existence of more than the recited, but prior art examples are excluded. “Simultaneous administration” used herein includes at least in part, overlap of duration.

For example, when two agents (for example, any agent or class of agents described herein having bioactivity) are administered simultaneously, their administration occurs within a certain desired time period. Administration of formulations may start and end on the same day. In addition, administration of one formulation may precede administration of a second formulation as long as the two agents are taken at least once on the same day. Similarly, administration of one formulation may be extended beyond administration of a second formulation as long as two formulations are taken at least once on the same day. To include simultaneous administration of a bioactive agent/a formulation, it is not necessary to take them at the same time each day.

“Effective amount” or “therapeutically effective amount” used herein is an amount sufficient to affect a desired biological effect such as a beneficial result including a clinical result. Thus, “effective amount” depends on the circumstances in which it is applied. An effective amount may vary depending on factors known in the art, such as the disease state, age, gender, and body weight of an individual being treated. Several divided doses may be administered daily, or the dose may be proportionally reduced as indicated by urgency of the therapeutic situation. In addition, the composition/formulation of the present disclosure may be administered as often as necessary to achieve a therapeutic amount.

The term “Intermittent administration” used herein includes a period during which a formulation is administered (this may be considered a “first administration period”), a subsequent period during which a formulation is not ingested or is ingested in a lower dose (this may be considered an “off-period”), and a subsequent period during which a formulation is administered again (this may be considered an “second administration period). In general, during the second administration period, the dose level of the formulation is consistent with that administered during the first administration period, but it may be increased or decreased as medically necessary.

“Liquid” used herein is an administration form consisting of a composition in a liquid state. Liquid may be spilled; and it flows and behaves in a container at a room temperature. Liquid exhibits Newton or pseudoplastic flow behavior.

In an embodiment, “semi-liquid” used herein may have the properties of both liquids and other formulation (i.e., suspension, emulsion, solution, cream, gel, jelly, etc.).

The term used herein “Ointment” may refer to a highly viscous liquid or semi-liquid formulation that may be used in the therapeutic treatment of a disease, syndrome or condition.

“Pharmaceutically acceptable carrier” used herein includes physiologically appropriate any and all solvents, dispersive media, coating, anti-microbial and anti-fungal agents, isotonic and absorption retardants, and the like. The type of the carrier may be selected on the basis of the intended administration route. The pharmaceutically acceptable carrier includes a sterile aqueous solution or dispersion and sterile powder for instant preparation of a sterile local solution or dispersion. The use of such media and agents for pharmaceutically active substances is well known in the art. As long as any conventional medium or agent is incompatible with the composition (for example, Chemical formula 1 described herein, a derivative or analogue of Chemical formula 1, or pharmaceutically acceptable salt, solvent, hydrate or polymorph thereof), its use is considered in the composition for the present disclosure.

“Pharmaceutical carrier” or “carrier” used herein may further include a pharmaceutically acceptable carrier, excipient or stabilizer which is non-toxic to cells or mammals in an adopted dose and concentration. The physiologically acceptable carrier is often an aqueous pH buffer solution. The example of the physiologically acceptable carrier includes a buffer such as phosphate, citrate and other organic acids; an anti-oxidant including ascorbic acid; a low molecular weight (less than about 10 residues of polypeptide); a protein such as serum albumin, gelatin or immunoglobulin; a hydrophilic polymer such as polyvinylpyrrolidone; an amino acid such as glycine, glutamine, asparagine, arginine or lysine; a monosaccharide, a disaccharide and other carbohydrate including glucose, mannose or dextrin; a chelating agent such as EDTA; a sugar-alcohol such as mannitol or sorbitol; a counter ion forming a salt such as sodium; and/or a nonionic surfactant such as Tween™, polyethylene glycol (PEG) and Pluronics™. Additionally, ‘pharmaceutically acceptable’ means that it is approved or may be approved by a federal or state government regulatory agency or a corresponding agency in a country other than the United States, or that it is listed in the United States Pharmacopoeia or other generally approved pharmacopoeia for use in animals, and more particularly, in humans.

The term “pharmaceutically acceptable salt or complex” refers to a salt or complex represented by the following specified Chemical formula 1. The example of this salt includes a base addition salt formed by a response of a compound represented by Chemical formula 1 which has an organic or inorganic base such as hydroxide, carbonate or bicarbonate of a metal cation as selected from the group consisting of alkali metals (for example, sodium, potassium or lithium) and alkali earth metals (for example, calcium or magnesium) or has primary, secondary or tertiary alkyl amine, but not limited thereto. An amine salt induced from methylamine, dimethylamine, trimethylamine, ethylamine, diethylamine, triethylamine, morpholine, N-methyl-D-glutamine, N,N′-bis(phenylmethyl)-1,2-ethanediamine, tromethamine, ethanolamine, diethanolamine, ethylene diamine, N-methylmorpholine, procaine, piperidine, piperazine, and the like is considered to be within the range of the present invention.

Furthermore, “salt” or “salt form” or “pharmaceutically acceptable salt” used herein may include a base addition salt (from with free carboxyl or other anionic groups) derived from an inorganic base such as for example, sodium, potassium, ammonium, calcium or ferric hydroxide, and an organic base such as for example, isopropylamine, trimethylamine, 2-ethylamino-ethanol, histidine, procaine, and the like. This salt is formed as an acid addition salt having any free cationic group, and for example, it is generally formed with an inorganic acid such as hydrochloric acid, sulfuric acid or phosphoric acid, or an organic acid such as formic acid, acetic acid, citric acid, p-toluenesulfonic acid, methanesulfonic acid, oxalic acid, tartaric acid, mandelic acid, and the like. The salt of the present disclosure may include an amine salt formed by protonation of an amino group having an inorganic acid such as hydrochloric acid, hydrobromic acid, hydriodic acid, sulfuric acid, phosphoric acid, and the like. In addition, the salt of the present disclosure includes an amine salt formed by protonation of an amino group having an appropriate organic acid such as p-toluenesulfonic acid, acetic acid, and the like.

The term used herein “pH agent” or “buffer” may refer to a compound or buffer useful as a pH adjusting agent. This may include a glycerol buffer, citrate buffer, borate buffer, acetate buffer, gluconate buffer, phosphate buffer or citrate-phosphate buffer, but not limited thereto. The pH agent or buffer may be used in an any appropriate amount.

The term used herein “preservative” may refer to a substance or chemical substance which prevents an undesirable change of a compound or composition or chemical formula described herein. The appropriate preservative may include for example, benzalkonium chloride, thimerosal, chlorobutanol, methyl paraben, propyl paraben, phenylethyl alcohol, edetate disodium sorbate, onamer m polyquat, cetyl bromide, cetyl pyridinium chloride, benzyl bromide, EDTA, phenyl mercury nitrate, phenyl mercury acetate, Merthiolate, acetate and phenyl mercury borate, polymyxin B sulfate, methyl and propyl paraben, tertiary ammonium chloride, sodium benzoate, sodium propionate and sodium perborate, and other agents known to those skilled in the art or a combination thereof. The preservative may be used in any appropriate amount.

The term used herein, “prevent”, “preventing” or “prevention” and other grammatical equivalents includes for reduction of incidence of a syndrome, as well as for preventing development, occurrence, interference or avoidance of the syndrome of a disease or condition. The prevention may be complete (i.e., no detectable symptoms) or partial, so that fewer symptoms may be observed than in the absence of treatment. The term further includes prophylactic benefits. To prevent a disease or condition, the composition may be administered to a patient at risk of developing a specific disease or a patient reporting one or more physiological syndromes of the disease, although not necessarily diagnosing the disease.

Ranges provided herein are understood to be shorthand for all values within the ranges. For example, a range of 1 to 10 is understood to include not only all intermediate decimal values between the aforementioned integers such as 1.1, 1.2, 1.3, 1.4, 1.5, 1.6, 1.7, 1.8 and 1.9 but also any number, combination of numbers of subranges from the group consisting of 1, 2, 3, 4, 5, 6, 7, 8, 9 or 10. With respect to subranges, “nested subranges” extending from one of the endpoints of the range are particularly considered. For example, overlapping subranges of the example range of 1 to 50 may include 1 to 10, 1 to 20, 1 to 30, and 1 to 40 in one direction, or may include 50 to 40, 50 to 30, 50 to 20, and 50 to 10 in the other direction. The range may be expressed herein as “about” one specific value and/or “about” other specific value. When such range is expressed, other aspect includes one specific value and/or other specific value. Similarly, when values are expressed as approximations using the antecedent “about”, it is understood that the specific value forms the other aspect. It is further understood that the endpoints of each range are significant in relation to the other endpoints and independently of the other endpoints. In addition, throughout the application, it is understood that data are provided in a number of different formats and these data represent endpoints and starting points and ranges for any combination of data points. For example, when a specific data point “10” and a specific data point “15” are disclosed, it is considered that between 10 and 15, as well as mor than, more than or equal to, less than, less than or equal to, and equal to are disclosed. Furthermore, it is understood that each unit between two specific units is disclosed. For example, when 10 and 15 are disclosed, 11, 12, 13 and 14 are disclosed.

Additional excipients considered for use in the practice of the present disclosure are those available to those skilled in the art.

“Semi-solid gel” according to the present disclosure is a semi-solid. The apparent viscosity of a semi-solid formulation may increase with concentration.

“Sequential administration” used herein includes that administration of two formulations (for example, a compound or composition described herein) occurs separately on the same day or does not occur on the same day (for example, occurs on consecutive days).

“Solution” according to the present disclosure may be a clear, homogeneous liquid administration form containing one or more chemical substances dissolved in a solvent or mixture of solvents that are miscible with one another. As molecules of a drug substance in a solution are uniformly dispersed, the used of the solution as an administration form generally provides assurance of a uniform dosage upon administration and good accuracy when the solution is diluted or otherwise mixed.

The term “solvent” used herein refers to an aqueous or non-aqueous liquid solvent. The selection of the solvent depends particularly on the solubility and mode of administration of a composition. The aqueous solvent may consist of only water or may consist of water and one or more of miscible solvents, and may contain dissolved solutes such as sugars, buffers, salts or other excipients. More commonly used non-aqueous solvents are short-chain organic alcohols such as methanol, ethanol and propanol, short-chain ketones such as acetone, and polyalcohols such as glycerol.

“Subject” or “patient” means a human or non-human animal such as a mammal. The “subject” may include any animal including horses, dogs, cats, pigs, goats, rabbits, hamsters, monkeys, guinea pigs, rats, mice, lizards, snakes, sheep, cattle, fish and birds. The human subject may refer to a patient.

“Suspension” used herein is a liquid administration form containing solid particles dispersed in a liquid vehicle.

“Viscosity” used herein refers to the flow resistance of a fluid. A viscosity agent may be used herein, and for example, it includes polyvinyl alcohol, polyvinyl pyrrolidone, methyl cellulose, hydroxy propyl methyl cellulose, hydroxyethyl cellulose, carboxymethyl cellulose, hydroxy propyl cellulose, other agents known to those skilled in the art or combinations thereof.

The term “weight percentage” or “% (w/w)” refers to a percentage of a component in a solution calculated on the basis of the weight of the component and solvent. For example, 1% (w/w) solution of the component may have the component of 1 g dissolved in a solvent of 100 g. The term “volume percentage” or “% (v/v)” refers to a percentage of a component in a solution calculated on the basis of the volume of the component and solvent. For example, 1% (v/v) solution of the component may have the component of 1 ml dissolved in a solvent of 100 ml. The term “weight/volume percentage” or “% (w/v)” refers to a percentage of a component in a solution calculated on the basis of the weight of the component and the volume of the solvent. For example, the 1.0% (w/v) solution of the component may have the component of 1 g dissolved in a solvent of 100 ml.

The term “syndrome” used herein refers to a condition characterized by a group of symptoms that occur continuously together or a series of related symptoms. The syndrome (for example, acute respiratory distress syndrome) may be a set of medical signs and symptoms that are interrelated and often associated with a specific disease. On the other hand, a disease may be a health condition with a clearly defined reason behind it. However, the syndrome (from a Greek word meaning ‘to run together’) may cause a number of symptoms without an identifiable cause. They may imply the likelihood of an underlying disease or the likelihood that a disease will develop.

The term “treat”, “treating” or “treatment” and other grammatical equivalents used herein include alleviation, attenuation, improvement or prevention of disease, condition (for example, acute respiratory distress syndrome) or symptoms, prevention of an additional symptom, improvement or prevention of fundamental metabolic causes of symptoms, inhibition of disease or condition, for example, development arrest of disease or condition, alleviation of disease or condition, regression of disease or condition, alleviation of condition caused by disease or condition, or stop of symptoms of disease or condition, and are intend to include prevention. The term further includes achieving a therapeutic benefit and/or prophylactic benefit. The therapeutic benefit means eradication or improvement of fundamental disorder being treated. In addition, since a therapeutic benefit is achieved by eradication or improvement of one or more of physiological symptoms related to the fundamental disorder, even though a patient may still suffer from the fundamental disorder, the improvement is observed in the patient.

The term “health functional food” refers to a food or food supplement prepared or processed with a raw material, functional ingredient, active pharmaceutical component or additive, useful for improving and/or nourishing and/or preserving the physiological functions of the human body.

The term ‘PDS’ can also be used as pendrin protein encoded by the gene SLC26A4 (PDS).

Unless otherwise limited by the definition of an individual substituent, all the substituents should be understood to be all optionally substituted.

### 2. Compound

One aspect of the present invention provides a compound represented by the following Chemical formula 1, optical isomer thereof, a mixture of two isomers thereof, precursor thereof, pharmaceutically acceptable salt thereof or solvate thereof:

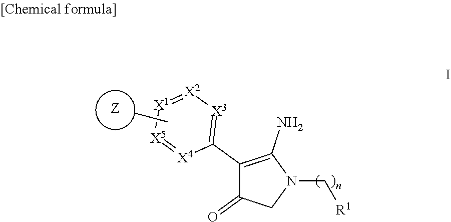

X is, independently at each occurrence, selected from CH, C—Z and N;
X² is, independently at each occurrence, selected from CH, C—Z and N;
X³ is, independently at each occurrence, selected from CH, C—Z and N;
X⁴ is, independently at each occurrence, selected from CH, C—Z and N;
X⁵ is, independently at each occurrence, selected from CH, C—Z and N;
n is, independently at each occurrence, selected from 0, 1 and 2;
R¹ is, at each occurrence, independently selected from the group consisting of hydrogen, C1-C6 alkyl, C1-C6 alkyl substituted with one or several of halogen, C1-C6 alkyl, ═O, C3-C10 cycloalkyl, C3-C10 heterocycloalkyl, C1-C4 haloalkyl, OR⁶ and NR⁶R⁷; C3-C10 cycloalkyl, C3-C10 cycloalkyl substituted with one or several of halogen, C1-C6 alkyl, ═O, C3-C10 cycloalkyl, C3-C10 heterocycloalkyl, C1-C4 haloalkyl, OR⁶ and NR⁶R⁷; C3-C10 heterocycloalkyl, C3-C10 heterocycloalkyl substituted with one or several of halogen, C1-C6 alkyl, ═O, C3-C10 cycloalkyl, C3-C10 heterocycloalkyl, C1-C4 haloalkyl, OR⁶ and NR⁶R⁷; C6-C12 aryl, C6-C12 aryl substituted with one or several of halogen, C1-C6 alkyl, C3-C10 cycloalkyl, C3-C10 heterocycloalkyl, C1-C4 haloalkyl, OR⁶ and NR⁶R⁷; C3-C12 heteroaryl, C3-C12 heteroaryl substituted with one or several of halogen, C1-C6 alkyl, C3-C10 cycloalkyl, C3-C10 heterocycloalkyl, C1-C4 haloalkyl, OR⁶ and NR⁶R⁷;
Z is any structure of the following group A;

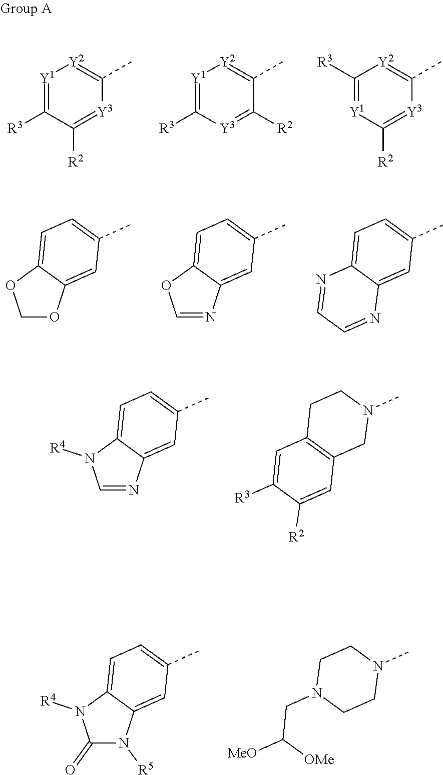

Y¹, Y² and Y³ are, independently at each occurrence, selected from CH and N;
R² and R³ are, at each occurrence, independently selected from the group consisting of hydrogen, halogen, C1-C6 alkyl, C3-C10 cycloalkyl, C3-C10 heterocycloalkyl, C1-C4 haloalkyl, OR⁶ and NR⁶R⁷, any of which is optionally substituted;
R⁴ and R⁵ are, are, at each occurrence, independently selected from the group consisting of hydrogen, C1-C6 alkyl, C3-C10 cycloalkyl, any of which is optionally substituted;
R⁶ and R⁷ are, at each occurrence, independently selected from the group consisting of hydrogen, C1-C4 haloalkyl, C1-C6 alkyl, C1-C6 alkyl substituted with one or several of halogen, C1-C6 alkyl, ═O, C3-C10 cycloalkyl, C3-C10 heterocycloalkyl, C1-C4 haloalkyl, OR⁸ and NR⁸R⁹; C3-C10 cycloalkyl, C3-C10 cycloalkyl substituted with one or several of halogen, C1-C6 alkyl, ═O, C1-C4 haloalkyl, OR⁸ and NR⁸R⁹; C3-C10 heterocycloalkyl, C3-C10 heterocycloalkyl substituted with one or several of halogen, C1-C6 alkyl, ═O, C1-C4 haloalkyl, OR⁸ and NR⁸R⁹;
R⁸ and R⁹ are, at each occurrence, independently selected from the group consisting of hydrogen, C1-C6 alkyl, C3-C10 cycloalkyl, C3-C10 heterocycloalkyl and C1-C4 haloalkyl, any of which is optionally substituted;
and pharmaceutically acceptable salts thereof.

In a preferred embodiment, in the Chemical Formula 1, Z is any structure of the following group B;

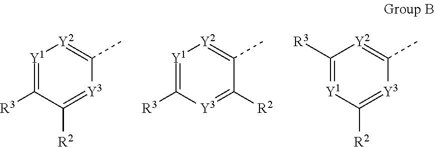

wherein, R² and R³ are as defined in claim 1; and
Y¹, Y² and Y³ are as defined in claim 1.

In a preferred embodiment, in the Chemical Formula 1, Z is

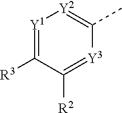

wherein R² and R³ are as defined above; and
Y¹, Y² and Y³ are as defined above.

In a preferred embodiment, the compound of the Chemical Formula 1 has the general formula II,

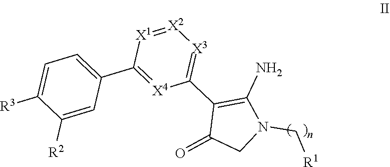

wherein, R¹, R² and R³ are as defined above; and
X¹, X², X³ and X⁴ are as defined above; and
n is as defined above.

In a preferred embodiment, in the Chemical Formula 1, R¹ is, at each occurrence, independently selected from the group consisting of hydrogen, C1-C6 alkyl, C1-C6 alkyl substituted with one or several of halogen, C1-C6 alkyl, ═O, C3-C10 cycloalkyl, C3-C10 heterocycloalkyl, C1-C4 haloalkyl, OR⁶ and NR⁶R⁷; C3-C10 cycloalkyl, C3-C10 cycloalkyl substituted with one or several of halogen, C1-C6 alkyl, ═O, C3-C10 cycloalkyl, C3-C10 heterocycloalkyl, C1-C4 haloalkyl, OR⁶ and NR⁶R⁷; C3-C10 heterocycloalkyl, C3-C10 heterocycloalkyl substituted with one or several of halogen, C1-C6 alkyl, ═O, C3-C10 cycloalkyl, C3-C10 heterocycloalkyl, C1-C4 haloalkyl, OR⁶ and NR⁶R⁷.

In a preferred embodiment, in the Chemical Formula 1, R¹ is, at each occurrence, independently selected from the group consisting of C6-C12 aryl, C6-C12 aryl substituted with one or several of halogen, C1-C6 alkyl, C3-C10 cycloalkyl, C3-C10 heterocycloalkyl, C1-C4 haloalkyl, OR⁶ and NR⁶R⁷; C3-C12 heteroaryl, C3-C12 heteroaryl substituted with one or several of halogen, C1-C6 alkyl, C3-C10 cycloalkyl, C3-C10 heterocycloalkyl, C1-C4 haloalkyl, OR⁶ and NR⁶R⁷.

In a preferred embodiment, in the Chemical Formula 1, R² and R³ are, at each occurrence, independently selected from the group consisting of OR⁶ and NR⁶R⁷.

In a preferred embodiment, the compound of the Chemical Formula 1 has one of the formulae 1)-120), as shown hereafter:

| # | |
|---|---|
| cpd | Structure |
| 1 | 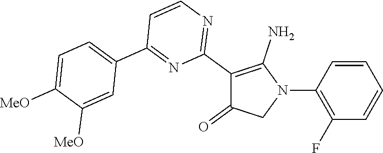 [SMILES: COc1ccc(-c2ccnc(C3=C(N)N(c4ccccc4F)CC3=O)n2)cc1OC] |
| 2 | 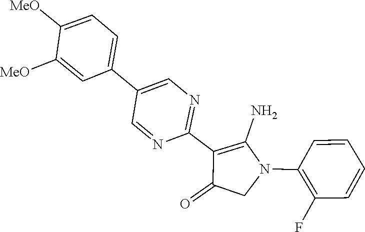 [SMILES: COc1ccc(-c2cnc(C3=C(N)N(c4ccccc4F)CC3=O)nc2)cc1OC] |
| 3 | 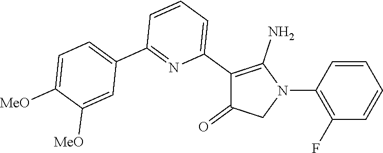 [SMILES: COc1ccc(-c2cccc(C3=C(N)N(c4ccccc4F)CC3=O)n2)cc1OC] |
| 4 | 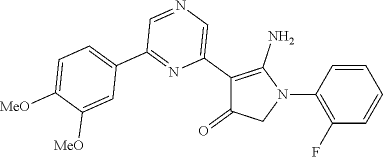 [SMILES: COc1ccc(-c2cncc(C3=C(N)N(c4ccccc4F)CC3=O)n2)cc1OC] |
| 5 | 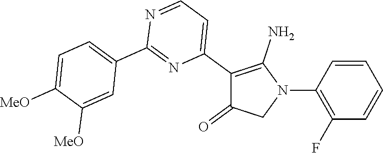 [SMILES: COc1ccc(-c2nccc(C3=C(N)N(c4ccccc4F)CC3=O)n2)cc1OC] |
| 6 | 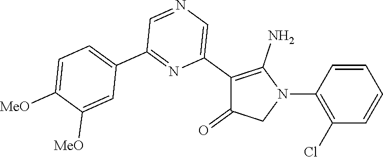 [SMILES: COc1ccc(-c2cncc(C3=C(N)N(c4ccccc4Cl)CC3=O)n2)cc1OC] |
| 7 | 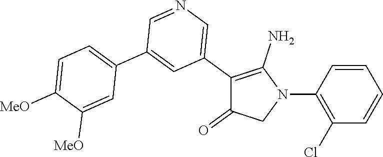 [SMILES: COc1ccc(-c2cncc(C3=C(N)N(c4ccccc4Cl)CC3=O)c2)cc1OC] |
| 8 | 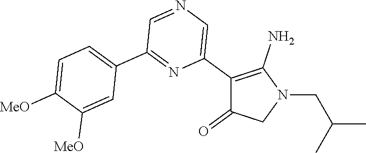 [SMILES: COc1ccc(-c2cncc(C3=C(N)N(CC(C)C)CC3=O)n2)cc1OC] |
| 9 | 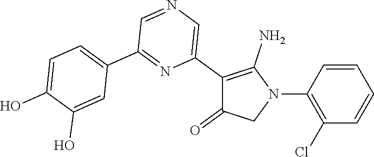 [SMILES: NC1=C(c2cncc(-c3ccc(O)c(O)c3)n2)C(=O)CN1c1ccccc1Cl] |
| 10 | 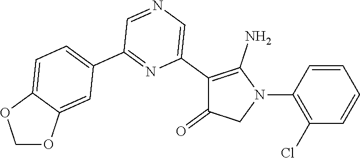 [SMILES: NC1=C(c2cncc(-c3ccc4c(c3)OCO4)n2)C(=O)CN1c1ccccc1Cl] |
| 11 | 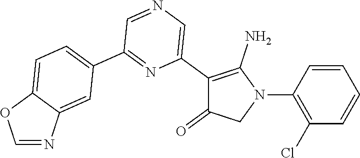 [SMILES: NC1=C(c2cncc(-c3ccc4ocnc4c3)n2)C(=O)CN1c1ccccc1Cl] |
| 12 | 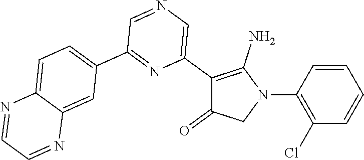 [SMILES: NC1=C(c2cncc(-c3ccc4nccnc4c3)n2)C(=O)CN1c1ccccc1Cl] |
| 13 | 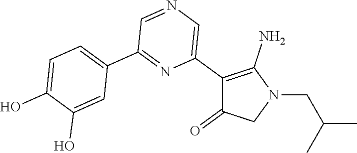 [SMILES: CC(C)CN1CC(=O)C(c2cncc(-c3ccc(O)c(O)c3)n2)=C1N] |
| 14 | 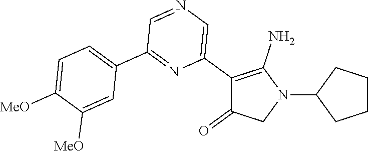 [SMILES: COc1ccc(-c2cncc(C3=C(N)N(C4CCCC4)CC3=O)n2)cc1OC] |
| 15 | 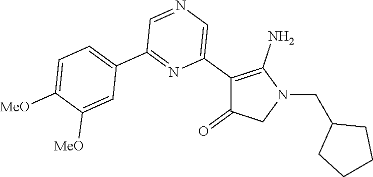 [SMILES: COc1ccc(-c2cncc(C3=C(N)N(CC4CCCC4)CC3=O)n2)cc1OC] |
| 16 | 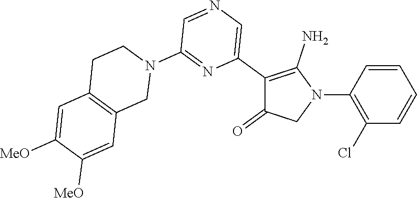 [SMILES: COc1cc2c(cc1OC)CN(c1cncc(C3=C(N)N(c4ccccc4Cl)CC3=O)n1)CC2] |
| 17 | 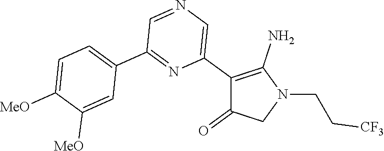 [SMILES: COc1ccc(-c2cncc(C3=C(N)N(CCC(F)(F)F)CC3=O)n2)cc1OC] |
| 18 | 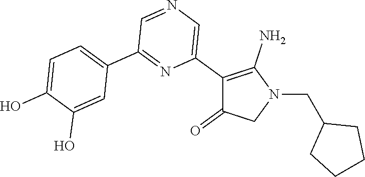 [SMILES: NC1=C(c2cncc(-c3ccc(O)c(O)c3)n2)C(=O)CN1CC1CCCC1] |
| 19 | 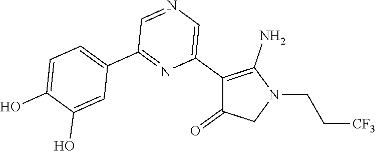 [SMILES: NC1=C(c2cncc(-c3ccc(O)c(O)c3)n2)C(=O)CN1CCC(F)(F)F] |
| 20 | 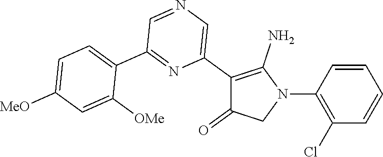 [SMILES: COc1ccc(-c2cncc(C3=C(N)N(c4ccccc4Cl)CC3=O)n2)c(OC)c1] |
| 21 | 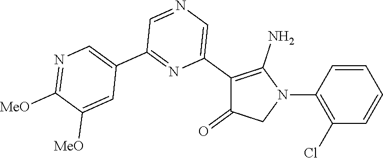 [SMILES: COc1cc(-c2cncc(C3=C(N)N(c4ccccc4Cl)CC3=O)n2)cnc1OC] |
| 22 | 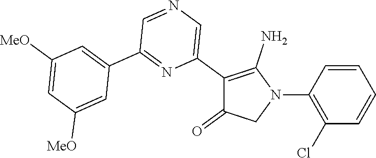 [SMILES: COc1cc(OC)cc(-c2cncc(C3=C(N)N(c4ccccc4Cl)CC3=O)n2)c1] |
| 23 | 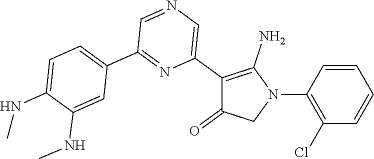 [SMILES: CNc1ccc(-c2cncc(C3=C(N)N(c4ccccc4Cl)CC3=O)n2)cc1NC] |
| 24 | 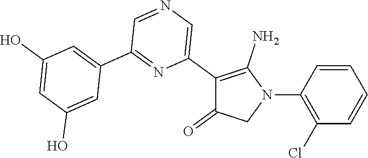 [SMILES: NC1=C(c2cncc(-c3cc(O)cc(O)c3)n2)C(=O)CN1c1ccccc1Cl] |
| 25 | 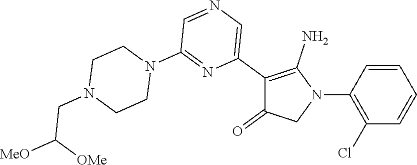 [SMILES: COC(CN1CCN(c2cncc(C3=C(N)N(c4ccccc4Cl)CC3=O)n2)CC1)OC] |
| 26 | 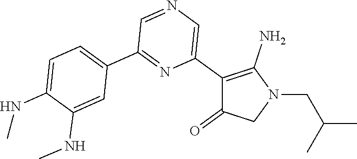 [SMILES: CNc1ccc(-c2cncc(C3=C(N)N(CC(C)C)CC3=O)n2)cc1NC] |
| 27 | 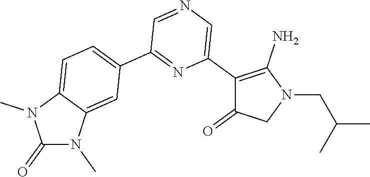 [SMILES: CC(C)CN1CC(=O)C(c2cncc(-c3ccc4c(c3)n(C)c(=O)n4C)n2)=C1N] |
| 28 | 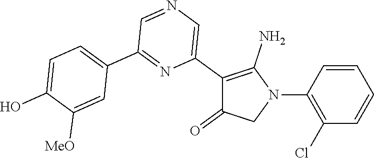 [SMILES: COc1cc(-c2cncc(C3=C(N)N(c4ccccc4Cl)CC3=O)n2)ccc1O] |
| 29 | 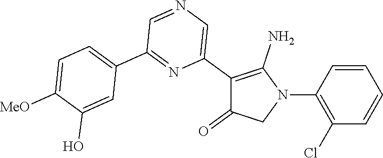 [SMILES: COc1ccc(-c2cncc(C3=C(N)N(c4ccccc4Cl)CC3=O)n2)cc1O] |
| 30 | 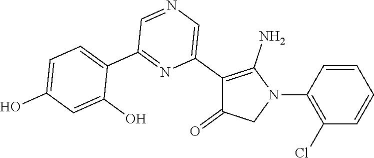 [SMILES: NC1=C(c2cncc(-c3ccc(O)cc3O)n2)C(=O)CN1c1ccccc1Cl] |
| 31 | 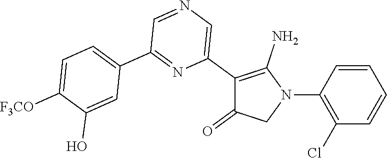 [SMILES: NC1=C(c2cncc(-c3ccc(OC(F)(F)F)c(O)c3)n2)C(=O)CN1c1ccccc1Cl] |
| 32 | 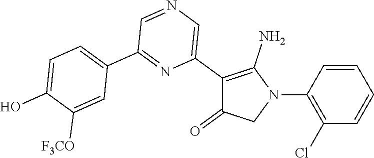 [SMILES: NC1=C(c2cncc(-c3ccc(O)c(OC(F)(F)F)c3)n2)C(=O)CN1c1ccccc1Cl] |
| 33 | 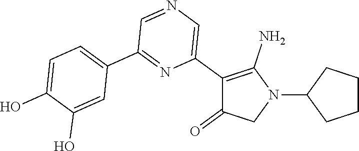 [SMILES: NC1=C(c2cncc(-c3ccc(O)c(O)c3)n2)C(=O)CN1C1CCCC1] |
| 34 | 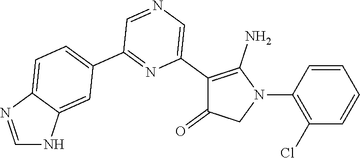 [SMILES: NC1=C(c2cncc(-c3ccc4nc[nH]c4c3)n2)C(=O)CN1c1ccccc1Cl] |
| 35 | 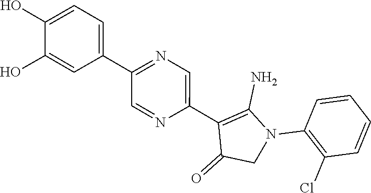 [SMILES: NC1=C(c2cnc(-c3ccc(O)c(O)c3)cn2)C(=O)CN1c1ccccc1Cl] |
| 36 | 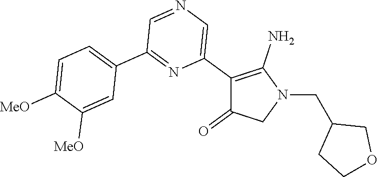 [SMILES: COc1ccc(-c2cncc(C3=C(N)N(CC4CCOC4)CC3=O)n2)cc1OC] |
| 37 | 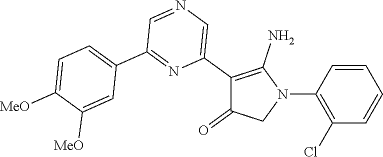 [SMILES: COc1ccc(-c2cncc(C3=C(N)N(c4ccccc4Cl)CC3=O)n2)cc1OC] |
| 38 | 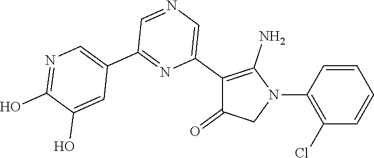 [SMILES: NC1=C(c2cncc(-c3cnc(O)c(O)c3)n2)C(=O)CN1c1ccccc1Cl] |
| 39 | 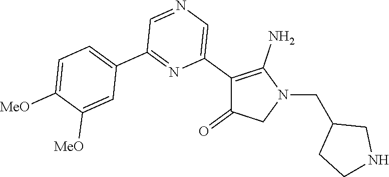 [SMILES: COc1ccc(-c2cncc(C3=C(N)N(CC4CCNC4)CC3=O)n2)cc1OC] |
| 40 | 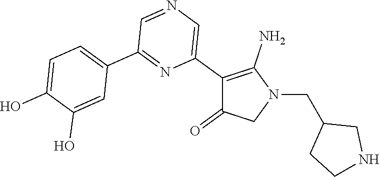 [SMILES: NC1=C(c2cncc(-c3ccc(O)c(O)c3)n2)C(=O)CN1CC1CCNC1] |
| 41 | 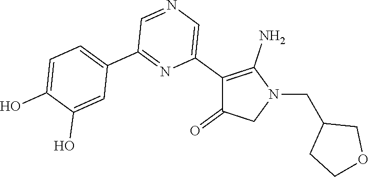 [SMILES: NC1=C(c2cncc(-c3ccc(O)c(O)c3)n2)C(=O)CN1CC1CCOC1] |
| 42 | 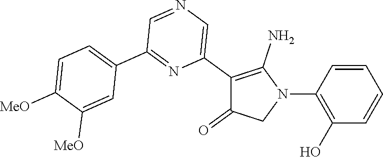 [SMILES: COc1ccc(-c2cncc(C3=C(N)N(c4ccccc4O)CC3=O)n2)cc1OC] |
| 43 | 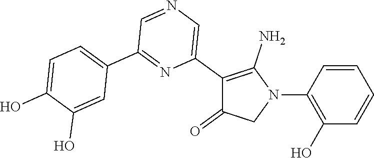 [SMILES: NC1=C(c2cncc(-c3ccc(O)c(O)c3)n2)C(=O)CN1c1ccccc1O] |
| 44 | 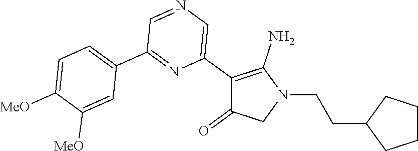 [SMILES: COc1ccc(-c2cncc(C3=C(N)N(CCC4CCCC4)CC3=O)n2)cc1OC] |
| 45 | 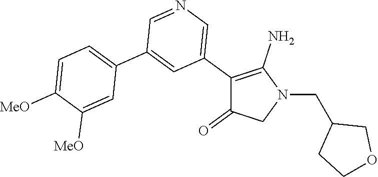 [SMILES: COc1ccc(-c2cncc(C3=C(N)N(CC4CCOC4)CC3=O)c2)cc1OC] |
| 46 | 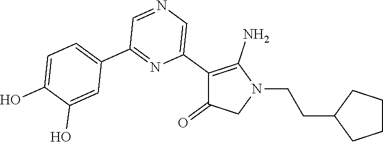 [SMILES: NC1=C(c2cncc(-c3ccc(O)c(O)c3)n2)C(=O)CN1CCC1CCCC1] |
| 47 | 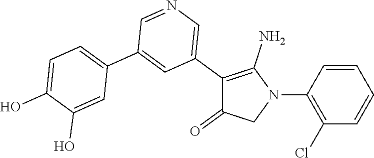 [SMILES: NC1=C(c2cncc(-c3ccc(O)c(O)c3)c2)C(=O)CN1c1ccccc1Cl] |
| 48 | 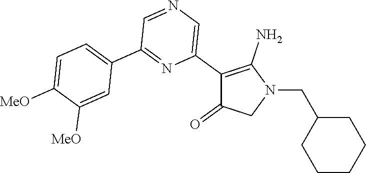 [SMILES: COc1ccc(-c2cncc(C3=C(N)N(CC4CCCCC4)CC3=O)n2)cc1OC] |
| 49 | 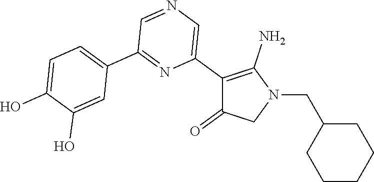 [SMILES: NC1=C(c2cncc(-c3ccc(O)c(O)c3)n2)C(=O)CN1CC1CCCCC1] |
| 50 | 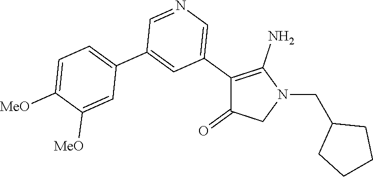 [SMILES: COc1ccc(-c2cncc(C3=C(N)N(CC4CCCC4)CC3=O)c2)cc1OC] |
| 51 | 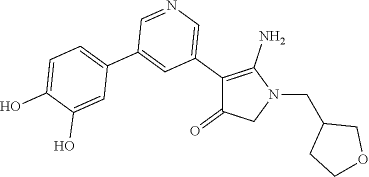 [SMILES: NC1=C(c2cncc(-c3ccc(O)c(O)c3)c2)C(=O)CN1CC1CCOC1] |
| 52 | 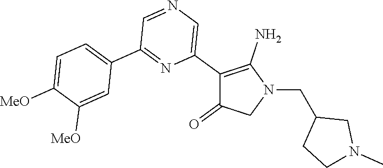 [SMILES: COc1ccc(-c2cncc(C3=C(N)N(CC4CCN(C)C4)CC3=O)n2)cc1OC] |
| 53 | 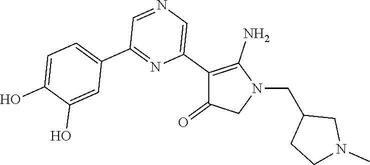 [SMILES: CN1CCC(CN2CC(=O)C(c3cncc(-c4ccc(O)c(O)c4)n3)=C2N)C1] |
| 54 | 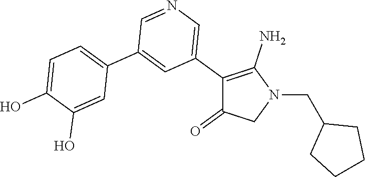 [SMILES: NC1=C(c2cncc(-c3ccc(O)c(O)c3)c2)C(=O)CN1CC1CCCC1] |
| 55 | 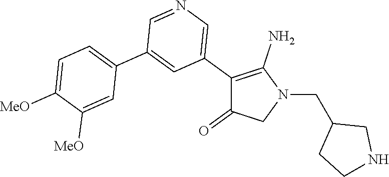 [SMILES: COc1ccc(-c2cncc(C3=C(N)N(CC4CCNC4)CC3=O)c2)cc1OC] |
| 56 | 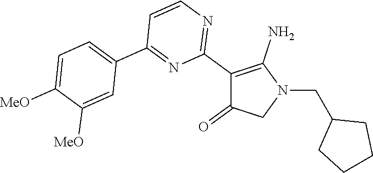 [SMILES: COc1ccc(-c2ccnc(C3=C(N)N(CC4CCCC4)CC3=O)n2)cc1OC] |
| 57 | 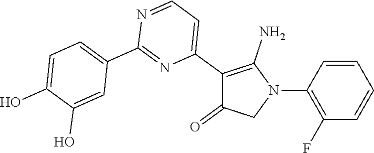 [SMILES: NC1=C(c2ccnc(-c3ccc(O)c(O)c3)n2)C(=O)CN1c1ccccc1F] |
| 58 | 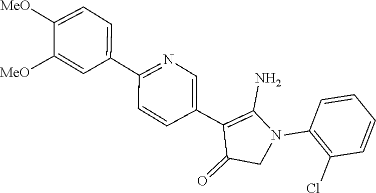 [SMILES: COc1ccc(-c2ccc(C3=C(N)N(c4ccccc4Cl)CC3=O)cn2)cc1OC] |
| 59 | 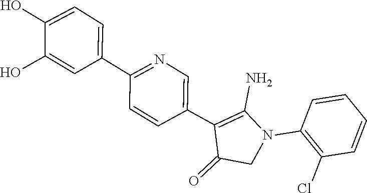 [SMILES: NC1=C(c2ccc(-c3ccc(O)c(O)c3)nc2)C(=O)CN1c1ccccc1Cl] |
| 60 | 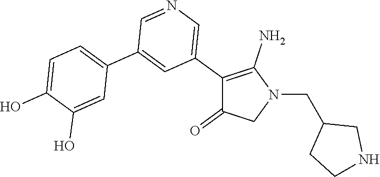 [SMILES: NC1=C(c2cncc(-c3ccc(O)c(O)c3)c2)C(=O)CN1CC1CCNC1] |
| 61 | 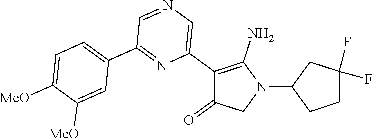 [SMILES: COc1ccc(-c2cncc(C3=C(N)N(C4CCC(F)(F)C4)CC3=O)n2)cc1OC] |
| 62 | 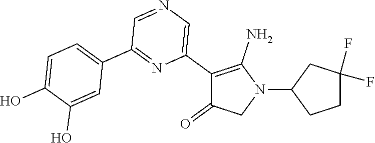 [SMILES: NC1=C(c2cncc(-c3ccc(O)c(O)c3)n2)C(=O)CN1C1CCC(F)(F)C1] |
| 63 | 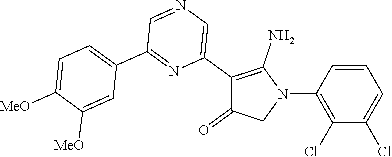 [SMILES: COc1ccc(-c2cncc(C3=C(N)N(c4cccc(Cl)c4Cl)CC3=O)n2)cc1OC] |
| 64 | 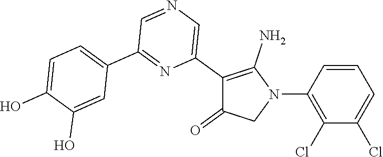 [SMILES: NC1=C(c2cncc(-c3ccc(O)c(O)c3)n2)C(=O)CN1c1cccc(Cl)c1Cl] |
| 65 | 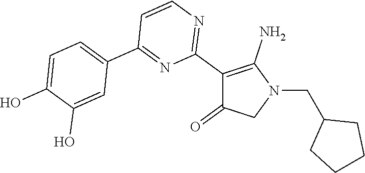 [SMILES: NC1=C(c2nccc(-c3ccc(O)c(O)c3)n2)C(=O)CN1CC1CCCC1] |
| 66 | 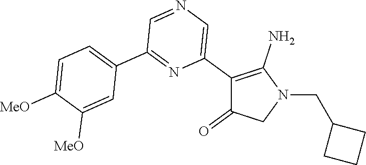 [SMILES: COc1ccc(-c2cncc(C3=C(N)N(CC4CCC4)CC3=O)n2)cc1OC] |
| 67 | 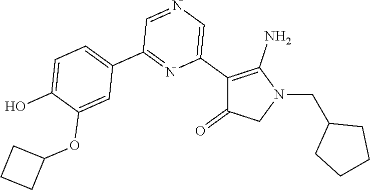 [SMILES: NC1=C(c2cncc(-c3ccc(O)c(OC4CCC4)c3)n2)C(=O)CN1CC1CCCC1] |
| 68 | 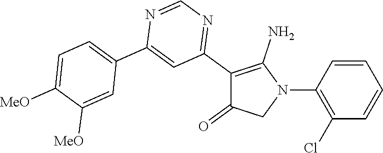 [SMILES: COc1ccc(-c2cc(C3=C(N)N(c4ccccc4Cl)CC3=O)ncn2)cc1OC] |
| 69 | 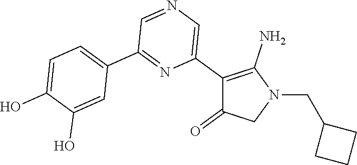 [SMILES: NC1=C(c2cncc(-c3ccc(O)c(O)c3)n2)C(=O)CN1CC1CCC1] |
| 70 | 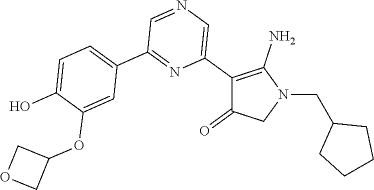 [SMILES: NC1=C(c2cncc(-c3ccc(O)c(OC4COC4)c3)n2)C(=O)CN1CC1CCCC1] |
| 71 | 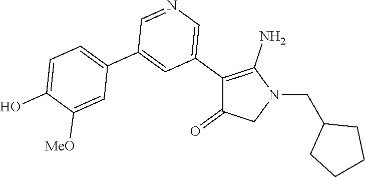 [SMILES: COc1cc(-c2cncc(C3=C(N)N(CC4CCCC4)CC3=O)c2)ccc1O] |
| 72 | 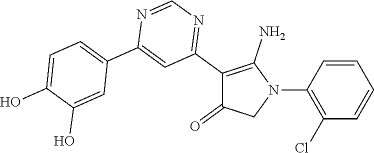 [SMILES: NC1=C(c2cc(-c3ccc(O)c(O)c3)ncn2)C(=O)CN1c1ccccc1Cl] |
| 73 | 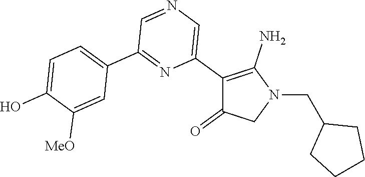 [SMILES: COc1cc(-c2cncc(C3=C(N)N(CC4CCCC4)CC3=O)n2)ccc1O] |
| 74 | 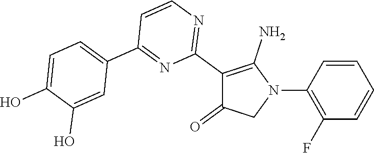 [SMILES: NC1=C(c2nccc(-c3ccc(O)c(O)c3)n2)C(=O)CN1c1ccccc1F] |
| 75 | 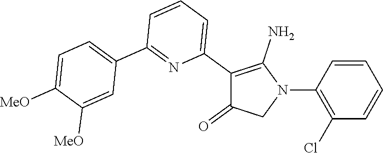 [SMILES: COc1ccc(-c2cccc(C3=C(N)N(c4ccccc4Cl)CC3=O)n2)cc1OC] |
| 76 | 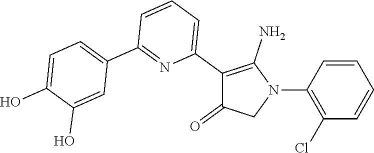 [SMILES: NC1=C(c2cccc(-c3ccc(O)c(O)c3)n2)C(=O)CN1c1ccccc1Cl] |
| 77 | 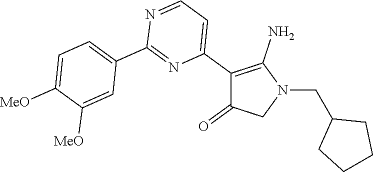 [SMILES: COc1ccc(-c2nccc(C3=C(N)N(CC4CCCC4)CC3=O)n2)cc1OC] |
| 78 | 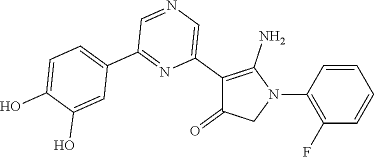 [SMILES: NC1=C(c2cncc(-c3ccc(O)c(O)c3)n2)C(=O)CN1c1ccccc1F] |
| 79 | 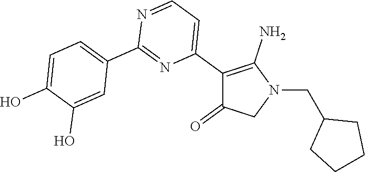 [SMILES: NC1=C(c2ccnc(-c3ccc(O)c(O)c3)n2)C(=O)CN1CC1CCCC1] |
| 80 | 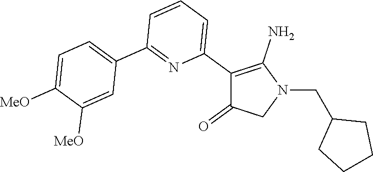 [SMILES: COc1ccc(-c2cccc(C3=C(N)N(CC4CCCC4)CC3=O)n2)cc1OC] |
| 81 | 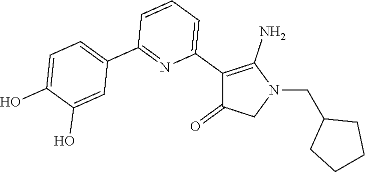 [SMILES: NC1=C(c2cccc(-c3ccc(O)c(O)c3)n2)C(=O)CN1CC1CCCC1] |
| 82 | 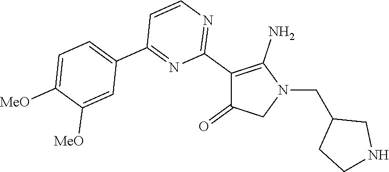 [SMILES: COc1ccc(-c2ccnc(C3=C(N)N(CC4CCNC4)CC3=O)n2)cc1OC] |
| 83 | 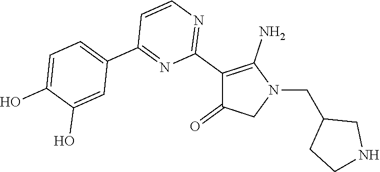 [SMILES: NC1=C(c2nccc(-c3ccc(O)c(O)c3)n2)C(=O)CN1CC1CCNC1] |
| 84 | 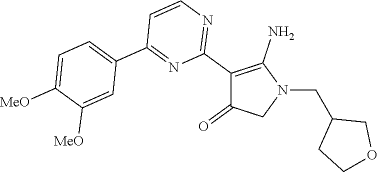 [SMILES: COc1ccc(-c2ccnc(C3=C(N)N(CC4CCOC4)CC3=O)n2)cc1OC] |
| 85 | 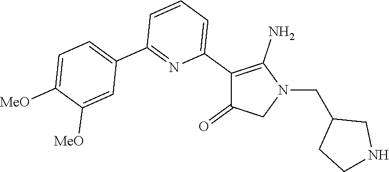 [SMILES: COc1ccc(-c2cccc(C3=C(N)N(CC4CCNC4)CC3=O)n2)cc1OC] |
| 86 | 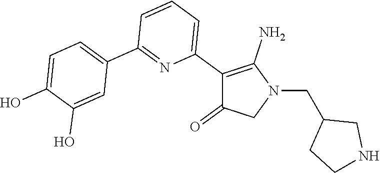 [SMILES: NC1=C(c2cccc(-c3ccc(O)c(O)c3)n2)C(=O)CN1CC1CCNC1] |
| 87 | 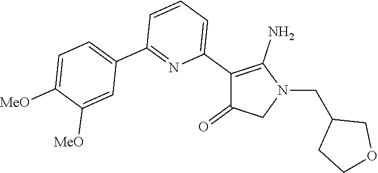 [SMILES: COc1ccc(-c2cccc(C3=C(N)N(CC4CCOC4)CC3=O)n2)cc1OC] |
| 88 | 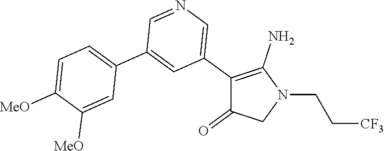 [SMILES: COc1ccc(-c2cncc(C3=C(N)N(CCC(F)(F)F)CC3=O)c2)cc1OC] |
| 89 | 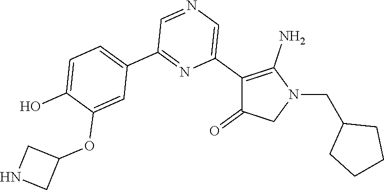 [SMILES: NC1=C(c2cncc(-c3ccc(O)c(OC4CNC4)c3)n2)C(=O)CN1CC1CCCC1] |
| 90 | 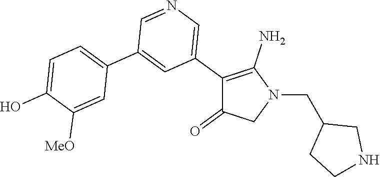 [SMILES: COc1cc(-c2cncc(C3=C(N)N(CC4CCNC4)CC3=O)c2)ccc1O] |
| 91 | 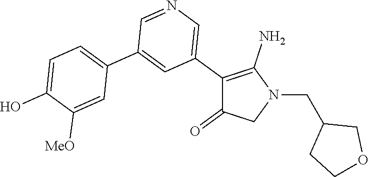 [SMILES: COc1cc(-c2cncc(C3=C(N)N(CC4CCOC4)CC3=O)c2)ccc1O] |
| 92 | 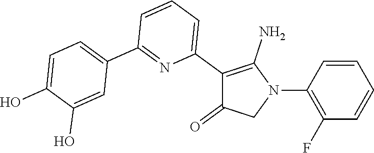 [SMILES: NC1=C(c2cccc(-c3ccc(O)c(O)c3)n2)C(=O)CN1c1ccccc1F] |
| 93 | 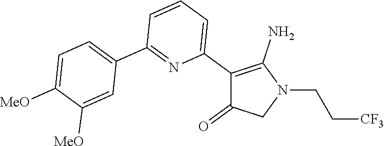 [SMILES: COc1ccc(-c2cccc(C3=C(N)N(CCC(F)(F)F)CC3=O)n2)cc1OC] |
| 94 | 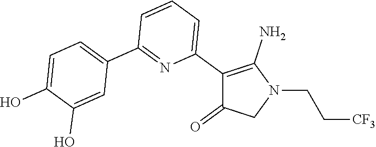 [SMILES: NC1=C(c2cccc(-c3ccc(O)c(O)c3)n2)C(=O)CN1CCC(F)(F)F] |
| 95 | 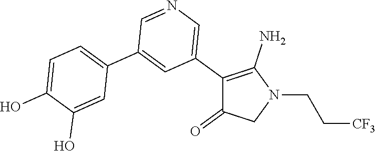 [SMILES: NC1=C(c2cncc(-c3ccc(O)c(O)c3)c2)C(=O)CN1CCC(F)(F)F] |
| 96 | 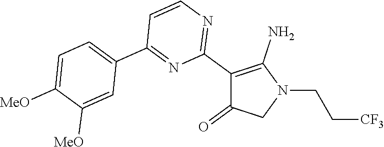 [SMILES: COc1ccc(-c2ccnc(C3=C(N)N(CCC(F)(F)F)CC3=O)n2)cc1OC] |
| 97 | 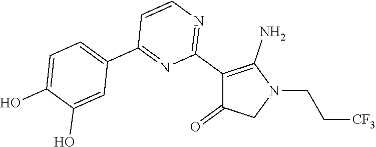 [SMILES: NC1=C(c2nccc(-c3ccc(O)c(O)c3)n2)C(=O)CN1CCC(F)(F)F] |
| 98 | 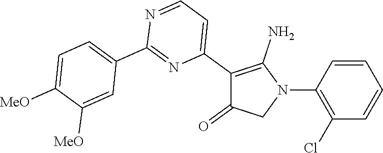 [SMILES: COc1ccc(-c2nccc(C3=C(N)N(c4ccccc4Cl)CC3=O)n2)cc1OC] |
| 99 | 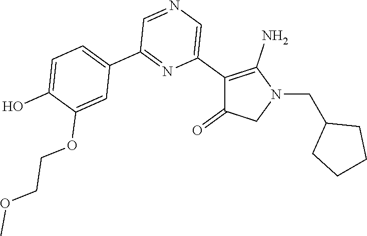 [SMILES: COCCOc1cc(-c2cncc(C3=C(N)N(CC4CCCC4)CC3=O)n2)ccc1O] |
| 100 | 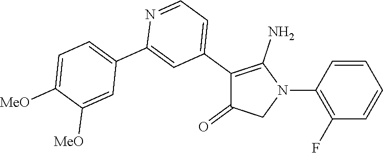 [SMILES: COc1ccc(-c2cc(C3=C(N)N(c4ccccc4F)CC3=O)ccn2)cc1OC] |
| 101 | 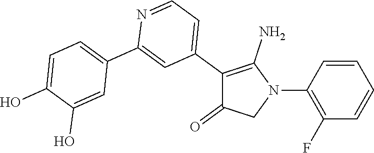 [SMILES: NC1=C(c2ccnc(-c3ccc(O)c(O)c3)c2)C(=O)CN1c1ccccc1F] |
| 102 | 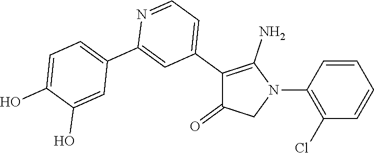 [SMILES: NC1=C(c2ccnc(-c3ccc(O)c(O)c3)c2)C(=O)CN1c1ccccc1Cl] |
| 103 | 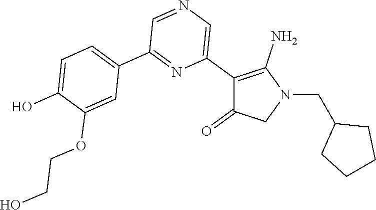 [SMILES: NC1=C(c2cncc(-c3ccc(O)c(OCCO)c3)n2)C(=O)CN1CC1CCCC1] |
| 104 | 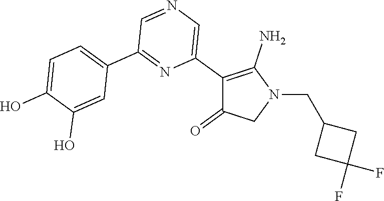 [SMILES: NC1=C(c2cncc(-c3ccc(O)c(O)c3)n2)C(=O)CN1CC1CC(F)(F)C1] |
| 105 | 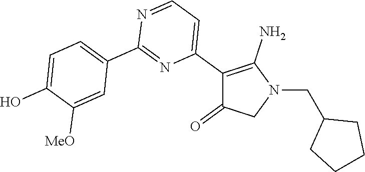 [SMILES: COc1cc(-c2nccc(C3=C(N)N(CC4CCCC4)CC3=O)n2)ccc1O] |
| 106 | 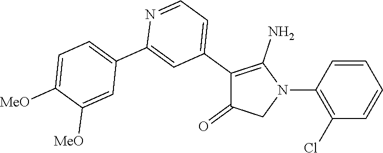 [SMILES: COc1ccc(-c2cc(C3=C(N)N(c4ccccc4Cl)CC3=O)ccn2)cc1OC] |
| 107 | 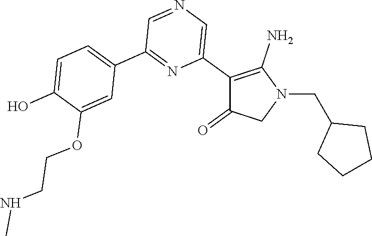 [SMILES: CNCCOc1cc(-c2cncc(C3=C(N)N(CC4CCCC4)CC3=O)n2)ccc1O] |
| 108 | 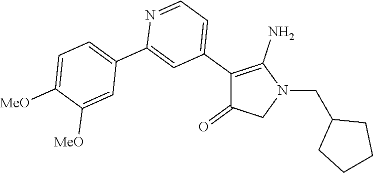 [SMILES: COc1ccc(-c2cc(C3=C(N)N(CC4CCCC4)CC3=O)ccn2)cc1OC] |
| 109 | 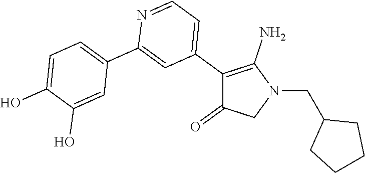 [SMILES: NC1=C(c2ccnc(-c3ccc(O)c(O)c3)c2)C(=O)CN1CC1CCCC1] |
| 110 | 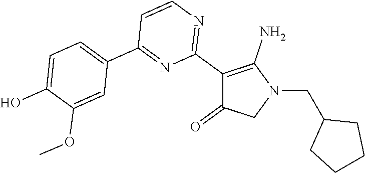 [SMILES: COc1cc(-c2ccnc(C3=C(N)N(CC4CCCC4)CC3=O)n2)ccc1O] |
| 111 | 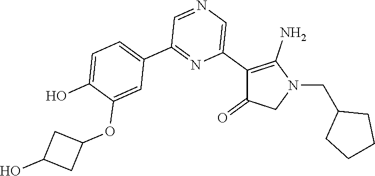 [SMILES: NC1=C(c2cncc(-c3ccc(O)c(OC4CC(O)C4)c3)n2)C(=O)CN1CC1CCCC1] |
| 112 | 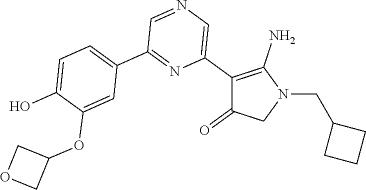 [SMILES: NC1=C(c2cncc(-c3ccc(O)c(OC4COC4)c3)n2)C(=O)CN1CC1CCC1] |
| 113 | 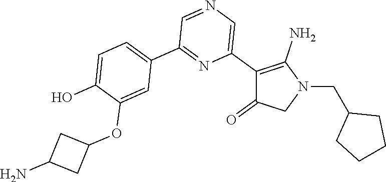 [SMILES: NC1=C(c2cncc(-c3ccc(O)c(OC4CC(N)C4)c3)n2)C(=O)CN1CC1CCCC1] |
| 114 | 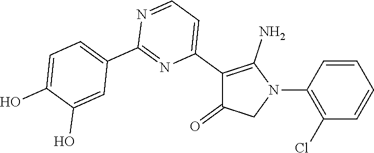 [SMILES: NC1=C(c2ccnc(-c3ccc(O)c(O)c3)n2)C(=O)CN1c1ccccc1Cl] |
| 115 | 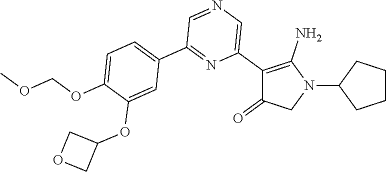 [SMILES: COCOc1ccc(-c2cncc(C3=C(N)N(C4CCCC4)CC3=O)n2)cc1OC1COC1] |
| 116 | 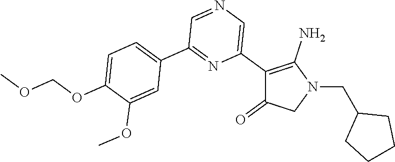 [SMILES: COCOc1ccc(-c2cncc(C3=C(N)N(CC4CCCC4)CC3=O)n2)cc1OC] |
| 117 | 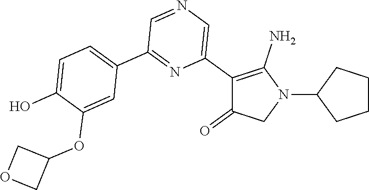 [SMILES: NC1=C(c2cncc(-c3ccc(O)c(OC4COC4)c3)n2)C(=O)CN1C1CCCC1] |
| 118 | 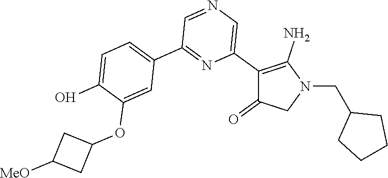 [SMILES: COC1CC(Oc2cc(-c3cncc(C4=C(N)N(CC5CCCC5)CC4=O)n3)ccc2O)C1] |
| 119 | 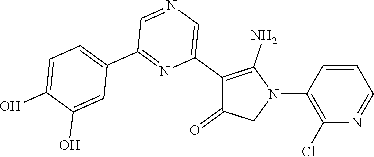 [SMILES: NC1=C(c2cncc(-c3ccc(O)c(O)c3)n2)C(=O)CN1c1cccnc1Cl] |
| 120 | 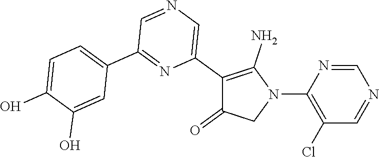 [SMILES: NC1=C(c2cncc(-c3ccc(O)c(O)c3)n2)C(=O)CN1c1ncncc1Cl] |

### 3. Composition/Formulation

In an another embodiment, the present invention provides a composition of Chemical formula 1 as an active ingredient, together with at least one pharmaceutically acceptable carrier, excipient and/or diluent.

In a preferred embodiment, the composition may be for use as a pharmaceutically active agent, preferably for use in a method of treating a disease.

The present invention includes the compound described herein and a pharmaceutical composition comprising a formulation suitable for administration of the compound described herein. Formulations of the pharmaceutical composition suitable for administration by any medically acceptable means are included. The pharmaceutical formulation may comprise a pharmaceutically acceptable additive or carrier suitable for means of administration and a pharmaceutically acceptable compound (composition).

The compound described herein may be a formulation (including a pharmaceutical composition) having an additive such as an excipient (for example, one or more excipients), an anti-oxidant (for example, one or more anti-oxidants), a stabilizer (for example, one or more stabilizers), a preservative (for example, one or more preservatives), a pH adjusting agent and/or buffers (for example, one or more pH adjusting agents and/or buffers), an isotonic adjusting agent (for example, one or more isotonic adjusting agents), a thickener (for example, one or more thickeners), a suspending agent (for example, one or more suspending agents), a binding agent (for example, one or more binding agents), a viscosity increasing agent (for example, one or more viscosity increasing agents), and the like, and is provided as a pharmaceutically acceptable additional ingredient for a particular condition to be treated. In some embodiments, the formulation may comprise a combination of the additional ingredient described herein (for example, 2, 3, 4, 5, 6, 7, 8 or more additional ingredients). In some embodiments, the additive may comprise for example, a treatment agent and a drug delivery modifier, and an enhancer such as calcium phosphate, magnesium stearate, talc, monosaccharides, disaccharides, starch, gelatin, cellulose, methyl cellulose, sodium carboxymethyl cellulose, dextrose, hydroxypropyl-beta-cyclodextrin, polyvinylpyrrolidone, low melting point wax and ion exchange resin, and a combination of any two or more thereof.

Other suitable pharmaceutically acceptable excipients are described in “Remington's Pharmaceutical Sciences” Mack Pub. Co., New Jersey (1991), and “Remington: The Science and Practice of Pharmacy”, Lippincott Williams & Wilkins, Philadelphia, 20th edition (2003) and 21st edition (2005) incorporated herein as a reference.

The formulation of the composition described herein may be appropriate for oral administration which may be composed of inhalation, nose spray, intravenous, intramuscular injection, intravitreal injection, ointment or solution, suspension, semiliquid, semisolid, gel, semisolid gel, jelly, emulsion, ointment, oil, tablet, liquid and cream. The tablet form may compose one or more of lactose, sucrose, mannitol, sorbitol, calcium phosphate, corn starch, potato starch, microcrystal cellulose, gelatin, colloidal silicon dioxide, talc, magnesium stearate, stearic acid and other excipient, a coloring agent, a filler, a binding agent, a diluent, a buffer, a moisturizer, a preservative, a flavoring agent, a dye, a disintegrating agent and a pharmaceutically suitable carrier. A capsule may contain an appropriate excipient with the compound or the compound may be used in a shell alone. All of these formulated compounds may be administered alone or co-administered, or intermittently administered, or sequentially or simultaneously administered.

### 4. Administration

The composition of the present invention includes one which may be administered by oral, parenteral, sublingual, dermal, rectal, transmucosal, local, transtympanic, intratympanic, intracochlear, through inhalation, buccal or intranasal administration, or be administered by any method of a combination thereof, but not limited thereto. The parenteral administration includes intravenous, intraperitoneal, subcutaneous, intramuscular, intrathecal and intra-arterial administration, but not limited thereto. In addition, the composition of the present invention may be administered as an implant, and this allows slow release of the composition as well as slowly controlled intravenous administration.

The dose administered to an individual in a single or multiple dose will vary depending on various factors including pharmacokinetic characteristics, patient condition and characteristics (gender, age, weight, health, size), severity of symptoms, concurrent treatment, treatment frequency and desired effect.

According to one embodiment of the present invention, the compound and pharmaceutical formulation thereof according to the present invention may be administered alone or with a useful adjuvant for treatment of respiratory disorder or disease. According to another embodiment of the present invention, the compound and pharmaceutical formulation thereof according to the present invention may be administered with radiotherapy.

The present invention comprises administration of the compound or pharmaceutical formulation thereof according to the present invention, and the compound and pharmaceutical formulation thereof according to the present invention is administered simultaneously or sequentially to a subject before other therapy or adjuvant (for example, polypharmacy) useful for treatment of pendred syndrome, in a therapeutically effective amount. The compound or pharmaceutical formulation according to the present invention administered with the adjuvant may be administered by the same or different administration route(s) in the same or different composition(s).

In one embodiment, the patient according to the present invention may be a patient suffering pendred syndrome or the related diseases thereof such as hearing loss, vesticular aqueduct dilation, goiter, hypertension, hypokalemia, hypothyroidism, hypochloremic alkalosis, renal tubular acidosis, volume depletion, hypovolemia, edema, cystic fibrosis, asthma, chronic obstructive pulmonary disease, rhinitis, sinusitis, cirrhosis, bone abnormality, malformation of the cochlea, chronic obstructive pulmonary disease, acute lung injury (ALI), acute respiratory distress syndrome (ARDS), bronchitis, speech impairment or rhinosinusitis.

### 5. Use According to the Present Invention

In other embodiment, the present invention provides a compound represented by Chemical formula 1 for preventing, improving or treating of pendred syndrome or the related diseases thereof, a mixture of the compound, or a use of a pharmaceutical composition thereof.

In other embodiment, the present invention provides a compound represented by Chemical formula 1 and a use of a pharmaceutical composition thereof as a pendrin corrector.

In other embodiment, the present invention provides a use for pendred syndrome-related diseases in one or more selected from the group consisting of hearing loss, vesticular aqueduct dilation, goiter, hypertension, hypokalemia, hypothyroidism, hypochloremic alkalosis, renal tubular acidosis, volume depletion, hypovolemia, edema, cystic fibrosis, asthma, chronic obstructive pulmonary disease, rhinitis, sinusitis, cirrhosis, bone abnormality, malformation of the cochlea, chronic obstructive pulmonary disease, acute lung injury (ALI), acute respiratory distress syndrome (ARDS), bronchitis, speech impairment, rhinosinusitis and the like.

In other embodiment, the present invention provides a compound represented by Chemical formula 1 for preventing or improving of pendred syndrome or the related diseases thereof, a mixture of the compound or a use of a pharmaceutical composition thereof, as an active ingredient in a health functional food.

In other embodiment, the present invention provides a use of a compound represented by Chemical formula 1 and a pharmaceutical composition thereof as a pendrin inhibitor for preventing or improving of pendred syndrome or the related diseases thereof, as an active ingredient in a health functional food.

Other aspects and advantages of the present invention will become apparent to those skilled in the art in consideration of the detailed description and drawings.

### MODE FOR INVENTION

### Example

Non-limitative examples of detailed experiments which do not limit the entire experiments are described herein. The description of the present invention described herein serves as an example, and a person with general knowledge of the technology related to the present invention should understand that it can easily change it to other specific areas or forms without changing the technical spirit or essential features of the present invention. The present invention described illustratively suggests the following examples, but not limited thereto.

The references cited herein are incorporated herein by reference in their entirety. The present invention is not limited in the scope to the specific embodiments described herein, which are intended as single examples of individual aspects of the present invention, and functionally equivalent methods and components are within the scope of the present invention. In practice, in addition to those illustrated and described herein, various modifications of the present invention will become apparent to those skilled in the art from the aforementioned description and attached drawings. Such modifications are intended to fall within the scope of the appended claims.

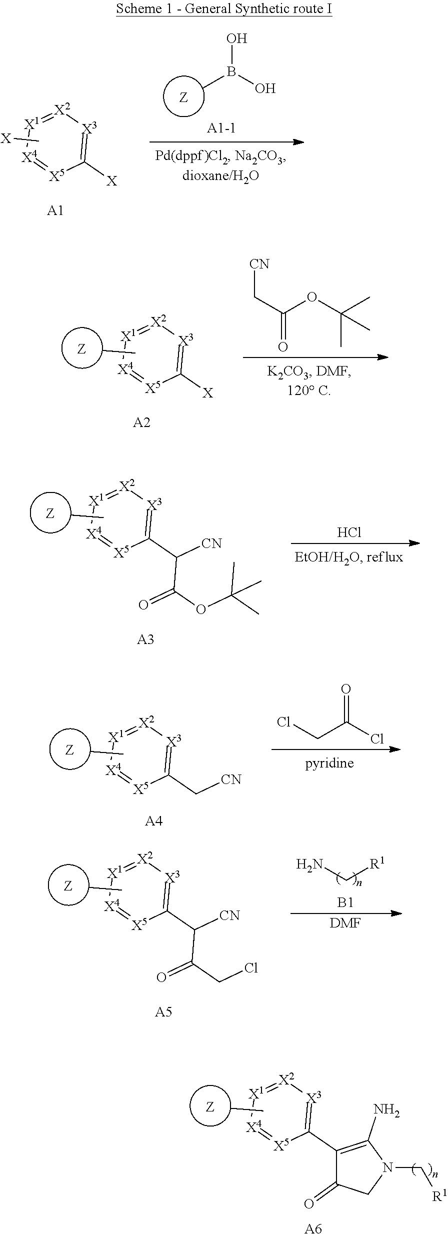

### General Procedure for Synthesis of A2

A mixture of A1, A1-1, Na₂CO₃, cyclopentyl(diphenyl)phosphane; dichloropalladium; iron in dioxane and H₂O was degassed and purged with N₂ for 3 times, and then the mixture was stirred at 100° C. for 2 h under N₂ atmosphere. The reaction mixture was poured into water and extracted with ethyl acetate, washed with brine, dried over Na₂SO₄ and concentrated under reduced pressure. The residue was purified by flash chromatography on silica gel to give A2.

### General Procedure for Synthesis of A3

To a solution A2 in DMF was added K₂CO₃ and tert-butyl 2-cyanoacetate. The mixture was stirred at 120° C. for 16 h. The reaction mixture was poured into water and extracted with ethyl acetate, washed with brine, dried over Na₂SO₄ and concentrated under reduced pressure to give A3.

### General Procedure for Synthesis of A4

A mixture of A3 in EtOH was added con. HCl and stirred at 80° C. for 40 min. The mixture was quenched with sat. NaHCO₃ to pH=7 and extracted with ethyl acetate. The combined organic layer was dried over Na₂SO₄. The filtrate was concentrated to give crude product which was purified by flash chromatography on silica gel A4.

### General Procedure for Synthesis of A5

To a solution of A4 in dioxane was added pyridine at 20° C. The reaction mixture was added dropwise 2-chloroacetyl chloride at 40-50° C. The mixture was stirred at 100° C. for 20 min. The reaction mixture was used for next step without further purification.

### General Procedure for Synthesis of A6

To a solution of A5 in dioxane from previous step was added DMF and B1. The mixture was stirred at 100° C. for 10 hr. The reaction mixture was concentrated under reduced pressure. The crude product was purified by Prep-HPLC and lyophilized to afford A6.

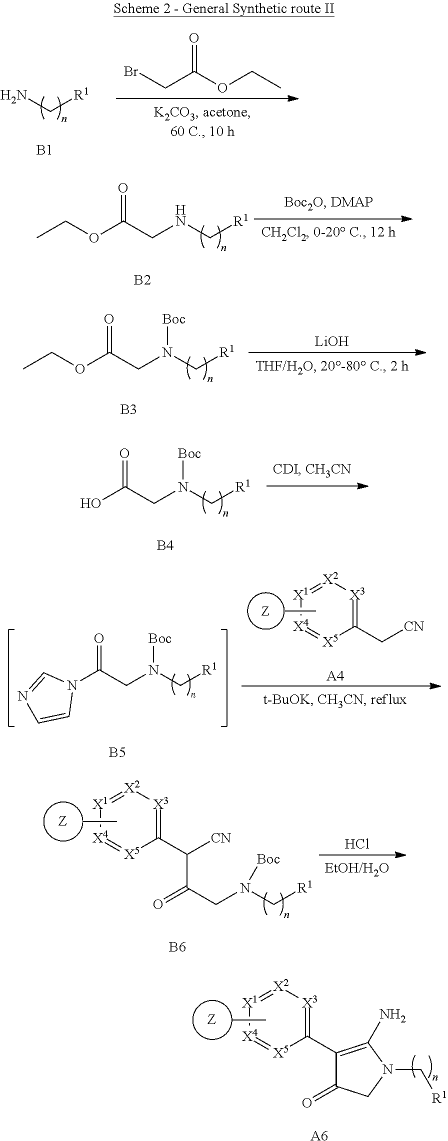

### General Procedure for Synthesis of B2

To a solution of B1 and K₂CO₃ in acetone was added ethyl 2-bromoacetate at 60° C. for 10 h. The reaction mixture was poured into water and extracted with EtOAc. The combined organic layers were washed with brine, dried over Na₂SO₄ and concentrated under reduced pressure. The filtrate was concentrated to give crude product which was purified by flash chromatography on silica gel to give B2.

### General Procedure for Synthesis of B3

To a solution of B2 in CH₂Cl₂ was added Boc₂O and DMAP at 0° C. The reaction mixture was poured into water and extracted with EtOAc. The combined organic layers were washed with brine, dried over Na₂SO₄ and concentrated under reduced pressure. The filtrate was concentrated to give crude product which was purified by flash chromatography on silica gel to give B3.

### General Procedure for Synthesis of B4

To a solution of B3 in THF and H₂O was added LiOH·H₂O and stirred at 20° C. for 1 h, then the mixture was stirred at 80° C. for 1 h. The reaction mixture was adjusted to pH=4 with HCl (1 mol/L), and then poured into water and extracted with EtOAc. The combined organic layers were washed with brine, dried over Na₂SO₄, then concentrated under reduced pressure to give B4.

### General Procedure for Synthesis of B6

To a solution of B4 in CH₃CN was added CDI, the reaction mixture was stirred at 20° C. for 40 min. TLC showed the B4 was consumed completely. The B5 was used for next step.

To a solution of B5 and A4 in CH₃CN was added t-BuOK and stirred at 80° C. for 1 h.

The reaction mixture was poured into water and extracted with EtOAc. The combined organic layers were washed with brine, dried over Na₂SO₄ then reaction mixture was concentrated in vacuum to give B6.

### General Procedure for Synthesis of A6

To a solution of B6 in EtOH was added 37% HCl. The reaction mixture was stirred at 80° C. for 1 h. The reaction solution was concentrated and the purified to afford A6.

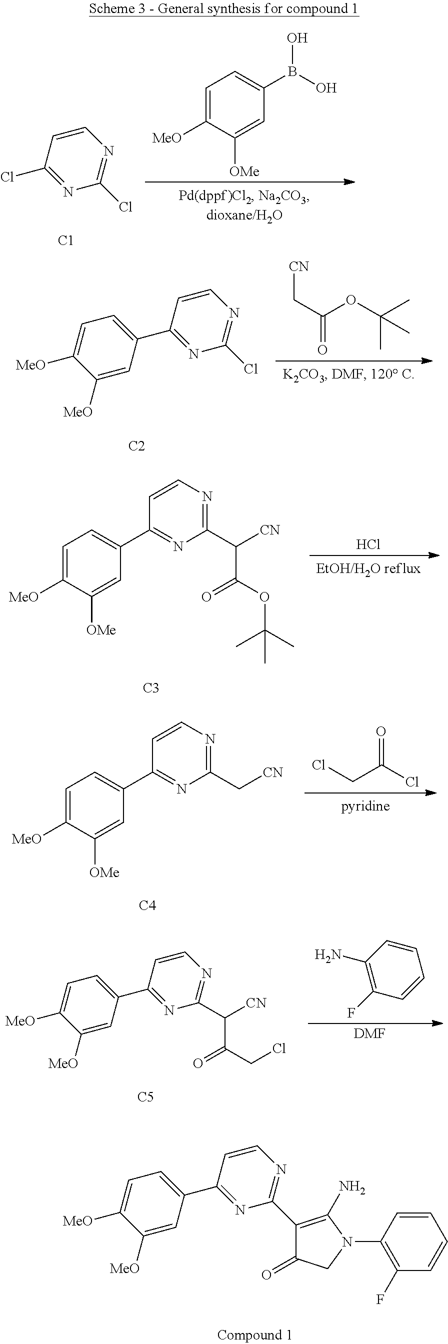

### General Procedure for Synthesis of C2

A mixture of C1 (3.68 g, 24.73 mmol), (3,4-dimethoxyphenyl)boronic acid (3 g, 16.49 mmol) Na₂CO₃ (3.49 g, 32.97 mmol), cyclopentyl(diphenyl)phosphane; dichloropalladium; iron (1.21 g, 1.65 mmol) in dioxane (10 mL) and H₂O (1 mL) was degassed and purged with N₂ for 3 times, and then the mixture was stirred at 100° C. for 2 h under N₂ atmosphere. The reaction mixture was poured into water (10 mL) and extracted with ethyl acetate (50 mL×3), washed with brine (50 mL×3), dried over Na₂SO₄ and concentrated under reduced pressure. The residue was purified by flash chromatography on silica gel (0-35% ethyl acetate in PE) to give C2 (2.57 g, 62.2% yield) as white solid.

### General Procedure for Synthesis of C3

*199To a solution C2 (1 g, 3.99 mmol) in DMF (10 mL) was added K₂CO₃ (1.10 g, 7.98 mmol) and tert-butyl 2-cyanoacetate (675.76 mg, 4.79 mmol). The mixture was stirred at 120° C. for 16 h. The reaction mixture was poured into water (10 mL) and extracted with ethyl acetate (10 mL×3), washed with brine (10 mL×3), dried over Na₂SO₄ and concentrated under reduced pressure to give C3 (1.5 g, crude, ˜93% purity) as a yellow solid.

### General Procedure for Synthesis of C4

A mixture of C3 (1.5 g, ˜93% purity, 3.94 mmol) in EtOH (25 mL) was added con. HCl (6 mL) and stirred at 80° C. for 40 min. The mixture was quenched with sat. NaHCO₃ to pH=7 and extracted with ethyl acetate (50 mL×3). The combined organic layer was dried over Na₂SO₄. The filtrate was concentrated to give crude product which was purified by flash chromatography on silica gel (0-50% ethyl acetate in petroleum ether) to give C4 (607 mg, 60.4% yield) as a yellow solid.

### General Procedure for Synthesis of C5

To a solution of C4 (50 mg, 195.87 umol) in dioxane (1 mL) was added pyridine (18.59 mg, 18.97 uL) at 20° C. The reaction mixture was added dropwise 2-chloroacetyl chloride (22.12 mg, 195.87 umol) at 40-50° C. The mixture was stirred at 100° C. for 20 min. The reaction mixture was used for next step without further purification.

### General Procedure for Synthesis of Compound 1

To a solution of C5 (64.98 mg, 195.87 umol) in dioxane from previous step was added DMF (1 mL) and 2-fluoroaniline (65.29 mg, 587.61 umol). The mixture was stirred at 100° C. for 10 hr. The reaction mixture was concentrated under reduced pressure. The crude product was purified by Prep-HPLC (column: Waters Xbridge 150*25 5u, table: 63-93% B (A=water (0.05% ammonia hydroxide v/v)), B=acetonitrile), flow rate: 25 mL/min, UV Detector 220 nm) and lyophilized to afford compound 1 (3 mg, 3.6% yield, 95% purity) as a brown solid.

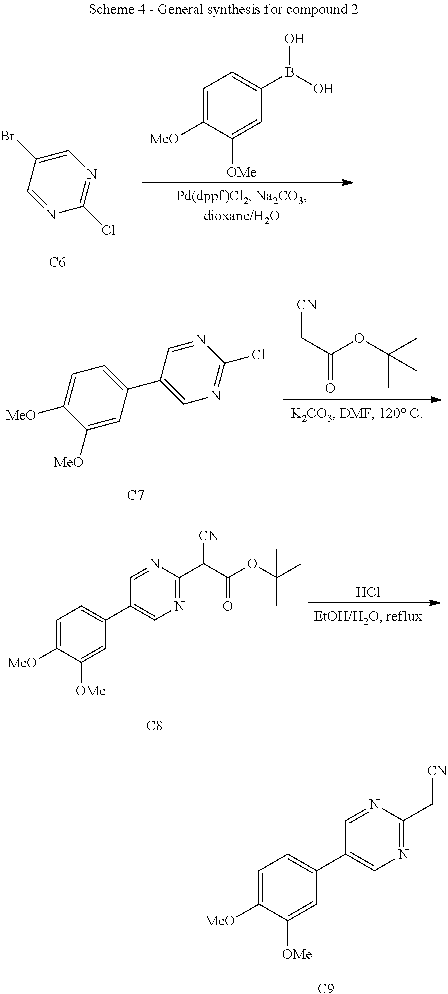

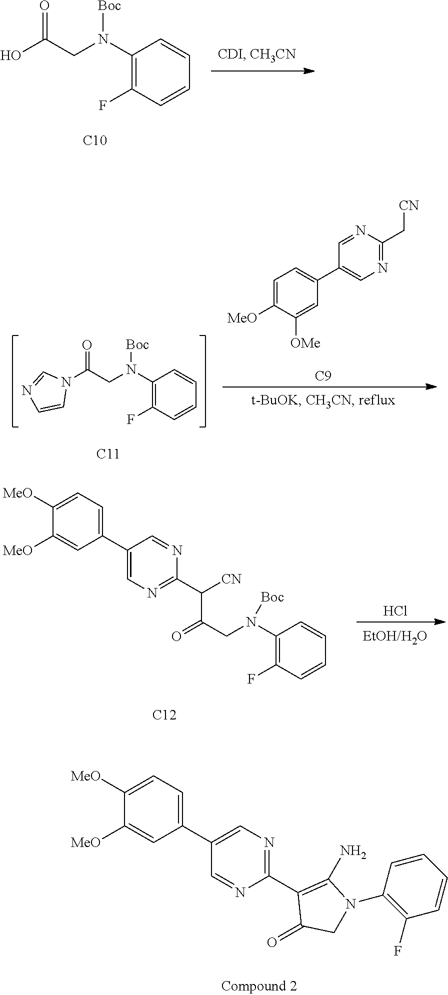

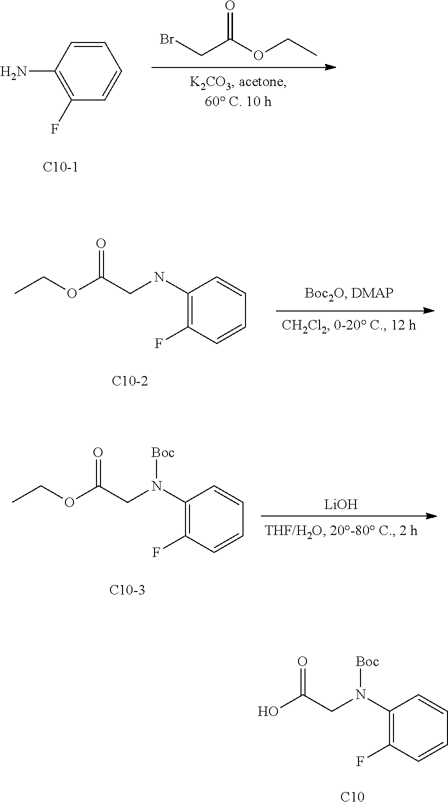

### General Procedure for Synthesis of C7

A mixture of C6 (3 g, 15.51 mmol), (3,4-dimethoxyphenyl)boronic acid (3 g, 16.49 mmol), Na₂CO₃ (3.29 g, 31.02 mmol), Pd(dppf)Cl₂ (1.13 g, 1.55 mmol) in dioxane (30 mL) and H₂O (5 mL) was degassed and purged with N₂ for 3 times, and then the mixture was stirred at 80° C. for 3 hr under N₂ atmosphere. The filtrate was concentrated to give crude product which was purified by flash chromatography on silica gel (0-40% EtOAc in petroleum ether) to give C7 (2.76 g, 71.0% yield) as off-white solid.

### General Procedure for Synthesis of C8

To a solution C7 (2.66 g, 10.61 mmol) in DMF (30 mL) was added K₂CO₃ (2.93 g, 21.22 mmol) and tert-butyl 2-cyanoacetate (1.80 g, 12.73 mmol, 1.82 mL). The mixture was stirred at 120° C. for 16 hr. The mixture was quenched with water (20 mL) and adjusted pH to about 4 with 1M HCl and then filtered. The filter cake was dried under reduced pressure to give C8 (3.4 g, 90.2% yield) as a brown solid

### General Procedure for Synthesis of C9

A mixture of C8 (3.4 g, 9.57 mmol) in HCl (3 mL) and EtOH (30 mL) was stirred at 90° C. for 40 min. The mixture was quenched with sat. NaHCO₃ to pH=8 and extracted with ethyl acetate (100 mL×3). The combined organic layer was dried over Na₂SO₄. The filtrate was concentrated to give crude product which was purified by flash chromatography on silica gel (0-50% EtOAc in petroleum ether) to give C9 (1.77 g, 72.5% yield) as a yellow solid.

### General Procedure for Synthesis of C10-2

To a solution of C10-1 (10 g, 90.00 mmol, 8.70 mL) and K₂CO₃ (17.41 g, 125.99 mmol) in acetone (20 mL) was added ethyl 2-bromoacetate (16.53 g, 99.00 mmol) at 60° C. for 10 h. The reaction mixture was poured into water (20 mL) and extracted with EtOAc (30 mL*3). The combined organic layers were washed with brine (30 mL*3), dried over Na₂SO₄ and concentrated under reduced pressure. The filtrate was concentrated to give crude product which was purified by flash chromatography on silica gel (0-10% EtOAc in petroleum ether) to give C10-2 (17.3 g, crude) as a brown liquid.

### General Procedure for Synthesis of C10-3

To a solution of C10-2 (10 g, 50.71 mmol) in CH₂Cl₂ (100 mL) was added Boc₂O (22.13 g, 101.42 mmol, 23.30 mL) and DMAP (9.29 g, 76.06 mmol) at 0° C. The reaction mixture was poured into water (20 mL) and extracted with EtOAc (30 mL*3). The combined organic layers were washed with brine (30 mL*3), dried over Na₂SO₄ and concentrated under reduced pressure. The filtrate was concentrated to give crude product which was purified by flash chromatography on silica gel (0-10% EtOAc in petroleum ether) to give C10-3 (2.84 g, 20.0% yield) as colorless oil.

### General Procedure for Synthesis of C10

To a solution of C10-3 (1.2 g, 4.04 mmol) in THF (6 mL) and H₂O (2 mL) was added LiOH·H₂O (338.73 mg, 8.07 mmol) and stirred at 20° C. for 1 h. then the mixture was stirred at 80° C. for 1 h. The reaction mixture was adjusted to pH=4 with HCl (1 mol/L), and then poured into water (10 mL) and extracted with EtOAc (50 mL*3). The combined organic layers were washed with brine (50 mL*3), dried over Na₂SO₄, then concentrated under reduced pressure to give C10 (887 mg, 2.96 mmol, 73.5% yield) as a brown solid.

### General Procedure for Synthesis of C12

To a solution of C10 (150 mg, 557.07 umol) in CH₃CN (2 mL) was added CDI (90.33 mg, 557.07 umol) and stirred at 20° C. for 40 min. TLC (PE:EtOAc=3:1) showed the C10 (150 mg, 557.07 umol) (Rf=0.3) was consumed completely. The C11 (177.89 mg crude) was used for next step. To a solution of C11 (177.89 mg, 554.29 umol) and C9 (141.49 mg, 554.29 umol) in CH₃CN (3 mL) was added t-BuOK (62.20 mg, 554.29 umol) and stirred at 80° C. for 1 h. The reaction mixture was poured into water (20 mL) and extracted with EtOAc (20 mL*3). The combined organic layers were washed with brine (30 mL*3), dried over Na₂SO₄ then reaction mixture was concentrated in vacuum to give C12 (358 mg crude) as a yellow solid.

### General Procedure for Synthesis of Compound 2

To a solution of C12 (350 mg, 690.98 umol) in EtOH (3 mL) was added HCl (0.3 mL). The reaction mixture was stirred at 80° C. for 1 h. The reaction mixture was concentrated and the crude product was purified by Prep-HPLC (column: Waters Xbridge 150*25 Su, table: 63-93% B (A=water (0.05% ammonia hydroxide v/v)), B=acetonitrile), flow rate: 25 mL/min, UV Detector 220 nm) to afford compound 2 (12.7 mg, 4.5% yield) as a yellow solid.

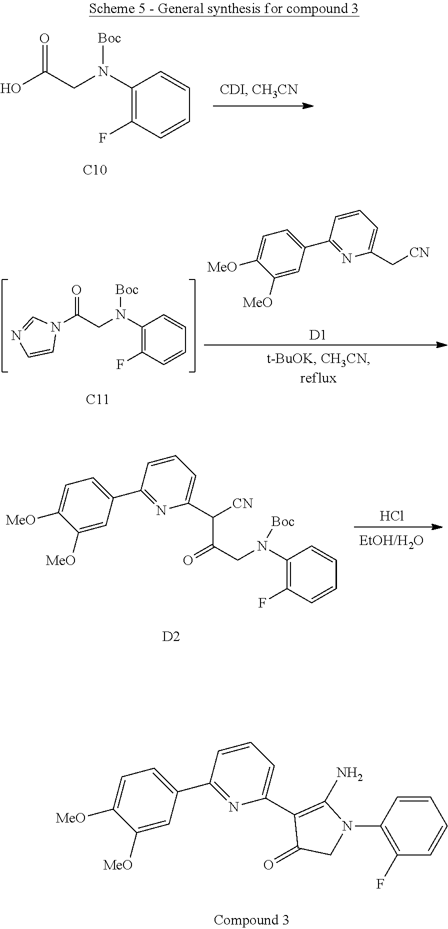

### General Procedure for Synthesis of D2

To a solution of C10 (100 mg, 371 umol) in CH₃CN (2 mL) was added CDI (60.22 mg, 371 umol), the reaction mixture was stirred at 20° C. for 40 min. TLC (PE:EtOAc=3:1) showed the C10 (Rf=0.3) was consumed completely.

Half of above reaction solution transferred to another bottle and D1 (47 mg, 184 umol) in CH₃CN (0.5 mL) was added, then added t-BuOK (20.73 mg, 185 umol) and warmed to 80° C. and stirred for 1 h. The reaction mixture was adjusted to pH=4 with HCl (1 mol/L), and then poured into water (10 mL) and extracted with EtOAc (10 mL*3). The combined organic layers were washed with brine (10 mL*3), dried over Na₂SO₄, then concentrated under reduced pressure to afford D2 (59 mg, crude, 63.17% yield), which was used in next step directly.

### General Procedure for Synthesis of Compound 3

To a solution of D2 (59 mg, 116.71 umol) in EtOH (0.5 mL) was added 37% HCl (0.05 mL). The reaction mixture was stirred at 80° C. for 1 h. The reaction solution was concentrated and the purified by Prep-HPLC (column: Waters Xbridge 150*25 5u, table: 63-93% B (A=water (0.05% ammonia hydroxide v/v)), B=acetonitrile), flow rate: 25 mL/min, UV Detector 220 nm) to afford compound 3 (22.3 mg, 44.77% yield, 95% purity)

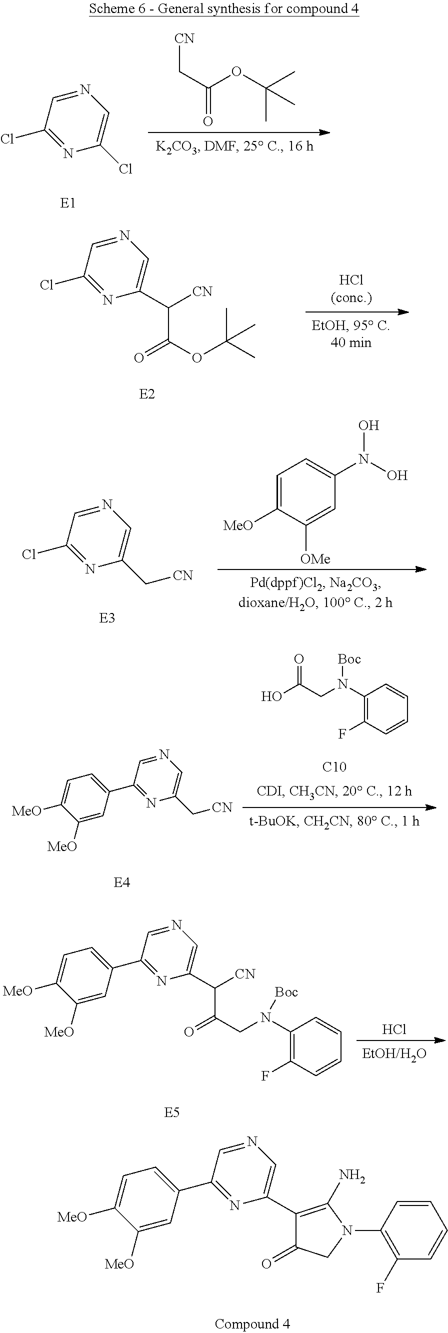

### General Procedure for Synthesis of E2

To a solution of E1 (1 g, 6.71 mmol) in DMF (15 mL) was added K₂CO₃ (2 g, 14.47 mmol) and tert-butyl 2-cyanoacetate (1 g, 7.08 mmol) under N₂. The mixture was stirred at 25° C. for 16 hr. The mixture was quenched with water (30 mL) and extracted with ethyl acetate (15 mL×3). The combined organic layer was dried over Na₂SO₄, filtered and concentrated to give crude E2 (2 g, 85% purity) as brown oil which was used for next step directly without further purification.

### General Procedure for Synthesis of E3

A mixture of E2 (2 g, 6.70 mmol, 85% purity) in HCl (3 mL) (conc.) and ethanol (15 mL) was stirred at 90° C. for 40 min. The mixture was quenched with sat. NaHCO₃ to pH=8 and extracted with ethyl acetate (20 mL×3). The combined organic layer was dried over Na₂SO₄, filtered and concentrated. The residue was purified by flash column chromatography on silica gel (30% ethyl acetate in PE) to give E3 (350 mg, 34.01% yield) as yellow oil.

### General Procedure for Synthesis of E4

To a solution of E3 (350 mg, 2.28 mmol) and (3,4-dimethoxyphenyl)boronic acid (414.75 mg, 2.28 mmol) in dioxane (5 mL) and H₂O (1 mL) was added Pd(dppf)Cl₂ (166.76 mg, 227.91 umol) and Na₂CO₃ (483.12 mg, 4.56 mmol). The mixture was stirred at 100° C. for 2 hr under N₂. The mixture was filtered. The filtrate was concentrated to give crude product which was purified by flash chromatography on silica gel (0-50% EtOAc in petroleum ether) to give E4 (425 mg, 73% yield) as a yellow solid.

### General Procedure for Synthesis of E5

To a solution of C10 (100 mg, 371.38 umol) in CH₃CN (2 mL) was added CDI (60.2 mg, 371.38 umol) and stirred at 20° C. for 12 h. To another solution of E4 (47.1 mg, 184.76 umol) in CH₃CN (1 mL) was added t-BuOK (20.7 mg, 184.76 umol) and half of the first reaction solution, then the mixture was stirred at 80° C. for 1 h. The reaction mixture was adjusted to pH=4 with HCl (1 mol/L), then poured into water (10 mL) and extracted with ethyl acetate (10 mL×3). The combined organic layers were washed with brine (10 mL×3), dried over Na₂SO₄, filtered and concentrated in vacuum to give E5 (93 mg crude) as a yellow solid.

### General Procedure for Synthesis of Compound 4

To a solution of E5 (93 mg, 183.60 umol) in EtOH (1 mL) was added HCl (0.1 mL). The reaction mixture was stirred at 80° C. for 1 h. The mixture was purified by Prep-HPLC (column: Boston Green ODS 150*30 mm*5 um, table: 43%-53% B (A=water (TFA), B=acetonitrile), flow rate: 25 mL/min, UV Detector 220 nm) and (column: Waters Xbridge 150*25 5u, table: 63-93% B (A=water (0.05% ammonia hydroxide v/v)), B=acetonitrile), flow rate: 25 mL/min, UV Detector 220 nm) to afford compound 4 (17.1 mg, 95% purity, 22.9% yield) as a yellow solid.

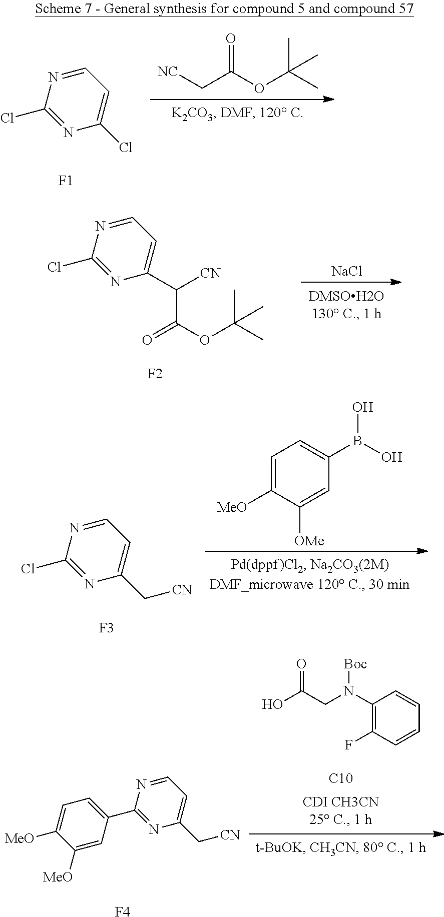

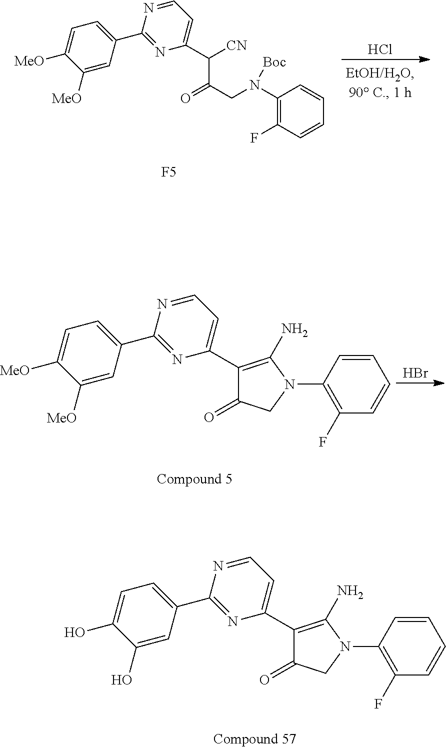 [SMILES: COc1ccc(-c2nccc(C(C#N)C(=O)CN(C(=O)OC(C)(C)C)c3ccccc3F)n2)cc1OC ; Cl ; "F5" ; COc1ccc(-c2nccc(C3=C(N)N(c4ccccc4F)CC3=O)n2)cc1OC ; Br ; NC1=C(c2ccnc(-c3ccc(O)c(O)c3)n2)C(=O)CN1c1ccccc1F]

### General Procedure for Synthesis of F2

To a solution of F1 (2 g, 13.42 mmol) in DMF (5 mL) was added tert-butyl 2-cyanoacetate (2.27 g, 16.11 mmol) and K₂CO₃ (3.71 g, 26.85 mmol). The resulting mixture was stirred at 120° C. for 12 hr. The reaction was diluted with water (10 mL) and adjusted with HCl (1M) to pH=6, then extracted with EtOAc (20 mL×3). The combined organic layer was washed with brine (30 mL), dried over anhydrous Na₂SO₄, filtered and concentrated in vacuum.

The residue was combined with another parallel batch, 2 g of F1 was conducted, the two batches were combined and purified by column chromatography (SiO2, DCM:MeOH=10:1) to give F2 (1.28 g, 6.87% yield, 78% purity) as a yellow solid and F2 (1.07 g, 18.54% yield, 59% purity) as a yellow solid.

### General Procedure for Synthesis of F3

To a solution of F2 (300 mg, 1.18 mmol 78% purity) in DMSO (2 mL) and H₂O (2 mL) was added NaCl (138 mg, 2.37 mmol). The mixture was stirred at 130° C. for 1 hr. The reaction mixture was diluted with H₂O (20 mL) and extracted with EtOAc (20 mL×3). The combined organic layer was washed with brine (20 mL×3), dried over anhydrous Na₂SO₄, filtered and concentrated under reduced pressure to give a residue. Another parallel reaction of 1.25 g of compound 2 was conducted, and the two batch reactions worked up together. The combined residue was purified by column chromatography (SiO2, DCM:MeOH=10:1) to give F3 (287.6 mg, 48.46% yield) as a yellow solid.

### General Procedure for Synthesis of F4

To a solution of F3 (100 mg, 0.65 mmol) and Na₂CO₃ (0.5 mL, 2M) in DME (1.5 mL) was added (3,4-dimethoxyphenyl) boronic acid (154.05 mg, 0.85 mmol), Pd(dppf)Cl₂ (23.82 mg, 0.03 mmol). The mixture was microwaved at 120° C. for 30 minutes. The reaction mixture was combined another 100 mg batch of parallel reaction and worked up together. The combined reaction mixture was diluted with water (10 mL), extracted with EtOAc (10 mL×3). The combined organic layer was washed with brine (10 mL×3), dried over anhydrous Na₂SO₄, filtered, and concentrated under reduced pressure to give a residue. The residue was purified by column chromatography (SiO2, Petroleum ether/Ethyl acetate=1/1) to give F4 (72 mg, 35.52% yield, 82% purity) as a yellow oil.

### General Procedure for Synthesis of F5

Solution A: To a solution of C10 (80 mg, 0.29 mmol) in CH₃CN (0.3 mL) was added CDI (48.17 mg, 0.29 mmol). The mixture was stirred at 25° C. for 5 min.

Solution B: To another solution of F4 (70 mg, 0.27 mmol) in CH₃CN (1 mL) was added t-BuOK (30.77 mg, 0.27 mmol), the mixture was stirred at 80° C. for 3 min.

The mixture A was added dropwise into a mixture B at 80° C., the resulting mixture was stirred at 80° C. for 15 min. The reaction mixture was quenched with H₂O (5 mL) and extracted with EtOAc (5 mL×3). The combined organic layers were dried over anhydrous Na₂SO₄, filtered and concentrated under reduced pressure to give a crude F5 (98 mg, crude) as a yellow oil.

### General Procedure for Synthesis of Compound 5

To a solution of F5 (98 mg, 0.19 mmol) in EtOH (1 mL) was added 37% HCl (0.2 mL). The mixture was stirred at 90° C. for 1 h. The reaction mixture was concentrated and then crude product was purified by prep-HPLC (column: Boston Green ODS 150*30 mm*5 um; mobile phase: [water(FA)-ACN]; B %: 22%-42%, 12 min) to give compound 5 (4.8 mg, 4.3% yield for 2 steps, 99.34% purity) as a white solid.

### General Procedure for Synthesis of Compound 57

A suspension of compound 5 (370 mg by another batch, 910.41 umol) in HBr (5 mL, 40% purity) was heated to 120° C. for 10 h. The crude product was purified by Prep-HPLC (column: Boston Prime C18 150*30 mm*5 um; mobile phase: [water (FA)-ACN]; B %: 12%-42%, 12 min) to give compound 57 (84.6 mg, 24% yield, 98% purity) as a gray solid.

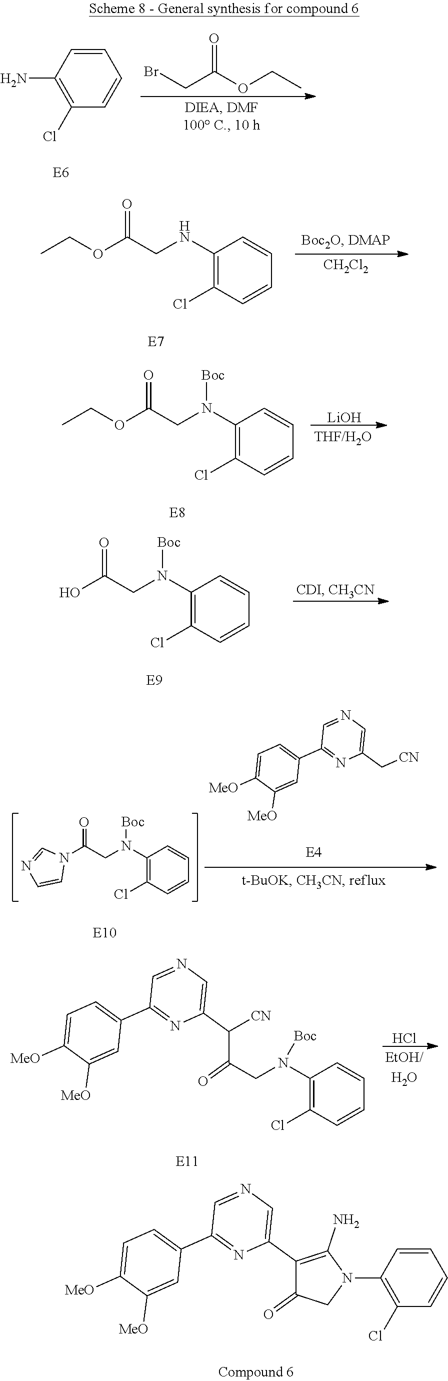

### General Procedure for Synthesis of E7

To a solution of E6 (5 g, 39.19 mmol) and DIEA (5.07 g, 39.19 mmol) in DMF (20 mL) was added ethyl 2-bromoacetate (1.64 g, 9.80 mmol) at 100° C. for 10 h. The reaction mixture was poured into water (20 mL) and extracted with EtOAc (30 mL×3), the combined organic layers were washed with brine (30 mL×3), dried over Na₂SO₄ and concentrated under reduced pressure. The filtrate was concentrated to give crude product which was purified by flash chromatography on silica gel (0-30% EtOAc in petroleum ether) to give E7 (1.4 g, 33% yield, crude) as yellow oil.

### General Procedure for Synthesis of E8

To a solution of E7 (1.4 g, 6.55 mmol) in CH₂Cl₂ (10 mL) was added Boc₂O (2.86 g, 13.10 mmol) and DMAP (1.20 g, 9.83 mmol) at 0° C. for 20 min, then add Boc₂O (4.29 g, 19.66 mmol) at 0° C. The reaction mixture was stirred at 20° C. for 12 h. The reaction mixture was poured into water (30 mL) and extracted with EtOAc (30 mL×3). The combined organic layers were washed with brine (30 mL×3), dried over Na₂SO₄. The organic layer was concentrated in vacuum. The residue was purified by flash column chromatography on silica gel (0-10 EtOAc in PE). Then the crude product was purified by Prep-HPLC (column: Waters Xbridge 150*25 Su, table: 6393 B (A=water (0.05% ammonia hydroxide v/v)), B=acetonitrile), flow rate: 25 mL/min, UV Detector 220 nm) to give E8 (217 mg, 10.34% yield) as a colorless oil.

### General Procedure for Synthesis of E9

To a solution of E8 (214 mg, 682.02 umol) in THF (2 mL) and H₂O (1 mL) was added LiOH·H₂O (57.24 mg, 1.36 mmol). The mixture was stirred at 20° C. for 10 hr. The reaction mixture was adjusted to pH=5 with HCl (1 mol/L), then poured into water (10 mL) and extracted with EtOAc (10 mL×3). The combined organic layers were washed with brine (10 mL×3), dried over Na₂SO₄, then concentrated under reduced pressure to give E9 (170 mg, 85.49% yield) as a yellow oil.

### General Procedure for Synthesis of E11

To a solution of E9 (80 mg, 279.99 umol) in CH₃CN (2 mL) was added CDI (54.48 mg, 335.99 umol) and stirred at 20° C. for 10 h. TLC (PE:EtOAc=3:1) showed the E9 (Rf=0.3) was consumed completely. The reaction mixture of E10 was used for next step.

To a solution of E4 (71.46 mg, 279.94 umol) in CH₃CN (3 mL) was added t-BuOK (47.12 mg, 419.91 umol) and stirred at 20° C. for 10 min, then the reaction mixture of E10 was added, the reaction was stirred at 80° C. for 1 h. The reaction mixture was adjusted to pH=4 with HCl (1 mol/L), and then poured into water (5 mL) and extracted with EtOAc (10 mL×3). The combined organic layers were washed with brine (10 mL×3), dried over Na₂SO₄, then concentrated under reduced pressure to give E11 (189 mg, crude) as a yellow solid.

### General Procedure for Synthesis of Compound 6

To a solution of E11 (189 mg, 361.39 umol) in EtOH (2 mL) was added HCl (0.2 mL, 12 M/L). The reaction mixture was stirred at 80° C. for 1 h. The crude product was purified by Prep-HPLC (column: Boston Prime C18 150*30 mm*5 um; mobile phase: [water (NH₃H₂O+NH₄HCO₃)-ACN]; B %: 41%-71%, 10 min) to afford. The reaction was lyophilized to give compound 6 (2.6 mg, 1.67% yield, 100% purity) as a white solid.

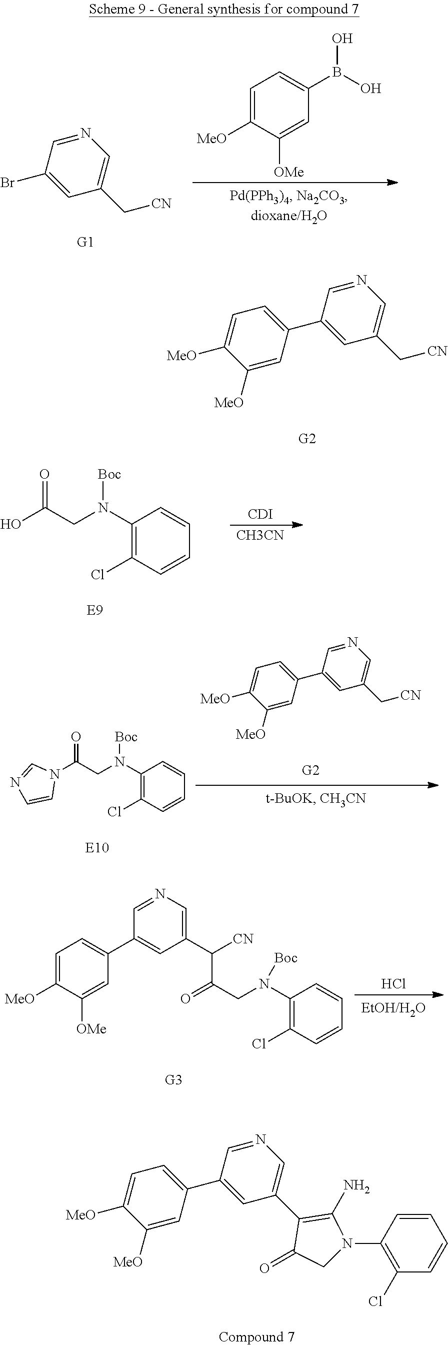

### General Procedure for Synthesis of G2

To a solution of (3,4-dimethoxyphenyl) boronic acid (930 mg, 5.11 mmol) in dioxane (10 mL) and H₂O (2 mL) was added G1 (1.01 g, 5.11 mmol), Na₂CO₃ (1.08 g, 10.22 mmol) and Pd (PPh₃)₄ (590.54 mg, 0.51 mmol), the mixture was stirred at 80° C. for 2 hr. The reaction mixture was diluted with H₂O (300 mL) and extracted with EtOAC (50 mL×3). The combined organic layers were washed with brine (50 mL×3), dried over anhydrous Na₂SO₄, filtered and concentrated under reduced pressure to give a residue. The residue was purified by column chromatography (SiO₂, Petroleum ether/Ethyl acetate=1/1) to give G2 (720 mg, 38.23% yield, 69% purity) as a yellow solid.

### General Procedure for Synthesis of G3

A: To a solution of E9 (350 mg, 1.22 mmol) in CH₃CN (3 mL) was added CDI (198.63 mg, 1.22 mmol). The mixture was stirred at 25° C. for 2 hr to give E10 as a yellow solution in CH₃CN.

B: To a solution of G2 (300 mg, 1.18 mmol) in CH₃CN (3 mL) was added t-BuOK (264.77 mg, 2.36 mmol) and E10 (396.15 mg, 1.18 mmol). The mixture was stirred at 80° C. for 1 hr. The reaction mixture combined with another batch of reaction mixture (100 mg of 2 was conducted), and worked up together. The combined reaction mixture was diluted with H₂O (30 mL) and extracted with EtOAc (30 mL×3). The combined organic layers were dried overanhydrous Na₂SO₄, filtered and concentrated under reduced pressure to give G3 (779 mg, crude) as a brown solid.

### General Procedure for Synthesis of Compound 7

To a solution of G3 (670 mg, 1.28 mmol) in EtOH (7 mL) was added 37% HCl (1 mL). The mixture was stirred at 90° C. for 1 hr. The reaction mixture combined with another batch of reaction mixture (from 100 mg of 3), the two-batch reaction mixture combined and worked up together. The reaction mixture was concentrated under reduced pressure to remove solvent and then purified by prep-HPLC (column: Boston Green ODS 150*30 mm*5 um; mobile phase: [water(FA)-ACN]; B %: 7%-37%, 12 min) to give compound 7 (237 mg, 43.33% yield, 99.74% purity) as a white solid.

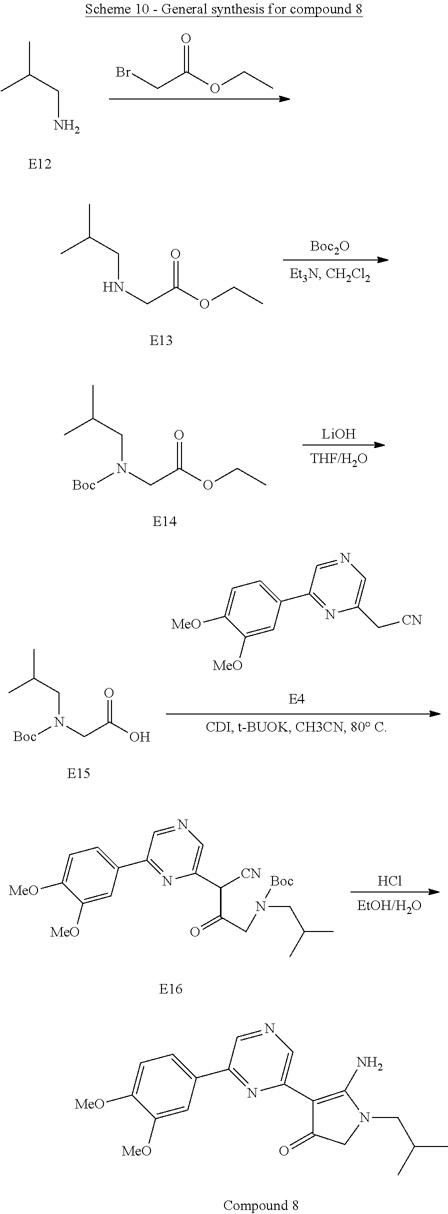

### General Procedure for Synthesis of E13

To a solution of E12 (4.38 g, 59.88 mmol) in CH₂Cl₂ (50 mL) was added ethyl 2-bromoacetate (5 g, 29.94 mmol), the mixture was stirred at 25° C. for 12 hr. The mixture was purified by column chromatography (SiO2, Petroleum ether/Ethyl acetate=1/1) to give E13 (3.02 g, 63.35% yield) as a yellow oil.

### General Procedure for Synthesis of E14

To a solution of E13 (2.92 g, 18.34 mmol) in CH₂Cl₂ (30 mL) was added Et₃N (2.78 g, 27.51 mmol) and Boc₂O (4.00 g, 18.34 mmol) at 0° C. The mixture was stirred at 25° C. for 12 hr. The mixture was purified by column chromatography (SiO2, Petroleum ether/Ethyl acetate=1/1 to 0/1) to give E14 (3.73 g, 78.43% yield) as a yellow oil.

### General Procedure for Synthesis of E15

To a solution of E14 (2 g, 7.71 mmol) in THF (20 mL) and H₂O (4 mL) was added LiOH·H₂O (1.62 g, 38.56 mmol), the mixture was stirred at 60° C. for 2 hr. The reaction mixture was diluted with H₂O (100 mL) and extracted with EtOAc (100 mL×3), the aqueous phase was adjusted pH to 5 with HCl (1M) and extracted with EtOAc (100 mL×3), The combined organic layer was washed with brine (100 mL×3), dried over anhydrous Na₂SO₄, filtered and concentrated under reduced pressure to give E15 (1.5 g, 84.10% yield) as a yellow oil.

### General Procedure for Synthesis of E16

To a solution of E15 (90.60 mg, 0.39 mmol) in CH₃CN (1 mL) was added CDI (76.22 mg, 0.47 mmol), the mixture was stirred at 25° C. for 2 hr. To another solution of E4 (100 mg, 0.39 mmol) in CH₃CN (1 mL) was added t-BuOK (87.92 mg, 0.78 mmol), the mixture was stirred at 80° C. for 5 min. Then the first solution was added dropwise at 80° C., The resulting mixture was stirred at 80° C. for 15 min. The mixture was added to water (20 mL) and extracted with EtOAc (20 mL×3). The combined organic layers were washed with brine (20 mL×3), dried over Na₂SO₄, filtered and concentrated under reduced pressure to give E16 (170 mg, 92.62% yield, 100% purity) as white solid

### General Procedure for Synthesis of Compound 8

To a solution of E16 (170 mg, 0.36 mmol) in EtOH (5 mL) was added 37% HCl (1 mL). The mixture was stirred at 80° C. for 0.5 hr. The mixture was quenched with sat. NaHCO₃ to pH=8 and extracted with ethyl acetate (30 mL×3). The combined organic layer was dried over Na₂SO₄, filtered and concentrated. The crude product was purified by prep-HPLC, [column: Boston Prime C18 150*30 mm*5 um; mobile phase: [water(NH3H2O+NH4HCO3)−ACN]; B %: 37%-67%, 10 min], then dried by lyophilization to get compound 8 (61.3 mg, 46% yield) as a white solid.

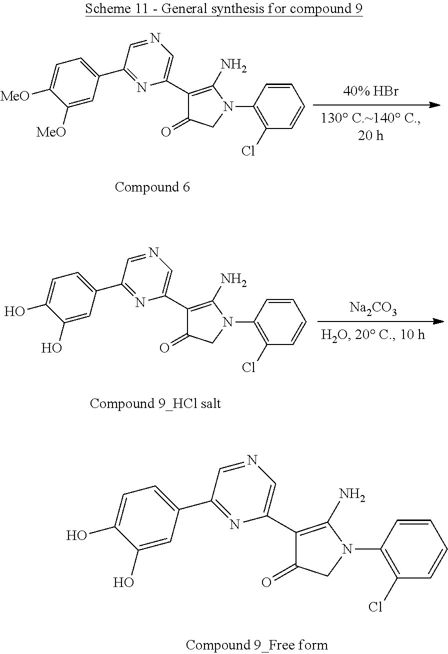

### General Procedure for Synthesis of Compound 9 HCl Salt

A solution of compound 6 (3 g, 7.09 mmol) in HBr (20 mL, 40% purity) was heated to 130° C. for 10 h, then stirred at 140° C. for 10 h. The mixture was filtered and washed with MeOH (20 mL). The filter cake was purified by Prep-HPLC (column: Boston Green ODS 150*30 mm*5 um; mobile phase: [water(HCl)-ACN]; B %: 23%-43%, 10 min) to give compound 9_HCl salt (1.64 g, 58.85% yield, 100% purity, HCl) as a yellow solid.

### General Procedure for Synthesis of Compound 9 Free Form

To a solution of compound 9_HCl salt (50 mg, 126.64 umol) in H₂O (5 mL) was added Na₂CO₃ (26.85 mg, 253.29 umol). The mixture was stirred at 20° C. for 10 h. The mixture was filtered and washed with H₂O (10 mL). The filter cake was collected and dried by lyophilization to give compound 9_free form (39 mg, 78.00% yield, 99% purity) as a yellow solid.

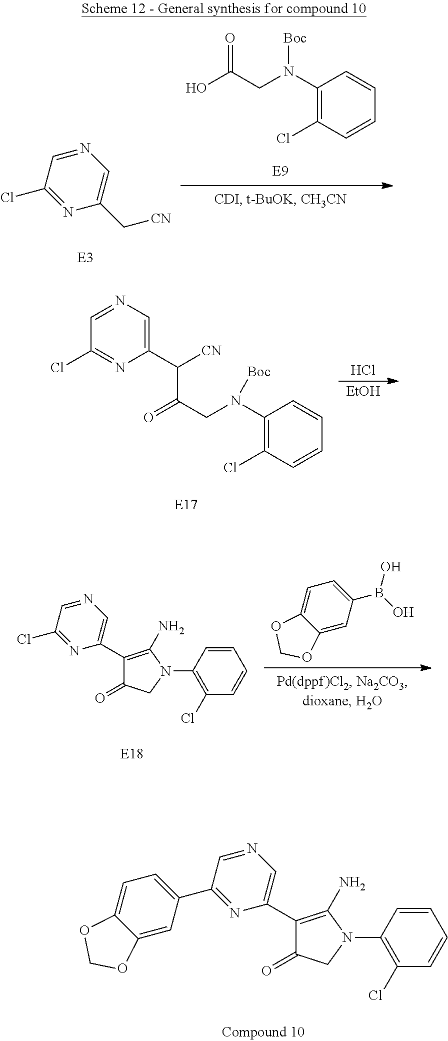

### General Procedure for Synthesis of E17

To a solution of E9 (1 g, 3.50 mmol) in CH₃CN (5 mL) was added CDI (567.50 mg, 3.50 mmol), the mixture was stirred at 25° C. for 2 hr. In another flask, a solution of E3 (500 mg, 3.26 mmol) in CH₃CN (5 mL) was added t-BuOK (730.69 mg, 6.51 mmol) and then the first solution was added. The mixture was stirred at 80° C. for 1 hr. The mixture was diluted with water (100 mL), extracted with EtOAc (100 mL×3). The combined organic layer was washed with brine (100 mL×3), dried over anhydrous Na₂SO₄, filtered and concentrated, filtered and concentrated under reduced pressure to give a E17 (1.54 g, crude), which was used into the next step without further purification.

### General Procedure for Synthesis of E18

To a solution of E17 (1 g, 2.37 mmol) in EtOH (10 mL) was added HCl (2.5 mL, 37% purity), the reaction was heated to 90° C. for 1 hr. The solvent was removed in vacuo and the residue was basified by NaHCO₃ aqueous solution to pH=8, the mixture was extracted with CH₂Cl₂ (30 mL×3). The organic layer was washes with brine (30 mL), dried over sodium sulfate and concentrated. The residue was triturated with a solution of PE/EtOAc (5/1, 10 mL) and then filtered. The solid was dried in vacuo to afford E18 (300 mg, 39% yield) as a black solid.

### General Procedure for Synthesis of Compound 10

To a solution of E18 (50 mg, 155.69 umol) and 1,3-benzodioxol-5-ylboronic acid (31 mg, 187 umol) in dioxane (1 mL) and H₂O (0.2 mL) was added Pd(dppf)Cl₂·CH₂Cl₂ (12.71 mg, 15.57 umol) and Na₂CO₃ (33 mg, 311.37 umo). The reaction was degassed and refilled N₂ for 3 times and then heated to 100° C. for 4 hr. The reaction mixture was diluted with water (10 mL) and extracted with EtOAc (20 mL×2). The combined organic layer was washed with brine (20 mL), dried over sodium sulfate and concentrated. The crude product was purified by prep-TLC (100% EtOAc) to afford a 30 mg crude product. The crude product was purified by prep-HPLC [column: O-Phenomenex C18 150*10 mm*5 um; mobile phase: [water(NH₃H₂O+NH₄HCO₃)-ACN]; B %: 33%-63%, 10 min] to afford compound 10 (4.3 mg, 6.3% yield, 93% purity) as a white solid.

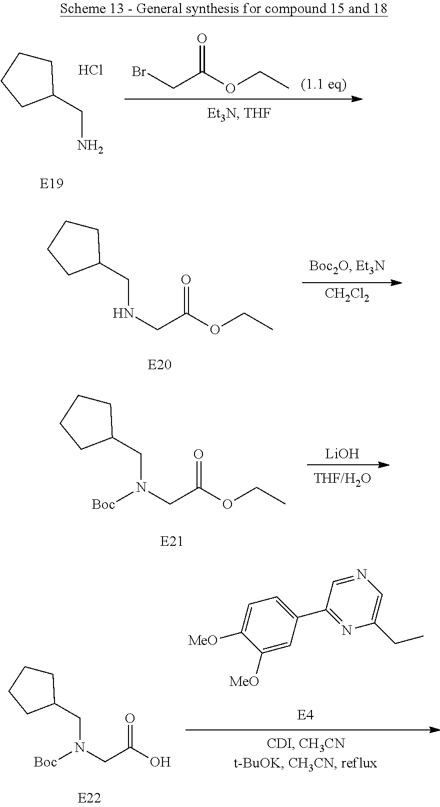

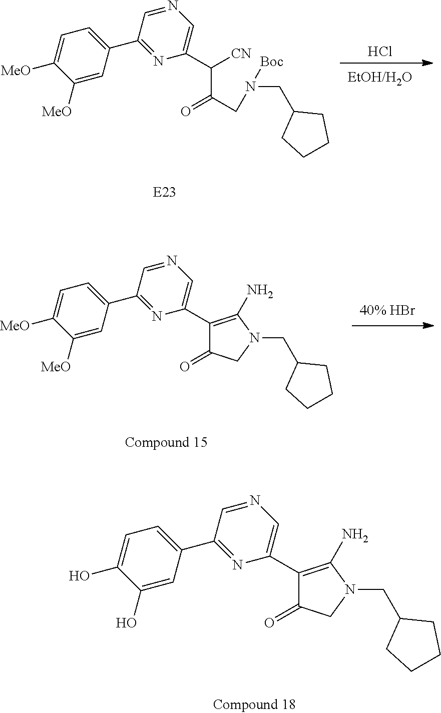 [SMILES: COc1ccc(-c2cncc(C(C#N)C(=O)CN(CC3CCCC3)C(=O)OC(C)(C)C)n2)cc1OC ; Cl ; COc1ccc(-c2cncc(C3=C(N)N(CC4CCCC4)CC3=O)n2)cc1OC ; NC1=C(c2cncc(-c3ccc(O)c(O)c3)n2)C(=O)CN1CC1CCCC1]

### General Procedure for Synthesis of E20

To a solution of E19 (500 mg, 3.69 mmol, HCl) in THF (10 mL) was added TEA (932.55 mg, 9.22 mmol) and ethyl 2-bromoacetate (677.19 mg, 4.06 mmol), then stirred at 25° C. for 12 hr, the reaction mixture was diluted with water (30 mL), and extracted with EtOAc (30 mL×3), The organic layer was washed with brine (30 mL), dried over anhydrous Na₂SO₄ and concentrated, the residue was purified by Combi Flash (SiO2, 50% to 60% EtOAc inPE) to give E20 (460 mg 67.36% yield) as colorless oil.

### General Procedure for Synthesis of E21

To a solution of E20 (460 mg, 2.48 mmol) in CH₂Cl₂ (10 mL) was added Et₃N (502.50 mg, 4.97 mmol) and Boc₂O (812.86 mg, 3.72 mmol), the mixture was stirred at 25° C. for 2 hr. The mixture was diluted with CH₂Cl₂ (50 mL), washed with 1N HCl (30 mL). The organic layer was dried over Na₂SO₄, filtered and concentrated to give E21 (700 mg, crude) as colorless oil.

### General Procedure for Synthesis of E22

To a solution of E21 (700 mg, 2.45 mmol) in THF (10 mL) was added a solution of LiOH·H₂O (154.38 mg, 3.68 mmol) in Water (5 mL), the mixture was stirred at 60° C. for 2 hr. Water (10 mL) was added to the mixture, and the mixture was treated with 1N HCl to adjust pH to 2, then extracted with EtOAc (20 mL×2). The combined organic layer was washed with brine (10 mL), dried over Na₂SO₄, filtered and concentrated to give E22 (450 mg, 71.29% yield) as colorless oil.

### General Procedure for Synthesis of E23

To a solution of E22 (110.88 mg, 0.43 mmol) in MeCN (2 mL) was added CDI (69.87 mg, 0.43 mmol) at 20° C., and stirred for 30 min. To another solution of E4 (100 mg, 0.39 mmol) in MeCN (2 mL) was added t-BuOK (43.96 mg, 0.39 mmol) at 20° C., and then heated to 80° C., and the first solution was added. The reaction mixture was stirred at 80° C. for 0.5 hr. The reaction was diluted with water (10 mL), acidified by 1N HCl to pH=5 and extracted with EtOAc (30 mL×3). The organic layer was washed with brine (20 mL), dried over Na₂SO₄, filtered and concentrated to give E23 (190 mg, crude) as a yellow solid.

### General Procedure for Synthesis of Compound 15

To a solution of E23 (190 mg, 0.38 mmol) in EtOH (2 mL) was added HCl (0.3 mL). The mixture was stirred at 80° C. for 1 hr. The reaction mixture was concentrated under reduced pressure to remove solvent. The residue was purified by prep-HPLC (column: Boston Green ODS 150*30 mm*5 um; mobile phase: [water (FA)-ACN]; B %: 40%-70%, 14 min) to give compound 15 (90 mg, 12.93% yield, 98.11% purity) as a yellow solid.

### General Procedure for Synthesis of Compound 18

A solution of compound 15 (35 mg, 0.09 mmol) in HBr (4 mL, 40% purity) was stirred at 120° C. for 16 hr. The reaction mixture was concentrated under reduced pressure to remove solvent. The residue was purified by prep-HPLC (column: Boston Green ODS 150*30 mm*5 um; mobile phase: [water (HCl)-ACN]; B %: 18%-38%, 10 min) to give compound 18 (5.3 mg, 15.00% yield, 92.78% purity) as a yellow solid.

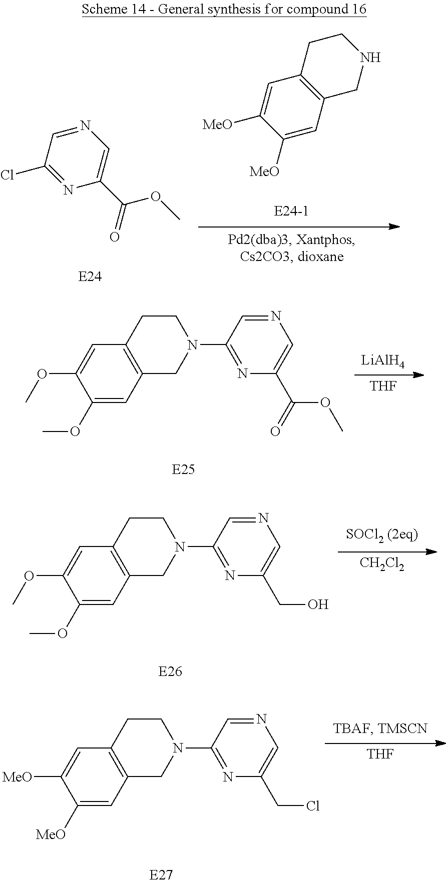

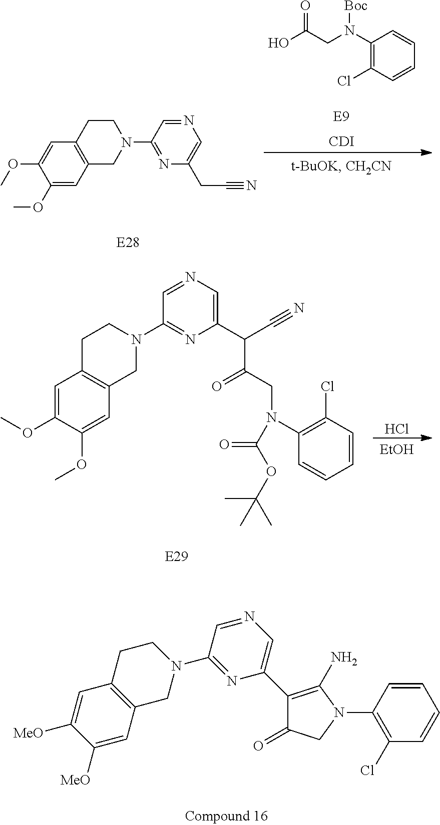

### General Procedure for Synthesis of E25

To a solution of E24 (500 mg, 2.90 mmol), E24-1 (560 mg, 2.90 mmol), Pd₂(dba)₃ (132.66 mg, 144.87 umol) and Xantphos (83.82 mg, 144.87 umol) in dioxane (5 mL) was added Cs₂CO₃ (1.42 g, 4.35 mmol). The mixture was degassed and refilled nitrogen for 3 times. The reaction was heated to 110° C. for 1 h. The reaction mixture was poured into water (50 mL) and extracted with EtOAc (50 mL×3). The combined organic layer was washes with brine (30 mL×3), dried over sodium sulfate and concentrated. The residue was purified by silica gel chromatography (PE/EtOAc=2/1) to afford E25 (340 mg, 33.85% yield) as a yellow solid.

### General Procedure for Synthesis of E26

To a suspension of LiAlH₄ (53.59 mg, 1.41 mmol) in THF (5 mL) was added a solution of E25 (310 mg, 941.25 umol) in THF (0.5 mL) at −10° C. for 0.5 hr. The reaction was quenched by adding water a drop of water, a drop of 15% NaOH solution, diluted with a mixture solution of DCM/methanol (5/1, 20 mL) and filtered. The filtrate was concentrated to afford the E26 (450 mg, 95.19% yield, 60% purity) as a yellow solid.

### General Procedure for Synthesis of E27

To a solution of E26 (50 mg, 165.93 umol) in DCM (1 mL) was added SOCl₂ (98.70 mg, 829.63 umol), the reaction mixture was stirred at 20° C. for 1 h. The reaction mixture was concentrated under reduced pressure to give E27 (51 mg, crude) as a brown solid.

### General Procedure for Synthesis of E28

To a solution of E27 (51 mg, 159.48 umol) and TMSCN (31.64 mg, 318.96 umol) in THF (1 mL) was added TBAF (1 M, 318.96 uL), the reaction mixture was stirred at 25° C. for 16 h. The reaction mixture was concentrated under reduced pressure to remove solvent. The residue was purified by flash silica gel chromatography (56% ethyl acetate in PE) to give E28 (23 mg, 46.47% yield) as a yellow solid

### General Procedure for Synthesis of E29

To a solution of E9 (23.29 mg, 81.52 umol) in CH₃CN (1 mL) was added CDI (13.22 mg, 81.52 umol) and the reaction stirred at 20° C. for 10 min.

To another solution of E28 (23 mg, 74.11 umol) in CH₃CN (1 mL) was added t-BuOK (9.15 mg, 81.52 umol), the reaction was heated to 80° C., then the first above solution was added. The reaction was stirred at 80° C. for 15 min. The reaction mixture was adjusted to pH=4 with HCl (1 mol/L), then poured into water (5 mL) and extracted with EtOAc (10 mL×3). The combined organic layers were washed with brine (10 mL×3), dried over Na₂SO₄, then concentrated under reduced pressure to give E29 (44 mg, crude) as a brown solid.

### General Procedure for Synthesis of Compound 16

To a solution of E29 (40 mg, 69.20 umol) in EtOH (5 mL) was added HCl (0.5 mL, 12 mol/L purity). The reaction mixture was stirred at 80° C. for 1 h. The crude product was purified by Prep-HPLC (column: Boston Green ODS 150*30 mm*5 um; mobile phase: [water(HCl)-ACN]; B %: 30%-50%, 10 min) to give compound 16 (4.9 mg, 13.49% yield, 100% purity, HCl) as a yellow solid.

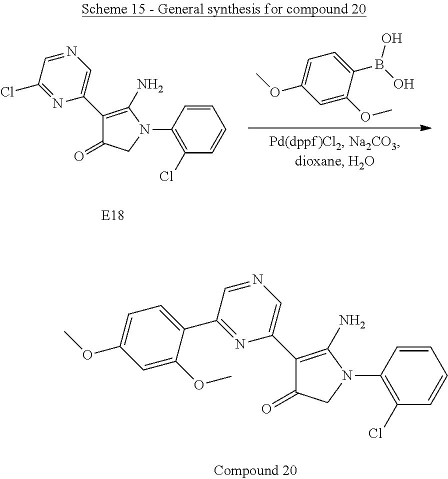 [SMILES: NC1=C(c2cncc(Cl)n2)C(=O)CN1c1ccccc1Cl ; COc1ccc(B(O)O)c(OC)c1 ; COc1ccc(-c2cncc(C3=C(N)N(c4ccccc4Cl)CC3=O)n2)c(OC)c1]

### General Procedure for Synthesis of Compound 20

To a solution of E18 (100 mg, 0.31 mmol) in dioxane (2 mL) and H₂O (0.5 mL) was added (2,4-dimethoxyphenyl)boronic acid (56.66 mg, 0.31 mmol), Pd(dppf)Cl₂ (22.78 mg, 0.03 mmol) and Na₂CO₃ (66.00 mg, 0.62 mmol), the mixture was stirred at 100° C. for 1 hr. The reaction mixture was diluted with H₂O (10 mL) and extracted with EtOAc (10 mL×3). The combined organic layers were washed with brine (10 mL×3), dried over anhydrous Na₂SO₄, filtered and concentrated under reduced pressure to give a residue. The residue was purified by prep-HPLC (column: Boston Green ODS 150*30 mm*5 um; mobile phase: [water(HCl)-ACN]; B %: 36%-56%, 10 min) to give compound 20 (50 mg, 34.96% yield, 100% purity, HCl) as a yellow solid.

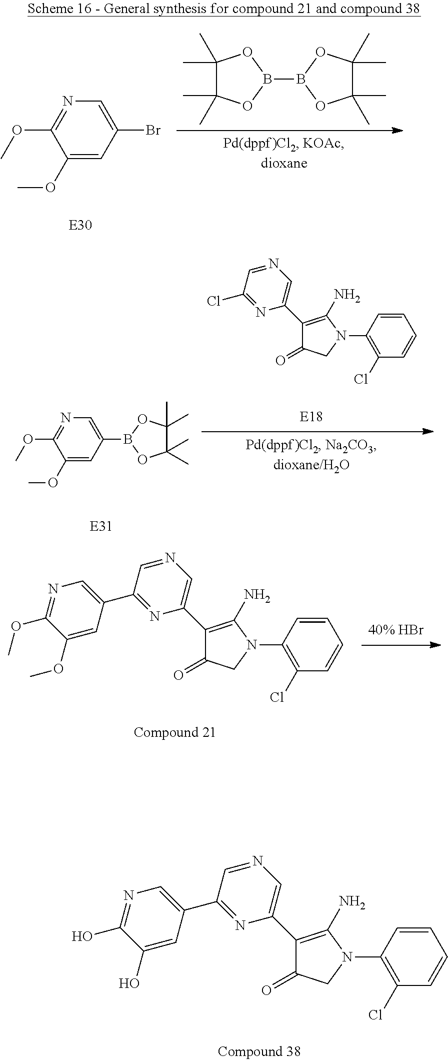

### General Procedure for Synthesis of Compound 21

To a solution of E30 (50 mg, 0.229 mmol) and 4,4,5,5-tetramethyl-2-(4,4,5,5-tetramethyl-1,3,2-dioxaborolan-2-yl)-1,3,2-dioxaborolane (87.3 mg, 0.343 mmol) in dioxane (2 mL) was added Pd(dppf)Cl₂ (16.78 mg, 0.023 mmol) and KOAc (45.01 mg, 0.459 mmol). The mixture was stirred at 100° C. for 1 hr. LC-MS showed E30 was consumed completely and E31 formed. To the reaction mixture was added E18 (73.64 mg, 0.229 mmol), Na₂CO₃ (48.61 mg, 0.459 mmol), Pd(dppf)Cl₂ (16.78 mg, 0.023 mmol) and H₂O (0.4 mL). The resulting mixture was degassed and refilled N2 for 3 times, and then stirred at 100° C. for 1 hr. LC-MS showed compound 2 was consumed completely and compound 21 formed. The reaction mixture was diluted with H₂O (10 mL) and extracted with EtOAc (10 mL×3). The combined organic layers were washed with brine (10 mL×3), dried over anhydrous Na₂SO₄, filtered and concentrated under reduced pressure to give a residue. The residue was purified by prep-HPLC (column: Boston Green ODS 150*30 mm*5 um; mobile phase: [water (HCl)-ACN]; B %: 50%-70%, min) to give compound 21 (14 mg, 98% purity, HCl salt, 13% yield) as a yellow solid.

### General Procedure for Synthesis of Compound 38

A solution of compound 21 (20 mg by another batch, 0.05 mmol) in 40% HBr (1.5 mL) was stirred at 80° C. for 16 hr. The reaction mixture was concentrated under reduced pressure to remove solvent. Another batch of parallel reaction (10 mg of compound 21) was conducted, and the two batch of crude product was combined and purified together. The residue was purified by prep-HPLC (column: Boston Green ODS 150*30 mm*5 um; mobile phase: [water(HCl)-ACN]; B %: 15%-36%, 10 min) to give compound 38 (3.8 mg, 19.33% yield, 95.73% purity) as a white solid.

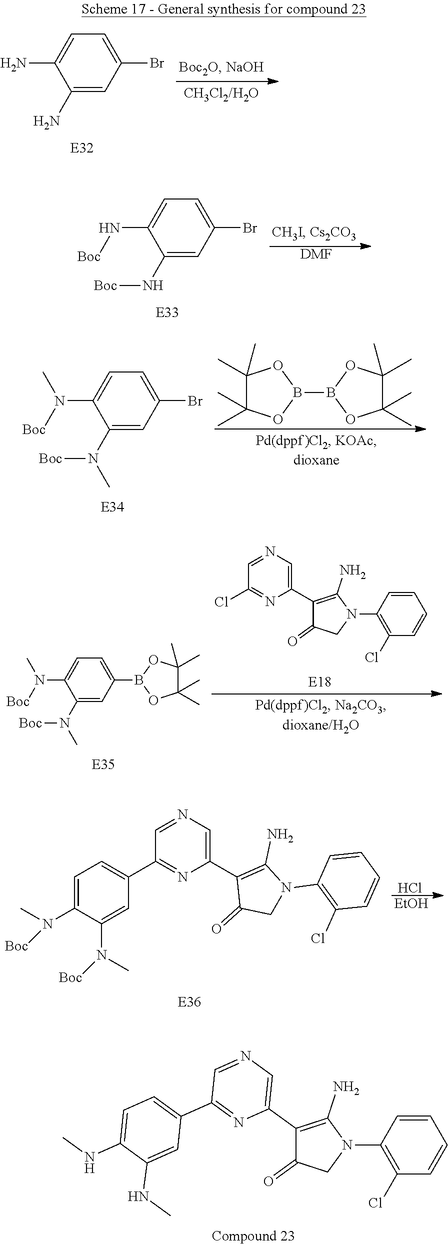

### General Procedure for Synthesis of E33

To a solution of E32 (2 g, 10.69 mmol), Boc₂O (11.67 g, 53.47 mmol) in DCM (30 mL) was added NaOH (13.4 mL, 2N in H₂O), the reaction mixture was stirred at 25° C. for 12 h. The reaction mixture was diluted with dichloromethane (70 mL) and brine (50 mL). The organic layer was dried over Na₂SO₄, filtered and concentrated under reduced pressure to give a residue. The residue was purified by silica gel chromatography (PE/EtOAc=5/1) to afford E33 (3.34 g, 81% yield) as a brown solid.

### General Procedure for Synthesis of E34

To a solution of Cs₂CO₃ (2.52 g, 7.75 mmol) in DMF (10 mL) was added E33 (1 g, 2.58 mmol) at 0° C., then CH₃I (1.83 g, 12.91 mmol) was added. The mixture was stirred at 20° C. for 2 h. The mixture was poured into water (20 mL) and extracted with EtOAc (20 mL×3). The combined organic layers were washed with brine (20 mL×3), dried over Na₂SO₄, then concentrated under reduced pressure to afford E34 (1.27 g, crude, 84% purity) as a brown solid.

### General Procedure for Synthesis of E36

To a solution of E34 (100 mg, 240.78 umol) and 4,4,5,5-tetramethyl-2-(4,4,5,5-tetramethyl-1,3,2-dioxaborolan-2-yl)-1,3,2-dioxaborolane (91.71 mg, 361.17 umol) in dioxane (2 mL) was added Pd(dppf)Cl₂ (17.62 mg, 24.08 umol) and KOAc (70.89 mg, 722.33 umol). The mixture was stirred at 100° C. for 2 h. LC-MS showed E35 was formed.

To the reaction mixture was added E18 (84.81 mg, 264.07 umol), Na₂CO₃ (76.33 mg, 720.18 umol), Pd(dppf)Cl₂ (17.57 mg, 24.01 umol) and H₂O (0.6 mL). The resulting mixture was degassed and refilled N₂ for 3 times, and then stirred at 100° C. for 1 h. LC-MS showed E36 was formed. The reaction mixture was diluted with H₂O (10 mL) and extracted with EtOAc (10 mL×3). The combined organic layers were washed with brine (10 mL×3), dried over anhydrous Na₂SO₄, filtered and concentrated under reduced pressure to give E36 (220 mg, crude) as a brown solid.

### General Procedure for Synthesis of Compound 23

To a solution of E36 (220 mg, 354.20 umol) in EtOH (3 mL) was added HCl (0.3 mL, 12M/L). The reaction mixture was stirred at 80° C. for 1 h. The reaction mixture was concentrated under reduced pressure to remove solvent. The crude product was purified by Prep-HPLC (column: Boston Prime C18 150*30 mm*5 um; mobile phase: [water(FA)-ACN]; B %: 17%-47%, 12 min) to give compound 23 (4.9 mg, 4.8% yield for 3 steps, 100% purity) as a yellow solid.

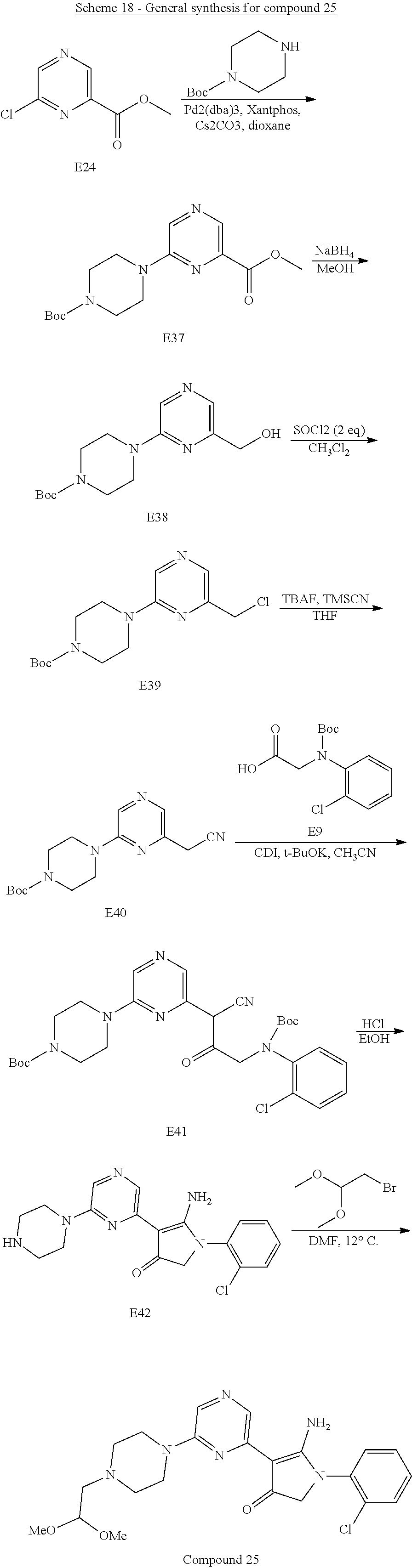

### General Procedure for Synthesis of E37

To a solution of E24 (3 g, 17.38 mmol) in dioxane (30 mL) was added tert-butyl piperazine-1-carboxylate (3.24 g, 17.38 mmol), Cs₂CO₃ (11.33 g, 34.77 mmol), Pd₂(dba)₃ (1.59 g, 1.74 mmol) and Xantphos (1.01 g, 1.74 mmol), the mixture was stirred at 110° C. for 1 hr. The reaction mixture was diluted with H₂O (50 mL) and extracted with EtOAc (50 mL×3). The combined organic layers were washed with brine (30 mL×3), dried over anhydrous Na₂SO₄, filtered and concentrated under reduced pressure to give a residue. The residue was purified by column chromatography (SiO₂, Petroleum ether/Ethyl acetate=3/1) to give E37 (1.84 g, 28.57% yield, 87% purity) as a brown solid

### General Procedure for Synthesis of E38

A solution of E37 (840 mg, 2.61 mmol) in THF (10 mL) was added dropwise into a solution of LiAlH₄ (197.80 mg, 5.21 mmol) in THF (15 mL) at −10° C., The resulting mixture was stirred at −10° C. for 30 min. The mixture was quenched with water (0.2 mL), 15% NaOH aqueous solution (0.2 mL) and water (0.6 mL). The mixture was diluted with CH₂C12/MeOH (10/1, 100 mL), then Na₂SO₄ was added until the aluminum salt was adsorbed, filtered and concentrated under reduced pressure to give a residue.

The reaction mixture was combined with another of crude product (1 g of E37 was conducted), and the combined crude product was purified by column chromatography (SiO₂, Petroleum ether/Ethyl acetate=1/1) to give E38 (365 mg, 21% yield, 90% purity) as a yellow solid.

### General Procedure for Synthesis of E39

To a solution of E38 (310 mg, 1.05 mmol) in CH₂Cl₂ (3.5 mL) was added SOCl₂ (250.59 mg, 2.11 mmol) at 0° C. The mixture was stirred at 0° C. for 1 hr. The reaction mixture was concentrated under reduced pressure to remove CH₂Cl₂. The residue was purified by column chromatography (SiO₂, Petroleum ether/Ethyl acetate=1/1) to give E39 (78 mg, 23.68% yield) as a yellow oil.

### General Procedure for Synthesis of E40

To a solution of E39 (78 mg, 0.25 mmol) in THF (1.5 mL) was added TMSCN (49.48 mg, 0.51 mmol) and TBAF (130.40 mg, 0.51 mmol). The mixture was stirred at 25° C. for 16 hr. The reaction mixture was diluted with H₂O (10 mL) and extracted with EtOAc (10 mL×3). The combined organic layers were washed with brine (10 mL×3), dried over anhydrous Na₂SO₄, filtered and concentrated under reduced pressure to give a residue. The residue was purified by column chromatography (SiO₂, Petroleum ether/Ethyl acetate=1/1) to give E40 (52 mg, 56.37% yield, 82% purity) as a yellow oil.

### General Procedure for Synthesis of E41

To a solution of E9 (53.87 mg, 0.19 mmol) in CH₃CN (1 mL) was added CDI (30.57 mg, 0.19 mmol), the mixture was stirred at 25° C. for 3 min. To another solution of E40 (52 mg, 0.17 mmol) in CH₃CN (1 mL) was added t-BuOK (19.23 mg, 0.17 mmol), the mixture was stirred at 80° C. for 2 min. and then the first solution was added dropwise at 80° C., the resulting mixture was stirred at 80° C. for 15 min. The reaction mixture was diluted with water (10 mL) and then adjusted pH to 4 by 1N HCl. The mixture was extracted with EtOAc (30 mL×3). The combined organic layer was washed with brine (30 mL×3), dried over anhydrous Na₂SO₄, filtered and concentrated to give a residue. The residue was purified by prep-HPLC (column: Boston Prime C18 150*30 mm*5 um; mobile phase: [water(NH₃H₂O+NH₄HCO₃)-ACN]; B %: 26%-56%, 10 min) to give E41 (20 mg, 20.23% yield, 99% purity) as a yellow solid.

### General Procedure for Synthesis of E42

To a solution of E41 (20 mg, 0.04 mmol) in EtOH (1 mL) was added 37% HCl (0.1 mL). The mixture was stirred at 80° C. for 1 hr. The reaction mixture was concentrated under reduced pressure to remove solvent to give E42 (13.6 mg, crude) as a yellow oil.

### General Procedure for Synthesis of Compound 25

To a solution of E42 (13.6 mg, 0.04 mmol) and 2-bromo-1,1-dimethoxyethane (18.60 mg, 0.11 mmol) in DMF (1.5 mL) was added K₂CO₃ (15.21 mg, 0.11 mmol). The mixture was stirred at 100° C. for 16 hr. The reaction mixture was purified by prep-HPLC (column: Boston Prime C18 150*30 mm*5 um; mobile phase: [water(NH₃H₂O+NH₄HCO₃)-ACN]; B %: 30%-60%, 10 min) to give compound 25 (3.3 mg, 19.41% yield, 99% purity) as a yellow solid.

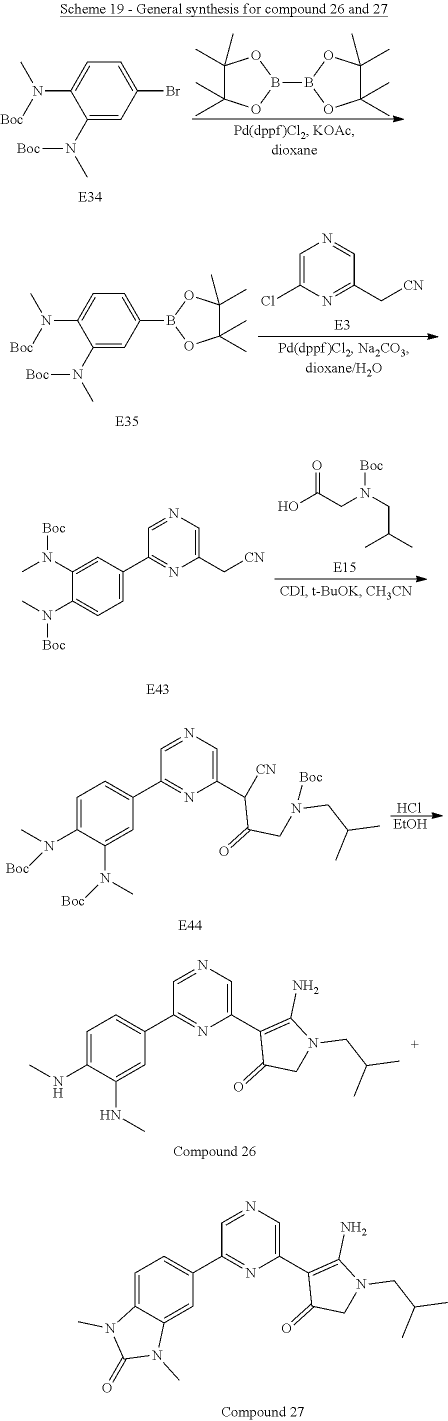

### General Procedure for Synthesis of E43

To a solution of E34 (200 mg, 481.56 umol) and 4,4,5,5-tetramethyl-2-(4,4,5,5-tetramethyl-1,3,2-dioxaborolan-2-yl)-1,3,2-dioxaborolane (183.43 mg, 722.33 umol) in dioxane (4 mL) was added Pd(dppf)Cl₂ (35.24 mg, 48.16 umol) and KOAc (141.78 mg, 1.44 mmol). The mixture was stirred at 100° C. for 2 h. To the reaction mixture was added E3 (81.10 mg, 528.13 umol), H₂O (0.6 mL), Pd(dppf)Cl₂ (35.13 mg, 48.01 umol) and Na₂CO₃ (152.66 mg, 1.44 mmol). The mixture was stirred at 100° C. for 1 h. The reaction mixture was diluted with H₂O (10 mL) and extracted with EtOAc (10 mL×3). The combined organic layer was washed with brine (10 mL×3), dried over anhydrous Na₂SO₄, filtered and concentrated under reduced pressure to give E43 (244.2 mg, crude, 52% purity) as a brown solid.

### General Procedure for Synthesis of E44

1) To a solution of E15 (136.88 mg, 591.80 umol) in CH₃CN (2 mL) was added CDI (95.96 mg, 591.80 umol) and the reaction stirred at 20° C. for 10 min.

2) A solution of E43 (244 mg, 538.00 umol) in CH₃CN (3 mL) was added t-BuOK (66.41 mg, 591.80 umol), the reaction was heated to 80° C., then the first solution was added. The reaction mixture was adjusted to pH=4 with HCl (1 mol/L), and then poured into water (10 mL) and extracted with EtOAc (10 mL×3). The combined organic layers were washed with brine (10 mL×3), dried over Na₂SO₄, then concentrated under reduced pressure to give E44 (454 mg, crude, 74% purity) as a brown solid.

General Procedure for Synthesis of Compound 26 and compound 27

To a solution of E44 (454 mg, 680.86 umol) in EtOH (3 mL) was added 37% HCl (0.3 mL). The reaction mixture was stirred at 80° C. for 1 h. The crude product was purified by Prep-HPLC (column: Boston Prime C18 150*30 mm*5 um; mobile phase: [water(FA)-ACN]; B %: 14%-34%, 14 min) to afford compound 26 (31.9 mg, 96.64% purity) as a yellow solid and compound 27 (9.0 mg, 100% purity) as a yellow solid.

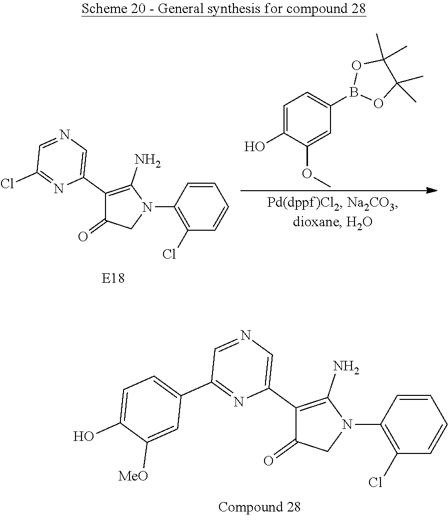 [SMILES: NC1=C(c2cncc(Cl)n2)C(=O)CN1c1ccccc1Cl ; COc1cc(B2OC(C)(C)C(C)(C)O2)ccc1O ; COc1cc(-c2cncc(C3=C(N)N(c4ccccc4Cl)CC3=O)n2)ccc1O]

### General Procedure for Synthesis of Compound 28

To a solution of 2-methoxy-4-(4,4,5,5-tetramethyl-1,3,2-dioxaborolan-2-yl)phenol (100 mg, 399.84 umol) and E18 (141.26 mg, 439.83 umol) in dioxane (3 mL) was added H₂O (0.6 mL), Pd(dppf)Cl₂ (29.26 mg, 39.98 umol) and Na₂CO₃ (127.14 mg, 1.20 mmol). The mixture was stirred at 100° C. for 1 h. The reaction mixture was diluted with H₂O (10 mL) and extracted with EtOAc (10 mL×3). The combined organic layer was washed with brine (10 mL×3), dried over anhydrous Na₂SO₄, filtered and concentrated under reduced pressure to give a residue. The crude product was purified by Prep-HPLC (column: Boston Prime C18 150*30 mm*5 um; mobile phase: [water(NH₃H₂O+NH₄HCO₃)-ACN]; B %: 26%-56%, 10 min) to give compound 28 (45.4 mg, 26.38% yield, 100% purity) as a yellow solid.

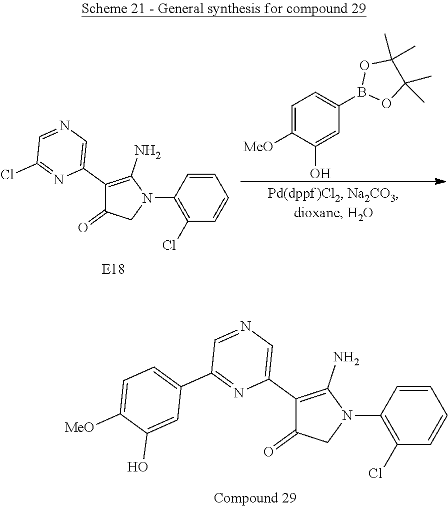 [SMILES: NC1=C(c2cncc(Cl)n2)C(=O)CN1c1ccccc1Cl ; COc1ccc(B2OC(C)(C)C(C)(C)O2)cc1O ; COc1ccc(-c2cncc(C3=C(N)N(c4ccccc4Cl)CC3=O)n2)cc1O]

### General Procedure for Synthesis of Compound 29

To a solution of 2-methoxy-5-(4,4,5,5-tetramethyl-1,3,2-dioxaborolan-2-yl)phenol (50 mg, 199.92 umol) and E18 (70.63 mg, 219.91 umol) in dioxane (1 mL) was added H₂O (0.2 mL), Pd(dppf)Cl₂ (14.63 mg, 19.99 umol) and Na₂CO₃ (63.57 mg, 599.77 umol). The mixture was stirred at 100° C. for 1 h. The reaction mixture was diluted with H₂O (10 mL) and extracted with EtOAc (10 mL×3). The combined organic layer was washed with brine (10 mL×3), dried over anhydrous Na₂SO₄, filtered and concentrated under reduced pressure to give a residue. The crude product was purified by Prep-HPLC (column: Boston Prime C18 150*30 mm*5 um; mobile phase: [water(NH₃H₂O+NH₄HCO₃)-ACN]; B %: 29%-59%, 10 min) to give compound 29 (17.2 mg, 21.04% yield, 100% purity) as a yellow solid.

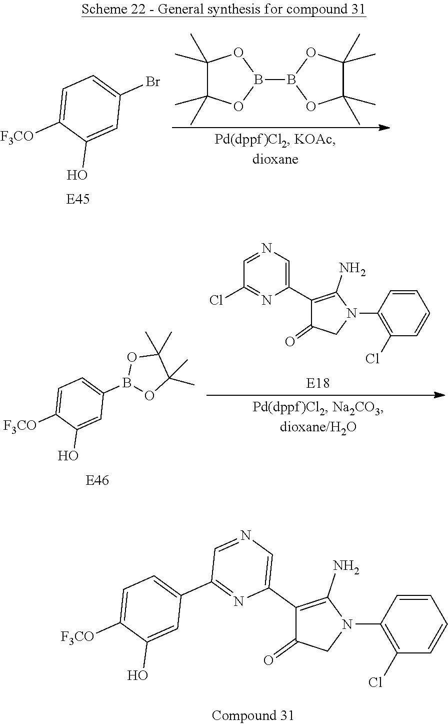

### General Procedure for Synthesis of Compound 31

To a solution of E45 (500 mg, 1.95 mmol) and 4,4,5,5-tetramethyl-2-(4,4,5,5-tetramethyl-1,3,2-dioxaborolan-2-yl)-1,3,2-dioxaborolane (741.05 mg, 2.92 mmol) in dioxane (5 mL) was added Pd(dppf)Cl₂ (142.35 mg, 0.19 mmol) and KOAc (381.87 mg, 3.89 mmol). The mixture was stirred at 100° C. for 1 hr. Take ⅓ of above reaction solution to another flask and then added E18 (187.44 mg, 0.58 mmol), Na₂CO₃ (144.34 mg, 1.36 mmol) and Pd(dppf)Cl₂ (42.71 mg, 0.06 mmol) and H₂O (1 mL) at 25° C. The mixture was stirred at 100° C. for 1 hr. The reaction mixture was diluted with H₂O (10 mL) and extracted with EtOAc (10 mL×3). The combined organic layers were washed with brine (10 mL×3), dried over anhydrous Na₂SO₄, filtered and concentrated under reduced pressure to give a residue. The residue was purified by prep-HPLC (column: Boston Prime C18 150*30 mm*5 um; mobile phase: [water(NH₃H₂O+NH₄HCO₃)-ACN]; B %: 38%-68%, 10 min) to give compound 31 (3.6 mg, 1.3% yield, 96.6% purity) as a white solid.

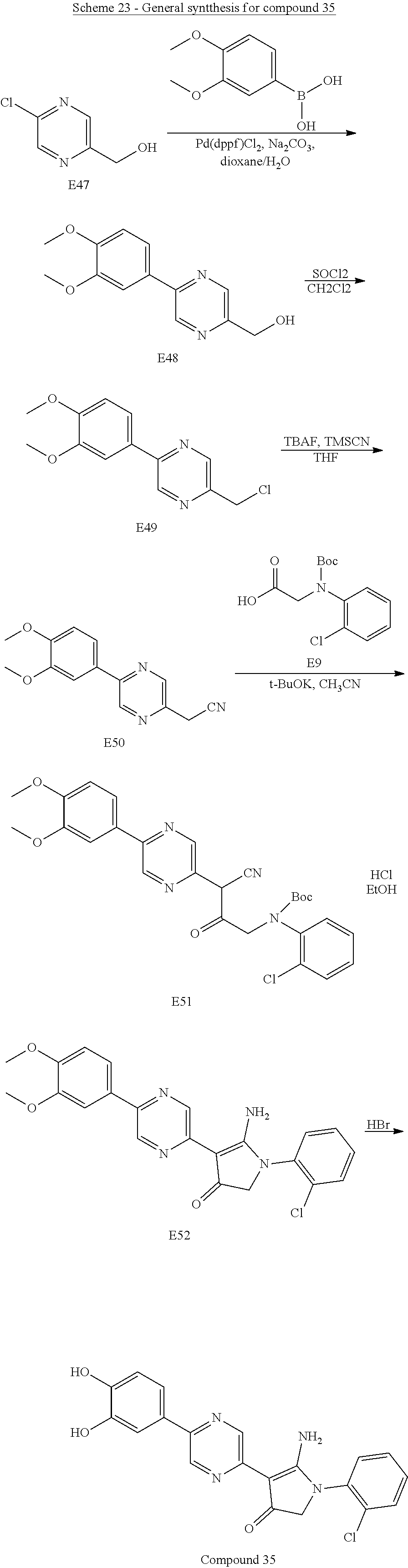

### General Procedure for Synthesis of E48

To a mixture of E47 (500 mg, 3.46 mmol) and (3,4-dimethoxyphenyl)boronic acid (692.38 mg, 3.80 mmol) in dioxane (5 mL) H₂O (1 mL) and was added Pd(dppf)Cl₂ (253.08 mg, 345.88 umol) and Na₂CO₃ (1.10 g, 10.38 mmol). The mixture was stirred at 100° C. for 1 h under N₂ atmosphere. The reaction mixture was diluted with H₂O (10 mL) and extracted with EtOAc (10 mL×3). The combined organic layer was washed with brine (10 mL×3), dried over anhydrous Na₂SO₄, filtered and concentrated under reduced pressure to give E48 (1.01 g, crude) as a brown solid.

### General Procedure for Synthesis of E49

To a solution of E48 (1.01 g, 4.10 mmol, crude) in CH₂Cl₂ (10 mL) was added SOCl₂ (595.04 uL, 8.20 mmol) at −10° C. The mixture was stirred at 0° C. for 1 h. The reaction mixture was concentrated under reduced pressure to give E49 (786 mg, 72% yield) as a yellow solid.

### General Procedure for Synthesis of E50

To a solution of E49 (786 mg, 2.97 mmol) and TMSCN (589.17 mg, 5.94 mmol) in THF (5 mL) was added TBAF (1 M, 5.94 mL), the reaction mixture was stirred at 25° C. for 16 h. The reaction mixture was concentrated under reduced pressure to remove solvent. The residue was purified by flash silica gel chromatography (PE:EtOAc=1:1) to give E50 (630 mg, 58% yield, 70% purity) as a yellow solid.

### General Procedure for Synthesis of E51

1) To a mixture of E9 (246.24 mg, 861.83 umol) in CH₃CN (2 mL) was added CDI (139.75 mg, 861.83 umol) and the reaction was stirred at 20° C. for 10 min.

2) To a solution of E50 (200 mg, 783.48 umol) in CH₃CN (3 mL) was added t-BuOK (96.71 mg, 861.83 umol), the reaction was heated to 80° C., then the first mixture solution was added. The reaction was stirred at 80° C. for 15 min. The reaction mixture was poured into water (10 mL) and adjusted to pH=4 with 1 M HCl, extracted with EtOAc (10 mL×3). The combined organic layers were washed with brine (10 mL×3), dried over Na₂SO₄, then concentrated under reduced pressure to give E51 (551 mg, crude, 60% purity) as a brown solid.

### General Procedure for Synthesis of E52

To a mixture of E51 (551 mg, 1.05 mmol) in EtOH (5 mL) was added concentrated HCl (0.5 mL). The reaction mixture was stirred at 80° C. for 1 h. The reaction mixture was poured into water (20 mL) and extracted with EtOAc (20 mL×3). The combined organic layers were washed with brine (20 mL×3), dried over Na₂SO₄, then concentrated under reduced pressure to give E52 (297 mg, 66% yield) as a yellow solid.

### General Procedure for Synthesis of Compound 35

A suspension of E52 (297 mg, 702.35 umol) in HBr (5 mL, 40% purity) was heated to 120° C. for 4 h. The crude product was purified by Prep-HPLC (column: Boston Prime C18 150*30 mm*5 um; mobile phase: [water (FA)-ACN]; B %: 20%-50%, 14 min) to give compound 35 (50 mg, 18% yield, 98% purity) as a yellow solid.

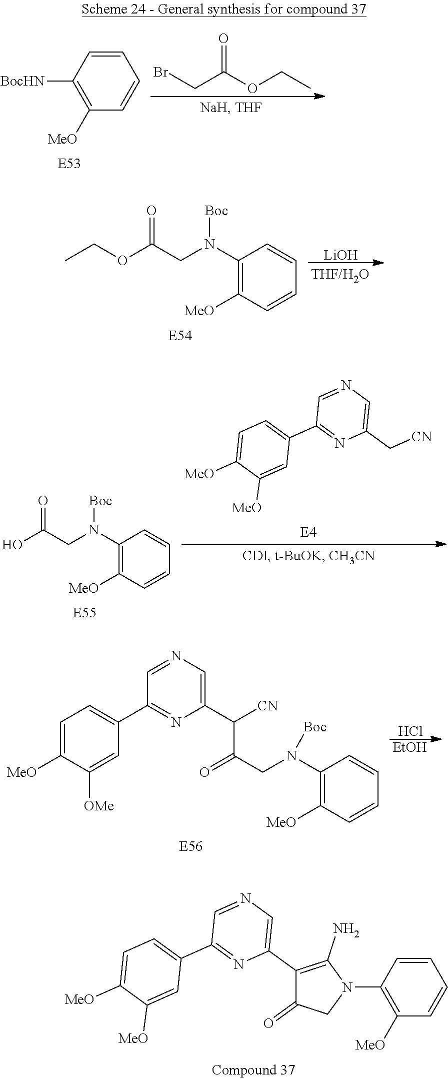

### General Procedure for Synthesis of E54

To a mixture of NaH (71.66 mg, 1.79 mmol, 60% purity) in THF (5 mL) was added E53 (400 mg, 1.79 mmol) and ethyl 2-bromoacetate (299.19 mg, 1.79 mmol). The mixture was stirred at 25° C. for 2 hr. The reaction mixture was quenched with H₂O (30 mL) and extracted with EtOAc (30 mL×3). The combined organic layers were dried over anhydrous Na₂SO₄, filtered and concentrated under reduced pressure to give a residue. Another batch from 100 mg of E53 was combined for purification. The residue was purified by column chromatography (SiO₂, Petroleum ether/Ethyl acetate=1/1) to give E54 (256 mg, crude) as a yellow oil.

### General Procedure for Synthesis of E55

To a solution of E54 (256 mg, 0.83 mmol, crude) in THF (2.5 mL) and H₂O (0.5 mL) was added LiOH·H₂O (173.63 mg, 4.14 mmol), the mixture was stirred at 60° C. for 12 hr. The reaction mixture was diluted with H₂O (10 mL) and extracted with EtOAc (20 mL). The aqueous phase was adjusted to pH=5 with 1 MHCI and extracted with EtOAc (10 mL×3), The combined organic layer was washed with brine (10 mL×3), dried over anhydrous Na₂SO₄, filtered and concentrated under reduced pressure to give E55 (147 mg, crude) as a yellow oil.

### General Procedure for Synthesis of E56

A: To a mixture of E55 (145.46 mg, 0.52 mmol) in CH₃CN (1 mL) was added CDI (83.85 mg, 0.52 mmol), the mixture was stirred at 25° C. for 3 min.

B: To a solution of E4 (120 mg, 0.47 mmol) in CH₃CN (1 mL) was added t-BuOK (52.75 mg, 0.47 mmol), the mixture stirred at 80° C. for 2 min, then the mixture from step A was added dropwise at 80° C. The resulting mixture was stirred at 80° C. for 15 min. The residue was adjusted with 1 M HCI to pH=4, and extracted with EtOAc (30 mL×3). The combined organic layer was washed with brine (30 mL×3), dried over anhydrous Na₂SO₄, filtered and concentrated to give E56 (220 mg, crude) as a yellow solid.

### General Procedure for Synthesis of Compound 37

To a solution of E56 (220 mg, 0.42 mmol) in EtOH (2 mL) was added concentrated HCl (0.2 mL). The mixture was stirred at 80° C. for 1 hr. The reaction mixture was concentrated under reduced pressure to remove solvent. The residue was purified by prep-HPLC (column: Boston Prime C18 150*30 mm*5 um; mobile phase: [water (NH₃H2O+NH₄HCO₃)-ACN]; B %: 36%-66%, 10 min) to give compound 37 (17.9 mg, 10% yield, 98% purity) as a yellow solid.

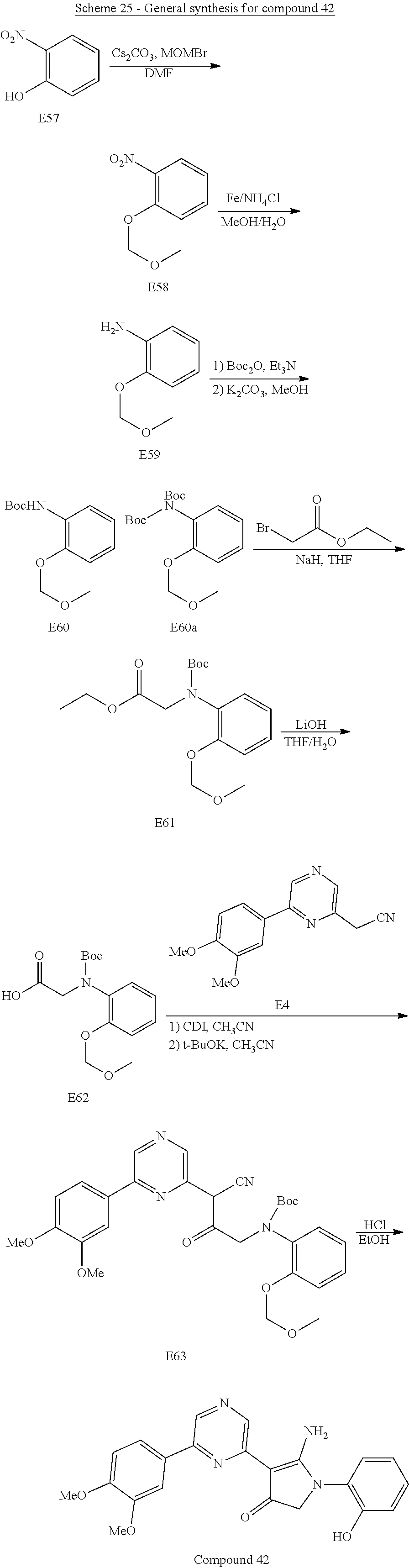

### General Procedure for Synthesis of E58

To a mixture of Cs₂CO₃ (23.42 g, 71.89 mmol) in DMF (20 mL) was added E57 (5 g, 35.94 mmol). The mixture was stirred at 0° C. for 10 min. Bromo(methoxy)methane (4.94 g, 39.54 mmol) was added to the mixture at 0° C. and stirred at 20° C. for 2 h. The mixture was quenched with saturated NH₄Cl (30 mL), diluted with water (30 mL) and extracted with EtOAc (30 mL×3). The combined organic layers were washed with brine (30 mL×3), dried over Na₂SO₄, concentrated under reduced pressure. he residue was purified by flash silica gel chromatography (PE:EtOAc=3:1) to give E58 (3.99 g, 60% yield) as a yellow oil.

### General Procedure for Synthesis of E59

To a mixture of E58 (1 g, 5.46 mmol) in MeOH (12 mL) and H₂O (4 mL) was added Fe (1.52 g, 27.30 mmol) and NH₄Cl (2.92 g, 54.60 mmol). The mixture was stirred at 60° C. for 1 h. The reaction mixture was filtered and washed with H₂O (20 mL). The filtrate was extracted with EtOAc (30 mL×3). The combined organic layers were washed with brine (30 mL×3), and then dried over anhydrous Na₂SO₄, filtered and concentrated in vacuum to give E59 (712 mg, 85% yield) as a brown oil.

### General Procedure for Synthesis of E60

1) To a solution of E59 (510 mg, 3.33 mmol) in Boc₂O (2.18 g, 9.99 mmol) was added K₂CO₃ (920.30 mg, 6.66 mmol), the mixture was stirred at 100° C. for 2 h.

The mixture was quenched with saturated NH₄Cl (20 mL), then poured into water (20 mL) and extracted with EtOAc (20 mL×3). The combined organic layers were washed with brine (20 mL×3), dried over Na₂SO₄, concentrated under reduced pressure. The residue was purified by flash silica gel chromatography (PE:EtOAc=5:1) to give E60 (193 mg, 93% purity) as a colorless oil and E60a (435 mg, 79% purity) as a yellow oil. 2) To a solution of E60a (435 mg, 1.23 mmol) in MeOH (10 mL) was added K₂CO₃ (510.34 mg, 3.69 mmol) and the reaction was stirred at 65° C. for 10 h. The mixture was quenched with saturated NH₄Cl (20 mL), diluted with water (20 mL) and extracted with EtOAc (20 mL×3). The combined organic layers were washed with brine (20 mL×3), dried over Na₂SO₄, concentrated under reduced pressure to give E60 (208 mg, 66% yield) as a brown oil.

### General Procedure for Synthesis of E61

To a suspension of NaH (189.49 mg, 4.74 mmol, 60% purity) in THF (10 mL) was added E60 (400 mg, 1.58 mmol) at 0° C., then ethyl 2-bromoacetate (290.10 mg, 1.74 mmol) was added. The mixture was stirred at 20° C. for 2 h. The mixture was quenched with saturated NH₄Cl (20 mL), diluted with water (20 mL) and extracted with EtOAc (20 mL×3). The combined organic layers were washed with brine (20 mL×3), dried over Na₂SO₄, concentrated under reduced pressure to give E61 (465 mg, 69% yield, 80% purity) as a colorless oil

### General Procedure for Synthesis of E62

To a solution of E61 (465 mg, 1.37 mmol) in THF (8 mL) and H₂O (6 mL) was added LiOH·H₂O (114.98 mg, 2.74 mmol). The mixture was stirred at 20° C. for 10 h. The reaction mixture was poured into water (10 mL) and adjusted to pH=5 with 1 M HCl, then extracted with EtOAc (20 mL×3). The combined organic layers were washed with brine (20 mL×3), dried over Na₂SO₄, concentrated under reduced pressure to give E62 (283 mg, 66% yield) as a yellow oil.

### General Procedure for Synthesis of E63

1) To a mixture of E62 (90 mg, 289.08 umol) in CH₃CN (2 mL) was added CDI (46.87 mg, 289.08 umol) and the reaction was stirred at 20° C. for 10 min.

2) To a mixture of E4 (67.09 mg, 262.80 umol) in CH₃CN (2 mL) was added t-BuOK (32.44 mg, 289.08 umol), the reaction was heated to 80° C., then the first reaction mixture was added and the reaction was stirred at 80° C. for another 15 min. The reaction mixture was poured into water (10 mL) and adjusted to pH=4 with 1 M HCl, extracted with EtOAc (10 mL×3). The combined organic layers were washed with brine (10 mL×3), dried over Na₂SO₄, then concentrated under reduced pressure to give E63 (183 mg, 66% yield, 52% purity) as a brown solid.

### General Procedure for Synthesis of Compound 42

To a mixture of E63 (180 mg, 328.12 umol) in EtOH (5 mL) was added concentrated HCl (0.5 mL). The reaction mixture was stirred at 80° C. for 1 h. The crude product was purified by Prep-HPLC (column: Boston Green ODS 150*30 mm*5 um; mobile phase: [water (HCl)-ACN]; B %: 28%-43%, 10 min) to give compound 42 (36.7 mg, 25. % yield, 100% purity, HCl salt) as a brick red solid.

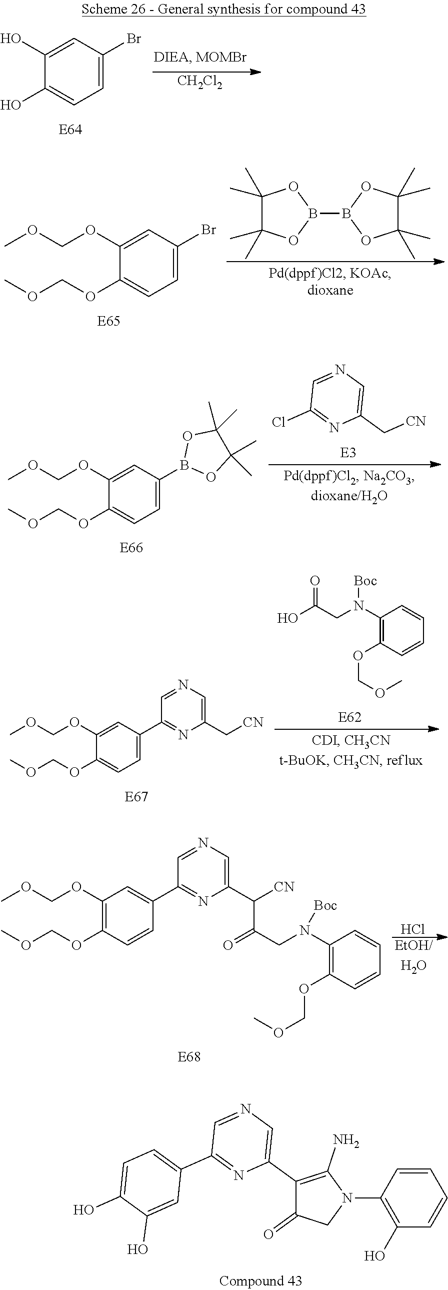

### General Procedure for Synthesis of E65

To a mixture of E64 (3 g, 15.87 mmol) in THF (10 mL) was added DIEA (8.21 g, 63.49 mmol, 11.1 mL) at 0° C. After stirred at 0° C. for 10 min, bromo(methoxy)methane (4.36 g, 34.92 mmol) was added to the mixture at 0° C. and stirred at 20° C. for 10 h. The mixture was quenched with saturated NH₄Cl (20 mL), diluted with water (20 mL) and extracted with EtOAc (20 mL×3). The combined organic layers were washed with brine (20 mL×3), dried over Na₂SO₄, concentrated under reduced pressure. The residue was purified by flash silica gel chromatography (PE:EtOAc=3:1) to give E65 (3.67 g, 83% yield) as a yellow oil.

### General Procedure for Synthesis of E66

To a mixture of E65 (1.8 g, 6.50 mmol) and 4,4,5,5-tetramethyl-2-(4,4,5,5-tetramethyl-1,3,2-dioxaborolan-2-yl)-1,3,2-dioxaborolane (2.47 g, 9.74 mmol) in dioxane (10 mL) was added Pd(dppf)Cl₂ (475.29 mg, 649.56 umol) and KOAc (1.91 g, 19.49 mmol). The mixture was stirred at 100° C. for 1 h under N₂ atmosphere. The mixture was poured into water (20 mL) and extracted with EtOAc (20 mL×3). The combined organic layers were washed with brine (20 mL×3), dried over Na₂SO₄, concentrated under reduced pressure. The residue was purified by flash silica gel chromatography (PE:EtOAc=3:1) to give E66 (1.68 g, 79% yield) as a yellow oil.

### General Procedure for Synthesis of E67

To a mixture of E66 (500 mg, 1.54 mmol) and E3 (260.55 mg, 1.70 mmol) in dioxane (10 mL) and H₂O (2 mL) was added Pd(dppf)Cl₂ (112.86 mg, 154.24 umol) and Na₂CO₃ (490.43 mg, 4.63 mmol). The mixture was stirred at 100° C. for 1 h. The reaction mixture was diluted with H₂O (10 mL) and extracted with EtOAc (10 mL×3). The combined organic layer was washed with brine (10 mL×3), dried over anhydrous Na₂SO₄, filtered and concentrated under reduced pressure to give a residue. The residue was purified by flash silica gel chromatography (PE EtOAc=3:1) to give E67 (378 mg, 71% yield, 92% purity) as a brown oil.

### General Procedure for Synthesis of E68

1) To a mixture of E62 (80.78 mg, 259.47 umol) in CH₃CN (2 mL) was added CDI (46.28 mg, 285.42 umol) and the reaction was stirred at 20° C. for 10 min.

2) To a mixture of E67 (90 mg, 285.42 umol) in CH₃CN (2 mL) was added t-BuOK (32.03 mg, 285.42 umol), the reaction was heated to 80° C., then the first reaction mixture was added. The reaction stirred at 80° C. for 15 min. The reaction mixture was poured into water (10 mL) and adjusted to pH=4 with 1 M HCl, extracted with EtOAc (10 mL×3). The combined organic layers were washed with brine (10 mL×3), dried over Na₂SO₄, then concentrated under reduced pressure to give E68 (190 mg, 75% yield, 63% purity) as a brown solid.

### General Procedure for Synthesis of Compound 43

To a mixture of E68 (190 mg, 312.17 umol) in EtOH (5 mL) was added concentrated HCl (0.5 mL). The reaction mixture was stirred at 80° C. for 1 h. The reaction mixture was concentrated under reduced pressure. The crude product was purified by Prep-HPLC (column: Boston Green ODS 150*30 mm*5 um; mobile phase: [water (FA)-ACN]; B %: 16%-36%, 10 min) to give compound 43 (25.5 mg, 19% yield, 96% purity, HCl salt) as a brick red solid.

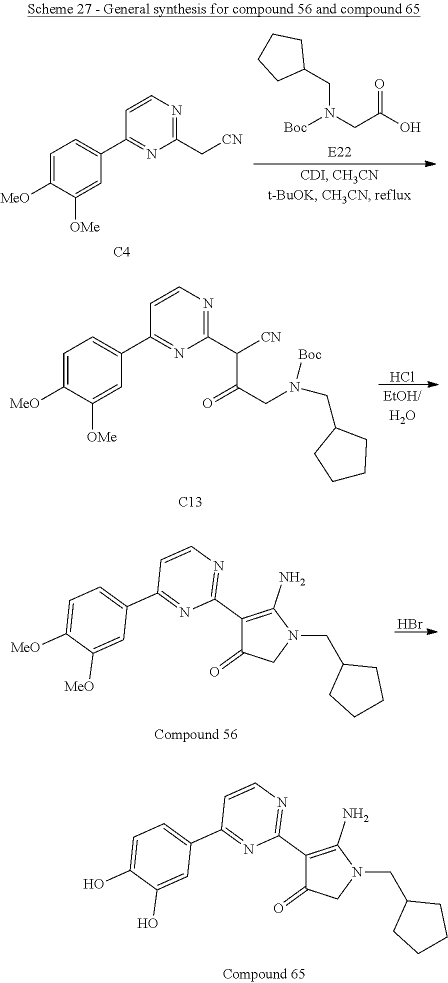

### General Procedure for Synthesis of C13

1) To a mixture of E22 (76.51 mg, 0.3 mmol) in CH₃CN (1 mL) was added CDI (48.21 mg, 0.3 mmol). The mixture was stirred at 25° C. for 3 min.

2) To a mixture of C4 (69 mg, 0.27 mmol) in CH₃CN (1 mL) was added t-BuOK (30.33 mg, 0.27 mmol). The mixture stirred at 80° C. for 2 min. The step one reaction mixture was added dropwise at 80° C. The resulting mixture was stirred at 80° C. for 15 min. The residue was adjusted with 1 M HCI to pH=4, extracted with EtOAc (10 mL×3). The combined organic layer was washed with brine (10 mL×3), dried over anhydrous Na₂SO₄, filtered and concentrated to give C13 (173 mg, crude) as an orange solid.

### General Procedure for Synthesis of Compound 56

To a solution of C13 (173 mg, 0.35 mmol) in EtOH (2 mL) was added concentrated HCl (0.5 mL). The mixture was stirred at 80° C. for 1 hr. The reaction mixture was concentrated under reduced pressure to remove solvent. The crude product was triturated with EtOAc at 25° C. for 30 min to give compound 56 (120 mg, crude) as a brown solid. The 60 mg of crude product was used into the next step without further purification. The rest 60 mg crude product was purified by prep-HPLC (column: Boston Green ODS 150*30 mm*5 um; mobile phase: [water (FA)-ACN]; B %: 12%-42%, 12 min) to give compound 56 (18.4 mg, 13% yield, 99% purity) as a yellow solid.

### General Procedure for Synthesis of Compound 65

A suspension of compound 56 (60 mg, 0.15 mmol) in HBr (2 mL, 40% purity) was stirred at 120° C. for 12 hr. The reaction mixture was concentrated under reduced pressure to remove solvent. The residue was purified by prep-HPLC (column: Boston Green ODS 150*30 mm*5 um; mobile phase: [water (FA)-ACN]; B %: 15%-45%, 12 min) to give compound 65 (15.8 mg, 28% yield, 98% purity) as a yellow solid.

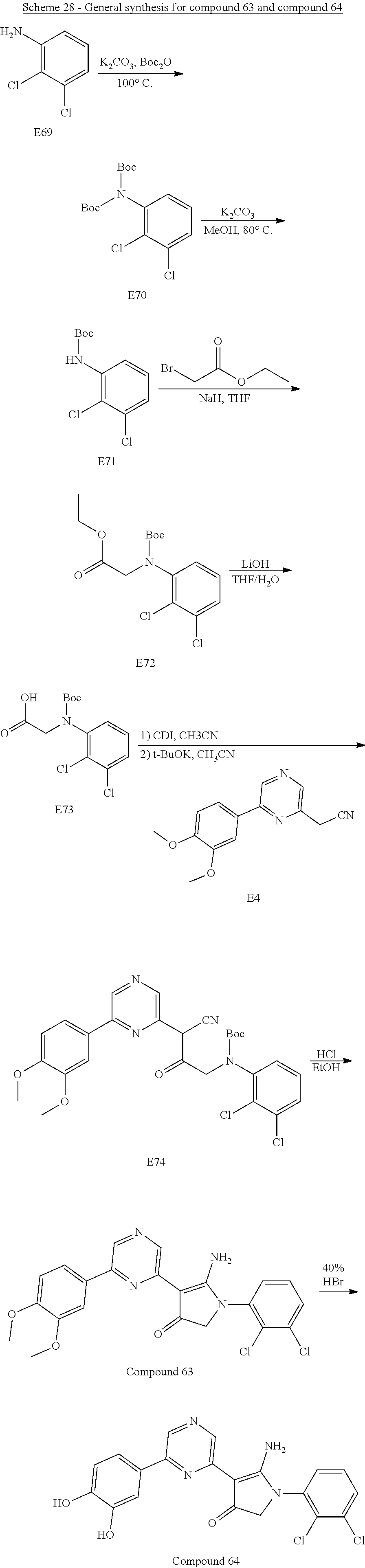

### General Procedure for Synthesis of E70

To a mixture of E69 (1 g, 6.17 mmol, 729.93 uL) in Boc₂O (4.04 g, 18.52 mmol, 4.25 mL) was added K₂CO₃ (1.71 g, 12.34 mmol). The mixture was stirred at 100° C. for 16 hr. The mixture was diluted with water (30 mL), extracted with EtOAc (30 mL×3). The combined organic layer was washed with brine (30 mL×3), dried over anhydrous Na₂SO₄, filtered and concentrated under reduced pressure. The residue was triturated with PE (30 mL) at 25° C. for 30 min to give E70 (1.71 g, 76% yield) as a white solid.

### General Procedure for Synthesis of E71

To a mixture of E70 (1.70 g, 4.69 mmol) in MeOH (20 mL) was added K₂CO₃ (1.30 g, 9.39 mmol). The mixture was stirred at 80° C. for 12 hr. The reaction mixture was diluted with H₂O (30 mL) and extracted with EtOAc (30 mL×3). The combined organic layers were washed with brine (30 mL×3), dried over anhydrous Na₂SO₄, filtered and concentrated under reduced pressure. The residue was purified by column chromatography (SiO2, Petroleum ether/Ethyl acetate=5/1) to give E71 (590 mg, 48% yield) as a yellow oil.

### General Procedure for Synthesis of E72

To a mixture of NaH (90.02 mg, 2.25 mmol, 60% purity) in THF (6 mL) was added E71 (590 mg, 2.25 mmol) and ethyl 2-bromoacetate (375.88 mg, 2.25 mmol, 248.93 uL). The mixture was stirred at 25° C. for 2 hr. The reaction mixture was diluted with H₂O (20 mL) and extracted with EtOAc (20 mL×3). The combined organic layers were washed with brine (20 mL×3), dried over anhydrous Na₂SO₄, filtered and concentrated under reduced pressure to give E72 (914 mg, crude) as a yellow oil.

### General Procedure for Synthesis of E73

To a mixture of E72 (914 mg, 2.62 mmol) in THF (10 mL) was added LiOH·H₂O (220.29 mg, 5.25 mmol) and H₂O (2 mL). The mixture was stirred at 25° C. for 12 hr. The reaction mixture was diluted with H₂O (10 mL) and extracted with EtOAc (20 mL). The aqueous phase was adjusted to pH=5 with 1 M HCI and extracted with EtOAc (30 mL×3). The combined organic layer was washed with brine (30 mL×3), dried over anhydrous Na₂SO₄, filtered and concentrated under reduced pressure to give E73 (643 mg, crude) as a yellow oil.

### General Procedure for Synthesis of E74

1) To a mixture of E73 (206.95 mg, 0.65 mmol) in CH₃CN (2 mL) was added CDI (104.81 mg, 0.65 mmol), the mixture was stirred at 25° C. for 3 min.

2) To a mixture of E4 (150 mg, 0.59 mmol) in CH₃CN (2 mL) was added t-BuOK (65.94 mg, 0.59 mmol), the mixture was stirred at 80° C. for 2 min. The step one mixture was added dropwise at 80° C., The resulting mixture was stirred at 80° C. for 15 min. The residue was adjusted with 1 M HCI to pH=4, and extracted with EtOAc (20 mL×3). The combined organic layer was washed with brine (20 mL×3), dried over anhydrous Na₂SO₄, filtered and concentrated to give E74 (307 mg, crude) as an orange solid.

### General Procedure for Synthesis of Compound 63

To a mixture of E74 (307 mg, 0.55 mmol) in EtOH (3 mL) was added concentrated HCl (0.6 mL). The mixture was stirred at 80° C. for 1 hr. The reaction mixture was concentrated under reduced pressure to remove solvent. The crude product was triturated with EtOAc at 25° C. for 30 min to give compound 63 (129 mg, crude) as a brown solid. Part 1: The 70 mg of crude product was used into the next step without further purification. Part 2: The rest 59 mg of crude product was purified by prep-HPLC (column: Boston Green ODS 150*30 mm*5 um; mobile phase: [water (FA)-ACN]; B %: 37%-67%, 12 min) to give compound 63 (19.6 mg, 8% yield, 99% purity) as an orange solid.

### General Procedure for Synthesis of Compound 64

A mixture of compound 63 (70 mg, 0.15 mmol) in HBr (2 mL, 40% purity) was stirred at 120° C. for 12 hr. The reaction mixture was concentrated under reduced pressure to remove solvent. The residue was purified by prep-HPLC (column: Boston Green ODS 150*30 mm*5 um; mobile phase: [water (FA)-ACN]; B %: 27%-57%, 12 min) to give compound 64 (23 mg, 34% yield, 98% purity) as a yellow solid.

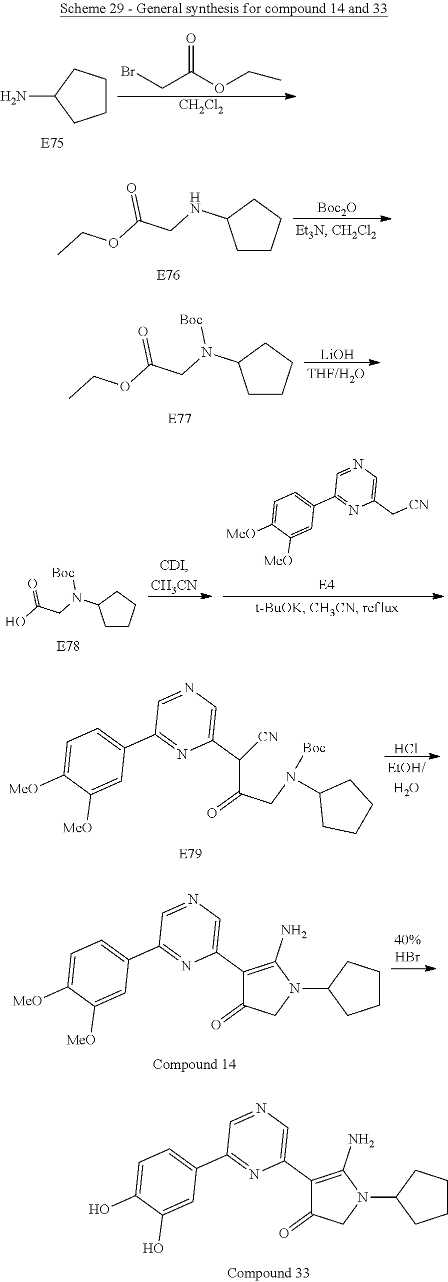

### General Procedure for Synthesis of E76

To a solution of E75 (5.10 g, 59.88 mmol) in CH₂Cl₂ (50 mL) was added ethyl 2-bromoacetate (5 g, 29.94 mmol). The mixture was stirred at 25° C. for 12 hr. The mixture was concentrated in vacuum. The residue was purified by column chromatography (SiO₂, Petroleum ether/Ethyl acetate=1/1) to afford E76 (3.33 g, 65% yield) was obtained as a yellow oil.

### General Procedure for Synthesis of E77

To a solution of E76 (3.33 g, 19.45 mmol) in CH₂Cl₂ (30 mL) was added Boc₂O (4.24 g, 19.45 mmol) and Et₃N (2.95 g, 29.17 mmol). The mixture was stirred at 25° C. for 12 hr. The mixture was concentrated in vacuum. The residue was purified by column chromatography (SiO2, petroleum ether/ethyl acetate=1/1 to 0/1) to afford E77 (1 g, 19% yield)

### General Procedure for Synthesis of E78

To a solution of E77 (500 mg, 1.84 mmol) in THF (6 mL) was added a solution of LiOH·H₂O (115.98 mg, 2.76 mmol) in water (2 mL), the mixture was stirred at 60° C. for 1 hr. Water (10 mL) was added to the mixture, and the mixture was treated with 1N HCl to adjust pH to 2, then extracted with EtOAc (20 mL×2). The combined organic layer was washed with brine (10 mL), dried over Na₂SO₄, filtered and concentrated to afford E78 (440 mg, 98% yield) as a yellow solid.

### General Procedure for Synthesis of E79

To a solution of E78 (104.84 mg, 430.91 umol) in MeCN (2 mL) was added CDI (69.87 mg, 430.91 umol) at 20° C., and stirred for 5 min. To second solution of E4 (100 mg, 391.74 umol) in MeCN (2 mL) was added t-BuOK (43.96 mg, 391.74 umol) at 20° C., and then heated to 80° C., and the first solution was added. The reaction mixture was stirred at 80° C. for 0.5 hr. The reaction was diluted with water (10 mL), acidified by 1N HCl to pH=5 and extracted with EtOAc (20 mL×2). The organic layer was washed with brine (20 mL), dried over Na₂SO₄, filtered and concentrated to afford E79 (200 mg, 62% yield, 58.5% purity) as a brown solid, which was used directly without purification.

### General Procedure for Synthesis of Compound 14

To a solution of E79 (200 mg, 243.47 umol, 58.5% purity) in EtOH (5 mL) was added HCl (0.5 mL, 37%). The reaction was heated to 80° C. for 1 hr. The reaction mixture was concentrated. The residue was purified by prep-HPLC (column: Boston Green ODS 150*30 mm*5 um; mobile phase: [water(HCl)-ACN]; B %: 30%-60%, 10 min) to afford compound 14 (60 mg, 63% yield, 100% purity) as an yellow solid.

### General Procedure for Synthesis of Compound 33

A mixture of compound 14 (20 mg, 0.05 mmol) in HBr (4 mL, 40% purity) was stirred at 125° C. for 12 hr. The reaction mixture was concentrated under reduced pressure to remove solvent. The residue was purified by prep-HPLC (column: Boston Green ODS 150*30 mm*5 um; mobile phase: [water(FA)-ACN]; B %: 8%-38%, 12 min) to give compound 33 (5.7 mg, 31% yield, 100% purity) as a yellow solid

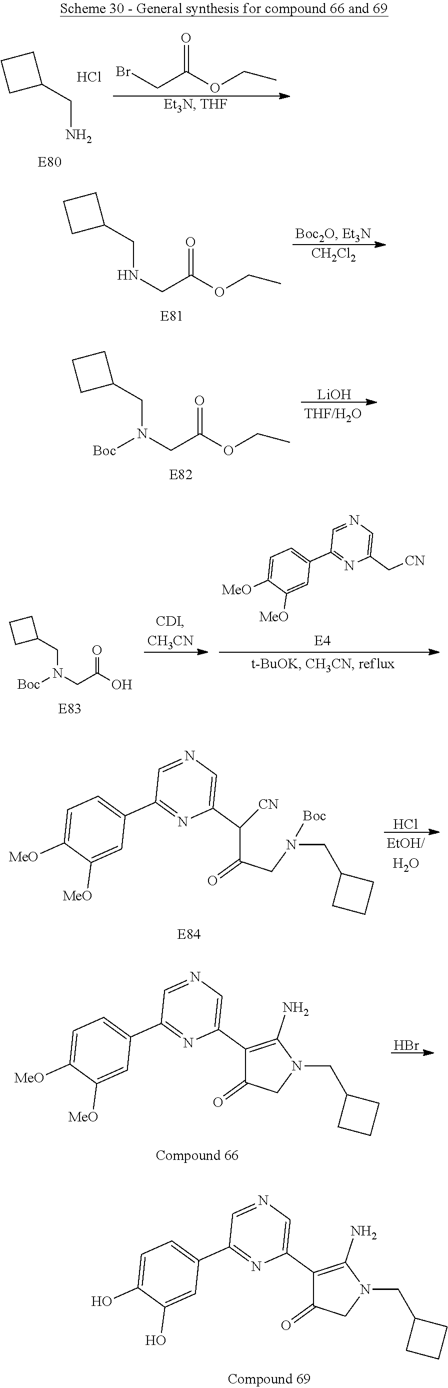

### General Procedure for Synthesis of E81

To a mixture of E80 (1 g, 8.22 mmol) in THF (10 mL) was added Et₃N (2.08 g, 20.56 mmol, 2.86 mL) and ethyl 2-bromoacetate (1.51 g, 9.05 mmol, 1.0 mL). The mixture was stirred at 25° C. for 12 h. The mixture was quenched with saturated NH₄Cl (30 mL), diluted with water (30 mL) and extracted with EtOAc (30 mL×3). The combined organic layers were washed with brine (30 mL×3), dried over Na₂SO₄, concentrated under reduced pressure to give E81 (654 mg, 26% yield, 56% purity) as a yellow oil.

### General Procedure for Synthesis of E82

To a mixture of E81 (630 mg, 3.68 mmol) in CH₂Cl₂ (10 mL) was added Boc₂O (1.61 g, 7.36 mmol) and Et₃N (558.43 mg, 5.52 mmol, 768 uL) at 0° C. The reaction mixture was stirred at 20° C. for 12 h. The reaction mixture was poured into water (30 mL) and extracted with EtOAc (30 mL×3). The combined organic layers were washed with brine (30 mL×3), dried over Na₂SO₄, filtered and concentrated under reduced pressure to give E82 (1.6 g, crude) as a brown oil.

### General Procedure for Synthesis of E83

To a mixture of E82 (1.6 g, 5.90 mmol) in THF (8 mL) and H₂O (6 mL) was added LiOH·H₂O (494.83 mg, 11.79 mmol). The mixture was stirred at 20° C. for 10 h. The reaction mixture was poured into water (10 mL) and adjusted to pH=5 with 1 M HCl, extracted with EtOAc (20 mL×3), the combined organic layers were washed with brine (20 mL×3), dried over Na₂SO₄, then concentrated under reduced pressure to give E83 (829 mg, 57% yield) as a yellow oil.

### General Procedure for Synthesis of E84

1) To a mixture of E83 (300 mg, 1.23 mmol) in CH₃CN (3 mL) was added CDI (219.93 mg, 1.36 mmol) and the reaction stirred at 20° C. for 10 min.

2) To a mixture of E4 (314.76 mg, 1.23 umol) in CH₃CN (3 mL) was added t-BuOK (152.20 mg, 1.36 umol). The reaction was heated to 80° C., then the step one reaction mixture was added and the resulting mixture reaction was stirred at 80° C. for 15 min. The reaction mixture was poured into water (10 mL) and adjusted to pH=4 with 1 M HCl, extracted with EtOAc (20 mL×3). The combined organic layers were washed with brine (20 mL×3), dried over Na₂SO₄, then concentrated under reduced pressure to give E84 (534 mg, 63% yield, 71% purity) as a yellow solid.

### General Procedure for Synthesis of Compound 66

To a mixture of E84 (534 mg, 1.11 mmol) in EtOH (5 mL) was added concentrated HCl (0.5 mL). The reaction mixture was stirred at 80° C. for 1 h. The reaction mixture was concentrated under reduced pressure. The residue was triturated with Ethyl acetate (5 mL) to give compound 66 (598 mg, crude) as a brown solid. 100 mg of crude product was purified by Prep-HPLC (column: Boston Green ODS 150*30 mm*5 um; mobile phase: [water (FA)-ACN]; B %: 26%-56%, 14 min) to give compound 66 (24.6 mg, 100% purity) as a yellow solid.

### General Procedure for Synthesis of Compound 69

A suspension of compound 66 (498 mg, 1.31 mmol) in HBr (5 mL, 40% purity) was stirred at 120° C. for 4 h. The reaction mixture was concentrated under reduced pressure. The crude product was purified by Prep-HPLC (column: Boston Green ODS 150*30 mm*5 um; mobile phase: [water (FA)-ACN]; B %: 16%-46%, 12 min) to give compound 69 (58.9 mg, 12% yield, 100% purity) as a gray solid.

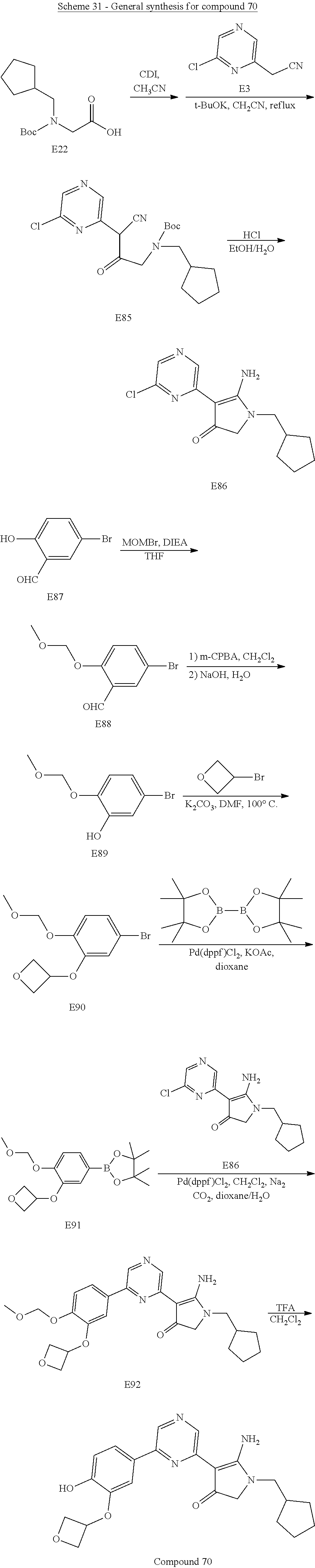

### General Procedure for Synthesis of E85

1) To a mixture of E22 (1.66 g, 6.45 mmol) in CH₃CN (10 mL) was added CDI (1.05 g, 6.45 mmol), the mixture was stirred at 25° C. for 3 min.

2) To a mixture of E3 (900 mg, 5.86 mmol) in CH₃CN (5 mL) was added t-BuOK (657.61 mg, 5.86 mmol), the mixture was stirred at 80° C. for 2 min. The step one reaction mixture was added dropwise at 80° C. The resulting mixture was stirred at 80° C. for 15 min. The residue was adjusted with 1 M HCI to pH=4, and extracted with EtOAc (20 mL×3). The combined organic layer was washed with brine (20 mL×3), dried over anhydrous Na₂SO₄, filtered and concentrated to give E85 (2.33 g, crude) as a brown solid.

### General Procedure for Synthesis of E86

To a mixture of E85 (2.33 g, 5.93 mmol) in EtOH (7.5 mL) was added concentrated HCl (1.5 mL). The mixture was stirred at 80° C. for 1 h. The reaction mixture was concentrated under reduced pressure to remove solvent. The crude product was triturated with EtOAc at 25° C. for 30 min to give E86 (1.3 g, 65% yield, 87% purity) as a yellow solid.

### General Procedure for Synthesis of E88

To a mixture of DIEA (12.86 g, 99.49 mmol) in THF (30 mL) was added E87 (5 g, 24.87 mmol) and stirred at 0° C. for 10 min. Then bromo(methoxy)methane (3.73 g, 29.85 mmol) was added to the mixture at 0° C. The mixture was stirred at 20° C. for 10 h. The mixture was quenched with saturated NH₄Cl (20 mL), then poured into water (50 mL) and extracted with EtOAc (50 mL×3). The combined organic layers were washed with brine (50 mL×3), dried over Na₂SO₄, concentrated under reduced pressure. The residue was purified by flash silica gel chromatography (PE:EtOAc=3:1) to give E88 (6.9 g, 84% yield, 75% purity) as a yellow oil.

### General Procedure for Synthesis of E89

To a mixture of E88 (2 g, 8.16 mmol) in CH₂Cl₂ (20 mL) was added m-CPBA (1.99 g, 9.79 mmol, 85% purity). The reaction was stirred at 20° C. for 16 h. NaOH (20 mL, 10% purity) was added to the mixture and stirred at 20° C. for 0.5 h. The reaction mixture was poured into water (20 mL) and adjusted to pH=4 with 1 M HCl, extracted with EtOAc (40 mL×3). The combined organic layers were washed with saturated NaHCO₃ (40 mL×3), dried over Na₂SO₄, concentrated under reduced pressure. The residue was purified by flash silica gel chromatography (PE:EtOAc=5:1) to give E89 (1.14 g, 60% yield) as a yellow oil.

### General Procedure for Synthesis of E90

To a mixture of E89 (500 mg, 2.15 mmol) in DMF (6 mL) was added 3-bromooxetane (1.47 g, 10.73 mmol) and K₂CO₃ (444.77 mg, 3.22 mmol). The mixture was stirred at 100° C. for 5 h. The reaction mixture was diluted with H₂O (20 mL) and extracted with EtOAc (20 mL×3). The combined organic layers were washed with brine (20 mL×3), dried over anhydrous Na₂SO₄, filtered and concentrated under reduced pressure. The residue was purified by column chromatography (SiO₂, Petroleum ether/Ethyl acetate=5/1) to give E90 (365 mg, 59% yield) as a yellow oil.

### General Procedure for Synthesis of E91

To a mixture of E90 (365 mg, 1.26 mmol) in dioxane (4 mL) was added 4,4,5,5-tetramethyl-2-(4,4,5,5-tetramethyl-1,3,2-dioxaborolan-2-yl)-1,3,2-dioxaborolane (480.87 mg, 1.89 mmol), Pd(dppf)Cl2 (92.37 mg, 0.13 mmol) and KOAc (247.79 mg, 2.52 mmol). The mixture was stirred at 100° C. for 2 h under N₂ atmosphere The reaction mixture was diluted with H₂O (20 mL) and extracted with EtOAc (20 mL×3). The combined organic layers were washed with brine (20 mL×3), dried over anhydrous Na₂SO₄, filtered and concentrated under reduced pressure. The residue was purified by column chromatography (SiO₂, Petroleum ether/Ethyl acetate=3/1) to give E91 (388 mg, crude) as a yellow oil.

### General Procedure for Synthesis of E92

To mixture of E86 (76 mg, 0.26 mmol) and E91 (100 mg, 0.30 mmol) in dioxane (5 mL) was added Pd(dppf)Cl₂·CH₂Cl₂ (21.20 mg, 0.02 mmol) and Na₂CO₃ (55.03 mg, 0.52 mmol) and H₂O (0.1 mL). The mixture was stirred at 100° C. for 3 h under N₂ atmosphere. The mixture was diluted with ethyl acetate (15 mL) and filtered. The organic layer was washed with water (10 mL×2), dried over Na₂SO₄, filtered and concentrated to give crude E92 (150 mg, 70% purity) as a brown gum.

### General Procedure for Synthesis of Compound 70

To a mixture of E92 (130 mg, 0.28 mmol) in CH₂Cl₂ (0.6 mL) was added TFA (2 mL). The mixture was stirred at 25° C. for 1 h. The reaction mixture was concentrated under reduced. The residue was adjusted with sat. NaHCO₃ to pH=7-8, and then extracted with DCM (10 mL×3). The combined organic layer was washed with brine (10 mL×3), dried over anhydrous Na₂SO₄, filtered and concentrated under reduced pressure. The residue was purified by prep-HPLC (column: C18-1 150*30 mm*5 um; mobile phase: [water (NH₄HCO₃)-ACN]; B %: 27%-47%, 13 min) to give compound 70 (6.1 mg, 5% yield, 97% purity) as an off-white solid.

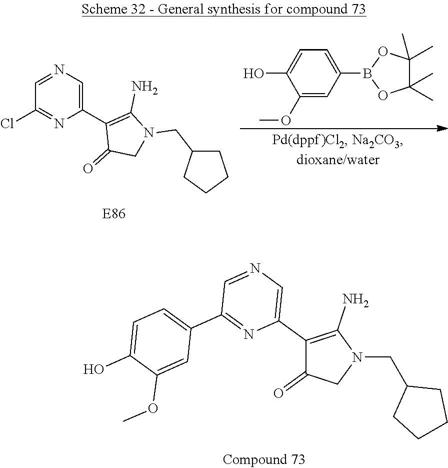 [SMILES: NC1=C(c2cncc(Cl)n2)C(=O)CN1CC1CCCC1 ; COc1cc(B2OC(C)(C)C(C)(C)O2)ccc1O ; COc1cc(-c2cncc(C3=C(N)N(CC4CCCC4)CC3=O)n2)ccc1O]

### General Procedure for Synthesis of Compound 73

To a mixture of E86 (60 mg, 0.2 mmol) in dioxane (1 mL) and H₂O (0.2 mL) was added 2-methoxy-4-(4,4,5,5-tetramethyl-1,3,2-dioxaborolan-2-yl) phenol (51.26 mg, 0.2 mmol), Pd(dppf)Cl₂ (15.00 mg, 0.02 mmol) and Na₂CO₃ (65.17 mg, 0.6 mmol). The mixture was stirred at 100° C. for 2 hr. The reaction mixture was concentrated under reduced pressure to remove solvent. The residue was purified by prep-HPLC (column: Boston Green ODS 150*30 mm*5 um; mobile phase: [water (FA)-ACN]; B %: 22%-52%, 12 min) to give compound 73 (0.9 mg, 1% yield, 100% purity) as a yellow solid.

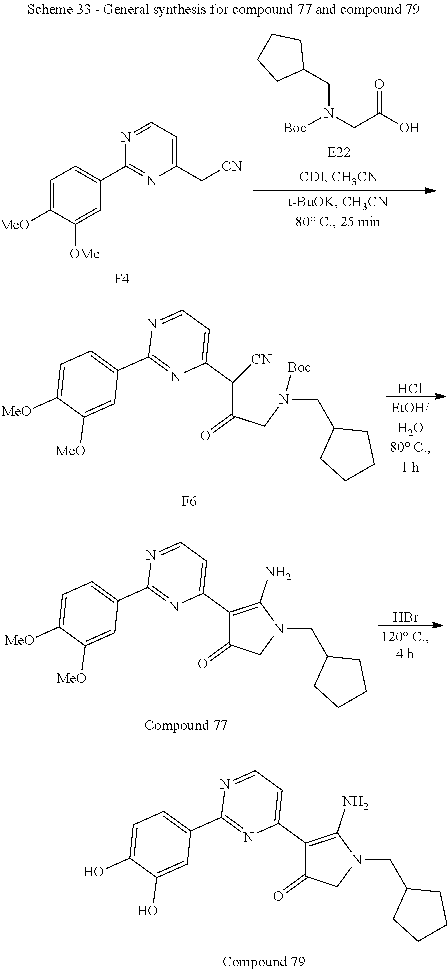

### General Procedure for Synthesis of F6

1) To a mixture of E22 (222.63 mg, 865.18 mmol) in CH₃CN (3 mL) was added CDI (140.29 mg, 865.18 mmol). The reaction was stirred at 20° C. for 10 min.

2) To a mixture of F4 (200.78 mg, 786.52 umol) in CH₃CN (3 mL) was added t-BuOK (97.08 mg, 865.18 umol). The reaction was heated to 80° C., then the step one reaction mixture was added. The reaction was stirred at 80° C. for 15 min. The reaction mixture was poured into water (10 mL) and adjusted to pH=4 with 1 M HCl, extracted with EtOAc (10 mL×3). The combined organic layers were washed with brine (10 mL×3), dried over Na₂SO₄, then concentrated under reduced pressure to give F6 (447 mg, 56% yield) as a brown solid.

### General Procedure for Synthesis of Compound 77

To a mixture of F6 (447 mg, 903.79 mol) in EtOH (5 mL) was added concentrated HCl (0.5 mL). The reaction mixture was stirred at 80° C. for 1 h. The reaction mixture was concentrated under reduced pressure to remove solvent. The crude product was triturated with Ethyl acetate (5 mL) to give compound 77 (478 mg, crude) as a brown solid. 378 mg of crude product was used into next step.

100 mg of crude product was purified by Prep-HPLC (column: Boston Green ODS 150*30 mm*5 um; mobile phase: [water (FA)-ACN]; B %: 18%-48%, 12 min) to give compound 77 (8.9 mg, 8.90% yield, 100% purity) as a yellow solid.

### General Procedure for Synthesis of Compound 79

A suspension of compound 77 (378 mg, 958.26 mol) in HBr (5 mL, 40% purity) was heated to 120° C. for 4 h. The reaction mixture was concentrated under reduced pressure. The crude product was purified by Prep-HPLC (column: Boston Green ODS 150*30 mm*5 um; mobile phase: [water (FA)-ACN]; B %: 8%-32%, 14 min) to give compound 79 (7.6 mg, 2% yield, 97% purity) as a yellow solid.

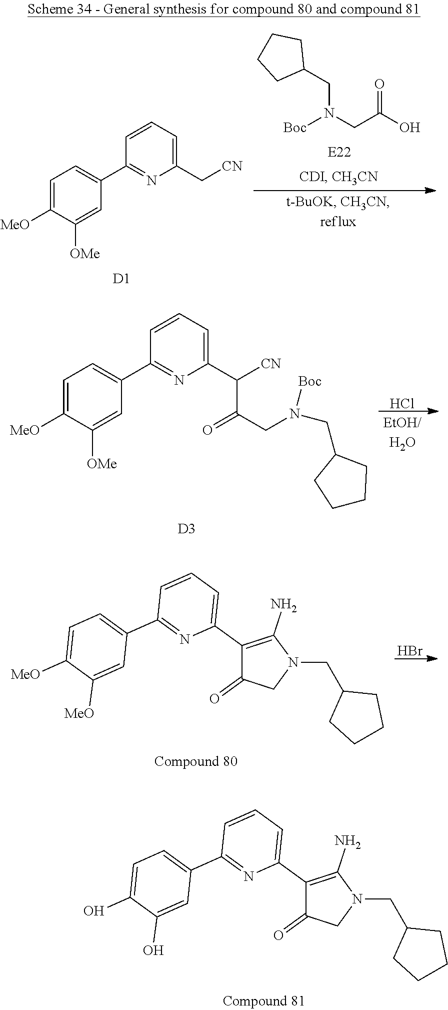

### General Procedure for Synthesis of D3

1) To a mixture of E22 (111.32 mg, 0.4 mmol) in CH₃CN (1 mL) was added CDI (70.14 mg, 0.43 mmol). The mixture was stirred at 25° C. for 3 min.

2) To a mixture of D1 (100 mg, 0.39 mmol) in CH₃CN (1 mL) was added t-BuOK (44.13 mg, 0.39 mmol). The mixture was stirred at 80° C. for 2 min. The step one reaction mixture was added dropwise at 80° C., The resulting mixture was stirred at 80° C. for 15 min. The residue was adjusted with 1M HCI to pH=4, and then extracted with EtOAc (20 mL×3). The combined organic layer was washed with brine (20 mL×3), dried over anhydrous Na₂SO₄, filtered and concentrated to give D3 (210 mg, crude) as an orange solid.

### General Procedure for Synthesis of Compound 80

To a mixture of D3 (210 mg, 0.44 mmol) in EtOH (2 mL) was added concentrated HCl (0.4 mL). The mixture was stirred at 80° C. for 1 h. The reaction mixture was concentrated under reduced pressure to remove solvent. The crude product was triturated with EtOAc (5 mL) at 25° C. for 30 min to give compound 80 (120 mg, crude) as a brown solid. 90 mg of the crude product was used into the next step without further purification. 30 mg of the crude product was purified by prep-HPLC (column: Boston Green ODS 150*30 mm*5 um; mobile phase: [water (FA)-ACN]; B %: 20%-50%, 12 min) to give compound 80 (12.5 mg, 100% purity) as a yellow solid.

### General Procedure for Synthesis of Compound 81

A solution of compound 80 (90 mg, 0.2 mmol) in HBr (1 mL, 40% purity) was stirred at 120° C. for 12 h. The reaction mixture was concentrated under reduced pressure to remove solvent. The residue was purified by prep-HPLC (column: Boston Green ODS 150*30 mm*5 um; mobile phase: [water (FA)-ACN]; B %: 15%-45%, 12 min) to give compound 81 (23.3 mg, 28% yield, 100% purity) as a yellow solid.

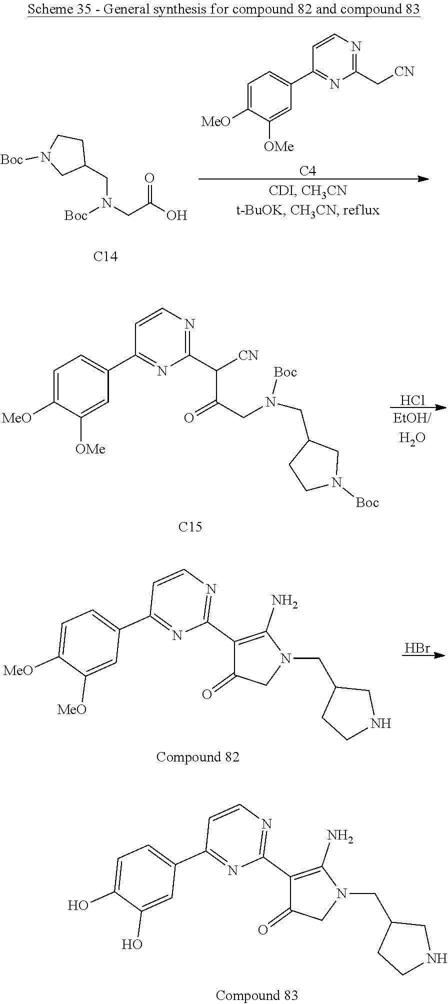

### General Procedure for Synthesis of C15

1) To a mixture of C14 (154.45 mg, 0.43 mmol) in CH₃CN (1 mL) was added CDI (69.87 mg, 0.43 mmol). The mixture was stirred at 25° C. for 3 min.

2) To a mixture of C4 (100 mg, 0.39 mmol) in CH₃CN (1 mL) was added t-BuOK (43.96 mg, 0.39 mmol). The mixture was stirred at 80° C. for 2 min. The step one reaction mixture was added dropwise at 80° C., The resulting mixture was stirred at 80° C. for 15 min. The mixture was adjusted with 1 M HCI to pH=4, extracted with EtOAc (10 mL×3). The combined organic layer was washed with brine (10 mL×3), dried over anhydrous Na₂SO₄, filtered and concentrated to give C15 (210 mg, crude) as an orange solid.

### General Procedure for Synthesis of Compound 82

To a mixture of C15 (210 mg, 0.35 mmol) in EtOH (2 mL) was added concentrated HCl (0.4 mL). The mixture was stirred at 80° C. for 1 h. The reaction mixture was concentrated under reduced pressure. The crude product was triturated with EtOAc (5 mL) at 25° C. for 30 min to give compound 82 (99 mg, crude) as a brown solid. 70 mg of the crude product was used into the next step without further purification. 29 mg of the crude product was purified by prep-HPLC (column: Boston Green ODS 150*30 mm*5 um; mobile phase: [water (FA)-ACN]; B %: 5%-35%, 10 min) to give compound 82 (9 mg, 100% purity) as a yellow solid.

### General Procedure for Synthesis of Compound 83

A suspension of compound 82 (70 mg, 0.17 mmol) in HBr (2 mL, 40% purity) was stirred at 120° C. for 12 h. The reaction mixture was concentrated under reduced pressure. The residue was purified by prep-HPLC (column: Boston Green ODS 150*30 mm*5 um; mobile phase: [water (FA)-ACN]; B %: 0%-22%, 10 min) to give compound 83 (13.1 mg, 20% yield, 99% purity) as an off-white solid.

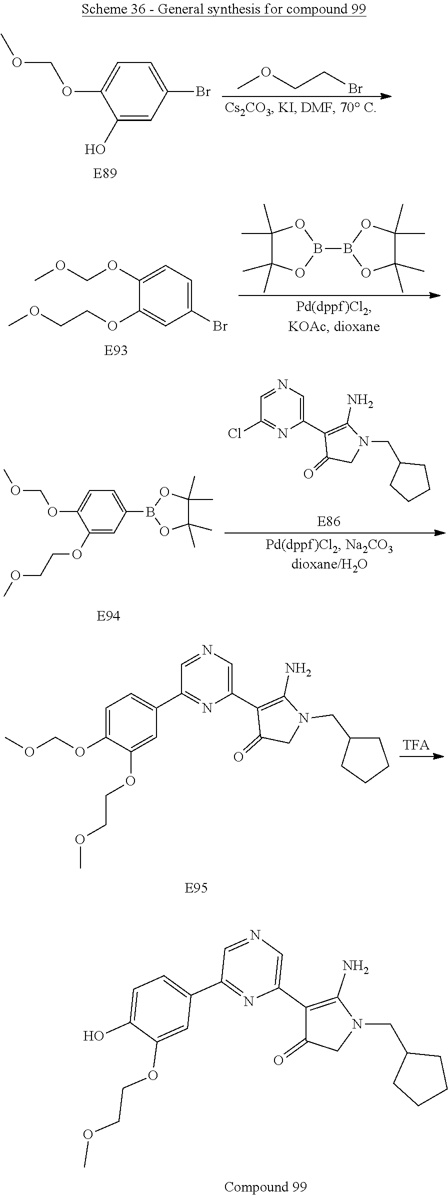

### General Procedure for Synthesis of E93

To a mixture of E89 (500 mg, 2.15 mmol) in DMF (5 mL) was added 1-bromo-2-methoxyethane (298.19 mg, 2.15 mmol, 202 L), Cs₂CO₃ (1.40 g, 4.29 mmol) and KI (356.14 mg, 2.15 mmol). The mixture was stirred at 70° C. for 12 h. The reaction mixture was diluted with H₂O (20 mL) and extracted with EtOAc (20 mL×3). The combined organic layer was washed with brine (20 mL×3), dried over anhydrous Na₂SO₄, filtered and concentrated. The residue was purified by column chromatography (SiO2, Petroleum ether/Ethyl acetate=1/1) to give E93 (430 mg, 69% yield) as a yellow oil.

### General Procedure for Synthesis of E94

To a mixture of E93 (430 mg, 1.48 mmol) in dioxane (4 mL) was added 4,4,4′,4′,5,5,5′,5′-octamethyl-2,2′-bi(1,3,2-dioxaborolane) (450.07 mg, 1.77 mmol), KOAc (434.85 mg, 4.43 mmol) and Pd(dppf)Cl₂ (108.07 mg, 0.15 mmol). The mixture was stirred at 100° C. for 2 h under N2 atmosphere. The reaction mixture of E94 (499 mg, crude) was used into the next step without further purification.

### General Procedure for Synthesis of E95

To a solution of E94 (499 mg, 1.48 mmol) in dioxane (5 mL) and H₂O (1 mL) was added E86 (431.96 mg, 1.48 mmol), Pd(dppf)Cl₂ (107.96 mg, 0.15 mmol) and Na₂CO₃ (312.77 mg, 2.95 mmol). The mixture was stirred at 100° C. for 2 h under N₂ atmosphere. The mixture was quenched with H₂O (20 mL) and extracted with EtOAc (20 mL×3). The combined organic layers were washed with brine (20 mL×3), dried over Na₂SO₄, concentrated under reduced pressure. The residue was purified by column chromatography (SiO₂, Petroleum ether/Ethyl acetate=1/1 to 0/1) to give E95 (170 mg, 22% yield, 90% purity) as a brown solid.

### General Procedure for Synthesis of Compound 99

To a solution of E95 (170 mg, 0.36 mmol) in CH₂Cl₂ (0.5 mL) was added TFA (2 mL). The mixture was stirred at 25° C. for 1 h. The reaction mixture was concentrated under reduced pressure to remove solvent. The residue was purified by prep-HPLC (column: Boston Green ODS 150*30 mm*5 um; mobile phase: [water (FA)-ACN]; gradient: 30%-60% B over 10 min) to give compound 99 (50.1 mg, 33% yield, 100% purity) as a yellow solid.

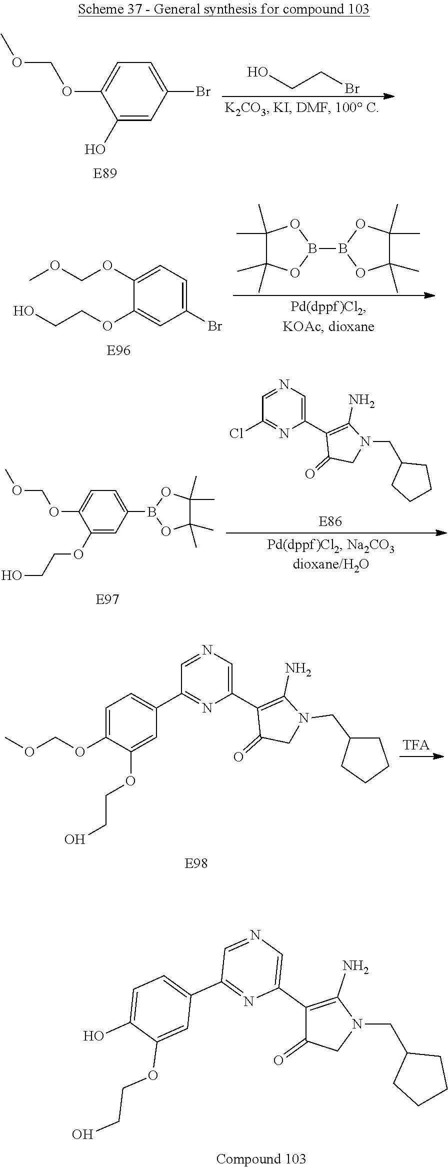

### General Procedure for Synthesis of E96

To a mixture of E89 (500 mg, 2.15 mmol) in DMF (5 mL) was added K₂CO₃ (593.01 mg, 4.29 mmol) and 2-bromoethan-1-ol (268.10 mg, 2.15 mmol, 152 L). The mixture was stirred at 100° C. for 2 h. The reaction mixture was diluted with H₂O (50 mL) and extracted with EtOAc (20 mL×3). The combined organic layers were washed with brine (20 mL×3), dried over anhydrous Na₂SO₄, filtered and concentrated under reduced pressure. The residue was purified by column chromatography (SiO₂, Petroleum ether/Ethyl acetate=1/1) to give E96 (263 mg, 44% yield) as a colorless oil.

### General Procedure for Synthesis of E97

To a mixture of E96 (263 mg, 0.95 mmol) in dioxane (3 mL) was added 4,4,4′,4′,5,5,5′,5′-octamethyl-2,2′-bi(1,3,2-dioxaborolane) (289.21 mg, 1.14 mmol), Pd(dppf)Cl₂ (69.44 mg, 0.09 mmol) and KOAc (279.43 mg, 2.85 mmol). The mixture was stirred at 100° C. for 2 h under N₂ atmosphere. The reaction mixture E97 (308 mg, crude) was used into the next step without further purification.

### General Procedure for Synthesis of E98

To a solution of E97 (308 mg, 0.95 mmol) in dioxane (3 mL) and H₂O (0.6 mL) was added E86 (278.15 mg, 0.95 mmol), Na₂CO₃ (201.40 mg, 1.90 mmol) and Pd(dppf)Cl₂ (69.52 mg, 0.09 mmol). The mixture was stirred at 100° C. for 2 h under N₂ atmosphere. The reaction mixture was diluted with H₂O (20 mL) and extracted with EtOAc (20 mL×3). The combined organic layer was washed with brine (20 mL×3), dried over anhydrous Na₂SO₄, filtered and concentrated. The residue was purified by column chromatography (SiO₂, Petroleum ether/Ethyl acetate=1/1) to give E98 (180 mg, 20% yield, 49% purity) as a brown solid.

### General Procedure for Synthesis of Compound 103

To a solution of E98 (180 mg, 0.39 mmol) in CH₂Cl₂ (0.5 mL) was added TFA (2 mL). The mixture was stirred at 25° C. for 1 h. The reaction mixture was concentrated under reduced pressure to remove solvent. The residue was purified by prep-HPLC (column: Boston Green ODS 150*30 mm*5 um; mobile phase: [water (FA)-ACN]; gradient: 14%-44% B over 12 min) to give compound 103 (8.8 mg, 5% yield, 96% purity) as a white solid.

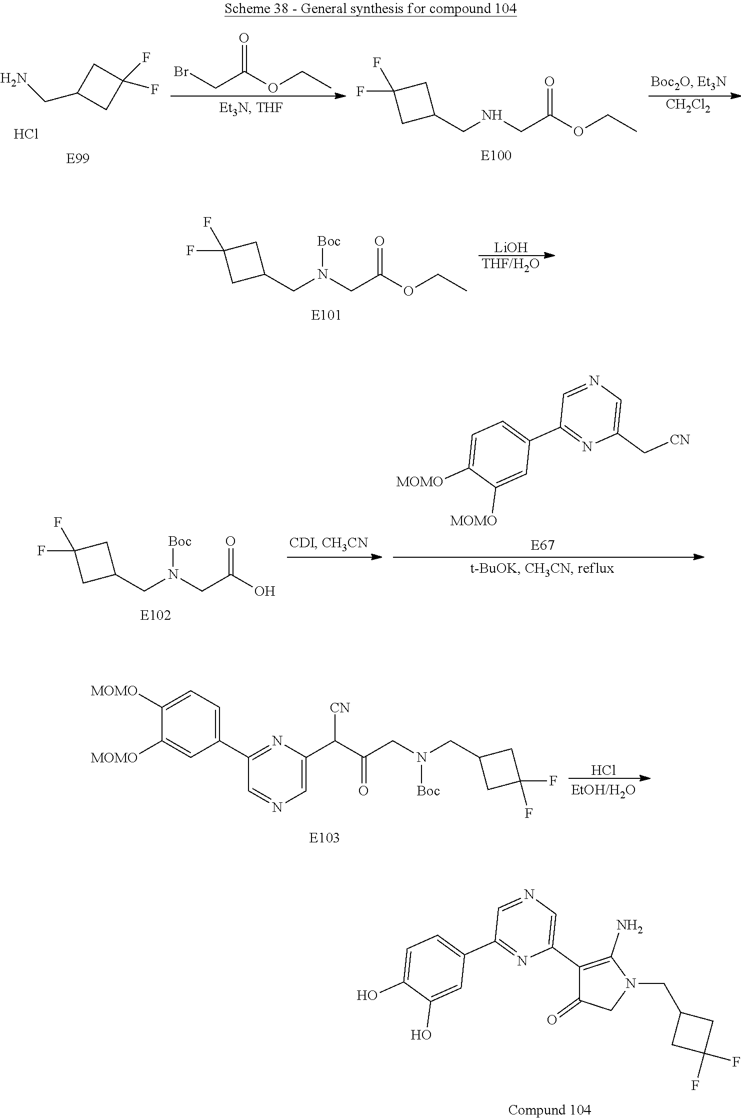

### General Procedure for Synthesis of E100

To a solution of E99 (500 mg, 3.17 mmol, HCl) in CH₂Cl₂ (10 mL) was added ethyl 2-bromoacetate (423.89 mg, 2.54 mmol, 280.72 L) and Et₃N (385.26 mg, 3.81 mmol, 529.94 L). The mixture was stirred at 25° C. for 12 hr. The reaction mixture was diluted with H₂O (20 mL) and extracted with EtOAc (20 mL×3). The combined organic layers were washed with brine (20 mL×3), dried over anhydrous Na₂SO₄, filtered and concentrated under reduced pressure. The residue was purified by column chromatography (SiO₂, Petroleum ether/Ethyl acetate=1/1) to give E100 (228 mg, 35% yield) as a yellow oil.

### General Procedure for Synthesis of E101

To a solution of E100 (228 mg, 1.10 mmol) in CH₂Cl₂ (3 mL) was added Boc₂O (240.14 mg, 1.10 mmol, 252.78 L) and Et₃N (167.01 mg, 1.65 mmol, 229.72 L). The mixture was stirred at 25° C. for 12 h. The reaction mixture was concentrated under reduced pressure. The residue was purified by column chromatography (SiO₂, Petroleum ether/Ethyl acetate=3/1 to 1/1) to give E101 (250 mg, 74% yield) as a colorless oil.

### General Procedure for Synthesis of E102

To a solution of E101 (250 mg, 0.81 mmol) in THF (3 mL) and H₂O (0.6 mL) was added LiOH·H₂O (170.68 mg, 4.07 mmol). The mixture was stirred at 25° C. for 12 hr. The reaction mixture was adjusted with 1N HCl to pH=4, and then extracted with EtOAc (20 mL×3). The combined organic layer was washed with brine (20 mL×3), dried over anhydrous Na₂SO₄, filtered and concentrated to give E102 (220 mg, crude) as a white solid.

### General Procedure for Synthesis of E103

1) To a mixture of E102 (219.21 mg, 0.78 mmol) in CH₃CN (2 mL) was added CDI (127.27 mg, 0.78 mmol), the mixture was stirred at 25° C. for 3 min.

2) To a mixture of E67 (225 mg, 0.71 mmol) in CH₃CN (2 mL) was added t-BuOK (80.07 mg, 0.71 mmol), the mixture was stirred at 80° C. for 2 min. The step one reaction mixture was added dropwise at 80° C. The resulting mixture was stirred at 80° C. for 15 min. The residue was adjusted with 1 M HCI to pH=4, and then extracted with EtOAc (20 mL×3). The combined organic layer was washed with brine (20 mL×3), dried over anhydrous Na₂SO₄, filtered and concentrated to give E103 (390 mg, crude) as a brown solid.

### General Procedure for Synthesis of Compound 104

To a solution of E103 (390 mg, 0.68 mmol) in EtOH (4 mL) was added concentrated HCl (1 mL). The mixture was stirred at 80° C. for 1 h. The reaction mixture was concentrated under reduced pressure to remove solvent. The residue was purified by prep-HPLC (column: Boston Green ODS 150*30 mm*5 um; mobile phase: [water (FA)-ACN]; gradient: 35%-65% B over 12 min) to give compound 104 (19.5 mg, 7% yield, 99% purity) as a brown solid.

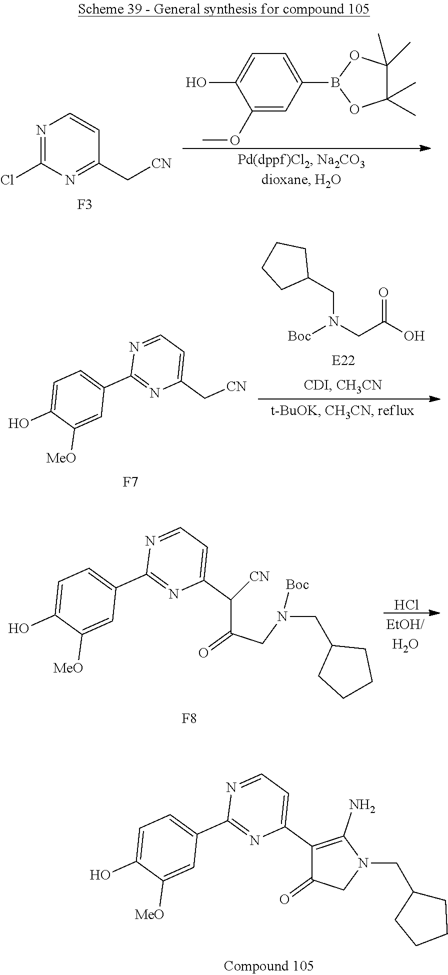

### General Procedure for Synthesis of F7

To a mixture of F3 (500 mg, 3.26 mmol) and 2-methoxy-4-(4,4,5,5-tetramethyl-1,3,2-dioxaborolan-2-yl) phenol (895.71 mg, 3.58 mmol) in dioxane (5 mL) was added H₂O (0.5 mL), Pd(dppf)Cl₂ (238.24 mg, 325.59 umol) and Na₂CO₃ (690.18 mg, 6.51 mmol). The mixture was stirred at 100° C. for 3 h under N₂ atmosphere. The reaction mixture was poured into water (15 mL) and extracted with EtOAc (20 mL×3). The combined organic layers were washed with brine (20 mL×3), dried over Na₂SO₄, concentrated under reduced pressure. The residue was purified by flash silica gel chromatography (PE:EtOAc=1:1) to give the crude. The crude product was purified by Prep-HPLC (column: Boston Prime C18 150*30 mm*5 um; mobile phase: [water (FA)-ACN]; gradient: 21%-51% B over 12 min) to give F7 (82 mg, 100% purity) as a yellow solid.

### General Procedure for Synthesis of F8

1) To a mixture of E22 (96.21 mg, 373.89 umol) in CH₃CN (2 mL) was added CDI (60.63 mg, 373.89 umol) and the reaction was stirred at 20° C. for 10 min.

2) Toa mixture of F7 (82 mg, 339.90 umol) in CH₃CN (2 mL) was added t-BuOK (41.95 mg, 373.89 umol), the reaction was heated to 80° C., then the first step reaction mixture was added and the reaction was stirred at 80° C. for 15 min. The reaction mixture was poured into water (20 mL) and adjusted to pH=4 with 1 M HCl, extracted with EtOAc (20 mL×3). The combined organic layers were washed with brine (20 mL×3), dried over Na₂SO₄, then concentrated under reduced pressure to give F8 (138 mg, 38% yield) as a brown solid.

### General Procedure for Synthesis of Compound 105

To a mixture of F8 (138 mg, 287.17 mol) in EtOH (10 mL) was added concentrated HCl (1 mL). The reaction mixture was stirred at 80° C. for 1 h. The reaction mixture was concentrated under reduced pressure to give a residue. The crude product was purified by Prep-HPLC (column: Boston Green ODS 150*30 mm*5 um; mobile phase: [water (FA)-ACN]; gradient: 15%-45% B over 12 min) to give compound 105 (25.5 mg, 22% yield, 96% purity) as a yellow solid.

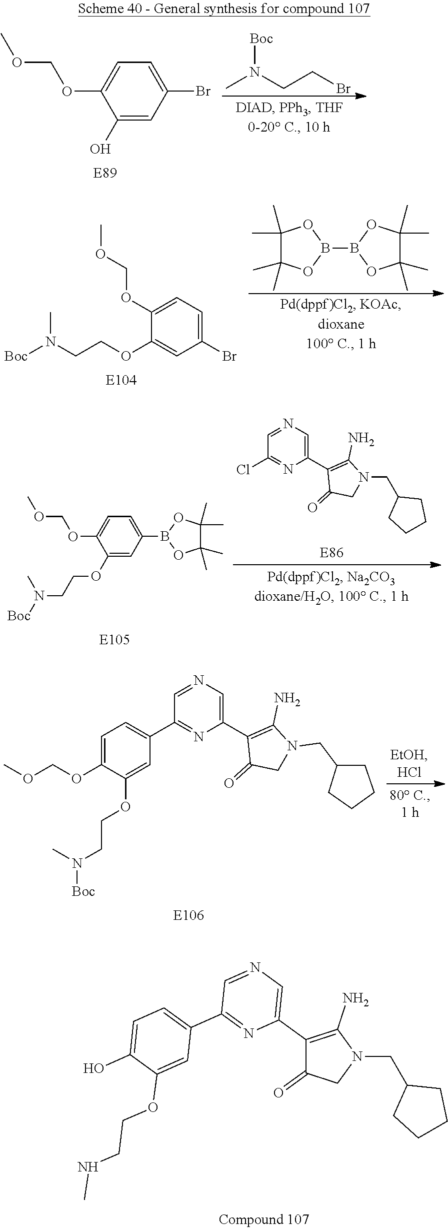

### General Procedure for Synthesis of E104

To a mixture of E89 (300 mg, 1.29 umol) in THF (10 mL) was added tert-butyl (2-hydroxyethyl)(methyl)carbamate (248.11 mg, 1.42 mmol) and PPh₃ (506.43 mg, 1.93 mmol), then DIAD (390.43 mg, 1.93 mmol) was added to the mixture at 0° C. and the mixture was stirred at 20° C. for 10 h. The mixture was diluted with ethyl acetate (15 mL) and filtered. The organic layer was washed with water (20 mL×2), dried over Na₂SO₄, filtered and concentrated to give a residue. The residue was purified by flash column chromatography on silica gel (0-30% EtOAc in PE) to give E104 (337 mg, 47% yield, 70% purity) as colorless oil.

### General Procedure for Synthesis of E105

A mixture of E104 (300 mg, 768.70 umol) and 4,4,4′,4′,5,5,5′,5′-octamethyl-2,2′-bi(1,3,2-dioxaborolane) (195.20 mg, 768.70 mol) in dioxane (3 mL) was added Pd(dppf)Cl₂ (56.25 mg, 76.87 mol) and KOAc (226.33 mg, 2.31 mmol). The mixture was stirred at 100° C. for 1 h. The mixture was diluted with ethyl acetate (15 mL) and filtered. The organic layer was washed with water (20 mL×2), dried over Na₂SO₄, filtered and concentrated to give E105 (115 mg, 24% yield, 70% purity) as a brown oil.

### *645General Procedure for Synthesis of E106

A mixture of E105 (115 mg, 262.96 mol) and E86 (84.68 mg, 289.25 mol) in dioxane (3 mL) was added Pd(dppf)Cl₂ (19.24 mg, 26.30 mol) and Na₂CO₃ (55.74 mg, 525.91 mol) and H₂O (0.3 mL). The mixture was stirred at 100° C. for 1 h under N₂ atmosphere. The mixture was diluted with ethyl acetate (15 mL) and filtered, the filtrate was concentrated under reduced pressure to give a residue. The crude was purified by flash column chromatography on silica gel (0-30% EtOAc in PE) to give E106 (39 mg, 18% yield, 70% purity) as a yellow oil.

### General Procedure for Synthesis of Compound 107

To a suspension of E106 (39 mg, 68.70 mol) in EtOH (5 mL) was added concentrated HCl (0.5 mL). The reaction mixture was stirred at 80° C. for 1 h. The reaction mixture was concentrated under reduced pressure to give a residue. The crude product was purified by Prep-HPLC (column: Boston Green ODS 150*30 mm*5 um; mobile phase: [water (FA)-ACN]; gradient: 10%-40% B over 12 min) to give compound 107 (2.4 mg, 8% yield, 100% purity) as a yellow solid.

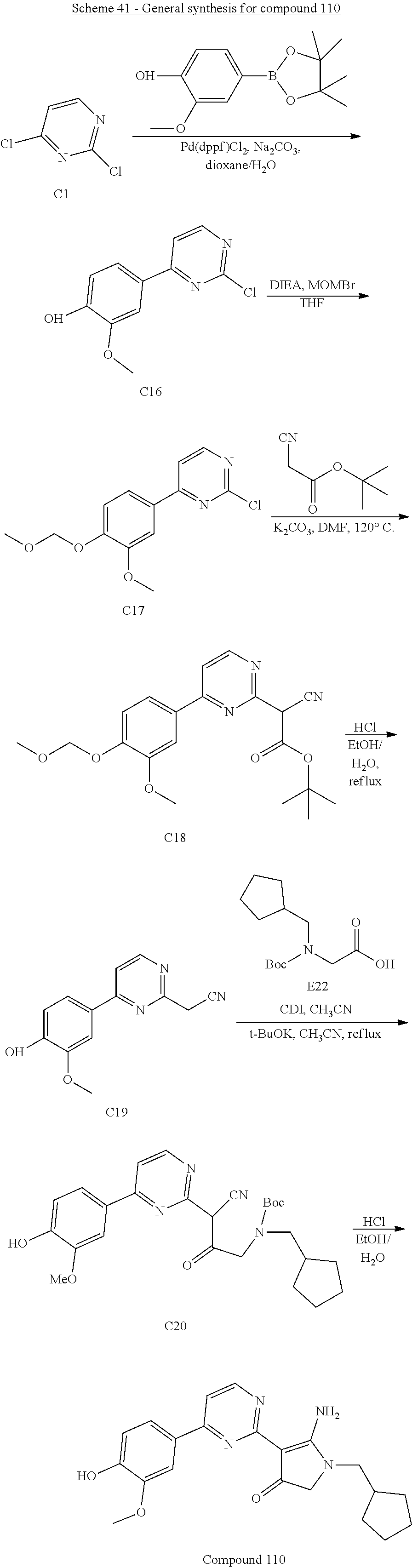

### General Procedure for Synthesis of C16

To a solution of C1 (2.46 g, 16.52 mmol) in dioxane (30 mL) and H₂O (6 mL) was added 2-methoxy-4-(4,4,5,5-tetramethyl-1,3,2-dioxaborolan-2-yl) phenol (3.18 g, 12.71 mmol), Na₂CO₃ (2.69 g, 25.42 mmol) and Pd(dppf)Cl₂ (929.85 mg, 1.27 mmol). The mixture was stirred at 100° C. for 2 h. The reaction mixture was diluted with H₂O (50 mL) and extracted with EtOAc (50 mL×3). The combined organic layers were washed with brine (50 mL×3), dried over anhydrous Na₂SO₄, filtered and concentrated under reduced pressure to give a residue. The residue was purified by column chromatography (SiO₂, Petroleum ether/Ethyl acetate=1/1) to give C16 (2.5 g, crude) as a white solid.

### General Procedure for Synthesis of C17

To a solution of C16 (1.5 g, 6.34 mmol) in THF (15 mL) was added DIEA (3.28 g, 25.35 mmol, 4.42 mL) and bromo(methoxy)methane (950.49 mg, 7.61 mmol, 620.83 L). The mixture was stirred at 20° C. for 12 hr. The mixture was quenched with sat. NaHCO₃ to pH=8 and extracted with ethyl acetate (30 mL×3). The combined organic layer was dried over Na₂SO₄, filtered and concentrated. The residue was purified by column chromatography (SiO₂, DCM:MeOH=10:1) to give C17 (785 mg, 17% yield, 38% purity) as a white solid.

### General Procedure for Synthesis of C18

To a solution of C17 (500 mg, 1.78 mmol) in DMF (5 mL) was tert-butyl 2-cyanoacetate (301.74 mg, 2.14 mmol, 305.71 L) and K₂CO₃ (984.73 mg, 7.12 mmol). The mixture was stirred at 120° C. for 16 h. The crude reaction mixture on notebook page ES20772-487 (735 mg scale) was combined to ES20772-488 for workup. The combined reaction mixture was poured into water (30 mL) and extracted with ethyl acetate (30 mL×3). The combined organic layer was washed with brine (20 mL×3), dried over anhydrous Na₂SO₄, filtered and concentrated to give a residue. The residue was purified by column chromatography (SiO₂, Petroleum ether/Ethyl acetate=1/1 to 0/1) to give C18 (940 mg, crude) as a yellow solid.

### General Procedure for Synthesis of C19

To a solution of C18 (940 mg, 2.44 mmol) in EtOH (10 mL) was added HCl (2 mL). The mixture was stirred at 80° C. for 1 h. The reaction mixture was concentrated under reduced pressure to remove solvent. The crude product was triturated with EtOAc (2 mL) at 25° C. for 30 min to give C19 (560 mg, 79.0% yield, 83% purity) as a yellow solid.

### General Procedure for Synthesis of C20

1) To a mixture of E22 (305.06 mg, 1.19 mmol) in CH₃CN (2 mL) was added CDI (192.23 mg, 1.19 mmol), the mixture was stirred at 25° C. for 3 min.

2) To a mixture of C19 (260 mg, 1.08 mmol) in CH₃CN (2 mL) was added t-BuOK (120.94 mg, 1.08 mmol), the mixture stirred at 80° C. for 2 min. The step one reaction was added dropwise at 80° C. The resulting mixture was stirred at 80° C. for 15 min. The residue was adjusted with 1 N HCl to pH=4, and then extracted with EtOAc (20 mL×3). The combined organic layer was washed with brine (20 mL×3), dried over anhydrous Na₂SO₄, filtered and concentrated to give C20 (670 mg, crude) as a brown solid.

### General Procedure for Synthesis of Compound 110

To a solution of C20 (670 mg, 1.39 mmol) in EtOH (5 mL) was added concentrated HCl (1 mL). The mixture was stirred at 80° C. for 1 h. The reaction mixture was concentrated under reduced pressure to remove solvent. The crude product on notebook page ES20772-493 was combined to ES20772-498 for further purification. The residue was purified by prep-HPLC (column: Boston Green ODS 150*30 mm*5 um; mobile phase: [water (FA)-ACN]; gradient: 10%-40% B over 12 min) to give compound 110 (54.5 mg, 10% yield, 96% purity) as a yellow solid.

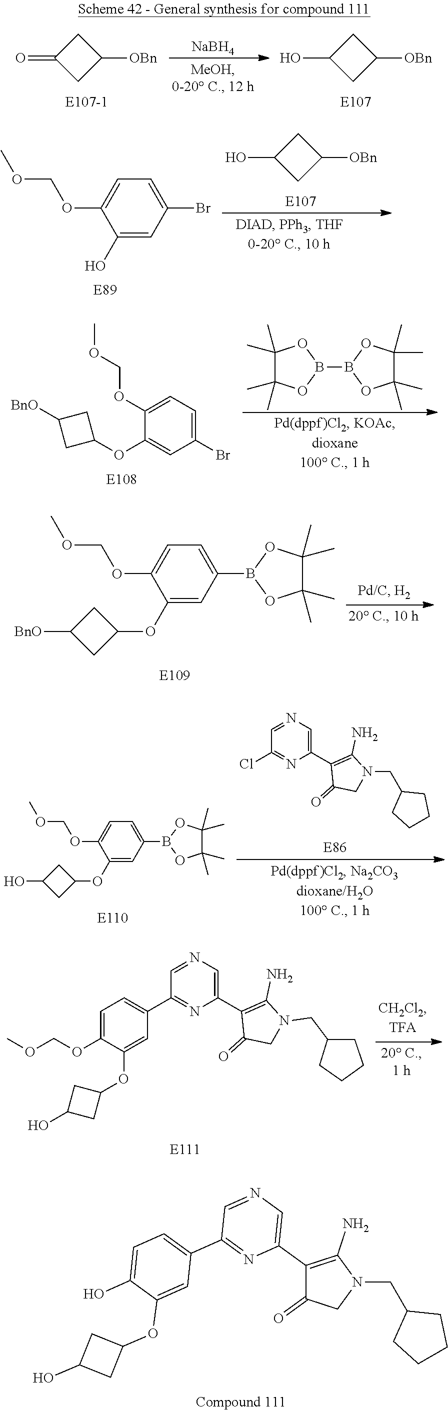

### General Procedure for Synthesis of E107

To a solution of E107-1 (1 g, 5.68 mmol) in MeOH (10 mL) was added NaBH₄ (322.05 mg, 8.51 mmol) at 0° C., the reaction was stirred at 20° C. for 12 h. The mixture was quenched with NH4C1 (30 mL), and then poured into water (30 mL) and extracted with EtOAc (30 mL×3). The organic layer was washed with brine (30 mL×3), dried over Na₂SO₄, filtered and concentrated to give E107 (974 mg, 96% yield) as a brown solid.

### General Procedure for Synthesis of E108

To a solution of E89 (1.1 g, 4.72 mmol) in THF (15 mL) was added E107 (925.32 mg, 5.19 mmol) and PPh₃ (1.86 g, 7.08 mmol), then DIAD (1.43 g, 7.08 mmol) was added to the mixture at 0° C. and the mixture was stirred at 20° C. for 10 h. The mixture was diluted with ethyl acetate (15 mL) and filtered. The organic layer was washed with water (20 mL×2), dried over Na₂SO₄, filtered and concentrated to give a residue. The residue was purified by flash column chromatography on silica gel (0-30% EtOAc in PE) to give E108 (617 mg, 33% yield) as a colorless oil.

### General Procedure for Synthesis of E109

To a solution of E108 (617 mg, 1.57 mmol) and 4,4,4′,4′,5,5,5′,5′-octamethyl-2,2′-bi(1,3,2-dioxaborolane) (438.24 mg, 1.73 mmol) in dioxane (5 mL) was added Pd(dppf)Cl₂ (114.80 mg, 156.89 mol) and KOAc (307.95 mg, 3.14 mmol). The mixture was stirred at 100° C. for 1 h under N₂. The mixture was diluted with ethyl acetate (15 mL) and filtered. The organic layer was washed with water (20 mL×2), dried over Na₂SO₄, filtered and concentrated to give a crude. The crude was purified by flash silica gel chromatography (PE:EA=5:1) to give E109 (495 mg, 71% yield) was obtained as a colorless oil.

### General Procedure for Synthesis of E110

To a mixture of E109 (495 mg, 1.12 mmol) in MeOH (10 mL) was added Pd/C (100 mg, 10% purity). The reaction mixture was stirred at 20° C. for 10 h under H₂ (15 psi). The reaction mixture was filtered, and the filtrate was concentrated under reduced pressure to give E110 (365 mg, 64% yield, 70% purity) as a colorless oil.

### General Procedure for Synthesis of E111

To a solution of E110 (100 mg, 285.54 mol) and E86 (83.60 mg, 285.54 mol) in dioxane (5 mL) was added Pd(dppf)Cl₂ (20.89 mg, 28.55 mol) and Na₂CO₃ (60.53 mg, 571.08 mol) and H₂O (0.5 mL). The mixture was stirred at 100° C. for 1 h under N₂. The mixture was diluted with ethyl acetate (15 mL) and filtered, the organic layer was washed with water (20 mL×2), dried over Na₂SO₄, filtered and concentrated to give E111 (199 mg, 58% yield, 40% purity) as a brown solid.

### General Procedure for Synthesis of Compound 111

To a solution of E111 (199.00 mg, 414.10 mol) in CH₂Cl₂ (5 mL) was added TFA (2 mL). The reaction mixture was stirred at 20° C. for 1 h. The reaction mixture was concentrated under reduced pressure to give a residue. The crude product was purified by Prep-HPLC (column: Boston Green ODS 150*30 mm*5 um; mobile phase: [water (FA)-ACN]; gradient: 22%-52% B over 12 min) to give compound 111 (4 mg, 2% yield, 100% purity) as a yellow solid.

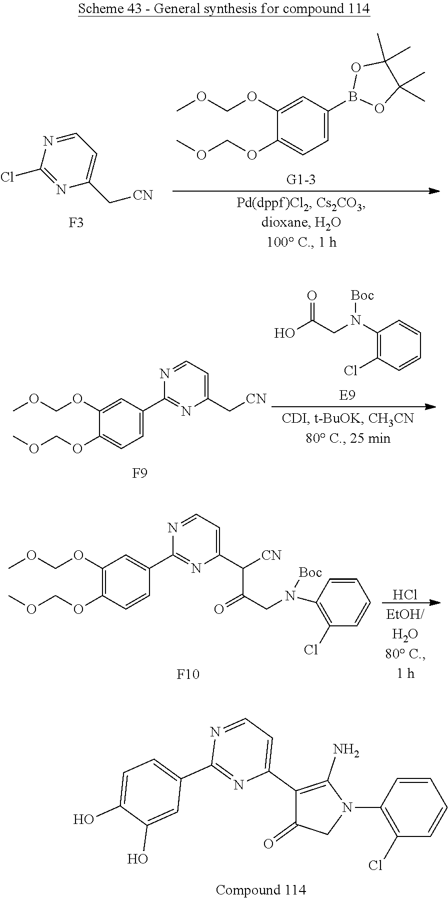

### General Procedure for Synthesis of F9

To a solution of F3 (100 mg, 651.17 mol) and G1-3 (232.20 mg, 716.29 mol) in dioxane (5 mL) was added Pd(dppf)Cl₂ (47.65 mg, 65.12 mol), Cs₂CO₃ (424.33 mg, 1.30 mmol) and H₂O (0.5 mL). The mixture was stirred at 100° C. for 1 h. The mixture was diluted with ethyl acetate (15 mL) and filtered. The organic layer was washed with water (20 mL×2), dried over Na₂SO₄, filtered and concentrated to give a residue. The residue was purified by flash silica gel chromatography (PE:EA=3:1) to give F9 (204 mg, crude) as a brown solid.

### General Procedure for Synthesis of F10

1) To a solution of E9 (203.33 mg, 711.65 umol) in CH₃CN (3 mL) was added CDI (115.39 mg, 711.65 umol) and the reaction stirred at 20° C. for 10 min.

2) To a solution of F9 (204 mg, 646.96 umol) in CH₃CN (3 mL) was added t-BuOK (79.86 mg, 711.65 umol), the reaction was heated to 80° C., then the first solution was added, and the reaction stirred at 80° C. for 15 min.

The reaction mixture was poured into water (10 mL) and adjusted to pH=4 with 1 N HCl, then extracted with EtOAc (10 mL×3). The combined organic layers were washed with brine (10 mL×3), dried over Na₂SO₄, then concentrated under reduced pressure to give F10 (565 mg, crude) as a brown solid.

### General Procedure for Synthesis of Compound 114

To a solution of F10 (565 mg, 135.67 umol) in EtOH (10 mL) was added concentrated HCl (1 mL). The reaction mixture was stirred at 80° C. for 1 h. The reaction mixture was concentrated under reduced pressure to give a residue. The crude product was purified by Prep-HPLC (column: Boston Green ODS 150*30 mm*5 um; mobile phase: [water (FA)-ACN]; gradient: 4%-34% B over 12 min) to give compound 114 (9.1 mg, 96% purity) as a yellow solid.

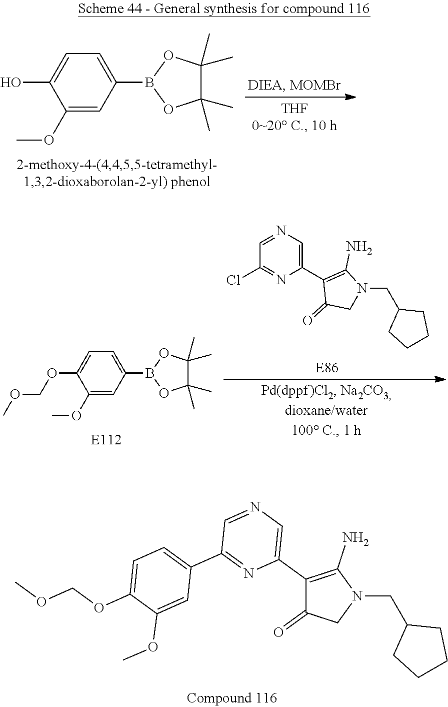

### General Procedure for Synthesis of E112

To a solution of 2-methoxy-4-(4,4,5,5-tetramethyl-1,3,2-dioxaborolan-2-yl) phenol (1.5 g, 6.00 mmol) in THF (30 mL) was added MOMBr (899.36 mg, 7.20 mmol) at 0° C. over 30 min. and then DIEA (3.10 g, 23.99 mmol) was added dropwise at 0° C. The resulting mixture was stirred at 20° C. for 10 hr. LCMS showed no desired product was detected. TLC indicated 2-methoxy-4-(4,4,5,5-tetramethyl-1,3,2-dioxaborolan-2-yl) phenol was consumed completely and one new spot formed.

The reaction mixture was quenched with NH₄Cl (20 mL), then poured into water (20 mL) and extracted with EtOAc (20 mL×3). The combined organic layers were washed with brine (20 mL×3), dried over Na₂SO₄, concentrated under reduced pressure to give a residue. The residue was purified by flash silica gel chromatography (PE:EtOAc=3:1) to give E112 (1.8 g, crude) as a colorless oil.

### General Procedure for Synthesis of Compound 116

To a solution of E112 (1.8 g, 6.12 mmol) and E86 (1.79 g, 6.12 mmol) in dioxane (10 mL) was added Pd(dppf)Cl₂ (447.76 mg, 611.93 mol) and Na₂CO₃ (1.30 g, 12.24 mmol) and H₂O (1 mL). The mixture was stirred at 100° C. for 1 hour under N₂.

The mixture was diluted with ethyl acetate (15 mL) and filtered. The combined organic layers were washed with water (20 mL×2), dried over Na₂SO₄, concentrated under reduced pressure to give compound 116 (3.88 g, 74.68% yield, 50% purity) as a brown solid.

The crude product (400 mg) was purified by Prep-HPLC (column: Boston Green ODS 150*30 mm*5 um; mobile phase: [water (FA)-ACN]; gradient: 36%-66% B over 12 min) to give compound 116 (52.8 mg, 100% purity) as a yellow solid.

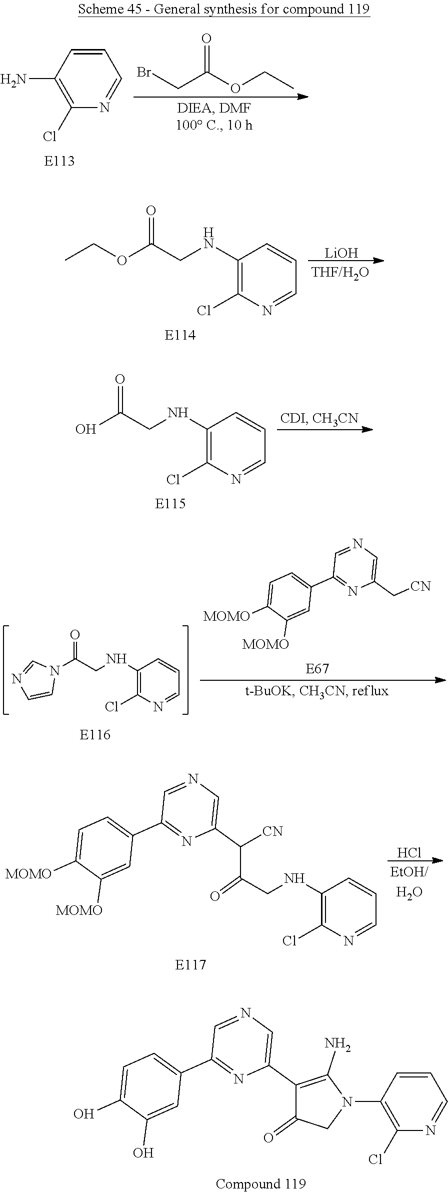

### General Procedure for Synthesis of E114

To a solution of E113 (10 g, 77.79 mmol) in DMF (150 mL) was added DIEA (30.16 g, 233.36 mmol, 40.65 mL) and ethyl 2-bromoacetate (14.29 g, 85.56 mmol, 9.47 mL). The mixture was stirred at 100° C. for 10 hour. The reaction mixture was diluted with H₂O (100 mL) and extracted with EtOAc (100 mL×3). The combined organic layers were washed with brine (100 mL×3), dried over anhydrous Na₂SO₄, filtered and concentrated under reduced pressure to give a residue. The residue was purified by column chromatography (SiO₂, Petroleum ether/Ethyl acetate=3/1 to 1/1) to give E1 14 (3.64 g, 19.6% yield, 90% purity) as a yellow oil.

### General Procedure for Synthesis of E115

To a solution of E114 (1 g, 4.66 mmol) in THF (15 mL) and H₂O (3 mL) was added LiOH·H₂O (977.50 mg, 23.29 mmol). The mixture was stirred at 25° C. for 12 hour. The reaction mixture was diluted with H₂O (20 mL) and extracted with EtOAc (50 mL). The aqueous phase was adjusted to pH=5 with HCl (1N) and extracted with EtOAc (20 mL×3), The combined organic layer was washed with brine (20 mL×3), dried over anhydrous Na₂SO₄, filtered and concentrated under reduced pressure to give E115 (320 mg, crude) as a white solid.

### General Procedure for Synthesis of E117

1) To a mixture of E115 (318.96 mg, 1.71 mmol) in CH₃CN (5 mL) was added CDI (277.17 mg, 1.71 mmol), the mixture was stirred at 25° C. for 3 min.

2) To a mixture of E67 (490 mg, 1.55 mmol) in CH₃CN (5 mL) was added t-BuOK (348.74 mg, 3.11 mmol), the mixture stirred at 80° C. for 2 min.

The step 1 reaction was added dropwise into step 2 reaction mixture at 80° C., The resulting mixture was stirred at 80° C. for 15 min. The residue was adjusted with 1N HCl to pH=4, and then extracted with EtOAc (50 mL×3). The combined organic layer was washed with brine (50 mL×3), dried over anhydrous Na₂SO₄, filtered and concentrated to give E117 (700 mg, crude) as a yellow solid.

### General Procedure for Synthesis of Compound 119

To a solution of E117 (700 mg, 1.45 mmol, crude) in EtOH (10 mL) was added 37% HCl (2 mL). The mixture was stirred at 80° C. for 1 hr. The reaction mixture was concentrated under reduced pressure to remove solvent. The residue was purified by prep-HPLC (column: Boston Green ODS 150*30 mm*5 um; mobile phase: [water(FA)-ACN]; gradient: 10%-40% B over 12 min) and then prep-HPLC (column: Phenomenex Gemini NX 150×30 mm, 5 μm; mobile phase: [water(NH₄HCO₃)-ACN]; gradient: 13%-43% B over 11 min) to give compound 119 (0.9 mg, 100% purity) as white solid

### Experimental Example 1—YFP Assay

Principle for YFP assay

Halide sensor YFP F46L/H148Q/I152L is quenched by halide ion like Iodide. When such YFP is expressed in a cell, it expresses in the cytoplasm.

To measure H723R-PDS activity, PANC-1 cells were stably transfected with halide sensors YFP-F46L/H148Q/I152L and H723R-PDS which has problems with membrane trafficking due to misfolding.

If the cell-treated drug is a corrector for H723R-PDS, H723R-PDS will translocate normally to the plasma membrane.

At this time, when treated with a high-concentration iodide solution outside the cell, iodide enters the cell due to the Cl−/I− exchange activity of pendrin, and the fluorescence of the halide sensor YFP decreases.

### Protocol for YFP Assay

### Cells for High-Throughput Screening

PANC-1 cells were cultured in Dulbecco's Modified Eagle's Medium with 10% fetal bovine serum, 2 mM glutamine, 100 units/ml penicillin and 100 g/ml streptomycin. For high-throughput screening, PANC-1 cells were stably transfected with human H723R-pendrin and halide sensor YFP-F46L/H148Q/I152L.

### YFP Fluorescence Quenching Assay Procedures

PANC-1 cells expressing human H723R-pendrin and YFP-F46L/H148Q/I152L were plated in 96-well microplates at a density of 2×10⁴ cells per well and grown for 20-24 hours at 37° C. (90% humidity 5% CO₂). Then these cells were treated with 50 μl growth medium containing test compounds and incubated for 20-24 hours. For YFP quenching assay, each well of the 96-well plate was washed 2 times with 200 μl of PBS, and it was filled with 50 μl of HEPES-buffered solution (140 mM NaCl, 5 mM KCl, 1 mM MgCl2, 1 mM CaCl₂, 10 mM D-glucose, 10 mM HEPES; pH 7.4 with NaOH). After 10 minutes of incubation at 37° C., the 96-well plate was moved on the FLUOstar Omega Microplate Reader (BMG Labtech, Ortenberg, Germany) for a fluorescence assay. Each well was assayed individually for H723R-pendrin mediated I⁻ influx by recording fluorescence (495±15 nm excitation, 520±20 nm emission) every 400 ms for 1 second (baseline). Then, 50 μL of NaI-substituted HEPES-buffered solution (NaI replacing NaCl) was added using a liquid injector at 1 second and YFP fluorescence was recorded every 400 ms for 10 seconds. The initial iodide influx rate was determined from the initial slope of fluorescence by nonlinear regression, after the infusion of iodide.

Table 1 shows measurement of Cl⁻/I⁻ exchange activity of pendrin for selected compounds of the invention. It is indicated as EC₅₀ with the following key: A=EC₅₀ less than 5 uM; B=EC₅₀ greater than 5 uM, but less than 10 uM; C=EC₅₀ greater than 10 uM.

TABLE 1
YFP assay

| | YFP assay |
|---|---|
| # cpds | (Panc-1, EC50, uM) |
| 1 | C |
| 2 | C |
| 3 | C |
| 4 | B |
| 5 | C |
| 6 | B |
| 7 | C |
| 8 | A |
| 9 | A |
| 10 | C |
| 11 | C |
| 12 | C |
| 13 | A |
| 14 | B |
| 15 | B |
| 16 | C |
| 17 | C |
| 18 | A |
| 19 | C |
| 20 | B |
| 21 | C |
| 22 | C |
| 23 | C |
| 24 | C |
| 25 | C |
| 26 | C |
| 27 | C |
| 28 | A |
| 29 | C |
| 30 | C |
| 31 | C |
| 32 | B |
| 33 | A |
| 34 | C |
| 35 | C |
| 36 | C |
| 37 | C |
| 38 | C |
| 39 | C |
| 40 | C |
| 41 | C |
| 42 | C |
| 43 | C |
| 44 | A |
| 45 | C |
| 46 | A |
| 47 | C |
| 48 | C |
| 49 | A |
| 50 | C |
| 51 | C |
| 52 | C |
| 53 | C |
| 54 | C |
| 55 | C |
| 56 | C |
| 57 | A |
| 58 | C |
| 59 | C |
| 60 | C |
| 61 | C |
| 62 | A |
| 63 | B |
| 64 | B |
| 65 | B |
| 66 | B |
| 67 | C |
| 68 | C |
| 69 | A |
| 70 | A |
| 71 | C |
| 72 | C |
| 73 | A |
| 74 | C |
| 75 | C |
| 76 | C |
| 77 | C |
| 78 | A |
| 79 | A |
| 80 | C |
| 81 | A |
| 82 | C |
| 83 | C |
| 84 | C |
| 85 | C |
| 86 | C |
| 87 | B |
| 88 | C |
| 89 | C |
| 90 | C |
| 91 | C |
| 92 | B |
| 93 | C |
| 94 | C |
| 95 | C |
| 96 | C |
| 97 | C |
| 98 | C |
| 99 | A |
| 100 | C |
| 101 | C |
| 102 | C |
| 103 | C |
| 104 | B |
| 105 | A |
| 106 | C |
| 107 | A |
| 108 | C |
| 109 | C |
| 110 | A |
| 111 | A |
| 112 | B |
| 113 | C |
| 114 | A |
| 115 | C |
| 116 | A |
| 117 | B |
| 118 | C |
| 119 | C |
| 120 | C |

Activity range: A Indicates <5 uM,
B indicates 5≤ EC50 <10 uM,
C indicates ≥10 uM

### Experimental Example 2—Surface Biotinylation Assay

### Principle for Surface Biotinylation Assay

Surface biotinylation is a commonly used technique to specifically isolate plasma membrane proteins using a modified biotin in a form that is easy to bind with proteins. Sulfo-NHS-SS-biotin is a generally used reagent in surface biotinylation due to its impermeability and water solubility. In cool conditions, the extracellular domain of membrane proteins is covalently binding with the reactive biotin ester. These proteins are separated from the whole lysate via a biotin-streptavidin response. In this process, we can validate the increasing rate of the expression level of mutant pendrin in the plasma membrane.

### Protocol for Surface Biotinylation Assay

Panc1^{parental}, Panc1^{hPDS-WT} and Panc1^{hPDS-H723R} cells were cultured in high glucose DMEM medium supplemented with 10% fetal bovine serum and 1% antibiotics. 24 hours after seeding in 6-well plates, cells were treated as indicated for 24 h. Cells were washed in PBS and incubated on ice with 0.3 mg/ml biotin in PBS. After 35 min, 1% BSA in PBS, D.W mixture (1:1) was added to cells, and cells were incubated for 10 min. Cells were washed and lysed in lysis buffer. The lysates were sonicated for 20 seconds, and not boiled. After centrifugated, the supernatants were collected and quantified via BCA analysis. The quantified supernatants were added to 10% avidin beads in PBS and incubated overnight at 4° C. After incubation, the below steps were repeated 4 times: i) centrifugation, ii) removing the supernatant, iii) washing with lysis buffer. After the last centrifugation and removing the supernatant, the sample buffer mixed with reducing buffer was added to biotin-avidin mixtures. The elution was accomplished in the 38° C. bioshaker for 40 min and the supernatant was collected. The supernatant was separated by SDS-PAGE and transferred to an Immobilon membrane. After incubation in 5% BSA solution to prevent nonspecific binding, the membrane was incubated with specific antibodies overnight. Then, the membrane was washed with TBST buffer and incubated with HRP-conjugated anti-rabbit or -mouse antibody for 1 hour. The ECL buffer and iBright were used for the visualization of protein bands. The respective protein band intensity was quantified by densitometric analysis using the NIH ImageJ program.

Table 2 shows expression percent of H723R-hPDS surface as the glycosylated form pendrin relative to wildtype hPDS for selected compounds of the invention. It is indicated as expression % with the following key: A=Expression % greater than 100%; B=Expression % greater than 50%, but less than 100%; C=Expression % less than 50%.

TABLE 2
Surface biotinylation assay

Surface expression % of H723R-hPDS
relative to WT-hPDS (Glycosylated
form pendrin)

| # cpds | 3 uM | 10 uM |
|---|---|---|
| 8 | B | A |
| 9 | A | A |
| 13 | A | A |
| 18 | A | A |
| 28 | A | A |
| 33 | A | A |
| 46 | B | A |
| 49 | A | A |
| 62 | B | A |
| 105 | A | A |

| | |
|---|---|
| Expression (%) | A = Greater than 100; B = Greater |
| range | than 50, but less than 100; C = Less than 50 |

### Experimental Example 3—PTI Assay

### Protocol for PTI Assay

Measurement of Cl⁻/HCO3⁻ Exchange Activity

Measurements of intracellular pH (pHᵢ) in PANC-1 cells were performed with the pH-sensitive fluorescent probe 2′,7′-bis-(2-carboxyethyl)-5-(and-6)-carboxyfluorescein (BCECF) according to previously reported protocols. Briefly, cells were incubated with 2 μM BCECF acetoxy-methylester for 5 min and then perfused with a HCO3⁻-buffered solution [containing 120 mM NaCl, 5 mM KCl, 1 mM MgCl2, 1 mM CaCl2, 10 mM d-glucose, 5 mM Hepes, and 25 mM NaHCO₃ (pH 7.4)].

BCECF fluorescence was recorded at excitation wavelengths of 490 and 440 nm at a resolution of 2/s on a recording setup. The Cl⁻/HCO₃⁻ exchange activities were estimated from the initial rate of pHᵢ increase as a result of Cl⁻ removal from the HCO₃⁻-containing buffer (25 mM HCO3⁻ with 5% CO₂).

The pHi calibration was performed with standard pH solutions containing 150 mM KCl and 5 μM nigericin. The intrinsic buffer capacity (βi) was calculated by measuring ΔpHi in response to 5 to 40 mM NH₄Cl pulses in Na⁺-free solutions. Because the βi values were not substantially affected by transfection with the plasmids encoding for WT-pendrin or H723R-pendrin, the Cl⁻/HCO₃⁻ exchange activities were expressed as ΔpH unit/min without compensating for the buffer capacity.

Table 3 shows measurement of Cl⁻/HCO₃ exchange activity of pendrin for selected compounds of the invention. It is indicated as exchange % with the following key: A=Exchange % greater than 50%; B=Exchange % less than 50%.

TABLE 3
PTI assay

Cl⁻/HCO₃⁻ exchange, % of WT-hPDS

| # cpds | 3 uM | 10 uM |
|---|---|---|
| 9 | A | A |
| 18 | A | A |
| 28 | B | A |
| 49 | A | A |
| 69 | B | A |
| 73 | A | A |
| 105 | A | A |

| | |
|---|---|
| Exchange (%) | A indicates ≥50, B indicates <50 |

range

### Experimental Example 4—Patient-Derived Cell-Based (PDC) Assay

### Principle for PDC Assay

The experimental approach is grounded in a multi-step process aimed at investigating the effects of drug treatments on nasal epithelial cells. Initially, nasal tissues are collected and processed to isolate epithelial cells through tissue lysis. These cells are then cultured and subjected to Air-Liquid Interface (ALI) conditions for an extended period. Following this, drug treatments involving human IL-4 and Pendrin corrector drugs are administered, and protein harvesting is conducted for subsequent analysis. The expression of the pendrin antibody ‘hR1 (YONSEI ENT Choi lab.)’ is confirmed through Western blot analysis after drug treatments. Additionally, immunofluorescence staining is performed on Transwell membranes from different drug concentrations, and the resulting cryosections are subjected to confocal microscopy using the primary antibody ‘hR2 (YONSEI ENT Choi lab.)’. This comprehensive methodology allows for the visualization and analysis of pendrin expression in both basal and apical regions of nasal epithelial cells, providing insights into the impact of drug treatment on the targeted cellular components.

### Protocol for PDC Assay

### Measurement of Pendrin Expression in Patient Nasal Epithelials Cells Through Western Blot and Conforcal Microscopy

1. Tissue Collection and Cell Isolation

Nasal tissue were collected from patients and placed in DMEM:F12 (Lonza Cat No. 12-719F) supplemented with 1% penicillin-streptomycin in Transfer media. After removing red blood cells with PBS, tissues were subjected to tissue lysis with 1% protease in transfer media at 37° C. for 1 hour, resulting in the isolation of epithelial cells.

2. Cell Culture

The obtained epithelial cells were conventionally cultured up to passage 1 in BEGM Bulletkit (Lonza Cat No. CC3170) supplemented with BSA 150 mg/ml and EGF (BD Cat No. 354001). Once reaching approximately 90% confluency, cells were detached using 0.25% Trypsin EDTA and seeded onto 12-well Transwell plates (Costar Cat No. 3450) in a 1:1 mixture of DMEM media (Lonza Cat No. 12-707F) and BEGM Bulletkit supplemented media for Air-Liquid Interface (ALI) culture.

3. Air-Liquid Interface Culture

ALI culture was initiated by adding 1 ml of media to the bottom and seeding 0.5 ml of cells on the membrane. When cells on the membrane reached 90% confluency, the bottom was filled with culture media supplemented with 50 nM Retinoic acid (RA, SIGMA Cat No. R2625), and ALI culture was performed for 7-14 days.

4. Drug Treatment and Protein Harvesting

Subsequent to ALI culture, cells were treated with human IL-4 at 10 g/ml for 24 hours, followed by treatment with ‘Compound 9, Compound 18 and Compound 105’ Pendrin corrector drugs at concentrations of 0.1, 0.3, 1, 3, 10 μM for 24 hours. After drug treatment, cells attached to the Transwell membrane were harvested by scraping into protein buffer (iNtRON Cat No. 17081). Protein quantification (30 g) was performed for each sample and the expression of the pendrin antibody ‘hR1 (YONSEI ENT custom antibody)’ was confirmed through Western blot analysis.

5. Immunofluorescence Staining

For immunofluorescence staining, Transwell membranes from each drug-treated concentration were fixed in 4% paraformaldehyde (PFA) at room temperature for 10 minutes. Subsequently, they were cut into three pieces using a microtome, embedded in OCT compound and processed in to 5 μm cryosections. Immunostaining using the primary antibody ‘hR2 (YONSEI ENT custom antibody)’ was conducted, and the pendrin expression in the basal and apical regions of epithelial cells was visualized using confocal microscopy (Carl Zeiss, LSM700).

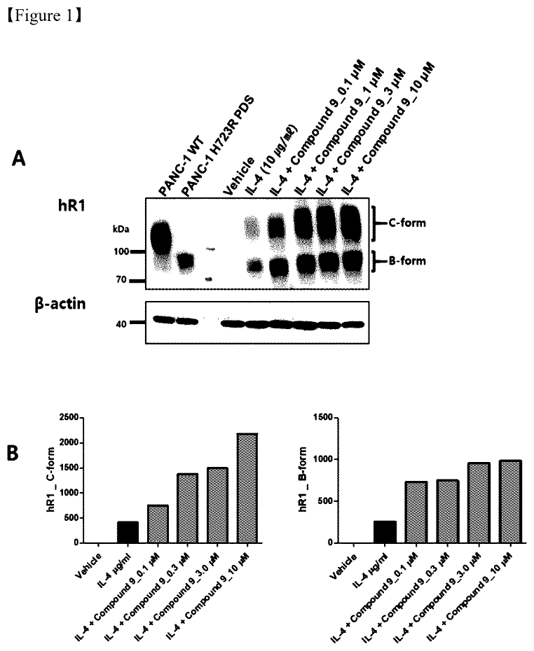

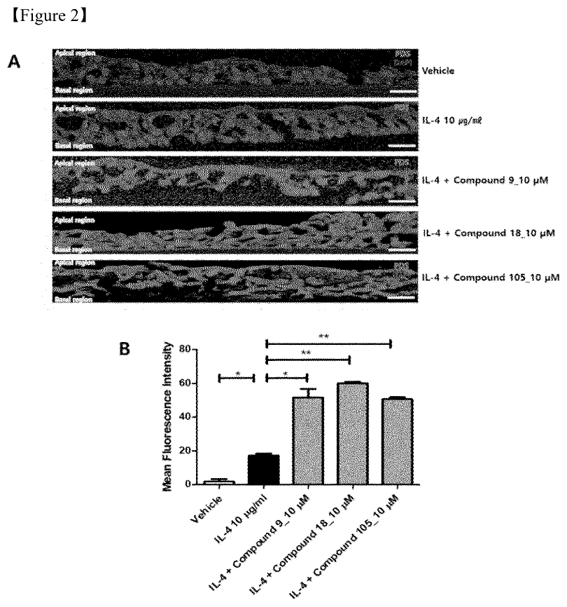

The results of the above experiments are shown in FIGS. 1 and 2.

FIG. 1 shows western blot analysis results of compound 9 treated patient epithelial cells inferred from patient nasal epithelial cells.

FIG. 1. (A, B) B-form, which represents a specific non-glycosylated precursor form of pendrin, showed few noticeable changes in response to Compound 9 treatment; however, in C-form, which represents a fully-glycosylated functional pendrin, a notable dose-dependent increase is observed, especially at a high concentration of 10 μM. These observations, extrapolated to patient-derived nasal epithelial cells, suggest a concentration-dependent effect of Compound 9 treatment on C-form expression in cells harboring the H724R mutation.

FIG. 2 shows immunofluorescence staining results of compound 9, 18 and 105

FIG. 2. (A) The figure illustrates immunofluorescence staining outcomes for epithelial cells under different conditions. In the vehicle and IL-4 groups, pendrin expression is absent, indicated by no fluorescence. Conversely, treatment with Compound 9, Compound 18 and Compound 105 at 10 μM exhibits a clear red fluorescence, signifying enhanced pendrin expression localized on the epithelial cell membrane. Scale bars=20 μm. (B) The graph is quantitative representation, comparing the mean fluorescence intensity levels between the vehicle, IL-4 and Compound 9, Compound 18 and Compound 105 (10 μM) groups. Statistical significance indicated a significant difference in pendrin expression intensity between experimental conditions. *p<0.05.

The specific compounds prepared by the general procedure as shown above, are described in below Table 4.

TABLE 4
Summarizes compounds 1-120 in terms of their structures and corresponding
characteristics.

| #cpd | Structure | Characterization Daia |
|---|---|---|
| 1 | 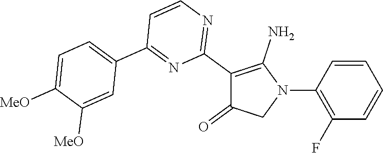 [SMILES: COc1ccc(-c2ccnc(C3=C(N)N(c4ccccc4F)CC3=O)n2)cc1OC] | Brown solid; ¹H NMR (400 MHz, DMSO-d6) δ = 9.43 (br. s, 1H), 8.68-8.53 (m, 1H), 8.04-7.88 (m, 1H), 7.86- 7.74 (m, 2H), 7.67-7.58 (m, 1H), 7.56-7.40 (m, 3H), 7.39-7.32 (m, 1H), 7.15-7.08 (m, 1H), 4.07 (br. s, 2H), 3.85 (s, 3H), 3.84 (s, 3H); LCMS: 100%, MS (ESI): m/z 407.2 [M + H]+. |
| 2 | 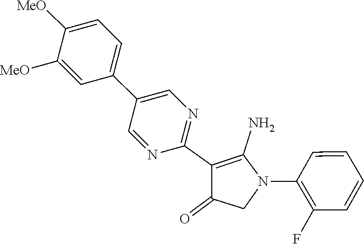 [SMILES: COc1ccc(-c2cnc(C3=C(N)N(c4ccccc4F)CC3=O)nc2)cc1OC] | Yellow solid; ¹H NMR (400 MHz, CHLOROFORM-d) δ = 9.21-8.95 (m, 2H), 7.53-7.38 (m, 2H), 7.30 (br s, 3H), 7.24-7.06 (m, 3H), 7.05-6.88 (m, 1H), 4.30 (br s, 2H), 3.95 (br d, J = 8.0 Hz, 6H); LCMS: 100%, MS (ESI): m/z 407.3 [M + H]+. |
| 3 | 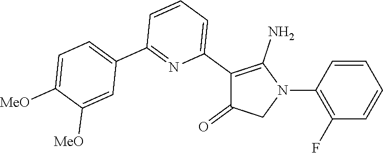 [SMILES: COc1ccc(-c2cccc(C3=C(N)N(c4ccccc4F)CC3=O)n2)cc1OC] | Yellow solid; ¹H NMR (400 MHz, CHLOROFORM-d) δ = 8.37 (brs, 1H), 8.10 (brs, 1H), 7.55-7.44 (m, 5H), 7.38-7.30 (m, 4H), 7.04 (d, J = 8.4 Hz, 1H), 4.39 (s, 2H), 4.01 (s, 3H), 3.98 (s, 3H); LCMS: 100%, MS (ESI): m/z 406.3 [M + H]+. |
| 4 | 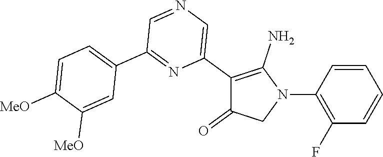 [SMILES: COc1ccc(-c2cncc(C3=C(N)N(c4ccccc4F)CC3=O)n2)cc1OC] | Yellow solid; ¹H NMR (400 MHz, DMSO-d6) δ = 9.52 (s, 1H), 9.30 (br. s, 1H), 8.72 (s, 1H), 8.13 (br. s, 1H), 7.64 (t, J = 8.0 Hz, 1H), 7.57-7.41 (m, 4H), 7.39-7.33 (m, 1H), 7.09 (d, J = 8.4 Hz, 1H), 4.19 (s, 2H), 3.82 (s, 6H); LCMS: 95%, MS (ESI): m/z 407.2 [M + H]+. |
| 5 | 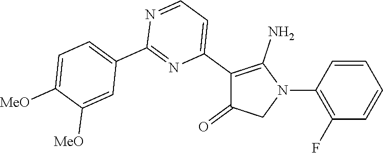 [SMILES: COc1ccc(-c2nccc(C3=C(N)N(c4ccccc4F)CC3=O)n2)cc1OC] | White solid; ¹H NMR (400 MHz, DMSO-d6) δ = 9.65 (s, 1H), 8.55 (d, J = 5.6 Hz, 1H), 8.29 (s, 1H), 8.19 (d, J = 5.6 Hz, 1H), 7.80 (dd, J = 2.0, 8.4 Hz, 1H), 7.73-7.62 (m, 2H), 7.56-7.42 (m, 2H), 7.41-7.34 (m, 1H), 7.07 (d, J = 8.8 Hz, 1H), 4.19 (s, 2H), 3.33 (s, 3H), 3.81 (s, 3H); LCMS: 99.3%, MS (ESI): m/z 407. 3 [M + H]+. |
| 6 | 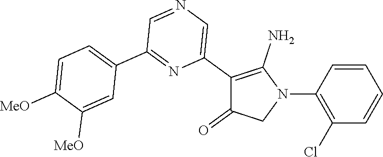 [SMILES: COc1ccc(-c2cncc(C3=C(N)N(c4ccccc4Cl)CC3=O)n2)cc1OC] | White solid; ¹H NMR (400 MHz, DMSO-d6) δ = 9.52 (s, 1H), 9.23 (br s, 1H), 8.72 (s, 1H), 8.02 (br s, 1H), 7.75-7.63 (m, 2H), 7.59-7.50 (m, 3H), 7.47 (d, J = 2.0 Hz, 1H), 7.09 (d, J = 8.5 Hz, 1H), 4.12 (br d, J = 3.8 Hz 2H), 3.82 (d, J = 1.5 Hz, 6H); LCMS: 100%, MS (ESI): m/z 423.2 [M + H]+. |
| 7 | 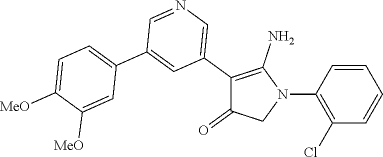 [SMILES: COc1ccc(-c2cncc(C3=C(N)N(c4ccccc4Cl)CC3=O)c2)cc1OC] | White solid; ¹H NMR (400 MHz, DMSO-d6) δ = 8.65 (d, J = 2.0 Hz, 1H), 8.56 (d, J = 2.1 Hz, 1H), 8.13 (s, 1H), 8.02 (t, J = 2.1 Hz, 1H), 7.68-7.60 (m, 1H), 7.60- 7.53 (m, 1H), 7.51-7.41 (m, 2H), 7.36-7.21 (m, 4H), 7.08 (d, J = 8.3 Hz, 1H), 4.09-3.93 (m, 2H), 3.85 (s, 3H), 3.80 (s, 3H); LCMS: 99.74%, MS (ESI): m/z 422.0 [M + H]+. |
| 8 | 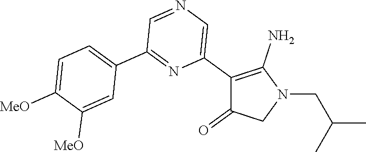 [SMILES: COc1ccc(-c2cncc(C3=C(N)N(CC(C)C)CC3=O)n2)cc1OC] | White solid; ¹H NMR (400 MHz, DMSO-d6) δ = 9.47 (s, 1H), 9.24 (br s, 1H), 8.62 (s, 1H), 8.16 (br s, 1H), 7.54 (dd, J = 2.1, 8.3 Hz, 1H), 7.50 (d, J = 2.0 Hz, 1H), 7.10 (d, J = 8.5 Hz, 1H), 3.88 (s, 3H), 3.84 (s, 3H), 3.80 (s, 2H), 3.26 (d, J = 7.6 Hz, 2H), 2.02 (td, J = 6.9, 13.6 Hz, 1H), 0.91 (d, J = 6.6 Hz, 6H); LCMS: 99.85%, MS (ESI): m/z 369.1 [M + H]+. |
| 9 | 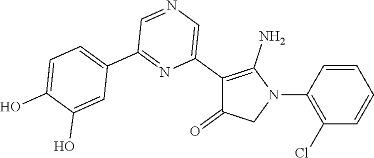 [SMILES: NC1=C(c2cncc(-c3ccc(O)c(O)c3)n2)C(=O)CN1c1ccccc1Cl] | White powder; ¹H NMR (400 MHz, DMSO-d6) δ = 9.46 (s, 1H), 9.18 (br s, 1H), 8.55 (s, 1H), 8.01 (br s, 1H), 7.76-7.60 (m, 2H), 7.58-7.43 (m, 2H), 7.33 (d, J = 2.1 Hz, 1H), 7.26 (dd, J = 2.2, 8.3 Hz, 1H), 6.87 (d, J = 8.2 Hz, 1H), 4.13 (br s, 2H); LCMS: 95.0%, MS (ESI): m/z 395.0 [M + H]+. |
| 10 | 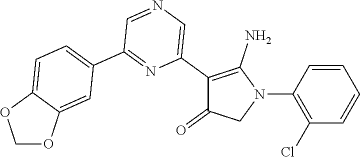 [SMILES: NC1=C(c2cncc(-c3ccc4c(c3)OCO4)n2)C(=O)CN1c1ccccc1Cl] | White solid; ¹H NMR (400 MHz, DMSO-d6) δ = 9.54 (s, 1H), 9.09 (br s, 1H), 8.65 (s, 1H), 7.95 (br s, 1H), 7.73-7.63 (m, 2H), 7.53-7.48 (m, 4H), 7.07 (d, J = 8.6 Hz, 1H), 6.11 (s, 2H), 4.12 (br s, 2H); LCMS: 93.0%, MS (ESI): m/z 407.0 [M + H]+. |
| 11 | 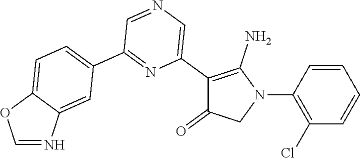 [SMILES: NC1=C(c2cncc(-c3ccc4ocnc4c3)n2)C(=O)CN1c1ccccc1Cl] | White solid; ¹H NMR (400 MHz, DMSO-d6) δ = 9.62 (s, 1H), 9.14 (br s, 1H), 8.85 (s, 1H), 8.80 (s, 1H), 8.37 (d, J = 1.6 Hz, 1H), 8.04 (dd, J = 1.8, 8.6 Hz, 2H), 7.94 (d, J = 8.6 Hz, 1H), 7.74-7.63 (m, 2H), 7.59-7.48 (m, 2H), 4.14 (br s, 2H); LCMS: 99.0%, MS (ESI), m/z 404.0 [M + H]+. |
| 12 | 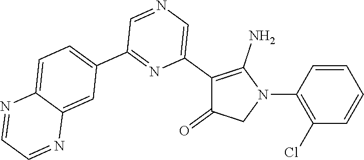 [SMILES: NC1=C(c2cncc(-c3ccc4nccnc4c3)n2)C(=O)CN1c1ccccc1Cl] | White solid; ¹H NMR (400 MHz, DMSO-d6) δ = 9.69 (s, 1H), 9.26-9.04 (t, 1H), 9.04-9.02 (m, 1H), 9.01- 9.00 (m, 1H), 8.98 (s, 1H), 8.66 (d, J = 1.9 Hz, 1H), 8.46 (dd, J = 2.0, 8.8 Hz, 1H), 8.25 (d, J = 8.8 Hz, 1H), 8.11 (br s, 1H), 7.70 (td, J = 3.2, 6.1 Hz, 2H), 7.58-7.47 (m, 2H), 4.17 (br d, J = 3.8 Hz, 2H); LCMS: 100%, MS (ESI): m/z 415.1 [M + H]+. |
| 13 | 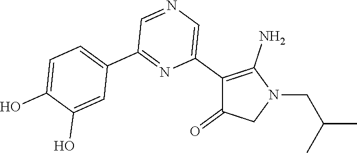 [SMILES: CC(C)CN1CC(=O)C(c2cncc(-c3ccc(O)c(O)c3)n2)=C1N] | Yellow solid; ¹H NMR (400 MHz, DMSO-d6) δ = 9.30- 9.10 (m, 1H), 8.75-8.57 (m, 1H), 7.39 (d, J = 2.1 Hz, 1H), 7.32 (dd, J = 2.1, 8.2 Hz, 1H), 6.91 (d, J = 8.2 Hz, 1H), 4.06 (d, J = 2.7 Hz, 2H), 3.35 (d, J = 7.5 Hz, 2H), 2.03 (td, J = 6.9, 13.6 Hz, 1H), 0.93 (s, 3H), 0.91 (s, 3H); LCMS: 100%, MS (ESI): m/z 341.1 [M + H]+. |
| 14 | 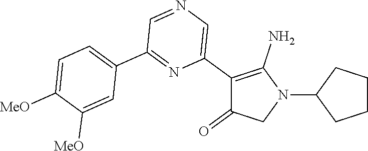 [SMILES: COc1ccc(-c2cncc(C3=C(N)N(C4CCCC4)CC3=O)n2)cc1OC] | Yellow solid; ¹H NMR (400 MHz, DMSO-d6) δ = 9.44 (s, 1 H), 9.31 (br s, 1 H), 8.65 (s, 1 H), 8.25 (br s, 1 H), 7.55 (dd, J = 2.0 Hz, 8.4 Hz, 1 H), 7.50 (d, J = 1.6 Hz, 1 H, 7.11 (d, J = 8.4 Hz, 1 H), 4.39-4.26 (m, 1 H), 3.88 (s, 3 H), 3.84 (s, 3 H), 3.82 (s, 2 H), 1.99-1.83 (m, 2 H), 1.79-1.68 (m, 2 H), 1.67-1.51 (m, 4 H); LCMS: 100%, MS (ESI): m/z 381.1 [M + H]+. |
| 15 | 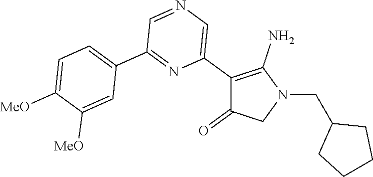 [SMILES: COc1ccc(-c2cncc(C3=C(N)N(CC4CCCC4)CC3=O)n2)cc1OC] | Yellow solid; ¹H NMR (400 MHz, DMSO-d6) δ = 9.46 (s, 1H), 9.36 (br s, 1H), 8.61 (s, 1H), 8.25 (br s, 1H), 7.58-2.45 (m, 2H), 7.10 (d, J = 8.3 Hz, 1H), 3.87 (s, 3H), 3.83 (s, 3H), 3.80 (s, 2H), 3.37 (d, J = 7.7 Hz, 2H), 1.79-1.69 (m, 2H), 1.68-1.60 (m, 2H), 1.58-1.49 (m, 2H), 1.26-1.38 (m, 2H); LCMS: 98.1%, MS (ESI): m/z 395.1 [M + H]+. |
| 16 | 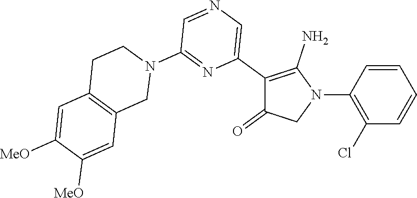 [SMILES: COc1cc2c(cc1OC)CN(c1cncc(C3=C(N)N(c4ccccc4Cl)CC3=O)n1)CC2] | Yellow solid; ¹H NMR (400 MHz, DMSO-d6) δ = 8.76 (s, 2H), 7.99 (s, 2H), 7.69-7.64 (m, 2H), 7.55-7.51 (m, 2H), 6.82 (s, 1H), 6.76 (s, 1H), 4.58 (s, 2H), 4.18-4.08 (m, 2H), 3.77-3.74 (m, 2H), 3.73 (s, 3H), 3.71 (s, 3H), 2.88-2.82 (m, 2H); LCMS: 100%, MS (ESI): m/z 478.1 [M + H]+. |
| 17 | 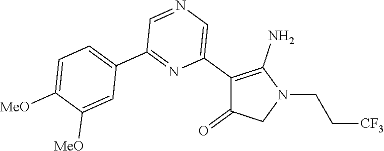 [SMILES: COc1ccc(-c2cncc(C3=C(N)N(CCC(F)(F)F)CC3=O)n2)cc1OC] | Yellow solid; ¹H NMR (400 MHz, DMSO-d6) δ = 9.45 (s, 1H), 9.30 (br s, 1H), 8.64 (s, 1H), 8.45 (br s, 1H), 7.57-7.48 (m, 2H), 7.10 (d, J = 8.5 Hz, 1H), 3.87 (s, 3H), 3.84 (s, 2H), 3.84-3.81 (m, 3H), 3.74 (t, J = 7.2 Hz, 2H), 2.76-2.65 (m, 2H); LCMS: 93.8%, MS (ESI): m/z 409.0 [M + H]+. |
| 18 | 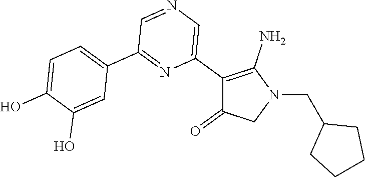 [SMILES: NC1=C(c2cncc(-c3ccc(O)c(O)c3)n2)C(=O)CN1CC1CCCC1] | Yellow solid; ¹H NMR (500 MHz, DMSO-d6) δ = 9.35 (s, 1H), 9.25 (br s, 1H), 8.48 (s, 1H), 8.35 (br s, 1H), 7.34 (d, J = 1.6 Hz, 1H), 7.27 (dd, J = 6.8, 1.6 Hz), 6.88 (d, J = 6.4 Hz, 1H), 3.89 (s, 2H), 3.40 (br d, J = 7.8 Hz, 2H), 2.30-2.21 (m, 1H), 1.79-1.68 (m, 2H), 1.67-1.59 (m, 2H), 1.58-1.47 (m, 2H), 1.26-1.37 (m, 2H); LCMS: 92.8%, MS (ESI): m/z 367.0 [M + H]+. |
| 19 | 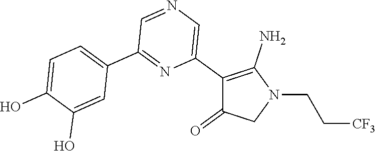 [SMILES: NC1=C(c2cncc(-c3ccc(O)c(O)c3)n2)C(=O)CN1CCC(F)(F)F] | Yellow solid; ¹H NMR (500 MHz, DMSO-d6) δ = 9.37 (s, 1H), 9.30 (br s, 1H), 8.82-8.13 (m, 2H), 7.40-7.24 (m, 2H), 6.88 (d, J = 8 2 Hz, 1H), 3.89 (s, 2H), 3.76 (br t, J = 7.2 Hz, 2H), 2.71 (td, J = 7.2, 11.2 Hz, 2H); LCMS: 94.7%, MS (ESI): m/z 381.0 [M + H]+. |
| 20 | 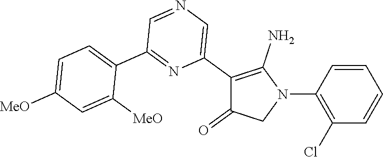 [SMILES: COc1ccc(-c2cncc(C3=C(N)N(c4ccccc4Cl)CC3=O)n2)c(OC)c1] | Yellow solid; ¹H NMR (400 MHz, DMSO-d6) δ = 9.41 (s, 1H), 9.24 (br s, 1H), 8.47 (s, 1H), 7.88 (br s, 1H), 7.71-7.62 (m, 2H), 7.57 (d, J = 8.5 Hz, 1H), 7.54-7.48 (m, 2H), 6.73 (d, J = 2.3 Hz, 1H), 6.69-6.64 (m, 1H), 4.12-4.10 (m, 2H), 3.83 (s, 3H), 3.82 (s, 3H); LCMS: 100% MS (ESI): m/z 423.0 [M + H]+. |
| 21 | 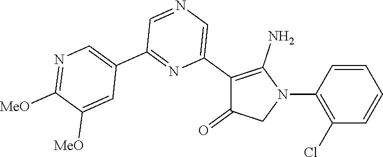 [SMILES: COc1cc(-c2cncc(C3=C(N)N(c4ccccc4Cl)CC3=O)n2)cnc1OC] | Yellow solid; ¹H NMR (500 MHz, DMSO-d6) δ = 9.57 (s, 1H), 9.06 (br s, 1H), 8.77 (s, 1H), 8.34 (d, J = 2.0 Hz, 1H), 7.95 (br s, 1H), 7.73-7.69 (m, 2H), 7.69-7.64 (m, 1H), 7.55-7.51 (m, 2H), 4.13 (br d, J = 6.1 Hz, 2H), 3.93 (s, 3H), 3.86 (s, 3H); LCMS: 98.0%, MS (ESI): m/z 424.0 [M + H]+. |
| 22 | 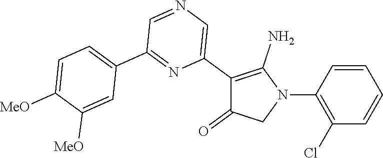 [SMILES: COc1ccc(-c2cncc(C3=C(N)N(c4ccccc4Cl)CC3=O)n2)cc1OC] | Yellow solid; ¹H NMR (400 MHz, DMSO-d6) δ = 9.58 (s, 1H), 9.27 (br s, 1H), 8.74 (s, 1H), 8.02 (br s, 1H), 7.73-7.64 (m, 2H), 7.56-7.48 (m, 2H), 7.08 (d, J = 2.1 Hz, 2H), 6.61 (s, 1H), 4.12 (br d, J = 2.7 Hz, 2H), 3.80 (s, 6H); LCMS: 100%, MS (ESI): m/z 423.1 [M + H]+. |
| 23 | 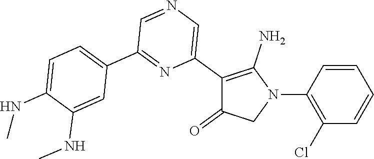 [SMILES: CNc1ccc(-c2cncc(C3=C(N)N(c4ccccc4Cl)CC3=O)n2)cc1NC] | Yellow solid; ¹H NMR (400 MHz, DMSO-d6) δ = 9.39 (s, 2H), 8.60 (s, 1H), 8.18-8.10 (m, 1H), 8.01 (br d, J = 0.8 Hz, 1H), 7.73-7.64 (m, 2H), 7.52 (dd, J = 3.6, 5.9 Hz, 2H), 7.27 (br d, J = 7.9 Hz, 1H), 6.95 (s, 1H), 6.49 (br d, J = 8.1 Hz, 1H), 5.10 (br s, 2H), 4.10 (br s, 2H), 2.77 (br d, J = 8.8 Hz, 6H); LCMS: 100%, MS (ESI): m/z 421.2 [M + H]+. |
| 24 | 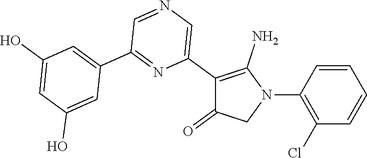 [SMILES: NC1=C(c2cncc(-c3cc(O)cc(O)c3)n2)C(=O)CN1c1ccccc1Cl] | Yellow solid; ¹H NMR (400 MHz, DMSO-d6) δ = 9.54 (d, J = 2.7 Hz, 3H), 9.14 (br s, 1H), 8.50 (s, 1H), 8.00 (br s, 1H), 7.67 (dt, J = 3.8, 6.2 Hz, 2H), 7.50 (br dd, J = 3.8, 5.7 Hz, 2H), 6.74 (d, J = 1.9 Hz, 2H), 6.34 (s, 1H), 4.11 (br s, 2H); LCMS: 100%, MS (ESI): m/z 395.0 [M + H]+. |
| 25 | 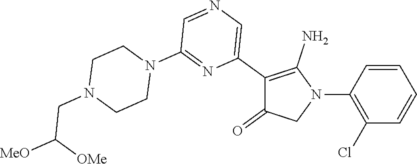 [SMILES: COC(CN1CCN(c2cncc(C3=C(N)N(c4ccccc4Cl)CC3=O)n2)CC1)OC] | Yellow solid; ¹H NMR (500 MHz, METHANOL-d4) δ = 8.92 (s, 1H), 7.84 (s, 1H), 7.67-7.62 (m. 1H), 7.60- 7.55 (m, 1H), 7.53-7.48 (m. 2H), 4.61 (s, 2H), 4.57 (t, J = 5.2 Hz, 1H), 4.26-4.18 (m, 1H), 4.15-4.07 (m, 1H), 3.54-3.49 (m, 4H), 3.37 (s, 6H), 2.71-2.67 (m, 4H), 2.58 (d, J = 5.2 Hz, 2H); LCMS: 99%, MS (ESI): m/z 459.1 [M + H]+. |
| 26 | 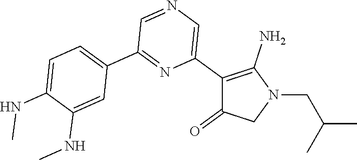 [SMILES: CNc1ccc(-c2cncc(C3=C(N)N(CC(C)C)CC3=O)n2)cc1NC] | Yellow solid; ¹H NMR (400 MHz, DMSO-d6) δ = 9.60- 9.39 (m, 1H), 9.33 (3, 1H), 8.51 (3, 1H), 8.14 (br s, 1H), 7.24 (dd, J = 1.8, 8.1 Hz, 1H), 6.95 (d, J = 1.7 Hz, 1H), 6.49 (d, J = 8.1 Hz, 1H), 5.10 (br d, J = 2.5 Hz, 1H), 4.94- 4.71 (m, 1H), 3.77 (s, 2H), 3.25 (br d, J = 7.5 Hz, 2H), 2.80 (br d, J = 12.5 Hz, 6H), 2.00 (s, 1H), 0.91 (d, J = 6.7 Hz, 6H); LCMS: 96.64%, MS (ESI): m/z 367.2 [M + H]+. |
| 27 | 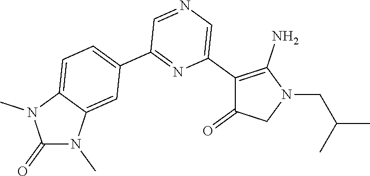 [SMILES: CC(C)CN1CC(=O)C(c2cncc(-c3ccc4c(c3)n(C)c(=O)n4C)n2)=C1N] | Yellow solid; ¹H NMR (400 MHz, DMSO-d6) δ = 9.49 (s, 1H), 9.40-8 98 (m, 1H), 8 65 (s, 1H), 8.48-7.90 (m, 1H), 7.73 (s, 1H), 7.69 (dd, J = 1.4, 8.1 Hz, 1H), 7.29 (d, J = 8.1 Hz, 1H), 3.79 (s, 2H), 3.42 (s, 3H), 3.38 (s, 3H), 3.27 (br d, J = 7.6 Hz, 2H), 2.13-1.91 (m, 1H), 0.91 (d, J = 6.6 Hz, 6H); LCMS: 100%, MS (ESI): m/z 393.1 [M + H]+. |
| 28 | 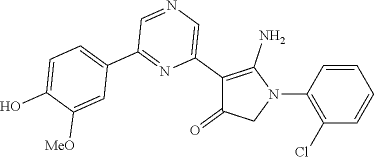 [SMILES: COc1cc(-c2cncc(C3=C(N)N(c4ccccc4Cl)CC3=O)n2)ccc1O] | Yellow solid; ¹H NMR (400 MHz, DMSO-d6) δ = 9.49 (br s, 1H), 9.48 (s, 1H), 9.27 (br s, 1H), 8.66 (s, 1H), 7.98 (br s, 1H), 7.73-7.64 (m, 2H), 7.57-7.49 (m, 2H), 7.47-7.40 (m, 2H), 6.89 (d, J = 8.2 Hz, 1H), 4.11 (br d, J = 2.6 Hz, 2H), 3.82 (s, 3H); LCMS: 100%, MS (ESI): m/z 409.1 [M + H]+. |
| 29 | 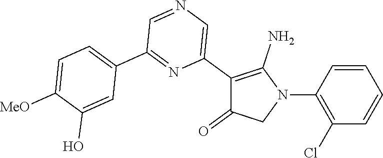 [SMILES: COc1ccc(-c2cncc(C3=C(N)N(c4ccccc4Cl)CC3=O)n2)cc1O] | Yellow solid; ¹H NMR (400 MHz, DMSO-d6) δ = 9.50 (s, 1H), 9.26 (s, 1H), 9.22-9.10 (m, 1H), 8.57 (s, 1H), 8.07-7.89 (m, 1H), 7.74-7.62 (m, 2H), 7.56-7.47 (m, 2H), 7.41-7.32 (m, 2H), 7.04 (d, J = 8.3 Hz, 1H), 4.11 (br d, J = 1.3 Hz, 2H), 3.83 (s, 3H); LCMS: 100%, MS (ESI): m/z 409.1 [M + H]+. |
| 30 | 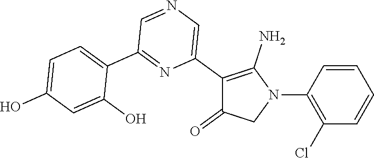 [SMILES: NC1=C(c2cncc(-c3ccc(O)cc3O)n2)C(=O)CN1c1ccccc1Cl] | Yellow solid; ¹H NMR (500 MHz, DMSO-d6) δ = 10.07 (s, 1H), 9.68 (s, 1H), 9.36 (s, 1H), 9.19 (br s, 1H), 8.58 (s, 1H), 7.84 (br s, 1H), 7.69-7.61 (m, 2H), 7.52-7.47 (m, 2H), 7.42 (d, J = 8.5 Hz, 1H), 6.44 (d, J = 2.1 Hz, 1H), 6.35 (dd, J = 2.3, 8.5 Hz, 1H), 4.08 (s, 2H), LCMS: 100%, MS (ESI): m/z 395.0 [M + H]+. |
| 31 | 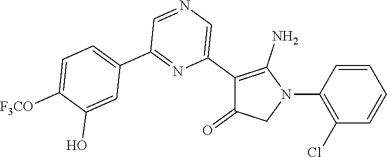 [SMILES: NC1=C(c2cncc(-c3ccc(OC(F)(F)F)c(O)c3)n2)C(=O)CN1c1ccccc1Cl] | White solid; ¹H NMR (400 MHz, DMSO-d6) δ = 10.51 (br s, 1H), 9.61 (s, 1H), 8.97 (s, 1H), 8.60 (s, 1H), 7.98 (s, 1H), 7.71-7.64 (m, 2H), 7.55 (s, 1H), 7.53-7.48 (m, 2H), 7.41 (s, 2H), 4.12 (br d, J = 2.0 Hz, 2H); LCMS: 96.6%, MS (ESI): m/z 463.1 [M + H]+. |
| 32 | 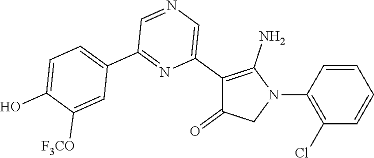 [SMILES: NC1=C(c2cncc(-c3ccc(O)c(OC(F)(F)F)c3)n2)C(=O)CN1c1ccccc1Cl] | Yellow solid; ¹H NMR (400 MHz, DMSO-d6) δ = 10.71 (br d, J = 3.2 Hz, 1H), 9.55 (s, 1H), 8.99 (br s, 1H), 8.66 (s, 1H), 8.19-7.93 (m, 1H), 7.84 (br d, J = 8.5 Hz, 1H), 7.79 (s, 1H), 7.73-7.62 (m, 2H), 7.56-7.46 (m, 2H), 7.15 (d, J = 8.5 Hz, 1H), 4.11 (br d, J = 3.3 Hz, 2H); LCMS: 100% MS (ESI): m/z 463.0 [M + H]+. |
| 33 | 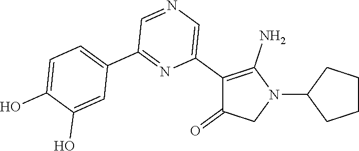 [SMILES: NC1=C(c2cncc(-c3ccc(O)c(O)c3)n2)C(=O)CN1C1CCCC1] | Yellow solid; ¹H NMR (500 MHz, DMSO-d6) δ = 9.50- 9.42 (m, 1H), 9.40 (s, 1H), 9.36-9.24 (m, 1H), 9.19 (s, 11), 8.44 (s, 1H), 8.26-8.00 (m, 1H), 7.32 (d, J = 2.5 Hz, 1H), 7.25 (d, J = 8.0 Hz, 1H), 6.87 (d, J = 8.0 Hz, 1H), 4.37-4.27 (m, 1H), 3.77 (s, 2H), 1.95-1.83 (m, 2H), 1.76-1.67 (m, 2H), 1.66-1.50 (m, 4H); LCMS: 100%, MS (ESI): m/z 353.1 [M + H]+. |
| 34 | 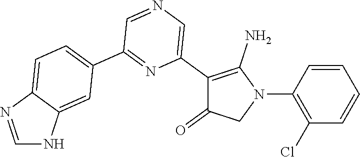 [SMILES: NC1=C(c2cncc(-c3ccc4nc[nH]c4c3)n2)C(=O)CN1c1ccccc1Cl] | Yellow solid; ¹H NMR (400 MHz, DMSO-d6) δ = 12.64 (br d, J = 19.2 Hz, 1H), 9.56 (d, J = 6.4 Hz, 1H), 9.45- 9.45 (m, 1H), 8.74 (d, J = 13.2 Hz, 1H), 8.31 (d, J = 1.6 Hz, 1H), 8.25-8.06 (m, 1H), 8.00 (br s, 1H), 7.82-7.55 (m, 4H), 7.53-7.48 (m, 2H), 4.13 (s, 2H); LCMS: 100%, MS (ESI): m/z 403.1 [M + H]+. |
| 35 | 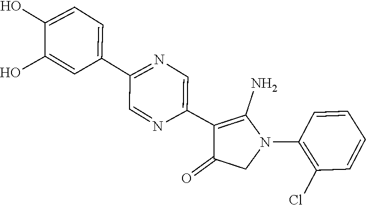 [SMILES: NC1=C(c2cnc(-c3ccc(O)c(O)c3)cn2)C(=O)CN1c1ccccc1Cl] | Yellow solid; ¹H NMR (400 MHz, DMSO-d6) δ = 9.58 (s, 1H), 9.36-9.09 (m, 2H), 9.04-8.92 (m, 1H), 8.78 (s, 1H), 7.84 (br s, 1H), 7.71-7.61 (m, 2H), 7.54-7.47 (m 3H), 7.34 (dd, J = 2.0, 8.4 Hz, 1H), 6.82 (d, J = 8.4 Hz, 1H), 4.08 (d, J = 4.8 Hz, 2H); LCMS: 98%, MS (ESI): m/z 395.0 [M + H]+. |
| 36 | 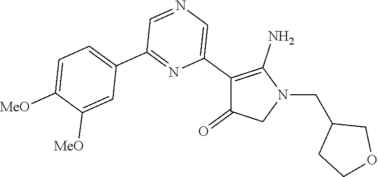 [SMILES: COc1ccc(-c2cncc(C3=C(N)N(CC4CCOC4)CC3=O)n2)cc1OC] | Yellow solid; ¹H NMR (400 MHz, DMSO-d6) δ = 9.39 (s, 1H), 9.35-9.10 (m, 1H), 8.71 (s, 1H), 8.66-8.42 (m, 1H), 7.56 (dd, J = 2.0, 8.4 Hz, 1H), 7.51 (d, J = 2.0 Hz, 1H), 7.11 (d, J = 8.8 Hz, 1H), 3.94 (s, 2H), 3.87 (s, 3H), 3.83 (s, 3H), 3.82-3.76 (m, 2H), 3.65 (d, J = 8.0 Hz, 1H), 3.50 (d, J = 7.6 Hz, 2H), 3.41 (dd, J = 2.4, 8.8 Hz, 1H), 2.71-2.58 (m, 1H), 2.09-1.94 (m, 1H), 1.65-1.51 (m, 1H); LCMS: 100%, MS (ESI): m/z 397.1 [M + H]+. |
| 37 | 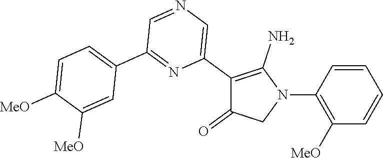 [SMILES: COc1ccc(-c2cncc(C3=C(N)N(c4ccccc4OC)CC3=O)n2)cc1OC] | Yellow solid; ¹H NMR (500 MHz, DMSO-d6) δ = 9.52 (s, 1H), 9.17 (br s, 1H), 8.67 (s, 1H), 7.71 (br s, 1H), 7.54 (dd, J = 2.0, 8.4 Hz, 1H), 7.47-7.42 (m, 3H), 7.22 (d, J = 6.4 Hz, 1H), 7.12-7.05 (m, 2H), 4.07 (s, 2H), 3.83 (s, 3H, 3.81 (s, 6H); LCMS: 98%, MS (ESI): m/z 419.1 [M + H]+. |
| 38 | 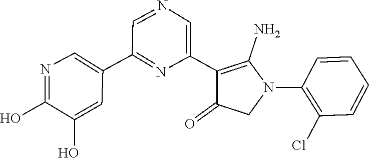 [SMILES: NC1=C(c2cncc(-c3cnc(O)c(O)c3)n2)C(=O)CN1c1ccccc1Cl] | Yellow solid; ¹H NMR (500 MHz, DMSO-d6) δ = 12.11 (br s, 1H), 9.47 (s, 1H), 9.30 (br s, 1H), 9.94 (br s, 1H), 8.57 (s, 1H), 8.05 (br s, 1H), 7.67 (ddd, J = 3.4, 6.1, 12.2 Hz, 2H), 7.59 (br s, 1H), 7.53-7.48 (m, 2H), 7.23 (d, J = 2.3 Hz, 1H), 4.17-4.02 (m, 2H); LCMS: 95.74%, MS (ESI): m/z 396.1 [M + H]+. |
| 39 | 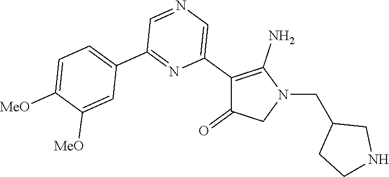 [SMILES: COc1ccc(-c2cncc(C3=C(N)N(CC4CCNC4)CC3=O)n2)cc1OC] | Orange solid; ¹H NMR (500 MHz, DMSO-d6) δ = 9.40 (s, 2H), 9.37-9.25 (m, 1H), 9.22-9.09 (m, 1H), 8.93- 8.74 (m, 1H), 8.71 (s, 1H), 7.56 (dd, J = 2.0, 8.0 Hz, 1H), 7.51 (d, J = 2.0 Hz, 1H), 7.12 (d, J = 8.5 Hz, 1H), 3.94 (d, J = 4.5 Hz, 2H), 3.87 (s, 3H), 3.83 (s, 3H), 3.55-3.51 (m, 2H), 3.39-3.24 (m, 2H), 3.21-3.06 (m, 1H), 3.00- 2.86 (m, 1H), 2.73-2.65 (m, 1H), 2.19-2.02 (m, 1H), 1.66 (dd, J = 7.5, 13.0 Hz, 1H); LCMS: 99%, MS (ESI): m/z 396.1 [M + H]+. |
| 40 | 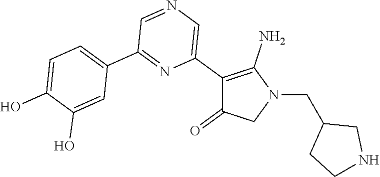 [SMILES: NC1=C(c2cncc(-c3ccc(O)c(O)c3)n2)C(=O)CN1CC1CCNC1] | Orange solid; ¹H NMR (500 MHz, DMSO-d6) δ = 9.53- 9.38 (m, 1H), 9.32 (s, 1H), 9.30-9.14 (m, 2H), 8.92- 8.70 (m, 1H), 8.57 (s, 1H), 7.37 (d, J = 2.0 Hz, 1H), 7.29 (dd, J = 2.0, 10.5 Hz, 1H), 6.90 (d, J = 8.0 Hz, 1H), 4.01 (s, 2H), 3.71 (dd, J = 6.5, 14.5 Hz, 1H), 3.59 (dd, J = 7.0, 14.5 Hz, 1H), 3.31 (d, J = 6.5 Hz, 2H), 3.18-3.07 (m, 1H), 2.97-2.87 (m, 1H), 2.74-2.64 (m, 1H), 2.15- 2.05 (m, 1H), 1.72-1.60 (m, 1H); LCMS: 99%, MS (ESI): m/z 368.1 [M + H]+. |
| 41 | 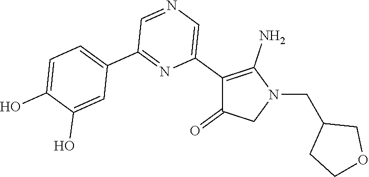 [SMILES: NC1=C(c2cncc(-c3ccc(O)c(O)c3)n2)C(=O)CN1CC1CCOC1] | Yellow solid; ¹H NMR (400 MHz, DMSO-d6) δ = 9.42 (s, 1H), 9.41 (s, 1H), 9.38-9.20 (m, 1H), 9.18 (s, 1H), 8.44 (s, 1H), 8.37-8.07 (m, 1H), 7.32 (d, J = 2.0 Hz, 1H), 7.25 (dd, J = 2.0, 8.4 Hz, 1H), 6.87 (d, J = 2.0 Hz, 1H), 3.84-3.75 (m, 4H), 3.65 (q, J = 7.6 Hz, 1H), 3.46 (d, J = 7.6 Hz, 2H), 3.43-3.39 (m, 1H), 2.64-2.59 (m, 1H), 2.02-1.98 (m, 1H), 1.61-1.52 (m, 1H); LCMS: 100%, MS (ESI): m/z 369.1 [M + H]+. |
| 42 | 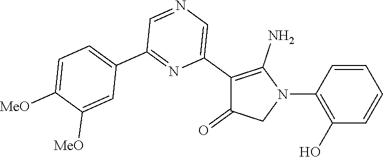 [SMILES: COc1ccc(-c2cncc(C3=C(N)N(c4ccccc4O)CC3=O)n2)cc1OC] | Brick red solid; ¹H NMR (400 MHz, DMSO-d6) δ = 10.12 (br s, 1H), 9.50 (s, 1H), 9.13 (br s, 1H), 8.70 (s, 1H), 7.79-7.64 (m, 1H), 7.56 (dd, J = 2.0, 8.4 Hz, 1H), 7.46 (d, J = 2.0 Hz, 1H), 7.36 (dd, J = 1.6, 7.6 Hz, 1H), 7.31-7.24 (m, 1H), 7.06 (d, J = 8.4 Hz, 1H), 7.06-7.01 (m, 1H), 6.96-6.90 (m, 1H), 4.09 (s, 2H), 3.82 (s, 6H); LCMS: 100%, MS (ESI): m/z 405.1 [M + H]+. |
| 43 | 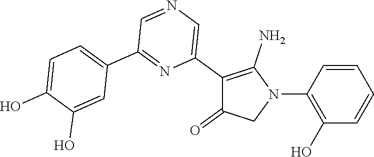 [SMILES: NC1=C(c2cncc(-c3ccc(O)c(O)c3)n2)C(=O)CN1c1ccccc1O] | Brick red solid; ¹H NMR (400 MHz, DMSO-d6) δ = 10.08 (br s, 1H), 9.45 (s, 1H), 9.08 (br s, 1H), 8.50 (s, 1H), 7.61 (br s, 1H), 7.35 (dd, J = 1.6, 7.6 Hz, 1H), 7.32 (d, J = 2.0 Hz, 1H), 7.29-7.22 (m, 2H), 7.02 (dd, J = 1.2, 8.4 Hz, 1H), 6.92 (t, J = 8.4 Hz, 1H), 6.85 (d, J = 8.4 Hz, 1H), 4.08 (s, 2H); LCMS: 96%, MS (ESI): m/z 377.1 [M + H]+. |
| 44 | 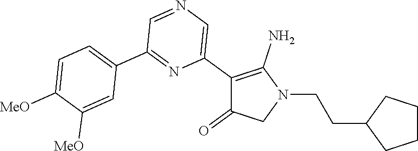 [SMILES: COc1ccc(-c2cncc(C3=C(N)N(CCC4CCCC4)CC3=O)n2)cc1OC] | Orange solid; ¹H NMR (400 MHz, DMSO-d6) δ = 9.45 (s, 1H), 9.36-9.06 (m, 1H), 8.61 (s, 1H), 8.33-8.00 (m, 1H), 7.53 (dd, J = 2.0, 8.4 Hz, 1H), 7.49 (d, J = 2.0 Hz, 1H), 7.10 (d, J = 8.4 Hz, 1H), 3.87 (s, 3H), 3.83 (s, 3H), 3.78 (s, 2H), 3.47-3.38 (m, 2H), 1.84-1.71 (m, 3H), 1.66-1.54 (m, 4H), 1.54-1.43 (m, 2H), 1.24-1.09 (m, 2H); LCMS: 100%, MS (ESI): m/z 409.2 [M + H]+. |
| 45 | 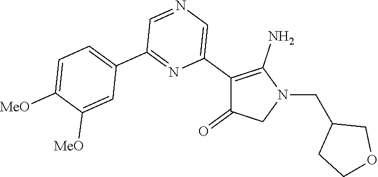 [SMILES: COc1ccc(-c2cncc(C3=C(N)N(CC4CCOC4)CC3=O)n2)cc1OC] | Yellow solid; ¹H NMR (400 MHz, DMSO-d6) δ = 8.57 (d, J = 1.6 Hz, 1H), 8.50 (d, J = 2.0 Hz, 1H), 7.95 (s, 1H), 7.34 (s, 2H), 7.27 (d, J = 2.0 Hz, 1H), 7.23 (dd, J = 2.0, 8.0 Hz, 1H), 7.06 (d, J = 8.4 Hz, 1H), 3.84 (s, 3H), 3.80 (s, 3H), 3.79-3.76 (m, 1H), 3.72 (s, 2H), 3.68- 3.62 (m, 1H), 3.43-3.38 (m, 4H), 2.57 (d, J = 7.6 Hz, 1H), 2.05-1.93 (m, 1H), 1.62-1.51 (m, 1H); LCMS: 100%, MS (ESI): m/z 396.1 [M + H]+. |
| 46 | 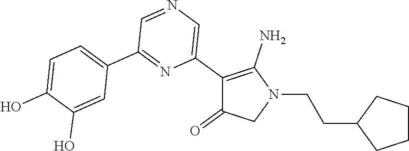 [SMILES: NC1=C(c2cncc(-c3ccc(O)c(O)c3)n2)C(=O)CN1CCC1CCCC1] | off-white solid; ¹H NMR (500 MHz, DMSO-d6) δ = 9.56-9.34 (m, 2H), 9.33-9.04 (m, 2H), 8.44 (s, 1H), 8.26-8.02 (m, 1H), 7.33 (d, J = 2.0 Hz, 1H), 7.25 (dd, J = 2.0, 8.0 Hz, 1H), 6.86 (d, J = 8.0 Hz, 1H), 3.77 (s, 2H), 3.46-3.40 (m, 2H), 1.81-1.71 (m, 3H), 1.64-1.54 (m, 4H), 1.53-1.43 (m, 2H), 1.15 (d, J = 3.5 Hz, 2H); LCMS: 100%, MS (ESI): m/z 381.1 [M + H]+. |
| 47 | 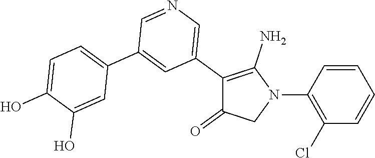 [SMILES: NC1=C(c2cncc(-c3ccc(O)c(O)c3)c2)C(=O)CN1c1ccccc1Cl] | Yellow solid; ¹H NMR (400 MHz, DMSO-d6) δ = 9.13 (br s, 1H), 9.10 (br s, 1H), 8.58 (d, J = 2.0 Hz, 1H), 8.44 (d, J = 2.0 Hz, 1H), 7.98 (t, J = 2.4 Hz, 1H), 7.64 (d, J = 9.6 Hz, 1H), 7.60-7.55 (m, 1H), 7.50-7.41 (m, 2H), 7.28 (br s, 2H), 7.10 (d, J = 2.4 Hz, 1H), 7.00 (dd, J = 2.4, 8.4 Hz, 1H), 6.85 (d, J = 8.0 Hz, 1H), 4.00 (br d, J = 9.6 Hz, 2H); LCMS: 100%, MS (ESI): m/z 394.0 [M + H]+. |
| 48 | 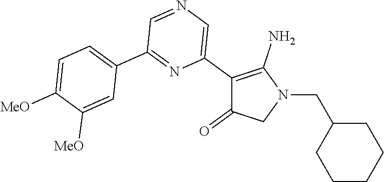 [SMILES: COc1ccc(-c2cncc(C3=C(N)N(CC4CCCCC4)CC3=O)n2)cc1OC] | Yellow sold; ¹H NMR (500 MHz, DMSO-d6) δ = 9.46 (s, 1H), 9.37-9.14 (m, 1H), 8.61 (s, 1H), 8.20-8.05 (m, 1H), 7.53 (dd, J = 2.0, 8.5 Hz, 1H), 7.49 (d, J = 2.0 Hz, 1H), 7.10 (d, J = 8.5 Hz, 1H), 3.87 (s, 3H), 3.83 (s, 3H), 3.78 (s, 2H), 3.27 (d, J = 7.0 Hz, 2H), 1.77-1.00 (m, 6H), 1.27-1.08 (m, 3H), 1.03-0.92 (m, 2H); LCMS: 100%, MS (ESI): m/z 409.3 [M + H]+. |
| 49 | 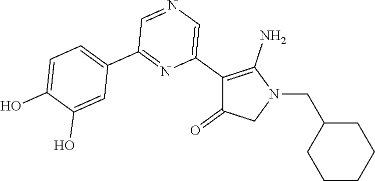 [SMILES: NC1=C(c2cncc(-c3ccc(O)c(O)c3)n2)C(=O)CN1CC1CCCCC1] | Yellow solid; ¹H NMR (500 MHz, DMSO-d6) δ = 9.41 (s, 2H), 9.35-9.20 (m, 1H), 9.18 (br s, 1H), 8.43 (s, 1H), 8.20-7.99 (m, 1H), 7.32 (d, J = 2.0 Hz, 1H), 7.25 (dd, J = 2.0, 8.0 Hz 1H), 6.86 (d, J = 8.5 Hz, 1H), 3.78 (s, 2H), 3.28 (d, J = 7.0 Hz, 2H), 1.73-1.59 (m, 6H), 1.24- 1.10 (m, 3H), 1.03-0.92 (m, 2H); LCMS: 100%, MS (ESI): m/z 381.2 [M + H]+. |
| 50 | 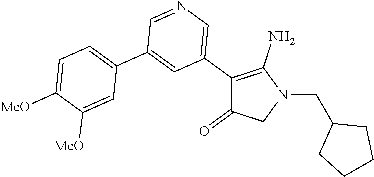 [SMILES: COc1ccc(-c2cncc(C3=C(N)N(CC4CCCC4)CC3=O)c2)cc1OC] | off-white solid; ¹H NMR (400 MHz, DMSO-d6) δ = 8.58 (br s, 1H), 8.49 (br s, 1H), 7.96 (s, 1H), 7.28 (s, 2H), 7.27-7.26 (m, 1H), 7.22 (dd, J = 2.0, 8.0 Hz, 1H), 7.06 (d, J = 8.4 Hz, 1H), 3.64 (s, 3H), 3.80 (s, 3H), 3.70 (s, 2H), 3.37-3.36 (m, 2H), 2.25-2.16 (m, 1H), 1.75- 1.69 (m, 2H), 1.66-1.58 (m, 2H), 1.55-1.48 (m, 2H), 1.22-1.14 (m, 2H); LCMS: 100%, MS (ESI): m/z 394.1 [M + H]+. |
| 51 | 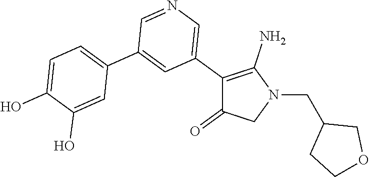 [SMILES: NC1=C(c2cncc(-c3ccc(O)c(O)c3)c2)C(=O)CN1CC1CCOC1] | Yellow solid; ¹H NMR (400 MHz, DMSO-d6) δ = 9.12 (br s, 1H), 9.09 (br s, 1H), 8.49 (d, J = 2.0 Hz, 1H), 8.38 (d, J = 2.0 Hz, 1H), 7.92 (t, J = 2.0 Hz, 1H), 7.31 (s, 2H), 7.06 (d, J = 2.0 Hz, 1H), 6.97 (dd, J = 2.4, 8.4 Hz, 1H), 6.83 (d, J = 8.0 Hz, 1H), 3.84-3.75 (m, 2H), 3.72 (s, 2H), 3.65 (q, J = 7.6 Hz, 1H), 3.41 (br d, J = 8.0 Hz, 3H), 2.60-2.54 (m, 1H), 2.01-1.95 (m, 1H), 1.59-1.52 (m, 1H); LCMS: 100%, MS (ESI): m/z 368.1 [M + H]+. |
| 52 | 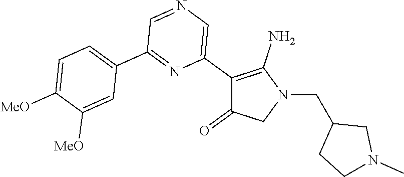 [SMILES: COc1ccc(-c2cncc(C3=C(N)N(CC4CCN(C)C4)CC3=O)n2)cc1OC] | Yellow solid; ¹H NMR (400 MHz, METHANOL-d4) δ = 9.43 (s, 1H), 8.51-8.45 (m, 1H), 7.50 (dd, J = 2.0, 8.4 Hz, 1H), 7.47 (d, J = 2.0 Hz, 1H), 7.08 (d, J = 8.4 Hz, 1H), 3.92 (d, J = 8.4 Hz, 8H), 3.58 (d, J = 7.2 Hz, 2H), 3.43-3.33 (m, 2H), 3.27-3.18 (m, 1H), 3.07-2.96 (m, 1H), 2.94-2.86 (m, 1H), 2.83 (s, 3H), 2.38-2.21 (m, 1H), 1.91-1.73 (m, 1H); LCMS: 96%, MS (ESI): m/z 410.3 [M + H]+. |
| 53 | 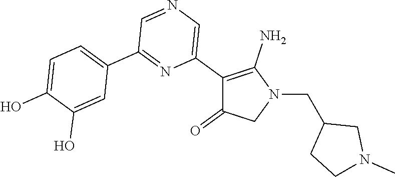 [SMILES: CN1CCC(CN2CC(=O)C(c3cncc(-c4ccc(O)c(O)c4)n3)=C2N)C1] | Yellow solid; ¹H NMR (500 MHz, METHANOL-d4) δ = 9.37 (s, 1H), 8.43 (s, 1H), 7.34 (d, J = 2.0 Hz, 1H), 7.27 (dd, J = 2.0, 8.0 Hz, 1H), 6.89 (d, J = 8.0 Hz, 1H), 3.91 (d, J = 3.5 Hz, 2H), 3.64-3.58 (m 2H), 3.53-3.47 (m, 1H), 3.46-3.39 (m, 1H), 3.37-3.33 (m, 1H), 3.11 (br s 1H), 2.90 (s, 3H), 2.98-2.87 (m, 1H), 2.38-2.27 (m, 1H), 1.93-1.81 (m, 1H), LCMS: 98%, MS (ESI): m/z 382.3 [M + H]+. |
| 54 | 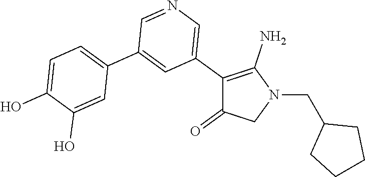 [SMILES: NC1=C(c2cncc(-c3ccc(O)c(O)c3)c2)C(=O)CN1CC1CCCC1] | Yellow solid; ¹H NMR (400 MHz, DMSO-d6) δ = 9.13 (s, 1H), 9.10 (s, 1H), 8.49 (d, J = 1.6 Hz, 1H), 8.38 (d, J = 2.0 Hz, 1H), 7.93 (s, 1H), 7.26 (s, 2H), 7.06 (d, J = 2.4 Hz, 1H), 6.97 (dd, J = 2.0, 8.0 Hz, 1H), 6.83 (d, J = 8.4 Hz, 1H), 3.70 (s, 2H), 3.32 (br s, 2H), 2.26-2.14 (m, 1H), 1.77-1.67 (m, 2H), 1.66-1.57 (m, 2H), 1.56-1.48 (m, 2H), 1.24-1.18 (m, 2H); LCMS: 100%, MS (ESI): m/z 366.1 [M + H]+. |
| 55 | 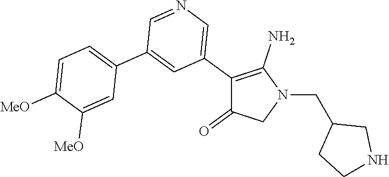 [SMILES: COc1ccc(-c2cncc(C3=C(N)N(CC4CCNC4)CC3=O)c2)cc1OC] | Yellow gum; ¹H NMR (400 MHz, DMSO-d6) δ = 8.57 (d, J = 2.0 Hz, 1H), 8.51 (d, J = 2.0 Hz, 1H), 7.95 (t, J = 4.0 Hz, 1H), 7.44 (br s, 2H), 7.27 (d, J = 2.0 Hz, 1H), 7.23 (dd, J = 2.0, 8.4 Hz, 1H), 7.06 (d, J = 8.0 Hz, 1H), 3.84 (s, 3H), 3.80 (s, 3H), 3.73 (d, J = 4.4 Hz, 2H), 3.50- 3.44 (m, 1H), 3.43-3.39 (m, 1H), 3.24-3.18 (m, 2H), 3.08-2.98 (m, 1H), 2.85-2.76 (m, 1H), 2.61-2.54 (m, 1H), 2.03-1.98 (m, 1H), 1.59-1.53 (m, 1H); LCMS: 95% MS (ESI): m/z 395.2 [M + H]+. |
| 56 | 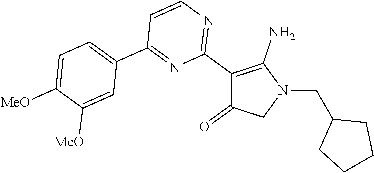 [SMILES: COc1ccc(-c2ccnc(C3=C(N)N(CC4CCCC4)CC3=O)n2)cc1OC] | Yellow solid; ¹H NMR (400 MHz, DMSO-d6) δ = 9.52- 9.18 (m, 1H), 8.53 (d, J = 5.2 Hz, 1H), 8.09-7.87 (m, 1H), 7.83 (d, J = 1.6 Hz, 1H), 7.75 (dd, J = 2.0, 8.4 Hz, 1H), 7.43 (d, J = 5.2 Hz, 1H), 7.09 (d, J = 8.8 Hz, 1H), 3.86 (s, 3H), 3.84 (s, 3H), 3.70 (s, 2H), 3.51-3.42 (m, 2H), 2.30-2.23 (m, 1H), 1.77-1.68 (m, 2H), 1.67-1.58 (m, 2H), 1.58-1.46 (m, 2H), 1.28-1.15 (m, 2H); LCMS: 99%, MS (ESI): m/z 395.1 [M + H]+. |
| 57 | 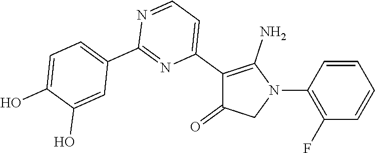 [SMILES: NC1=C(c2ccnc(-c3ccc(O)c(O)c3)n2)C(=O)CN1c1ccccc1F] | Gray solid; ¹H NMR (400 MHz, DMSO-d6) δ = 9.66 (br s, 1H), 9.43 (br s, 1H), 9.16 (br s, 1H), 8.50 (d, J = 5.6 Hz, 1H), 8.22 (br s, 1H), 8.16-8 11 (m, 1H), 7.73-7.59 (m, 2H), 7.53-7.41 (m, 3H), 7.35 (dt, J = 1.2, 7.6 Hz, 1H), 6.82 (d, J = 8.0 Hz, 1H), 4.18 (s, 2H); LCMS: 98%, MS (ESI): m/z 379.1 [M + H]+. |
| 58 | 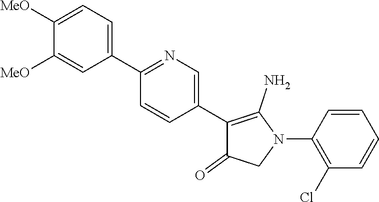 [SMILES: COc1ccc(-c2ccc(C3=C(N)N(c4ccccc4Cl)CC3=O)cn2)cc1OC] | Yellow solid; ¹H NMR (400 MHz, DMSO-d6) δ = 8.75 (d, J = 2.0 Hz, 1H), 7.97 (d, J = 10.8 Hz, 1H), 7.87 (d, J = 8.4 Hz, 1H), 7.72 (d, J = 1.6 Hz, 1H), 7.67-7.60 (m, 2H), 7.59-7.54 (m, 1H), 7.51-7.42 (m, 2H), 7.30-7.20 (m, 2H), 7.05 (d, J = 8.8 Hz, 1H), 4.00 (d, J = 10.0 Hz, 2H), 3.85 (s, 3H), 3.81 (s, 3H); LCMS: 90%, MS (ESI): m/z 422.1 [M + H]+. |
| 59 | 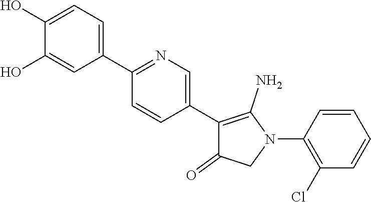 [SMILES: NC1=C(c2ccc(-c3ccc(O)c(O)c3)nc2)C(=O)CN1c1ccccc1Cl] | Yellow solid; ¹H NMR (400 MHz, DMSO-d6) δ = 9.10 (br s, 1H), 9.04 (br s, 1H), 8.69 (d, J = 1.6 Hz, 1H), 7.90 (dd, J = 2.4, 8.4 Hz, 1H), 7.69 (d, J = 8.0 Hz, 1H), 7.66- 7.60 (m, 1H), 7.59-7.52 (m, 2H), 7.47-7.43 (m, 2H), 7.35 (dd, J = 2.4, 8.0 Hz, 1H), 7.22 (br s, 2H), 6.80 (d, J = 8.4 Hz, 1H), 3.98 (d, J = 10.0 Hz, 2H); LCMS: 100%, MS (ESI): m/z 394.0 [M + H]+. |
| 60 | 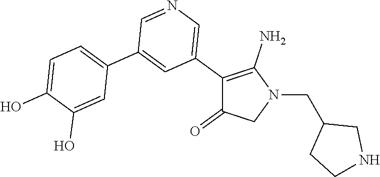 [SMILES: NC1=C(c2cncc(-c3ccc(O)c(O)c3)c2)C(=O)CN1CC1CCNC1] | Yellow solid; ¹H NMR (400 MHz, METHANOL-d4) δ = 8.90 (s, 1H), 8.83 (s, 1H), 8.79 (d, J = 2.0 Hz, 1H), 7.28 (d, J = 2.0 Hz, 1H), 7.22 (dd, J = 2.4, 8.0 Hz, 1H), 6.98 (d, J = 8.0 Hz, 1H), 4.42 (s, 2H), 3.79 (d, J = 6.8 Hz, 2H), 3.69-3.61 (m, 1H), 3.56-3.50 (m, 1H), 3.41- 3.34 (m, 1H), 3.15-3.06 (m, 1H), 2.98-2.84 (m, 1H), 2.45-2.31 (m, 1H), 1.98-1.84 (m, 1H); LCMS: 94%, MS (ESI): m/z 367.1 [M + H]+. |
| 61 | 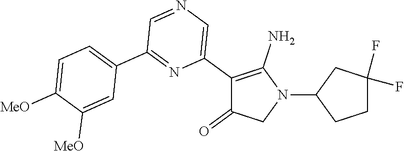 [SMILES: COc1ccc(-c2cncc(C3=C(N)N(C4CCC(F)(F)C4)CC3=O)n2)cc1OC] | Yellow solid; ¹H NMR (500 MHz, DMSO-d6) δ = 9.45 (s, 1H), 9.42-9.15 (m, 1H), 8.63 (s, 1H), 8.48-8.23 (m, 1H), 7.53 (dd, J = 2.0, 8.0 Hz, 1H), 7.48 (d, J = 2.0 Hz, 1H), 7.10 (d, J = 8.5 Hz, 1H), 4.68-4.51 (m, 1H), 3.87 (s, 3H), 3.83 (s, 5H), 2.49-2.41 (m, 1H), 2.40-2.21 (m, 2H), 2.17-2.01 (m, 2H), 2.01-1.88 (m, 1H); LCMS: 100%, MS (ESI): m/z 417.2 [M + H]+. |
| 62 | 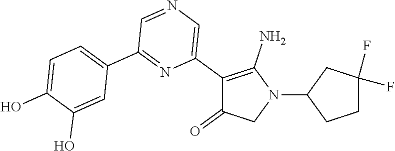 [SMILES: NC1=C(c2cncc(-c3ccc(O)c(O)c3)n2)C(=O)CN1C1CCC(F)(F)C1] | Yellow solid; ¹H NMR (400 MHz, DMSO-d6) δ = 9.40 (s, 2H), 9.32-9.08 (m, 1H), 8.46 (s, 1H), 8.38-8.20 (m, 1H), 7.32 (d, J = 1.6 Hz, 1H), 7.28-7.21 (m, 1H), 6.87 (d, J = 8.4 Hz, 1H), 4.68-4.52 (m, 1H), 3.83 (s, 2H), 2.47-2.41 (m, 1H), 2.37-2.26 (m, 2H), 2.17-2.01 (m, 2H), 2.00-1.90 (m, 1H); LCMS: 100%, MS (ESI): m/z 389.1 [M + H]+. |
| 63 | 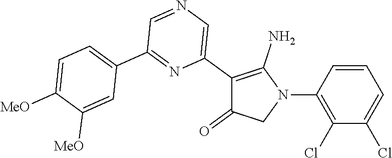 [SMILES: COc1ccc(-c2cncc(C3=C(N)N(c4cccc(Cl)c4Cl)CC3=O)n2)cc1OC] | Orange solid; ¹H NMR (500 MHz, DMSO-d6) δ = 9.51 (s, 1H), 9.24 (br s, 1H), 8.72 (s, 1H), 8.13 (br s, 1H), 7.79 (dd, J = 2.0, 8.0 Hz, 1H), 7.68 (d, J = 8.0 Hz, 1H), 7.57-7.51 (m, 2H), 7.47 (d, J = 2.0 Hz, 1H), 7.09 (d, J = 8.0 Hz, 1H), 4.13 (d, J = 5.5 Hz, 2H), 3.82 (s, 6H); LCMS: 99%, MS (ESI): m/z 457.0 [M + H]+. |
| 64 | 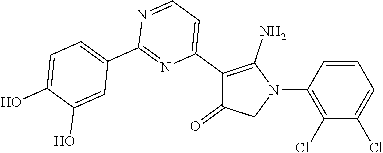 [SMILES: NC1=C(c2ccnc(-c3ccc(O)c(O)c3)n2)C(=O)CN1c1cccc(Cl)c1Cl] | Yellow solid; ¹H NMR (500 MHz, DMSO-d6) δ = 9.52- 9.37 (m, 2H), 9.20 (br s, 2H), 6.53 (s, 1H), 8.09 (s, 1H), 7.77 (dd, J = 1.5, 8.0 Hz, 1H), 7.67 (d, J = 1.5, 7.5 Hz, 1H), 7.57-7.46 (m, 1H), 7.32 (d, J = 2.0 Hz, 1H), 7.25 (dd, J = 2.0, 8.0 Hz, 1H), 6.85 (d, J = 8.0 Hz, 1H), 4.12 (d, J = 6.5 Hz, 2H); LCMS: 98%, MS (ESI): m/z 429.0 [M + H]+. |
| 65 | 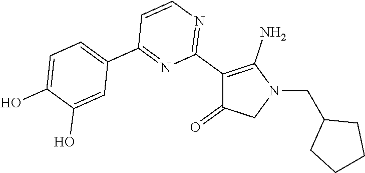 [SMILES: NC1=C(c2nccc(-c3ccc(O)c(O)c3)n2)C(=O)CN1CC1CCCC1] | Yellow sold; ¹H NMR (500 MHz, DMSO-d6) δ = 9.89- 9.48 (m, 1H), 9.46-9.01 (m, 2H), 8.49 (br s, 1H), 8.26- 8.01 (m, 1H), 7.57 (s, 1H), 7.49 (dd, J = 2.0, 9.0 Hz, 1H), 7.32 (br s, 1H), 6.87 (d, J = 8.5 Hz, 1H), 3.75 (br s, 2H), 3.39 (br s, 2H), 2.30-2.23 (m, 1H), 1.77-1.67 (m, 2H), 1.67-1.59 (m, 2H), 1.57-1.46 (m, 2H), 1.27-1.15 (m, 2H); LCMS: 98%, MS (ESI): m/z 367.1 [M + H]+. |
| 66 | 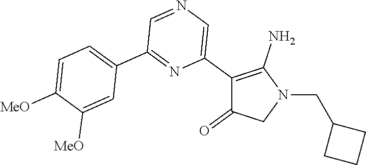 [SMILES: COc1ccc(-c2cncc(C3=C(N)N(CC4CCC4)CC3=O)n2)cc1OC] | Yellow solid; ¹H NMR (400 MHz, DMSO-d6) δ = 9.45 (s, 1H) 9.37-9.04 (m, 1H), 8.61 (s, 1H), 8.37-8.16 (m, 1H), 7.52 (d, J = 8.4 Hz, 1H), 7.49 (s, 1H), 7.09 (d, J = 8.4 Hz, 1H), 3.87 (s, 3H), 3.83 (s, 3H), 3.74 (s, 2H), 3.46 (d, J = 7.6 Hz, 2H), 2.72-2.60 (m, 1H), 2.09-1.98 (m, 2H), 1.94-1.82 (m, 2H), 1.81-1.69 (m, 2H); LCMS: 100%, MS (ESI): m/z 381.2 [M + H]+. |
| 67 | 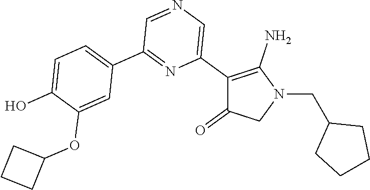 [SMILES: NC1=C(c2cncc(-c3ccc(O)c(OC4CCC4)c3)n2)C(=O)CN1CC1CCCC1] | Yellow solid; ¹H NMR (500 MHz, DMSO-d6) δ = 9.43 (s, 1H), 9.41 (br s, 1H), 9.31-9.10 (m, 1H), 8.51 (s, 1H), 8.37-8.03 (m, 1H), 7.37 (dd, J = 2.0, 10 Hz, 1H), 7.28 (s, 1H), 6.91 (d, J = 8.0 Hz, 1H), 4.77 (t, J = 7.0 Hz, 1H), 3.79 (s, 2H), 3.38 (br s, 2H), 2.52 (d, J = 2.0 Hz, 1H), 2.46-2.40 (m, 2H), 2.30-2.20 (m, 1H), 2.18-2.08 (m, 2H), 1.86-1.78 (m, 1H), 1.75-1.70 (m, 2H), 1.67-1.61 (m, 2H), 1.57-1.49 (m, 2H), 1.25-1.16 (m, 2H); LCMS: 96%, MS (ESI): m/z 421.2 [M + H]+. |
| 68 | 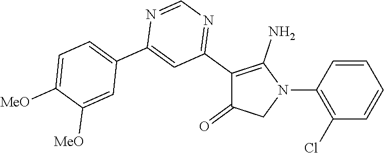 [SMILES: COc1ccc(-c2cc(C3=C(N)N(c4ccccc4Cl)CC3=O)ncn2)cc1OC] | Yellow solid; ¹H NMR (500 MHz, DMSO-d6) δ = 9.37 (br s, 1H), 8.89 (s, 1H), 8.80 (d, J = 1.5 Hz, 1H), 8.00 (br s, 1H), 7.72 (d, J = 2.0 Hz, 1H), 7.71-7.68 (m, 1H), 7.68-7.64 (m, 1H), 7.61 (dd, J = 2.0, 8.5 Hz, 1H), 7.55- 7.50 (m, 2H), 7.15 (d, J = 8.5 Hz, 1H), 4.11 (br d, J = 19.0 Hz, 2H), 3.86 (s, 3H), 3.84 (s, 3H); LCMS: 99%, MS (ESI): m/z 423.0 [M + H]+. |
| 69 | 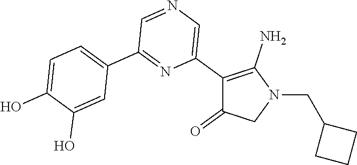 [SMILES: NC1=C(c2cncc(-c3ccc(O)c(O)c3)n2)C(=O)CN1CC1CCC1] | Gray solid; ¹H NMR (400 MHz, DMSO-d6) δ = 9.40 (s, 1H), 9.37-9.10 (m, 2H), 8.43 (s, 1H), 8.38-7.98 (m, 1H), 7.32 (d, J = 2.0 Hz, 1H), 7.24 (dd, J = 2.0, 8.0 Hz, 1H), 6.86 (d, J = 8.4 Hz, 1H), 3.74 (s, 2H), 3.47 (d, J = 7.2 Hz, 2H), 2.70-2.59 (m, 1H), 2.11-1.97 (m, 2H), 1.93-1.83 (m, 2H), 1.81-1.70 (m, 2H); LCMS: 100%, MS (ESI): m/z 353.1 [M + H]+. |
| 70 | 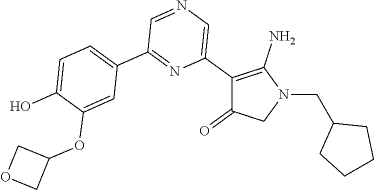 [SMILES: NC1=C(c2cncc(-c3ccc(O)c(OC4COC4)c3)n2)C(=O)CN1CC1CCCC1] | off-white solid; ¹H NMR (400 MHz, DMSO-d6) δ = 9.46 (s, 1H), 9.31-9.03 (m, 1H), 8.55 (s, 1H), 8.38- 7.97 (m, 1H), 7.47-7.42 (m, 2H), 7.02 (d, J = 8.0 Hz, 1H), 5.10 (t, J = 5.2 Hz, 1H), 4.40 (dd, J = 1.6, 11.2 Hz, 1H), 4.26-4.19 (m, 1H), 4.07 (dd, J = 7.6, 11.6 Hz, 1H), 3.80 (s, 2H), 3.74-3.60 (m, 2H), 3.36 (d, J = 8.0 Hz, 2H), 2.30-2.19 (m, 1H), 1.77-1.67 (m, 2H), 1.67-1.59 (m, 2H), 1.58-1.46 (m, 2H), 1.26-1.16 (m, 2H); LCMS: 97%, MS (ESI): m/z 423.3 [M + H]+. |
| 71 | 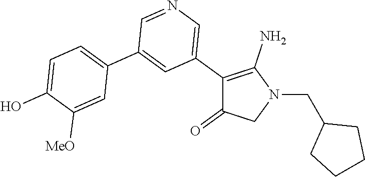 [SMILES: COc1cc(-c2cncc(C3=C(N)N(CC4CCCC4)CC3=O)c2)ccc1O] | Yellow solid; ¹H NMR (400 MHz, DMSO-d6) δ = 9.18 (br s, 1H), 8.55 (d, J = 2.0 Hz, 1H), 8.47 (d, J = 2.0 Hz, 1H), 7.93 (t, J = 2.0 Hz, 1H), 7.27 (s, 2H), 7.24 (d, J = 2.0 Hz, 1H), 7.10 (dd, J = 2.0, 8.2 Hz, 1H), 6.88 (d, J = 8.4 Hz, 1H), 3.85 (s, 3H), 3.70 (s, 2H), 3.32-3.31 (m, 2H), 2.27-2.14 (m, 1H), 1.77-1.67 (m, 2H), 1.67-1.59 (m, 2H), 1.53-1.50 (m, 2H), 1.26-1.19 (m, 2H); LCMS: 96%, MS (ESI): m/z 380.1 [M + H]+. |
| 72 | 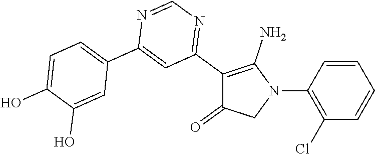 [SMILES: NC1=C(c2cc(-c3ccc(O)c(O)c3)ncn2)C(=O)CN1c1ccccc1Cl] | Yellow solid; ¹H NMR (400 MHz, DMSO-d6) δ = 9.40 (br s, 2H), 8.83 (s, 1H), 8.71 (s, 1H), 7.94 (br s, 1H), 7.74-7.61 (m, 2H), 7.56 (d, J = 2.0 Hz, 1H), 7.52 (dd, J = 4.0. 5.6 Hz, 2H), 7.44 (dd, J = 2.0, 8.4 Hz, 1H), 6.87 (d, J = 8.0 Hz, 1H), 4.10 (d, J = 10.8 Hz, 2H); LCMS: 99%, MS (ESI): m/z 395.0 [M + H]+. |
| 73 | 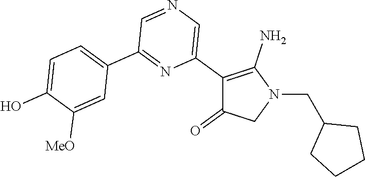 [SMILES: COc1cc(-c2cncc(C3=C(N)N(CC4CCCC4)CC3=O)n2)ccc1O] | Yellow solid; ¹H NMR (500 MHz, DMSO-d6) δ = 9.42 (s, 1H), 9.39-9.17 (m, 1H), 8.57 (s, 1H), 8.36-8.02 (m, 1H), 7.47 (d, J = 2.0 Hz, 1H), 7.41 (dd, J = 2.0, 8.0 Hz, 1H), 6.91 (d, J = 8.0 Hz, 1H), 3.88 (s, 3H), 3.79 (s, 2H), 3.37 (br s, 2H), 2.30-2.20 (m, 1H), 1.79-1.69 (m, 2H), 1.68-1.59 (m, 2H), 1.58-1.46 (m, 2H), 1.26-1.18 (m, 2H); LCMS: 100%, MS (ESI): m/z 381.2 [M + H]+. |
| 74 | 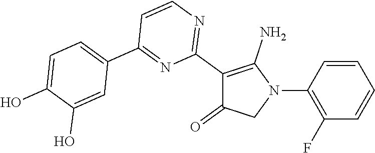 [SMILES: NC1=C(c2nccc(-c3ccc(O)c(O)c3)n2)C(=O)CN1c1ccccc1F] | Orange solid; ¹H NMR (400 MHz, DMSO-d6) δ = 9.55 (br s, 1H), 9.21 (br s, 1H), 8.55 (d, J = 5.6 Hz, 1H), 7.65- 7.58 (m, 2H), 7.52-7.39 (m, 3H), 7.37-7.30 (m, 2H), 6.85 (d, J = 8.4 Hz, 1H), 4.05 (s, 2H); LCMS: 79%, MS (ESI): m/z 379.1 [M + H]+. |
| 75 | 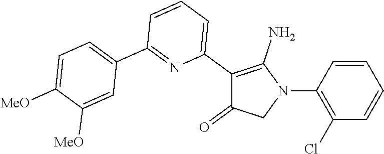 [SMILES: COc1ccc(-c2cccc(C3=C(N)N(c4ccccc4Cl)CC3=O)n2)cc1OC] | Yellow solid; ¹H NMR (400 MHz, DMSO-d6) δ = 9.91 (br s, 1H), 8.37 (d, J = 7.6 Hz, 1H), 7.85 (br s, 1H), 7.73 (t, J = 8.0 Hz, 1H), 7.71-7.61 (m, 2H), 7.55-7.48 (m, 2H), 7.48-7.39 (m, 3H), 7.04 (d, J = 8.4 Hz, 1H), 4.05 (s, 2H), 3.80 (s, 6H); LCMS: 99%, MS (ESI): m/z 422.1 [M + H]+. |
| 76 | 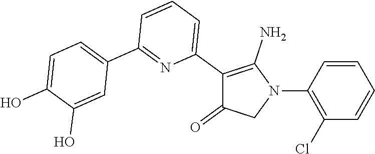 [SMILES: NC1=C(c2cccc(-c3ccc(O)c(O)c3)n2)C(=O)CN1c1ccccc1Cl] | Yellow solid; ¹H NMR (400 MHz, DMSO-d6) δ = 9.91 (br s, 1H), 9.51-8.80 (m, 2H), 8.32 (d, J = 8.0 Hz, 1H), 7.75 (br s, 1H), 7.72-7.58 (m, 3H), 7.53-7.41 (m, 2H), 7.32-7.21 (m, 2H), 7.15 (dd, J = 2.0, 8.0 Hz, 1H), 6.81 (d, J = 8.0 Hz, 1H), 4.04 (s, 2H); LCMS: 94%, MS (ESI): m/z 394.1 [M + H]+. |
| 77 | 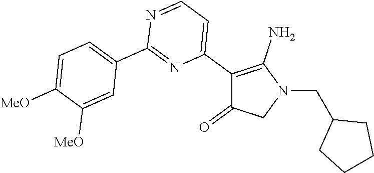 [SMILES: COc1ccc(-c2nccc(C3=C(N)N(CC4CCCC4)CC3=O)n2)cc1OC] | Yellow solid; ¹H NMR (400 MHz, DMSO-d6) δ = 9.69- 9.49 (m, 1H), 8.44 (d, J = 5.6 Hz, 1H), 8.38-8.26 (m, 1H), 8.11 (d, J = 5.6 Hz, 1H), 7.78 (dd, J = 2.0, 8.4 Hz, 1H), 7.75 (d, J = 1.6 Hz, 1H), 7.08 (d, J = 8.8 Hz, 1H), 3.86 (s, 3H), 3.84 (s, 3H), 3.81 (s, 2H), 3.38 (d, J = 8.0 Hz, 2H), 2.30-2.19 (m, 1H), 1.78-1.68 (m, 2H), 1.63- 1.59 (m, 2H), 1.58-1.47 (m, 2H), 1.27-1.19 (m, 2H); LCMS: 100%, MS (ESI): m/z 395.1 [M + H]+. |
| 78 | 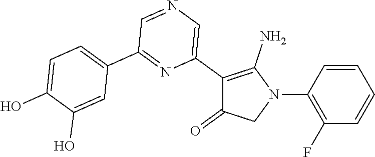 [SMILES: NC1=C(c2cncc(-c3ccc(O)c(O)c3)n2)C(=O)CN1c1ccccc1F] | Brown solid; ¹H NMR (500 MHz, DMSO-d6) δ = 9.48 (s, 1H), 9.45-9.08 (m, 2H), 8.53 (s, 1H), 8.21-7.98 (m, 1H), 7.63 (dt, J = 1.5, 8.0 Hz, 1H), 7.51-7.46 (m, 1H), 7.45-7.40 (m, 1H), 7.37-7.33 (m, 1H), 7.33-7.31 (m, 1H), 7.25 (dd, J = 2.0, 8.0 Hz, 1H), 6.86 (d, J = 8.5 Hz, 1H), 4.17 (s, 2H); LCMS: 96%, MS (ESI): m/z 379.1 [M + H]+. |
| 79 | 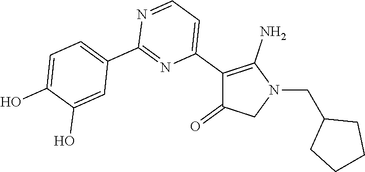 [SMILES: NC1=C(c2ccnc(-c3ccc(O)c(O)c3)n2)C(=O)CN1CC1CCCC1] | Yellow solid; ¹H NMR (400 MHz, DMSO-d6) δ = 9.74- 9.51 (m, 1H), 9.45-9.31 (m, 1H), 9.14 (br s, 1H), 8.40 (d, J = 5.6 Hz, 1H), 8.35-8.22 (m, 1H), 8.06 (d, J = 5.6 Hz, 1H), 7.65 (d, J = 2.0 Hz, 1H), 7.50 (dd, J = 2.0, 8.4 Hz, 1H), 6.83 (d, J = 8.4 Hz, 1H), 3.79 (s, 2H), 3.38 (d, J = 8.0 Hz, 2H), 2.31-2.18 (m, 1H), 1.78-1.68 (m, 2H), 1.68-1.59 (m, 2H), 1.58-1.48 (m, 2H), 1.28-1.17 (m, 2H); LCMS: 97%, MS (ESI): m/z 367.1 [M + H]+. |
| 80 | 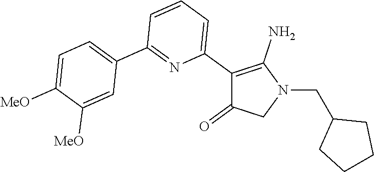 [SMILES: COc1ccc(-c2cccc(C3=C(N)N(CC4CCCC4)CC3=O)n2)cc1OC] | Yellow solid; ¹H NMR (400 MHz, DMSO-d6) δ = 10.09- 9.85 (m, 1H), 8.31 (d, J = 8.0 Hz, 1H), 8.13-7.91 (m, 1H), 7.65 (t, J = 8.0 Hz, 1H), 7.47-7.40 (m, 2H), 7.37 (d, J = 7.6 Hz, 1H), 7.06 (d, J = 8.8 Hz, 1H), 3.86 (s, 3H), 3.82 (s, 3H), 3.23 (s, 2H), 3.41-3.38 (m, 2H), 2.25 (td, J = 7.6, 15.6 Hz, 1H), 1.79-1.68 (m, 2H), 1.67- 1.58 (m, 2H), 1.58-1.46 (m, 2H), 1.27-1.15 (m, 2H); LCMS: 100%, MS (ESI): m/z 394.1 [M + H]+. |
| 81 | 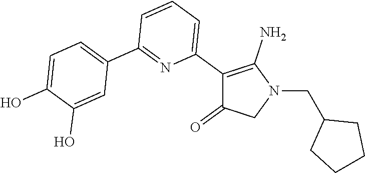 [SMILES: NC1=C(c2cccc(-c3ccc(O)c(O)c3)n2)C(=O)CN1CC1CCCC1] | Yellow solid; ¹H NMR (400 MHz, DMSO-d6) δ = 10.13- 9.85 (m, 1H), 9.38-9.21 (m, 1H), 9.14-9.01 (m, 1H), 8.26 (d, J = 8.0 Hz, 1H), 8.07-7.90 (m, 1H), 7.61 (t, J = 8.0 Hz, 1H), 7.27 (s, 1H), 7.22-7.11 (m, 2H), 6.83 (d, J = 8.4 Hz, 1H), 3.72 (s, 2H), 3.35 (br s, 2H), 2.30-2.19 (m, 1H), 1.78-1.58 (m, 4H), 1.56-1.49 (m, 2H), 1.26- 1.17 (m, 2H); LCMS: 100%, MS (ESI): m/z 366.1 [M + H]+. |
| 82 | 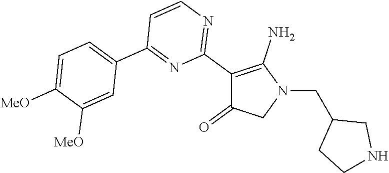 [SMILES: COc1ccc(-c2ccnc(C3=C(N)N(CC4CCNC4)CC3=O)n2)cc1OC] | Yellow solid; ¹H NMR (400 MHz, DMSO-d6) δ = 9.56- 9.33 (m, 1H), 8.54 (d, J = 5.2 Hz, 1H), 8.35 (s, 1H), 7.84 (d, J = 2.0 Hz, 1H), 7.76 (dd, J = 2.0, 8.8 Hz, 1H), 7.44 (d, J = 5.2 Hz, 1H), 7.09 (d, J = 8.4 Hz, 1H), 3.86 (s, 3H), 3.83 (s, 3H), 3.71 (d, J = 3.2 Hz, 2H), 3.22-3.15 (m, 3H), 3.11-3.01 (m, 2H), 2.88-2.83 (m, 1H), 2.67- 2.60 (m, 1H), 2.09-1.97 (m, 1H), 1.66-1.54 (m, 1H); LCMS: 100%, MS (ESI): m/z 396.1 [M + H]+. |
| 83 | 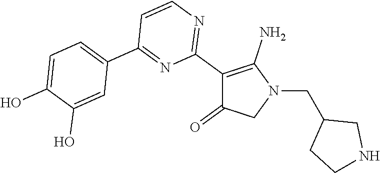 [SMILES: NC1=C(c2nccc(-c3ccc(O)c(O)c3)n2)C(=O)CN1CC1CCNC1] | off-white solid; ¹H NMR (400 MHz, DMSO-d6) δ = 9.66-9.39 (m, 1H), 8.49 (d, J = 5.6 Hz, 1H), 8.32 (s, 2H), 7.60 (d, J = 2.0 Hz, 1H), 7.45 (dd, J = 2.0, 8.4 Hz, 1H), 7.26 (d, J = 5.2 Hz, 1H), 6.85 (d, J = 8.0 Hz, 1H), 3.71 (br s, 2H), 3.56-3.50 (m, 2H), 3.29-3.24 (m, 2H), 3.16-3.05 (m, 1H), 2.94-2.83 (m, 1H), 2.70-2.60 (m, 1H), 2.10-2.00 (m, 1H), 1.73-1.54 (m, 1H); LCMS: 99% MS (ESI): m/z 368.1 [M + H]+. |
| 84 | 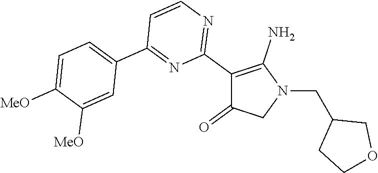 [SMILES: COc1ccc(-c2ccnc(C3=C(N)N(CC4CCOC4)CC3=O)n2)cc1OC] | Orange solid; ¹H NMR (500 MHz, METHANOL-d4) δ = 8.55 (br s, 1H), 7.74-7.63 (m, 2H), 7.47 (d, J = 3.5 Hz, 1H), 7.09 (d, J = 8.0 Hz, 1H), 4.67-4.48 (m, 1H), 4.01-3.93 (m, 6H), 3.92 (s, 3H), 3.88 (dd, J = 7.2, 8.5 Hz, 1H), 3.78 (q, J = 7.5 Hz, 1H), 3.57 (dd, J = 5.5, 8.5 Hz, 1H), 3.52 (dd, J = 3.0, 7.0 Hz, 2H), 2.76-271 (m, 1H), 2.21-2.07 (m, 1H), 1.77-1.64 (m, 1H); LCMS: 100%, MS (ESI): m/z 397.1 [M + H]+. |
| 85 | 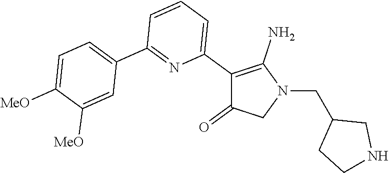 [SMILES: COc1ccc(-c2cccc(C3=C(N)N(CC4CCNC4)CC3=O)n2)cc1OC] | off-white solid: ¹H NMR (400 MHz, METHANOL-d4) δ = 8.28 (d, J = 8.0 Hz, 1H), 7.70 (t, J = 8.0 Hz, 1H), 7.51-7.43 (m, 2H), 7.40 (d, J = 7.6 Hz, 1H), 7.06 (d, J = 8.4 Hz, 1H), 3.96-3.86 (m, 8H), 3.59 (d, J = 7.2 Hz, 2H), 3.51-3.48 (m, 1H), 3.46-3.38 (m, 1H), 3.27-3.25 (m, 1H), 3.02-2.94 (m, 1H), 2.80-2.75 (m, 1H), 2.34- 2.20 (m, 1H), 1.83-1.75 (m, 1H); LCMS: 94%, MS (ESI): m/z 395.2 [M + H]+. |
| 86 | 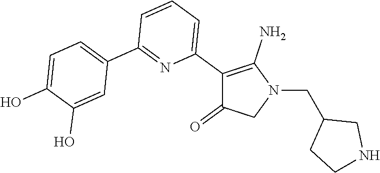 [SMILES: NC1=C(c2cccc(-c3ccc(O)c(O)c3)n2)C(=O)CN1CC1CCNC1] | Yellow solid; ¹H NMR (400 MHz, METHANOL-d4) δ = 8.23 (d, J = 8.0 Hz, 1H), 7.67 (t, J = 8.0 Hz, 1H), 7.35- 7.27 (m, 2H), 7.22 (dd, J = 2.0, 8.4 Hz, 1H), 6.85 (d, J = 8.0 Hz, 1H), 3.90 (s, 2H), 3.59 (d, J = 7.2 Hz, 2H), 3.55-3.50 (m, 1H), 3.49-3.41 (m, 1H), 3.31-3.29 (m, 1H), 3.04-2.97 (m, 1H), 2.87-2.74 (m, 1H), 2.35-2.22 (m, 1H), 1.85-1.77 (m, 1H); LCMS: 96%, MS (ESI): m/z 367.1 [M + H]+. |
| 87 | 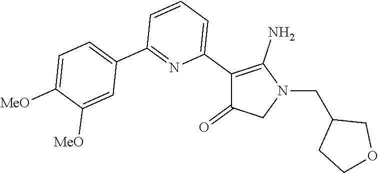 [SMILES: COc1ccc(-c2cccc(C3=C(N)N(CC4CCOC4)CC3=O)n2)cc1OC] | Yellow solid; ¹H NMR (500 MHz, METHANOL-d4) δ = 8.26 (d, J = 8.0 Hz, 1H), 7.68 (t, J = 7.5 Hz, 1H), 7.47 (d, J = 2.0 Hz, 1H), 7.44 (dd, J = 2.0, 8.0 Hz, 1H), 7.38 (d, J = 7.5 Hz, 1H), 7.04 (d, J = 8.5 Hz, 1H), 3.97-3.93 (m, 1H), 3.92 (s, 3H), 3.90-3.87 (m, 5H), 3.87-3.83 (m, 1H), 3.77 (q, J = 7.5 Hz, 1H), 3.56 (dd, J = 5.5, 8.5 Hz, 1H), 3.51-3.41 (m, 2H), 2.76-2.65 (m, 1H), 2.15- 2.09 (m, 1H), 1.74-1.63 (m, 1H); LCMS: 98%, MS (ESI): m/z 396.1 [M + H]+. |
| 88 | 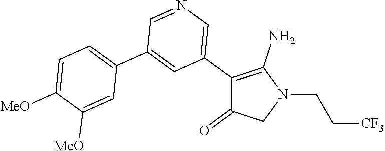 [SMILES: COc1ccc(-c2cncc(C3=C(N)N(CCC(F)(F)F)CC3=O)c2)cc1OC] | Yellow solid; ¹H NMR (400 MHz, DMSO-d6) δ = 8.57- 8.51 (m, 2H), 7.94 (br s, 1H), 7.44 (br s, 2H), 7.27 (br s, 1H), 7.23 (d, J = 8.4 Hz, 1H), 7.07 (d, J = 8.4 Hz, 1H), 3.84 (s, 3H), 3.80 (s, 3H), 3.75 (s, 2H), 3.69 (br s, 2H), 2.68-2.60 (m, 2H); LCMS: 100%, MS (ESI): m/z 408.1 [M + H]+. |
| 89 | 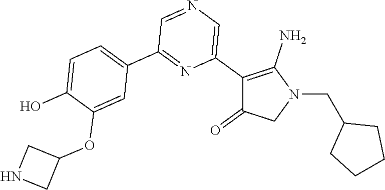 [SMILES: NC1=C(c2cncc(-c3ccc(O)c(OC4CNC4)c3)n2)C(=O)CN1CC1CCCC1] | Yellow solid; ¹H NMR (400 MHz, DMSO-d6) δ = 9.45 (s, 1H), 9.37-9.06 (m, 1H), 8.55 (s, 1H), 8.37-8.09 (m, 1H), 7.50-7.41 (m, 2H), 7.01 (d, J = 9.2 Hz, 1H), 4.49- 4.39 (m, 1H), 4.17-3.99 (m, 2H), 3.80 (s, 2H), 3.46- 3.43 (m, 2H), 2.90-2.74 (m, 2H), 2.31-2.16 (m, 1H), 1.77-1.67 (m, 2H), 1.67-1.58 (m, 2H), 1.58-1.44 (m, 2H), 1.28-1.12 (m, 2H); LCMS: 100%, MS (ESI) m/z 422.2 [M + H]+. |
| 90 | 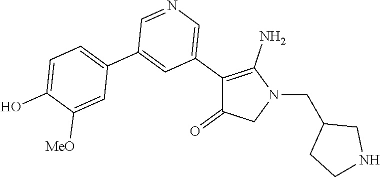 [SMILES: COc1cc(-c2cncc(C3=C(N)N(CC4CCNC4)CC3=O)c2)ccc1O] | Yellow solid; ¹H NMR (400 MHz, METHANOL-d4) δ = 8.54 (s, 1H), 8.49-8.40 (m, 2H), 8.06-7.97 (m, 1H), 7.26 (d, J = 2.0 Hz, 1H), 7.14 (dd, J = 2.0, 8.0 Hz, 1H), 6.91 (d, J = 8.4 Hz, 1H), 3.97 (s, 2H), 3.94 (s, 3H), 3.63- 3.57 (m, 2H), 3.57-3.50 (m, 1H), 3.49-3.44 (m, 1H), 3.04-2.93 (m, 1H), 2.86-2.72 (m, 1H), 2.35-2.22 (m, 1H), 1.86-1.74 (m, 1H), 1.37-1.25 (m, 1H); LCMS: 100%, MS (ESI): m/z 381.2 [M + H]+. |
| 91 | 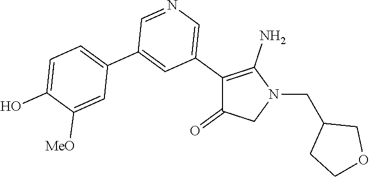 [SMILES: COc1cc(-c2cncc(C3=C(N)N(CC4CCOC4)CC3=O)c2)ccc1O] | Yellow solid; ¹H NMR (400 MHz, METHANOL-d4) δ = 8.53 (d, J = 2.0 Hz, 1H), 8.46 (d, J = 2.0 Hz, 1H), 8.05 (t, J = 2.0 Hz, 1H), 7.27 (d, J = 2.0 Hz, 1H), 7.15 (dd, J = 2.0, 8.4 Hz, 1H), 6.91 (d, J = 8.4 Hz, 1H), 3.96 (d, J = 2.4 Hz, 2H), 3.94 (s, 3H), 3.91 (d, J = 8.4 Hz, 1H), 3.87 (d, J = 7.2 Hz, 1H), 3.82-3.72 (m, 1H), 3.59-3.55 (m, 1H), 3.52-3.47 (m, 2H), 2.81-2.66 (m, 1H), 2.21-2.06 (m, 1H), 1.77-1.63 (m, 1H); LCMS: 100%, MS (ESI): m/z 382.2 [M + H]+. |
| 92 | 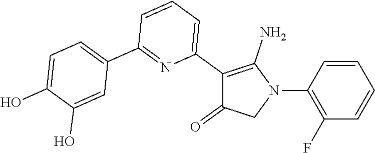 [SMILES: NC1=C(c2cccc(-c3ccc(O)c(O)c3)n2)C(=O)CN1c1ccccc1F] | Yellow solid; ¹H NMR (400 MHz, DMSO-d6) δ = 10.24- 9.54 (m, 1H), 9.48-9.24 (m, 1H), 9.23-8.98 (m, 1H), 8.28 (d, J = 7.6 Hz, 1H), 8.07-7.82 (m, 1H), 7.75 (t, J = 8.0 Hz, 1H), 7.62 (dt, J = 2.0, 8.0 Hz, 1H), 7.52-7.38 (m, 2H), 7.37-7.30 (m, 2H), 7.28 (d, J = 2.0 Hz, 1H), 7.18 (dd, J = 2.0, 8.0 Hz, 1H), 6.84 (d, J = 8.0 Hz, 1H), 4.15 (s, 2H); LCMS: 100%, MS (ESI): m/z 378.1 [M + H]+. |
| 93 | 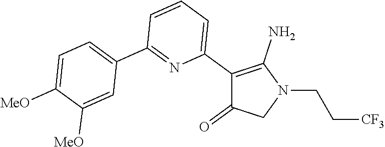 [SMILES: COc1ccc(-c2cccc(C3=C(N)N(CCC(F)(F)F)CC3=O)n2)cc1OC] | White solid; ¹H NMR (400 MHz, DMSO-d6) δ = 10.15- 9.92 (m, 1H), 8.34-8.27 (m, 1H), 8.26-8.10 (m, 1H), 7.67 (t, J = 8.0 Hz, 1H), 7.48-7.42 (m, 2H), 7.42-7.35 (m, 1H), 7.07 (d, J = 8.8 Hz, 1H), 3.86 (s, 3H), 3.82 (s, 3H), 3.77 (s, 2H), 3.71 (t, J = 7.2 Hz, 2H), 2.77-2.62 (m, 2H); LCMS: 98%, MS (ESI): m/z 408.1 [M + H]+. |
| 94 | 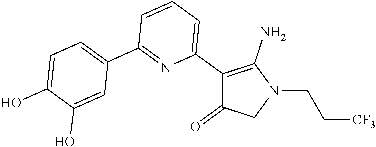 [SMILES: NC1=C(c2cccc(-c3ccc(O)c(O)c3)n2)C(=O)CN1CCC(F)(F)F] | Brown solid; ¹H NMR (400 MHz, DMSO-d6) δ = 10.31- 9.84 (m, 1H) 9.45-9.18 (m, 1H), 9.18-8.91 (m, 1H), 8.25 (d, J = 8.0 Hz, 1H), 8.22-8.17 (m, 1H), 7.62 (t, J = 8.0 Hz, 1H), 7.30-7.19 (m, 2H), 7.17 (dd, J = 2.0, 8.4 Hz, 1H), 6.83 (d, J = 8.4 Hz, 1H), 3.76 (s, 2H), 3.71 (t, J = 7.2 Hz, 2H), 2.77-2.60 (m, 2H); LCMS: 94%, MS (ESI): m/z 380.1 [M + H]+. |
| 95 | 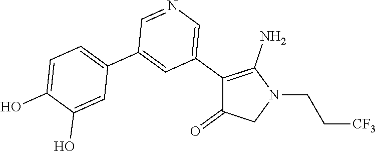 [SMILES: NC1=C(c2cncc(-c3ccc(O)c(O)c3)c2)C(=O)CN1CCC(F)(F)F] | Yellow solid; ¹H NMR (400 MHz, DMSO-d6) δ = 9.13- 9.08 (m, 2H), 8.48 (s, 1H), 8.39 (s, 1H), 7.90 (s, 1H), 7.41 (s, 2H), 7.06 (s, 1H), 7.00-6.93 (m, 1H), 6.83 (d, J = 8.4 Hz, 1H), 3.74 (s, 2H), 3.72-3.63 (m, 2H), 2.69- 2.58 (m, 2H); LCMS: 94%, MS (ESI): m/z 380.1 [M + H]+. |
| 96 | 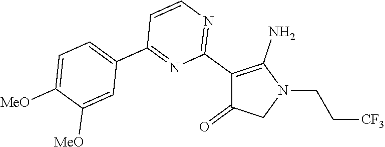 [SMILES: COc1ccc(-c2ccnc(C3=C(N)N(CCC(F)(F)F)CC3=O)n2)cc1OC] | Yellow solid; ¹H NMR (400 MHz, DMSO-d6) δ = 9.67- 9.16 (m, 1H), 8.55 (br s, 1H), 8.35-8.01 (m, 1H), 7.95- 3.63 (m, 2H), 7.46 (br s, 1H), 7.09 (br s, 1H), 3.85 (br d, J = 9.6 Hz, 6H), 3.73 (br s, 4H), 2.69 (d, J = 2.4 Hz, 2H); LCMS: 99%, MS (ESI): m/z 409.1 [M + H]+. |
| 97 | 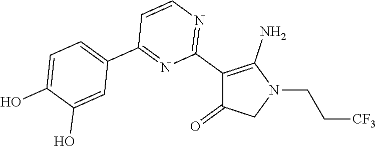 [SMILES: NC1=C(c2nccc(-c3ccc(O)c(O)c3)n2)C(=O)CN1CCC(F)(F)F] | Orange solid; ¹H NMR (400 MHz, METHANOL-d4) δ = 8.59-8.47 (m, 1H), 7.54-7.46 (m, 2H), 7.41 (d, J = 3.6 Hz, 1H), 6.92 (d, J = 8.0 Hz, 1H), 3.98 (br s, 2H), 3.84 (t, J = 7.2 Hz, 2H), 2.75-2.59 (m, 2H); LCMS: 100%, MS (ESI): m/z 381.0 [M + H]+. |
| 98 | 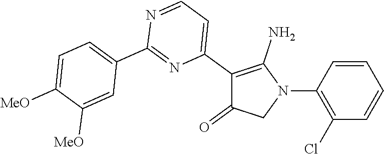 [SMILES: COc1ccc(-c2nccc(C3=C(N)N(c4ccccc4Cl)CC3=O)n2)cc1OC] | Yellow solid; ¹H NMR (400 MHz, DMSO-d6) δ = 9.55 (br s, 1H), 8.54 (d, J = 5.6 Hz, 1H), 8.18 (d, J = 5.6 Hz, 2H), 7.79 (dd, J = 1.6, 8.4 Hz, 1H), 7.74-7.65 (m, 3H), 7.58-7.47 (m, 2H), 7.07 (d, J = 8.4 Hz, 1H), 4.12 (br d, J = 7.2 Hz, 2H), 3.82 (s, 3H), 3.80 (s, 3H), LCMS: 96%, MS (ESI): m/z 423.1 [M + H]+. |
| 99 | 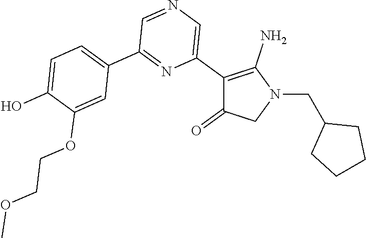 [SMILES: COCCOc1cc(-c2cncc(C3=C(N)N(CC4CCCC4)CC3=O)n2)ccc1O] | Yellow solid; ¹H NMR (400 MHz, DMSO-d6) δ = 9.62- 9.45 (m, 1H), 9.42 (s, 1H), 9.38-9.01 (m, 1H), 8.56 (s, 1H), 8.44-7.91 (m, 1H), 7.47 (d, J = 2.0 Hz, 1H), 7.41 (dd, J = 2.0, 8.4 Hz, 1H), 6.94 (d, J = 8.4 Hz, 1H), 4.19 (t, J = 4.4 Hz, 2H), 3.81 (s, 2H), 3.72 (t, J = 4.4 Hz, 2H), 3.38 (d, J = 7.2 Hz, 2H), 3.34 (s, 3H), 2.29-2.21 (m, 1H), 1.79-1.68 (m, 2H), 1.67-1.59 (m, 2H), 1.58- 1.49 (m, 2H), 1.29-1.14 (m, 2H); LCMS: 100%, MS (ESI): m/z 425.1 [M + H]+. |
| 100 | 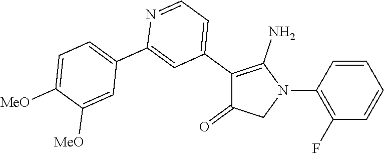 [SMILES: COc1ccc(-c2cc(C3=C(N)N(c4ccccc4F)CC3=O)ccn2)cc1OC] | Yellow solid; ¹H NMR (400 MHz, DMSO-d6) δ = 8.45 (d, J = 5.2 Hz, 1H), 8.03 (s, 1H), 7.67 (d, J = 2.0 Hz, 1H), 7.55 (dd, J = 2.0, 8.4 Hz, 2H), 7.51-7.26 (m, 6H), 7.05 (d, J = 8.4 Hz, 1H), 4.06 (s, 2H), 3.83 (s, 3H), 3.81 (s, 3H); LCMS: 100%, MS (ESI): m/z 406.1 [M + H]+. |
| 101 | 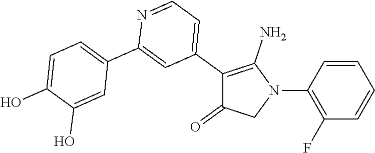 [SMILES: NC1=C(c2ccnc(-c3ccc(O)c(O)c3)c2)C(=O)CN1c1ccccc1F] | Yellow solid; ¹H NMR (400 MHz, DMSO-d6) δ = 9.11- 9.07 (m, 1H), 9.06-9.02 (m, 1H), 8.40 (d, J = 5.2 Hz, 1H), 7.98 (s, 1H), 7.56 (dt, J = 1.2, 8.0 Hz, 1H), 7.50 (d, J = 2.0 Hz, 1H), 7.47-7.27 (m, 7H), 6.80 (d, J = 8.4 Hz, 1H), 4.06 (s, 2H); LCMS: 100%, MS (ESI): m/z 378.1 [M + H]+. |
| 102 | 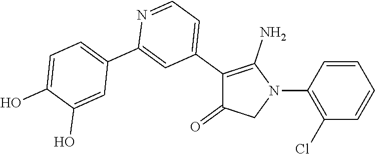 [SMILES: NC1=C(c2ccnc(-c3ccc(O)c(O)c3)c2)C(=O)CN1c1ccccc1Cl] | Yellow solid; ¹H NMR (400 MHz, DMSO-d6) δ = 9.19- 9.07 (m, 1H), 9.05 (br s, 1H), 8.39 (d, J = 5.2 Hz, 1H), 8.00 (s, 1H), 7.69-7.62 (m, 1H), 7.61-7.55 (m, 1H), 7.52-7.43 (m, 3H), 7.42-7.25 (m, 4H), 6.80 (d, J = 8.0 Hz, 1H), 4.00 (s, 2H); LCMS: 100%, MS (ESI): m/z 394.1 [M + H]+. |
| 103 | 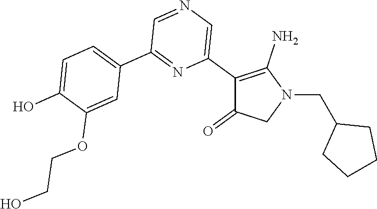 [SMILES: NC1=C(c2cncc(-c3ccc(O)c(OCCO)c3)n2)C(=O)CN1CC1CCCC1] | White solid; ¹H NMR (400 MHz, DMSO-d6) δ = 9.43 (s, 1H), 9.37-9.07 (m, 1H), 8.56 (s, 1H), 8.30-8.02 (m, 1H), 7.47 (s, 1H), 7.41 (d, J = 8.0 Hz, 1H), 6.93 (d, J = 8.0 Hz, 1H), 5.19-4.77 (m, 1H), 4.08 (t, J = 4.8 Hz, 2H), 3.87-3.72 (m, 4H), 3.58-3.44 (m, 2H), 2.28-2.21 (m, 1H), 1.75-1.65 (m, 2H), 1.67-1.58 (m, 2H), 1.57- 1.44 (m, 2H), 1.24-1.16 (m, 2H); LCMS: 96%, MS (ESI): m/z 411.2 [M + H]+. |
| 104 | 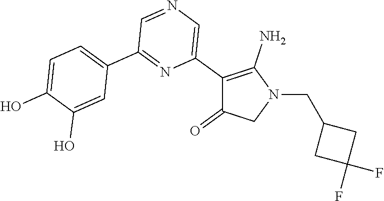 [SMILES: NC1=C(c2cncc(-c3ccc(O)c(O)c3)n2)C(=O)CN1CC1CC(F)(F)C1] | Brown solid; ¹H NMR (400 MHz, DMSO-d6) δ = 9.40 (s, 2H), 9.32-8.99 (m, 2H), 8.45 (s, 1H), 8.39-8.19 (m, 1H), 7.33 (s, 1H), 7.25 (d, J = 8.0 Hz, 1H), 6.87 (d, J = 8.0 Hz, 1H), 3.80 (s, 2H), 3.59 (d, J = 6.8 Hz, 2H), 2.84- 2.59 (m, 3H), 2.43-2.31 (m, 2H); LCMS: 99%, MS (ESI): m/z 389.1 [M + H]+. |
| 105 | 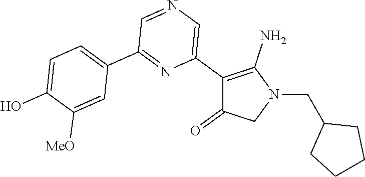 [SMILES: COc1cc(-c2cncc(C3=C(N)N(CC4CCCC4)CC3=O)n2)ccc1O] | Yellow solid; ¹H NMR (400 MHz, DMSO-d6) δ = 9.67- 9.58 (m, 1H), 9.56 (s, 1H), 8.42 (d, J = 5.6 Hz, 1H), 8.39-8.30 (m, 1H), 8.08 (d, J = 5.6 Hz, 1H), 7.73 (s, 1H), 7.65 (d, J = 8.4 Hz, 1H), 6.89 (dd, J = 3.0, 8.4 Hz, 1H), 3.86 (s, 3H), 3.80 (s, 2H), 3.38 (br s, 2H), 2.30- 2.20 (m, 1H), 1.79-1.68 (m, 2H), 1.68-1.59 (m, 2H), 1.58-1.42 (m, 2H), 1.29-1.16 (m, 2H); LCMS: 96%, MS (ESI): m/z 381.1 [M + H]+. |
| 106 | 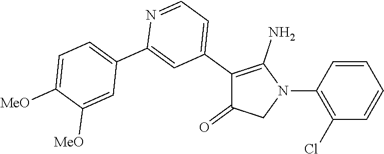 [SMILES: COc1ccc(-c2cc(C3=C(N)N(c4ccccc4Cl)CC3=O)ccn2)cc1OC] | Yellow solid; ¹H NMR (400 MHz, DMSO-d6) δ = 8.45 (d, J = 5.6 Hz, 1H), 8.16 (s, 1H), 8.05 (s, 1H), 7.70-7.61 (m, 2H), 7.61-7.52 (m, 2H), 7.51-7.44 (m, 3H), 7.42 (br s, 1H), 7.06 (d, J = 8.4 Hz, 1H), 4.01 (s, 2H), 3.83 (s, 3H), 3.81 (s, 3H); LCMS: 93%, MS (ESI): m/z 422.1 [M + H]+. |
| 107 | 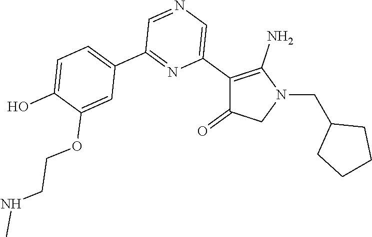 [SMILES: CNCCOc1cc(-c2cncc(C3=C(N)N(CC4CCCC4)CC3=O)n2)ccc1O] | Yellow solid; ¹H NMR (400 MHz, DMSO-d6) δ = 9.42 (s, 1H), 9.37-9.09 (m, 1H), 8.55 (s, 1H), 8.43-8.31 (m, 1H), 8.30-8.01 (m, 1H), 7.51 (s, 1H), 7.46 (dd, J = 1.6, 8.4 Hz, 1H), 6.91 (d, J = 8.4 Hz, 1H), 4.30-4.04 (m, 4H), 3.79 (s, 3H), 3.37 (br d, J = 7.6 Hz, 2H), 3.03 (br s, 2H), 2.30-2.20 (m, 1H), 1.78-1.67 (m, 2H), 1.66-1.58 (m, 2H), 1.57-1.44 (m, 2H), 1.27-1.16 (m, 2H); LCMS: 100%, MS (ESI): m/z 424.2 [M + H]+. |
| 108 | 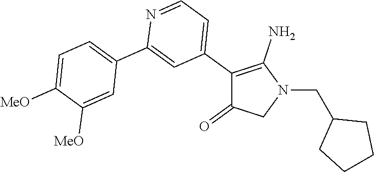 [SMILES: COc1ccc(-c2cc(C3=C(N)N(CC4CCCC4)CC3=O)ccn2)cc1OC] | Red solid; ¹H NMR (400 MHz, DMSO-d6) δ = 8.39 (d, J = 5.2 Hz, 1H), 8.00 (s, 1H), 7.66 (d, J = 2.0 Hz, 1H), 7.53 (dd, J = 2.0, 8.4 Hz, 1H), 7.46-7.25 (m, 3H), 7.04 (d, J = 8.4 Hz, 1H), 3.83 (s, 3H), 3.80 (s, 3H), 3.72 (s, 2H), 3.35 (br d, J = 7.6 Hz, 2H), 2.28-2.13 (m, 1H), 1.77-1.67 (m, 2H), 1.67-1.58 (m, 2H), 1.58-1.46 (m, 2H) 1.26-1.13 (m, 2H); LCMS: 100%, MS (ESI): m/z 394.2 [M + H]+. |
| 109 | 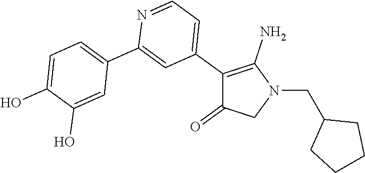 [SMILES: NC1=C(c2ccnc(-c3ccc(O)c(O)c3)c2)C(=O)CN1CC1CCCC1] | Yellow solid; ¹H NMR (400 MHz, DMSO-d6) δ = 9.34- 8.76 (m, 1H), 8.34 (d, J = 5.2 Hz, 1H), 8.17 (s, 1H), 7.96 (s, 1H), 7.47 (d, J = 2.0 Hz, 1H), 7.35-7.26 (m, 3H), 6.79 (d, J = 8.0 Hz, 1H), 3.71 (s, 2H), 3.35 (br s, 2H), 2.26-2.15 (m, 1H), 1.78-1.67 (m, 2H), 1.67-1.59 (m, 2H), 1.58-1.47 (m, 2H), 1.26-1.16 (m, 2H); LCMS: 100%, MS (ESI): m/z 366.1 [M + H]+. |
| 110 | 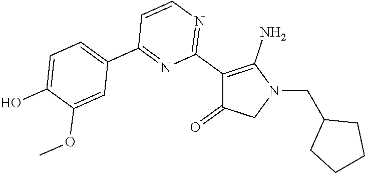 [SMILES: COc1cc(-c2ccnc(C3=C(N)N(CC4CCCC4)CC3=O)n2)ccc1O] | Yellow solid; ¹H NMR (400 MHz, DMSO-d6) δ = 8.51 (br s, 1H), 8.37 (s, 1H), 7.69 (s, 1H), 7.63 (br d, J = 8.4 Hz, 1H), 7.51 (br d, J = 5.6 Hz, 1H), 6.95 (d, J = 8.4 Hz, 1H), 3.99 (br s, 2H), 3.98 (s, 3H), 3.46 (d, J = 8.0 Hz, 2H), 2.29-2.23 (m, 1H), 1.92-1.79 (m, 2H), 1.79-1.69 (m, 2H), 1.69-1.57 (m, 2H), 1.38-1.24 (m. 2H); LCMS: 96%, MS (ESI): m/z 381.1 [M + H]+. |
| 111 | 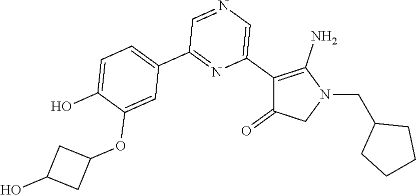 [SMILES: NC1=C(c2cncc(-c3ccc(O)c(OC4CC(O)C4)c3)n2)C(=O)CN1CC1CCCC1] | Yellow solid; ¹H NMR (400 MHz, DMSO-d6) δ = 9.44 (s, 1H), 9.38 (br s, 1H), 9.29-9.07 (m, 1H), 8.50 (s, 1H), 8.36-7.99 (m, 1H), 7.38 (br d, J = 8.0 Hz, 1H), 7.19 (s, 1H), 6.92 (br d, J = 8.4 Hz, 1H), 5.20 (br d, J = 4.4 Hz, 1H), 4.90 (br d, J = 2.8 Hz, 1H), 4.51-4.36 (m, 1H), 3.79 (s, 2H), 3.39 (br d, J = 7.6 Hz, 2H), 2.42-2.36 (m, 2H), 2.35-2.29 (m, 2H), 2.28-2.20 (m, 1H), 1.79-1.70 (m, 2H), 1.69-1.61 (m, 2H), 1.58-1.45 (m, 2H), 1.25- 1.18 (m, 2H); LCMS: 100%, MS (ESI): m/z 437.2 [M + H]+. |
| 112 | 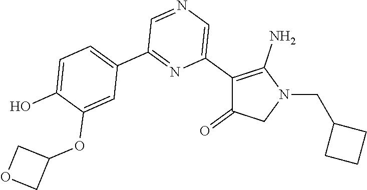 [SMILES: NC1=C(c2cncc(-c3ccc(O)c(OC4COC4)c3)n2)C(=O)CN1CC1CCC1] | Yellow solid; ¹H NMR (400 MHz, DMSO-d6) δ = 9.45 (s, 1H), 9.33-9.04 (m, 1H), 8.55 (s, 1H), 8.44-8.14 (m, 1H), 7.50-7.37 (m, 2H), 7.02 (d, J = 8.8 Hz, 1H), 5.13 (br t, J = 5.2 Hz, 1H), 4.40 (dd, J = 2.0, 11.2 Hz, 1H), 4.27-4.18 (m, 1H), 4.11-4.04 (m, 1H), 3.75 (s, 2H), 3.71-3.63 (m, 2H), 3.46 (d, J = 7.6 Hz, 2H), 2.71-2.60 (m, 1H), 2.09-1.97 (m, 2H), 1.92-1.82 (m, 2H), 1.80- 1.71 (m, 2H); LCMS: 100%, MS (ESI): m/z 409.2 [M + H]+. |
| 113 | 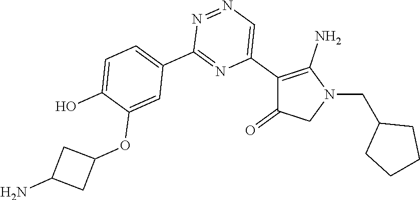 [SMILES: NC1=C(c2cnnc(-c3ccc(O)c(OC4CC(N)C4)c3)n2)C(=O)CN1CC1CCCC1] | Yellow solid; ¹H NMR (400 MHz, DMSO-d6) δ = 9.42 (s, 1H), 8.53 (s, 1H), 8.44 (s, 1H), 7.43 (dd, J = 2.0, 8.4 Hz, 1H), 7.30 (d, J = 2.0 Hz, 1H), 6.97 (d, J = 8.4 Hz, 1H), 4.72 (q, J = 6.8 Hz, 1H), 3.91 (s, 2H), 3.57 (q, J = 8.0 Hz, 1H), 3.42 (d, J = 7.6 Hz, 2H), 3.09-2.94 (m, 2H), 2.44-2.26 (m, 3H), 1.92-1.79 (m, 2H), 1.79-1.69 (m, 2H), 1.69-1.57 (m, 2H), 1.38-1.22 (m, 2H); LCMS: 99%, MS (ESI): m/z 436.2 [M + H]+. |
| 114 | 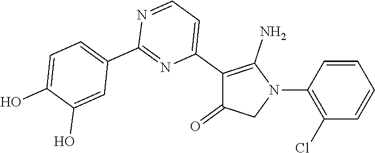 [SMILES: NC1=C(c2ccnc(-c3ccc(O)c(O)c3)n2)C(=O)CN1c1ccccc1Cl] | Yellow solid; ¹H NMR (400 MHz, DMSO-d6) δ = 8.47 (d, J = 5.6 Hz, 1H), 8.21 (d, J = 5.6 Hz, 1H), 7.70-7.64 (m, 1H), 7.64-7.57 (m, 2H), 7.55-7.46 (m, 3H), 6.84 (d, J = 8.4 Hz, 1H), 4.33-4.07 (m, 2H); LCMS: 96%, MS (ESI): m/z 395.0 [M + H]+. |
| 115 | 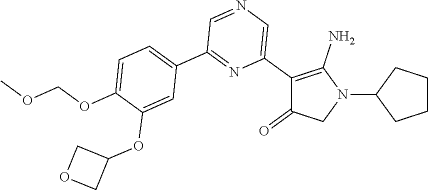 [SMILES: COCOc1ccc(-c2cncc(C3=C(N)N(C4CCCC4)CC3=O)n2)cc1OC1COC1] | Black brown solid; ¹H NMR (400 MHz, DMSO-d6) δ = 9.49 (s, 1H), 9.23-8.97 (m, 1H), 8.57 (s, 1H), 8.32- 8.06 (m, 1H), 7.54 (dd, J = 2.0, 8.4 Hz, 1H), 7.26 (d, J = 8.4 Hz, 1H), 7.11 (d, J = 2.0 Hz, 1H), 5.39 (q, J = 5.2 Hz, 1H), 5.28 (s, 2H), 4.98 (t, J = 6.8 Hz, 2H), 4.64 (dd, J = 5.2, 7.2 Hz, 2H), 4.35 (q, J = 7.2 Hz, 1H), 3.77 (s, 2H), 3.44 (s, 3H), 1.97-1.83 (m, 2H), 1.77-1.67 (m, 2H), 1.65-1.54 (m, 4H); LCMS: 99%, MS (ESI): m/z 453.3 [M + H]+. |
| 116 | 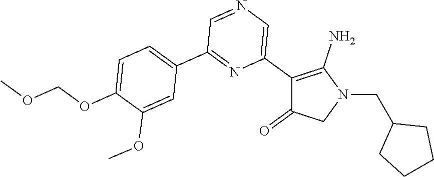 [SMILES: COCOc1ccc(-c2cncc(C3=C(N)N(CC4CCCC4)CC3=O)n2)cc1OC] | Yellow solid; ¹H NMR (400 MHz, DMSO-d6) δ = 9.48 (s, 1H), 9.39-9.07 (m, 1H), 8.61 (s, 1H), 8.39-8.02 (m, 1H), 7.53 (d, J = 1.6 Hz, 1H), 7.52-7.47 (m, 1H), 7.20 (d, J = 8.4 Hz, 1H), 5.23 (s, 2H), 3.89 (s, 3H), 3.80 (s, 2H), 3.41 (s, 3H), 3.37 (br d, J = 7.6 Hz, 2H), 2.25 (br t, J = 7.6 Hz, 1H), 1.78-1.68 (m, 2H), 1.68-1.59 (m, 2H), 1.58-1.47 (m, 2H), 1.27-1.18 (m, 2H); LCMS: 100%, MS (ESI): m/z 425.1 [M + H]+. |
| 117 | 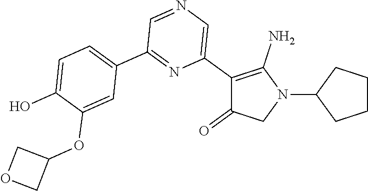 [SMILES: NC1=C(c2cncc(-c3ccc(O)c(OC4COC4)c3)n2)C(=O)CN1C1CCCC1] | Yellow solid; ¹H NMR (400 MHz, DMSO-d6) δ = 9.45 (s, 1H), 9.36-9.07 (m, 1H), 8.55 (s, 1H), 8.31-8.16 (m, 1H), 8.15 (s, 1H), 7.49-7.40 (m, 2H), 7.02 (d, J = 9.0 Hz, 1H), 5.10 (br s, 1H), 4.40 (dd, J = 2.0, 11.5 Hz, 1H), 4.34-4.19 (m, 2H), 4.07 (dd, J = 7.5, 11.5 Hz, 1H), 3.76 (s, 2H), 3.72-3.61 (m, 2H), 1.95-1.83 (m, 2H), 1.77- 1.67 (m, 2H), 1.65-1.52 (m, 4H); LCMS: 100%, MS (ESI): m/z 409.1 [M + H]+. |
| 118 | 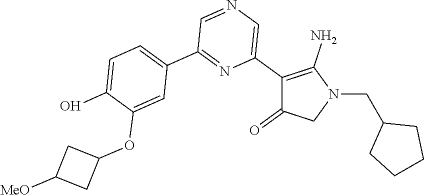 [SMILES: COC1CC(Oc2cc(-c3cncc(C4=C(N)N(CC5CCCC5)CC4=O)n3)ccc2O)C1] | Yellow solid; ¹H NMR (400 MHz, DMSO-d6) δ = 9.43 (s, 2H), 9.34-8.93 (m, 1H), 8.52 (s, 1H), 8.40-7.93 (m, 1H), 7.39 (br d, J = 8.4 Hz, 1H), 7.22 (s, 1H), 6.93 (d, J = 8.0 Hz, 1H), 4.92 (d, J = 4.3 Hz, 1H), 4.25-4.03 (m, 1H), 3.80 (s, 2H), 3.39 (br d, J = 7.2 Hz, 2H), 3.18 (s, 3H), 2.40 (d, J = 3.6 Hz, 4H), 2.30-2.17 (m, 1H), 1.61- 1.68 (m, 2H), 1.64 (br s, 2H), 1.58-1.41 (m, 2H), 1.24- 1.18 (m, 2H); LCMS: 100%, MS (ESI): m/z 451.2 [M + H]+. |
| 119 | 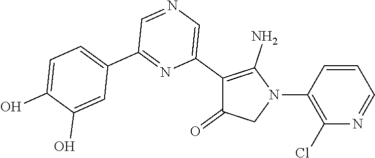 [SMILES: NC1=C(c2cncc(-c3ccc(O)c(O)c3)n2)C(=O)CN1c1cccnc1Cl] | White solid; ¹H NMR (400 MHz, DMSO-d6) δ = 9.47 (s, 1H), 9.30-9.14 (m, 1H), 8.54 (s, 1H), 8.51 (dd, J = 1.5, 4.5 Hz, 1H), 8.30-8.18 (m, 1H), 8.15 (dd, J = 1.5, 7.5 Hz, 1H), 7.61 (dd, J = 5.0, 7.5 Hz, 1H), 7.33 (d, J = 2.0 Hz, 1H), 7.25 (dd, J = 2.5, 8.0 Hz, 1H), 6.86 (d, J = 8.0 Hz, 1H), 4.12 (s, 2H); LCMS: 100%, MS (ESI): m/z 396.0 [M + H]+. |
| 120 | 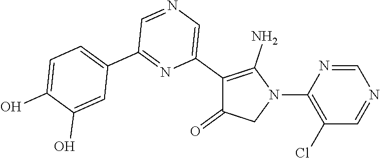 [SMILES: NC1=C(c2cncc(-c3ccc(O)c(O)c3)n2)C(=O)CN1c1ncncc1Cl] | Brown solid; ¹H NMR (400 MHz, DMSO-d6) δ = 9.08 (t, J = 5.5 Hz, 1H), 8.74 (d, J = 11.0 Hz, 2H), 8.66 (s, 1H), 8.60 (s, 1H), 7.36-7.28 (m, 2H), 7.03-6.96 (m, 1H), 4.68 (d, J = 6.0 Hz, 2H); LCMS: 100%, MS (ESI): m/z 397.0 [M+ H]+. |

1. A compound having the general formula I, optical isomer thereof, a mixture of two isomers thereof, precursor thereof, pharmaceutically acceptable salt thereof or solvate thereof: 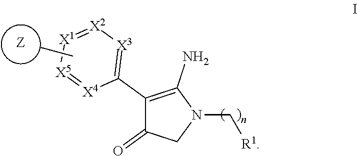
X¹ is, independently at each occurrence, selected from CH, C—Z and N;
X² is, independently at each occurrence, selected from CH, C—Z and N;
X³ is, independently at each occurrence, selected from CH, C—Z and N;
X⁴ is, independently at each occurrence, selected from CH, C—Z and N;
X⁵ is, independently at each occurrence, selected from CH, C—Z and N;
n is, independently at each occurrence, selected from 0, 1 and 2;
R¹ is, at each occurrence, independently selected from the group consisting of hydrogen, C1-C6 alkyl, C1-C6 alkyl substituted with one or several of halogen, C1-C6 alkyl, ═O, C3-C10 cycloalkyl, C3-C10 heterocycloalkyl, C1-C4 haloalkyl, OR⁶ and NR⁶R⁷; C3-C10 cycloalkyl, C3-C10 cycloalkyl substituted with one or several of halogen, C1-C6 alkyl, ═O, C3-C10 cycloalkyl, C3-C10 heterocycloalkyl, C1-C4 haloalkyl, OR⁶ and NR⁶R⁷; C3-C10 heterocycloalkyl, C3-C10 heterocycloalkyl substituted with one or several of halogen, C1-C6 alkyl, ═O, C3-C10 cycloalkyl, C3-C10 heterocycloalkyl, C1-C4 haloalkyl, OR⁶ and NR⁶R⁷; C6-C12 aryl, C6-C12 aryl substituted with one or several of halogen, C1-C6 alkyl, C3-C10 cycloalkyl, C3-C10 heterocycloalkyl, C1-C4 haloalkyl, OR⁶ and NR⁶R⁷; C3-C12 heteroaryl, C3-C12 heteroaryl substituted with one or several of halogen, C1-C6 alkyl, C3-C10 cycloalkyl, C3-C10 heterocycloalkyl, C1-C4 haloalkyl, OR⁶ and NR⁶R⁷;
Z is any structure of the following group A; 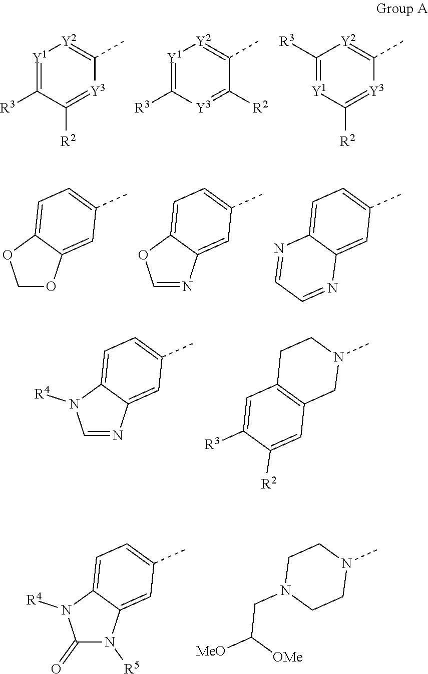
Y¹, Y² and Y³ are, independently at each occurrence, selected from CH and N;
R² and R³ are, at each occurrence, independently selected from the group consisting of hydrogen, halogen, C1-C6 alkyl, C3-C10 cycloalkyl, C3-C10 heterocycloalkyl, C1-C4 haloalkyl, OR⁶ and NR⁶R⁷, any of which is optionally substituted;
R⁴ and R⁵ are, are, at each occurrence, independently selected from the group consisting of hydrogen, C1-C6 alkyl, C3-C10 cycloalkyl, any of which is optionally substituted;
R⁶ and R⁷ are, at each occurrence, independently selected from the group consisting of hydrogen, C1-C4 haloalkyl, C1-C6 alkyl, C1-C6 alkyl substituted with one or several of halogen, C1-C6 alkyl, ═O, C3-C10 cycloalkyl, C3-C10 heterocycloalkyl, C1-C4 haloalkyl, OR⁸ and NR⁸R⁹; C3-C10 cycloalkyl, C3-C10 cycloalkyl substituted with one or several of halogen, C1-C6 alkyl, ═O, C1-C4 haloalkyl, OR⁸ and NR⁸R⁹; C3-C10 heterocycloalkyl, C3-C10 heterocycloalkyl substituted with one or several of halogen, C1-C6 alkyl, ═O, C1-C4 haloalkyl, OR⁸ and NR⁸R⁹;
R⁸ and R⁹ are, at each occurrence, independently selected from the group consisting of hydrogen, C1-C6 alkyl, C3-C10 cycloalkyl, C3-C10 heterocycloalkyl and C1-C4 haloalkyl, any of which is optionally substituted.

2. The compound, optical isomer thereof, a mixture of two isomers thereof, precursor thereof, pharmaceutically acceptable salt thereof or solvate thereof according to claim 1, wherein, Z is any structure of the following group B; 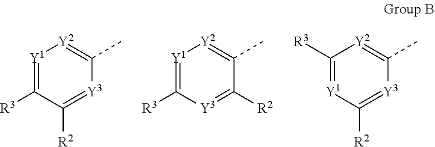
Wherein, R² and R³ are as defined in claim 1; and
Y¹, Y² and Y³ are as defined in claim 1.

3. The compound, optical isomer thereof, a mixture of two isomers thereof, precursor thereof, pharmaceutically acceptable salt thereof or solvate thereof according to claim 1,
wherein,
Z is 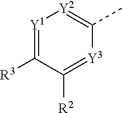
R² and R³ are as defined in claim 1; and
Y¹, Y² and Y³ are as defined in claim 1.

4. The compound, optical isomer thereof, a mixture of two isomers thereof, precursor thereof, pharmaceutically acceptable salt thereof or solvate thereof according to claim 1, having the general formula II, 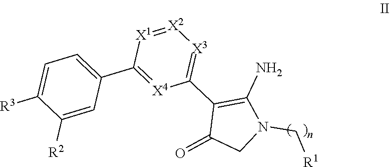
wherein, R¹, R² and R³ are as defined in claim 1; and
X¹, X², X³ and X⁴ are as defined in claim 1; and
n is as defined in claim 1.

5. The compound, optical isomer thereof, a mixture of two isomers thereof, precursor thereof, pharmaceutically acceptable salt thereof or solvate thereof according to claim 1, wherein R¹ is, at each occurrence, independently selected from the group consisting of hydrogen, C1-C6 alkyl, C1-C6 alkyl substituted with one or several of halogen, C1-C6 alkyl, ═O, C3-C10 cycloalkyl, C3-C10 heterocycloalkyl, C1-C4 haloalkyl, OR⁶ and NR⁶R⁷; C3-C10 cycloalkyl, C3-C10 cycloalkyl substituted with one or several of halogen, C1-C6 alkyl, ═O, C3-C10 cycloalkyl, C3-C10 heterocycloalkyl, C1-C4 haloalkyl, OR⁶ and NR⁶R⁷; C3-C10 heterocycloalkyl, C3-C10 heterocycloalkyl substituted with one or several of halogen, C1-C6 alkyl, ═O, C3-C10 cycloalkyl, C3-C10 heterocycloalkyl, C1-C4 haloalkyl, OR⁶ and NR⁶R⁷.

6. The compound, optical isomer thereof, a mixture of two isomers thereof, precursor thereof, pharmaceutically acceptable salt thereof or solvate thereof according to claim 1, wherein R¹ is, at each occurrence, independently selected from the group consisting of C6-C12 aryl, C6-C12 aryl substituted with one or several of halogen, C1-C6 alkyl, C3-C10 cycloalkyl, C3-C10 heterocycloalkyl, C1-C4 haloalkyl, OR⁶ and NR⁶R⁷; C3-C12 heteroaryl, C3-C12 heteroaryl substituted with one or several of halogen, C1-C6 alkyl, C3-C10 cycloalkyl, C3-C10 heterocycloalkyl, C1-C4 haloalkyl, OR⁶ and NR⁶R⁷.

7. The compound, optical isomer thereof, a mixture of two isomers thereof, precursor thereof, pharmaceutically acceptable salt thereof or solvate thereof according to claim 1, wherein, R² and R³ are, at each occurrence, independently selected from the group consisting of OR⁶ and NR⁶R⁷.

8. The compound, optical isomer thereof, a mixture of two isomers thereof, precursor thereof, pharmaceutically acceptable salt thereof or solvate thereof according to claim 1, having one of the formulae 1-120, as shown hereafter:

| # | |
|---|---|
| cpd | Structure |
| 1 | 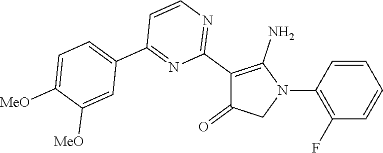 [SMILES: COc1ccc(-c2ccnc(C3=C(N)N(c4ccccc4F)CC3=O)n2)cc1OC] |
| 2 | 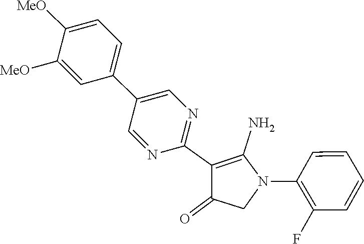 [SMILES: COc1ccc(-c2cnc(C3=C(N)N(c4ccccc4F)CC3=O)nc2)cc1OC] |
| 3 | 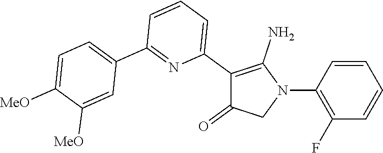 [SMILES: COc1ccc(-c2cccc(C3=C(N)N(c4ccccc4F)CC3=O)n2)cc1OC] |
| 4 | 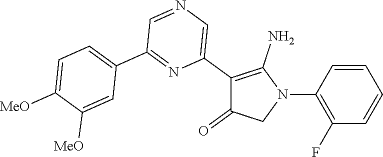 [SMILES: COc1ccc(-c2cncc(C3=C(N)N(c4ccccc4F)CC3=O)n2)cc1OC] |
| 5 | 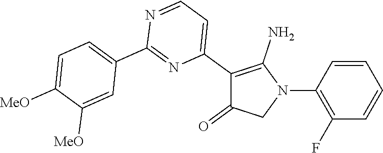 [SMILES: COc1ccc(-c2nccc(C3=C(N)N(c4ccccc4F)CC3=O)n2)cc1OC] |
| 6 | 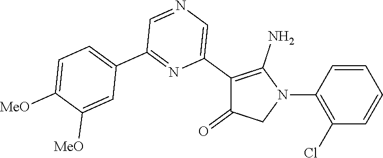 [SMILES: COc1ccc(-c2cncc(C3=C(N)N(c4ccccc4Cl)CC3=O)n2)cc1OC] |
| 7 | 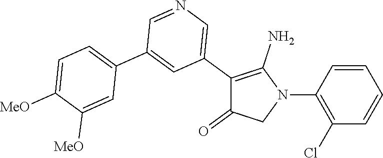 [SMILES: COc1ccc(-c2cncc(C3=C(N)N(c4ccccc4Cl)CC3=O)c2)cc1OC] |
| 8 | 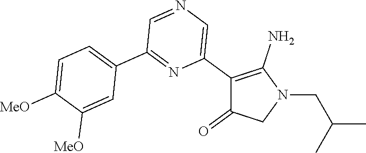 [SMILES: COc1ccc(-c2cncc(C3=C(N)N(CC(C)C)CC3=O)n2)cc1OC] |
| 9 | 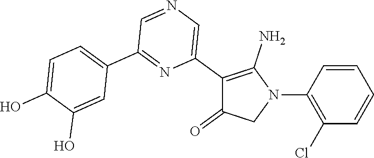 [SMILES: NC1=C(c2cncc(-c3ccc(O)c(O)c3)n2)C(=O)CN1c1ccccc1Cl] |
| 10 | 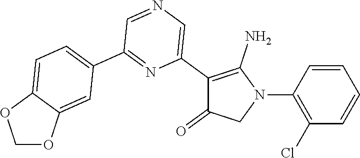 [SMILES: NC1=C(c2cncc(-c3ccc4c(c3)OCO4)n2)C(=O)CN1c1ccccc1Cl] |
| 11 | 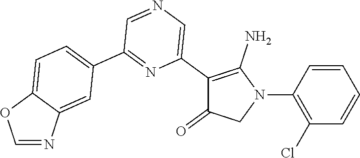 [SMILES: NC1=C(c2cncc(-c3ccc4ocnc4c3)n2)C(=O)CN1c1ccccc1Cl] |
| 12 | 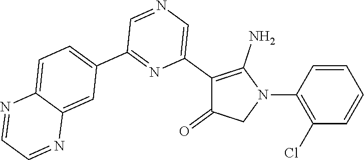 [SMILES: NC1=C(c2cncc(-c3ccc4nccnc4c3)n2)C(=O)CN1c1ccccc1Cl] |
| 13 | 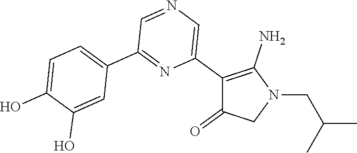 [SMILES: CC(C)CN1CC(=O)C(c2cncc(-c3ccc(O)c(O)c3)n2)=C1N] |
| 14 | 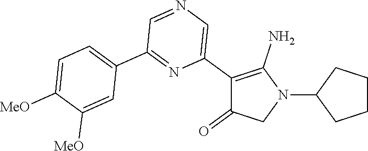 [SMILES: COc1ccc(-c2cncc(C3=C(N)N(C4CCCC4)CC3=O)n2)cc1OC] |
| 15 | 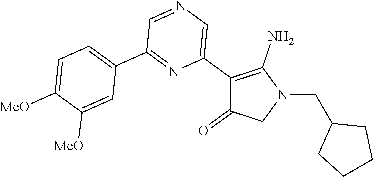 [SMILES: COc1ccc(-c2cncc(C3=C(N)N(CC4CCCC4)CC3=O)n2)cc1OC] |
| 16 | 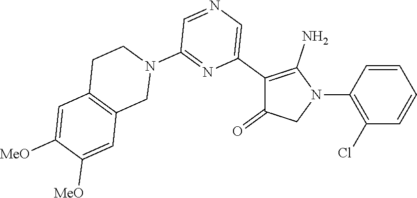 [SMILES: COc1cc2c(cc1OC)CN(c1cncc(C3=C(N)N(c4ccccc4Cl)CC3=O)n1)CC2] |
| 17 | 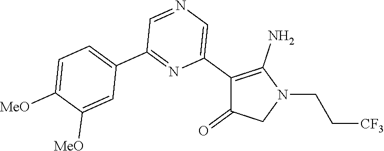 [SMILES: COc1ccc(-c2cncc(C3=C(N)N(CCC(F)(F)F)CC3=O)n2)cc1OC] |
| 18 | 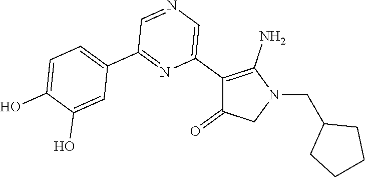 [SMILES: NC1=C(c2cncc(-c3ccc(O)c(O)c3)n2)C(=O)CN1CC1CCCC1] |
| 19 | 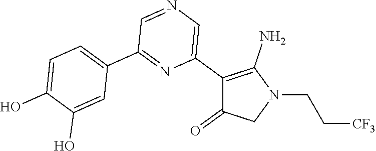 [SMILES: NC1=C(c2cncc(-c3ccc(O)c(O)c3)n2)C(=O)CN1CCC(F)(F)F] |
| 20 | 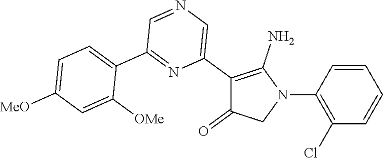 [SMILES: COc1ccc(-c2cncc(C3=C(N)N(c4ccccc4Cl)CC3=O)n2)c(OC)c1] |
| 21 | 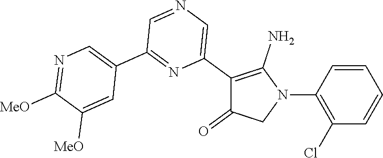 [SMILES: COc1cc(-c2cncc(C3=C(N)N(c4ccccc4Cl)CC3=O)n2)cnc1OC] |
| 22 | 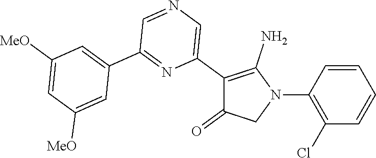 [SMILES: COc1cc(OC)cc(-c2cncc(C3=C(N)N(c4ccccc4Cl)CC3=O)n2)c1] |
| 23 | 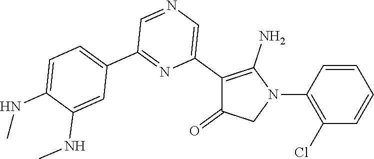 [SMILES: CNc1ccc(-c2cncc(C3=C(N)N(c4ccccc4Cl)CC3=O)n2)cc1NC] |
| 24 | 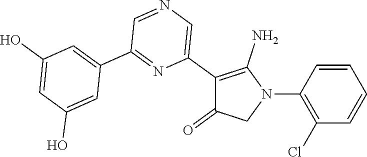 [SMILES: NC1=C(c2cncc(-c3cc(O)cc(O)c3)n2)C(=O)CN1c1ccccc1Cl] |
| 25 | 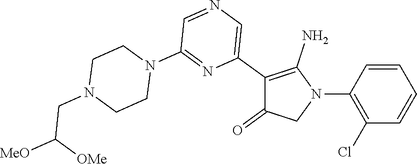 [SMILES: COC(CN1CCN(c2cncc(C3=C(N)N(c4ccccc4Cl)CC3=O)n2)CC1)OC] |
| 26 | 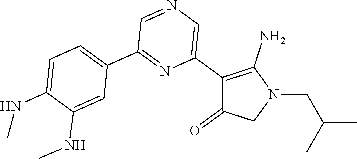 [SMILES: CNc1ccc(-c2cncc(C3=C(N)N(CC(C)C)CC3=O)n2)cc1NC] |
| 27 | 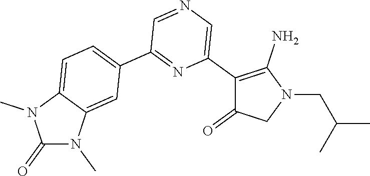 [SMILES: CC(C)CN1CC(=O)C(c2cncc(-c3ccc4c(c3)n(C)c(=O)n4C)n2)=C1N] |
| 28 | 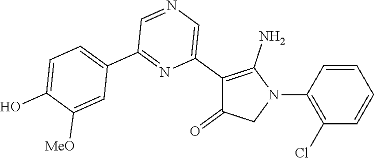 [SMILES: COc1cc(-c2cncc(C3=C(N)N(c4ccccc4Cl)CC3=O)n2)ccc1O] |
| 29 | 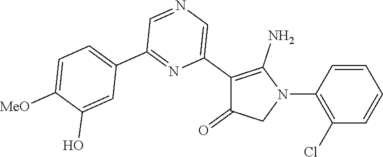 [SMILES: COc1ccc(-c2cncc(C3=C(N)N(c4ccccc4Cl)CC3=O)n2)cc1O] |
| 30 | 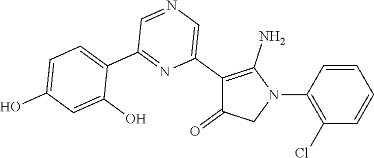 [SMILES: NC1=C(c2cncc(-c3ccc(O)cc3O)n2)C(=O)CN1c1ccccc1Cl] |
| 31 | 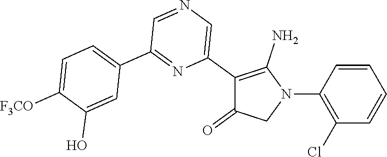 [SMILES: NC1=C(c2cncc(-c3ccc(OC(F)(F)F)c(O)c3)n2)C(=O)CN1c1ccccc1Cl] |
| 32 | 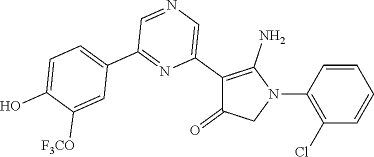 [SMILES: NC1=C(c2cncc(-c3ccc(O)c(OC(F)(F)F)c3)n2)C(=O)CN1c1ccccc1Cl] |
| 33 | 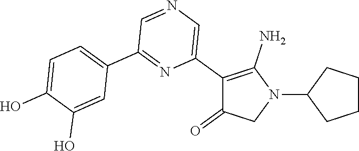 [SMILES: NC1=C(c2cncc(-c3ccc(O)c(O)c3)n2)C(=O)CN1C1CCCC1] |
| 34 | 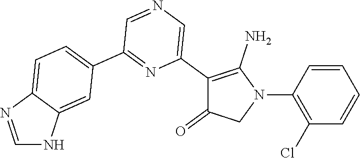 [SMILES: NC1=C(c2cncc(-c3ccc4nc[nH]c4c3)n2)C(=O)CN1c1ccccc1Cl] |
| 35 | 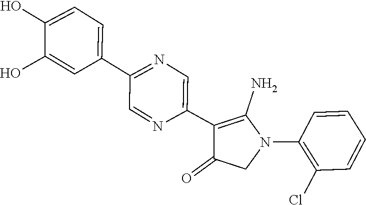 [SMILES: NC1=C(c2cnc(-c3ccc(O)c(O)c3)cn2)C(=O)CN1c1ccccc1Cl] |
| 36 | 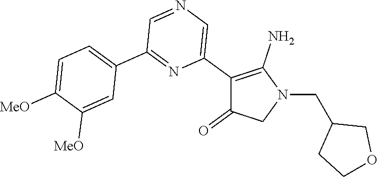 [SMILES: COc1ccc(-c2cncc(C3=C(N)N(CC4CCOC4)CC3=O)n2)cc1OC] |
| 37 | 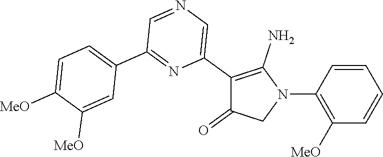 [SMILES: COc1ccc(-c2cncc(C3=C(N)N(c4ccccc4OC)CC3=O)n2)cc1OC] |
| 38 | 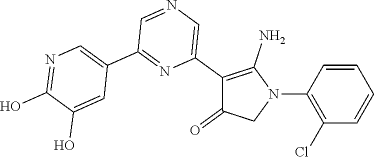 [SMILES: NC1=C(c2cncc(-c3cnc(O)c(O)c3)n2)C(=O)CN1c1ccccc1Cl] |
| 39 | 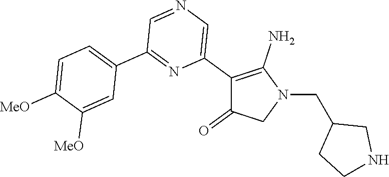 [SMILES: COc1ccc(-c2cncc(C3=C(N)N(CC4CCNC4)CC3=O)n2)cc1OC] |
| 40 | 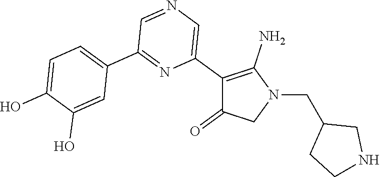 [SMILES: NC1=C(c2cncc(-c3ccc(O)c(O)c3)n2)C(=O)CN1CC1CCNC1] |
| 41 | 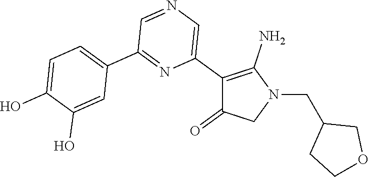 [SMILES: NC1=C(c2cncc(-c3ccc(O)c(O)c3)n2)C(=O)CN1CC1CCOC1] |
| 42 | 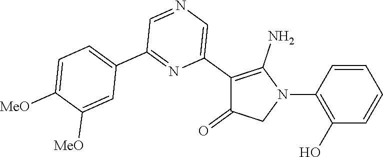 [SMILES: COc1ccc(-c2cncc(C3=C(N)N(c4ccccc4O)CC3=O)n2)cc1OC] |
| 43 | 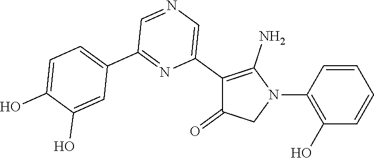 [SMILES: NC1=C(c2cncc(-c3ccc(O)c(O)c3)n2)C(=O)CN1c1ccccc1O] |
| 44 | 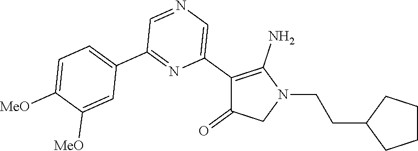 [SMILES: COc1ccc(-c2cncc(C3=C(N)N(CCC4CCCC4)CC3=O)n2)cc1OC] |
| 45 | 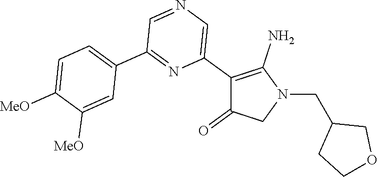 [SMILES: COc1ccc(-c2cncc(C3=C(N)N(CC4CCOC4)CC3=O)n2)cc1OC] |
| 46 | 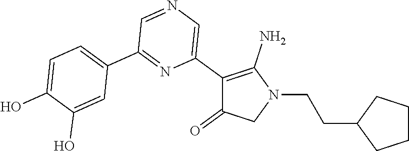 [SMILES: NC1=C(c2cncc(-c3ccc(O)c(O)c3)n2)C(=O)CN1CCC1CCCC1] |
| 47 | 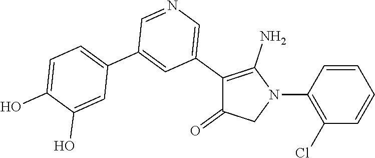 [SMILES: NC1=C(c2cncc(-c3ccc(O)c(O)c3)c2)C(=O)CN1c1ccccc1Cl] |
| 48 | 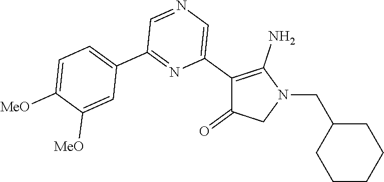 [SMILES: COc1ccc(-c2cncc(C3=C(N)N(CC4CCCCC4)CC3=O)n2)cc1OC] |
| 49 | 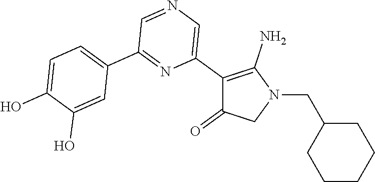 [SMILES: NC1=C(c2cncc(-c3ccc(O)c(O)c3)n2)C(=O)CN1CC1CCCCC1] |
| 50 | 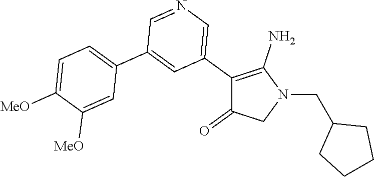 [SMILES: COc1ccc(-c2cncc(C3=C(N)N(CC4CCCC4)CC3=O)c2)cc1OC] |
| 51 | 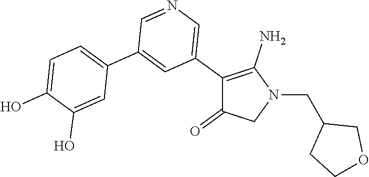 [SMILES: NC1=C(c2cncc(-c3ccc(O)c(O)c3)c2)C(=O)CN1CC1CCOC1] |
| 52 | 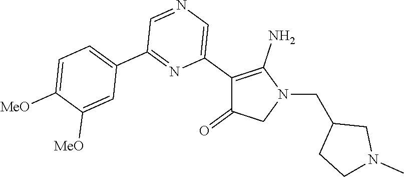 [SMILES: COc1ccc(-c2cncc(C3=C(N)N(CC4CCN(C)C4)CC3=O)n2)cc1OC] |
| 53 | 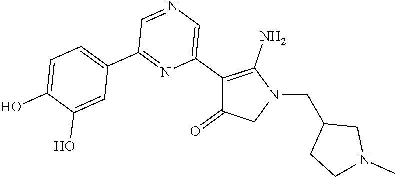 [SMILES: CN1CCC(CN2CC(=O)C(c3cncc(-c4ccc(O)c(O)c4)n3)=C2N)C1] |
| 54 | 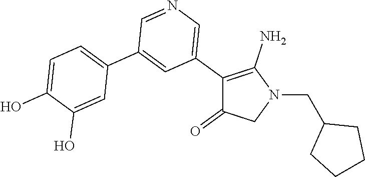 [SMILES: NC1=C(c2cncc(-c3ccc(O)c(O)c3)c2)C(=O)CN1CC1CCCC1] |
| 55 | 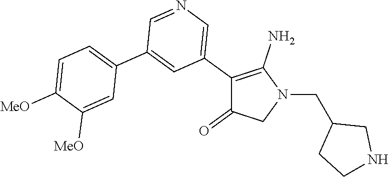 [SMILES: COc1ccc(-c2cncc(C3=C(N)N(CC4CCNC4)CC3=O)c2)cc1OC] |
| 56 | 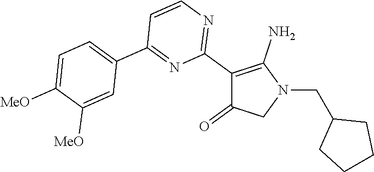 [SMILES: COc1ccc(-c2ccnc(C3=C(N)N(CC4CCCC4)CC3=O)n2)cc1OC] |
| 57 | 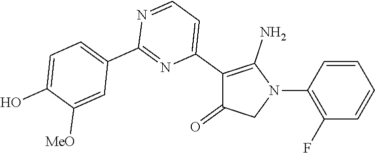 [SMILES: COc1cc(-c2nccc(C3=C(N)N(c4ccccc4F)CC3=O)n2)ccc1O] |
| 58 | 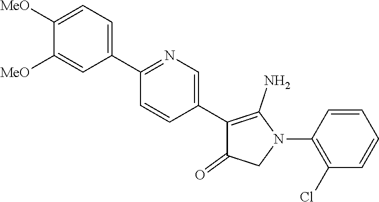 [SMILES: COc1ccc(-c2ccc(C3=C(N)N(c4ccccc4Cl)CC3=O)cn2)cc1OC] |
| 59 | 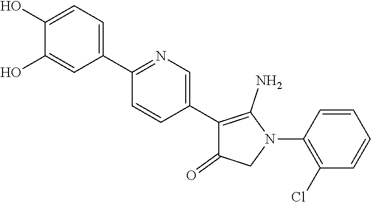 [SMILES: NC1=C(c2ccc(-c3ccc(O)c(O)c3)nc2)C(=O)CN1c1ccccc1Cl] |
| 60 | 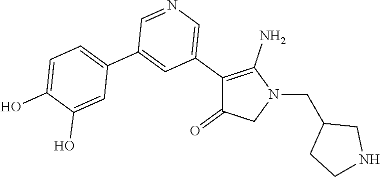 [SMILES: NC1=C(c2cncc(-c3ccc(O)c(O)c3)c2)C(=O)CN1CC1CCNC1] |
| 61 | 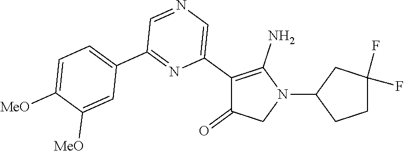 [SMILES: COc1ccc(-c2cncc(C3=C(N)N(C4CCC(F)(F)C4)CC3=O)n2)cc1OC] |
| 62 | 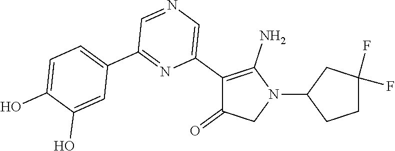 [SMILES: NC1=C(c2cncc(-c3ccc(O)c(O)c3)n2)C(=O)CN1C1CCC(F)(F)C1] |
| 63 | 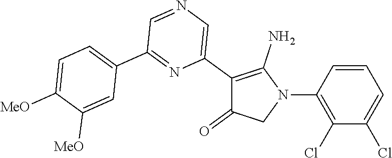 [SMILES: COc1ccc(-c2cncc(C3=C(N)N(c4cccc(Cl)c4Cl)CC3=O)n2)cc1OC] |
| 64 | 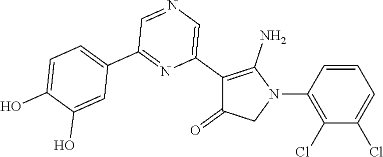 [SMILES: NC1=C(c2cncc(-c3ccc(O)c(O)c3)n2)C(=O)CN1c1cccc(Cl)c1Cl] |
| 65 | 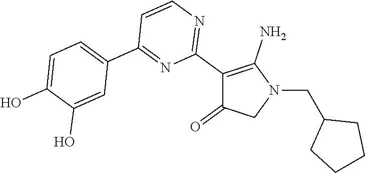 [SMILES: NC1=C(c2nccc(-c3ccc(O)c(O)c3)n2)C(=O)CN1CC1CCCC1] |
| 66 | 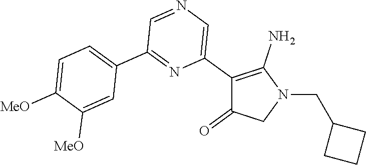 [SMILES: COc1ccc(-c2cncc(C3=C(N)N(CC4CCC4)CC3=O)n2)cc1OC] |
| 67 | 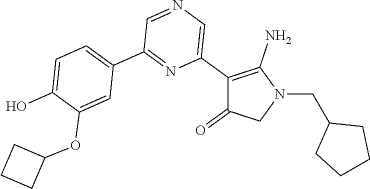 [SMILES: NC1=C(c2cncc(-c3ccc(O)c(OC4CCC4)c3)n2)C(=O)CN1CC1CCCC1] |
| 68 | 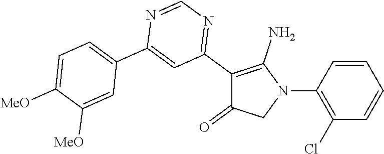 [SMILES: COc1ccc(-c2cc(C3=C(N)N(c4ccccc4Cl)CC3=O)ncn2)cc1OC] |
| 69 | 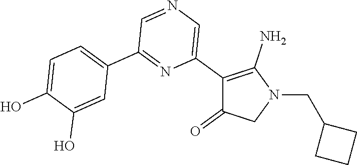 [SMILES: NC1=C(c2cncc(-c3ccc(O)c(O)c3)n2)C(=O)CN1CC1CCC1] |
| 70 | 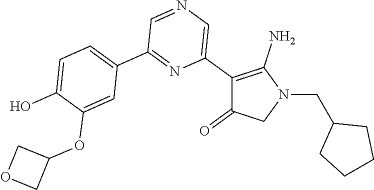 [SMILES: NC1=C(c2cncc(-c3ccc(O)c(OC4COC4)c3)n2)C(=O)CN1CC1CCCC1] |
| 71 | 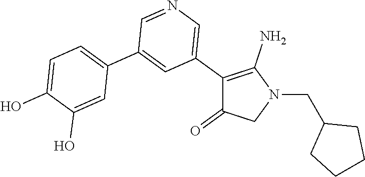 [SMILES: NC1=C(c2cncc(-c3ccc(O)c(O)c3)c2)C(=O)CN1CC1CCCC1] |
| 72 | 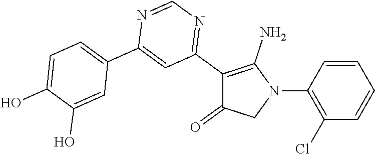 [SMILES: NC1=C(c2cc(-c3ccc(O)c(O)c3)ncn2)C(=O)CN1c1ccccc1Cl] |
| 73 | 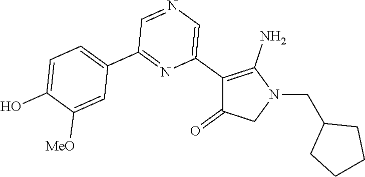 [SMILES: COc1cc(-c2cncc(C3=C(N)N(CC4CCCC4)CC3=O)n2)ccc1O] |
| 74 | 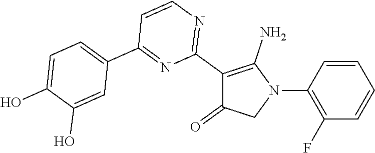 [SMILES: NC1=C(c2nccc(-c3ccc(O)c(O)c3)n2)C(=O)CN1c1ccccc1F] |
| 75 | 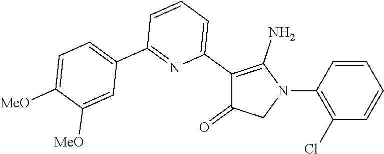 [SMILES: COc1ccc(-c2cccc(C3=C(N)N(c4ccccc4Cl)CC3=O)n2)cc1OC] |
| 76 | 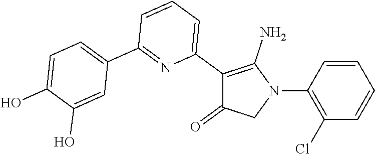 [SMILES: NC1=C(c2cccc(-c3ccc(O)c(O)c3)n2)C(=O)CN1c1ccccc1Cl] |
| 77 | 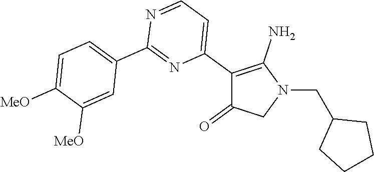 [SMILES: COc1ccc(-c2nccc(C3=C(N)N(CC4CCCC4)CC3=O)n2)cc1OC] |
| 78 | 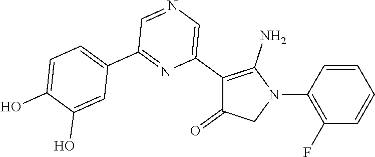 [SMILES: NC1=C(c2cncc(-c3ccc(O)c(O)c3)n2)C(=O)CN1c1ccccc1F] |
| 79 | 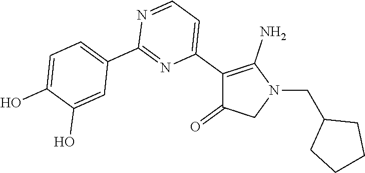 [SMILES: NC1=C(c2ccnc(-c3ccc(O)c(O)c3)n2)C(=O)CN1CC1CCCC1] |
| 80 | 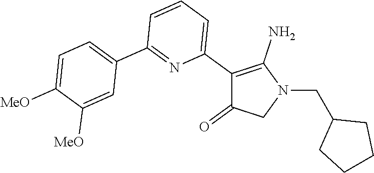 [SMILES: COc1ccc(-c2cccc(C3=C(N)N(CC4CCCC4)CC3=O)n2)cc1OC] |
| 81 | 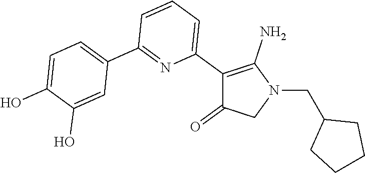 [SMILES: NC1=C(c2cccc(-c3ccc(O)c(O)c3)n2)C(=O)CN1CC1CCCC1] |
| 82 | 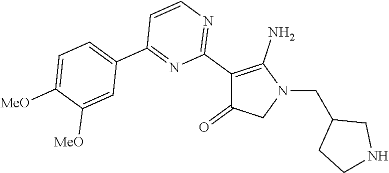 [SMILES: COc1ccc(-c2ccnc(C3=C(N)N(CC4CCNC4)CC3=O)n2)cc1OC] |
| 83 | 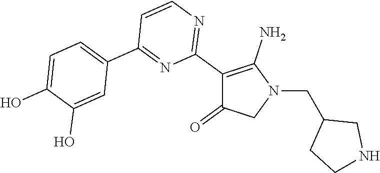 [SMILES: NC1=C(c2nccc(-c3ccc(O)c(O)c3)n2)C(=O)CN1CC1CCNC1] |
| 84 | 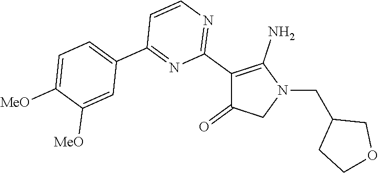 [SMILES: COc1ccc(-c2ccnc(C3=C(N)N(CC4CCOC4)CC3=O)n2)cc1OC] |
| 85 | 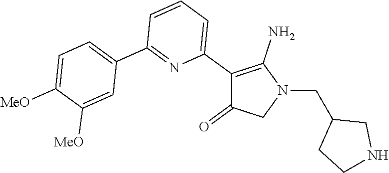 [SMILES: COc1ccc(-c2cccc(C3=C(N)N(CC4CCNC4)CC3=O)n2)cc1OC] |
| 86 | 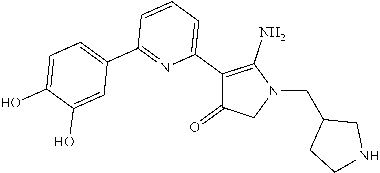 [SMILES: NC1=C(c2cccc(-c3ccc(O)c(O)c3)n2)C(=O)CN1CC1CCNC1] |
| 87 | 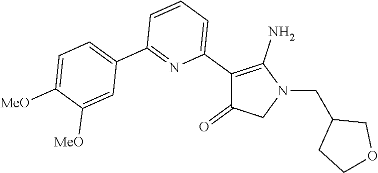 [SMILES: COc1ccc(-c2cccc(C3=C(N)N(CC4CCOC4)CC3=O)n2)cc1OC] |
| 88 | 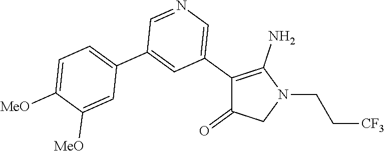 [SMILES: COc1ccc(-c2cncc(C3=C(N)N(CCC(F)(F)F)CC3=O)c2)cc1OC] |
| 89 | 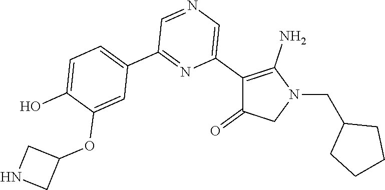 [SMILES: NC1=C(c2cncc(-c3ccc(O)c(OC4CNC4)c3)n2)C(=O)CN1CC1CCCC1] |
| 90 | 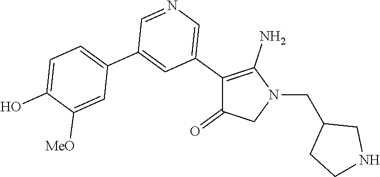 [SMILES: COc1cc(-c2cncc(C3=C(N)N(CC4CCNC4)CC3=O)c2)ccc1O] |
| 91 | 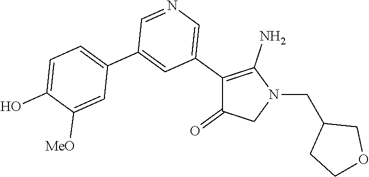 [SMILES: COc1cc(-c2cncc(C3=C(N)N(CC4CCOC4)CC3=O)c2)ccc1O] |
| 92 | 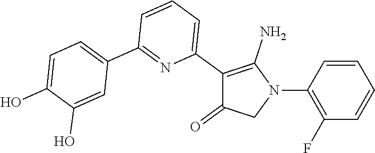 [SMILES: NC1=C(c2cccc(-c3ccc(O)c(O)c3)n2)C(=O)CN1c1ccccc1F] |
| 93 | 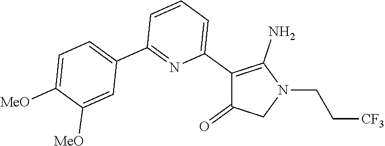 [SMILES: COc1ccc(-c2cccc(C3=C(N)N(CCC(F)(F)F)CC3=O)n2)cc1OC] |
| 94 | 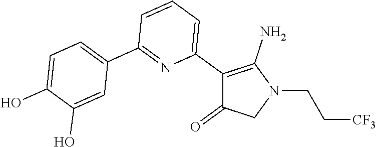 [SMILES: NC1=C(c2cccc(-c3ccc(O)c(O)c3)n2)C(=O)CN1CCC(F)(F)F] |
| 95 | 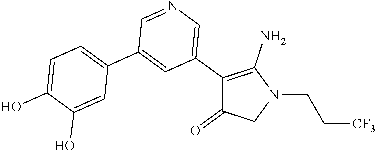 [SMILES: NC1=C(c2cncc(-c3ccc(O)c(O)c3)c2)C(=O)CN1CCC(F)(F)F] |
| 96 | 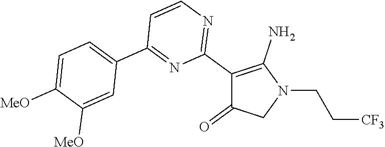 [SMILES: COc1ccc(-c2ccnc(C3=C(N)N(CCC(F)(F)F)CC3=O)n2)cc1OC] |
| 97 | 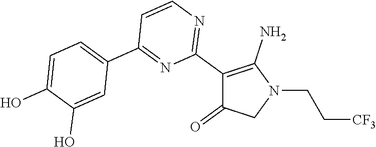 [SMILES: NC1=C(c2nccc(-c3ccc(O)c(O)c3)n2)C(=O)CN1CCC(F)(F)F] |
| 98 | 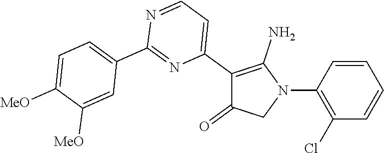 [SMILES: COc1ccc(-c2nccc(C3=C(N)N(c4ccccc4Cl)CC3=O)n2)cc1OC] |
| 99 | 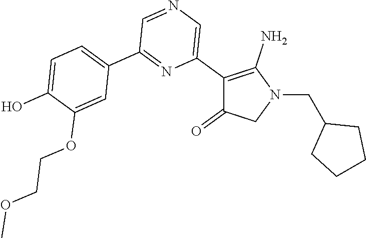 [SMILES: COCCOc1cc(-c2cncc(C3=C(N)N(CC4CCCC4)CC3=O)n2)ccc1O] |
| 100 | 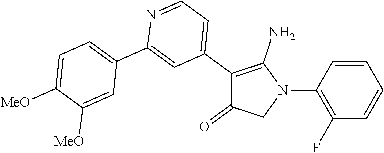 [SMILES: COc1ccc(-c2cc(C3=C(N)N(c4ccccc4F)CC3=O)ccn2)cc1OC] |
| 101 | 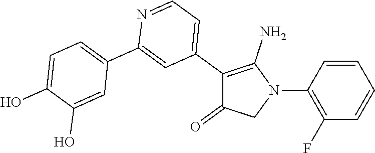 [SMILES: NC1=C(c2ccnc(-c3ccc(O)c(O)c3)c2)C(=O)CN1c1ccccc1F] |
| 102 | 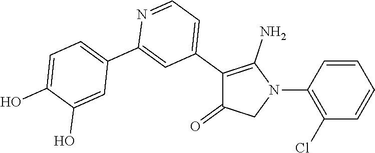 [SMILES: NC1=C(c2ccnc(-c3ccc(O)c(O)c3)c2)C(=O)CN1c1ccccc1Cl] |
| 103 | 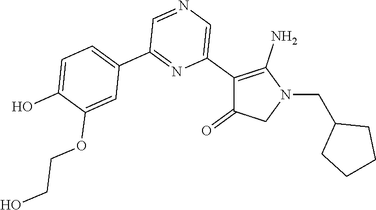 [SMILES: NC1=C(c2cncc(-c3ccc(O)c(OCCO)c3)n2)C(=O)CN1CC1CCCC1] |
| 104 | 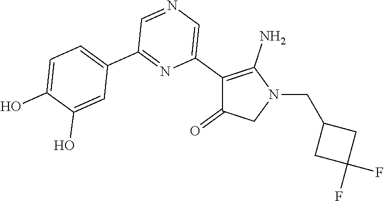 [SMILES: NC1=C(c2cncc(-c3ccc(O)c(O)c3)n2)C(=O)CN1CC1CC(F)(F)C1] |
| 105 | 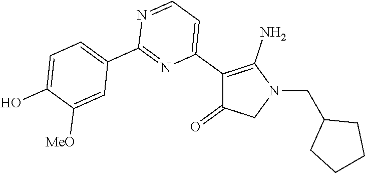 [SMILES: COc1cc(-c2nccc(C3=C(N)N(CC4CCCC4)CC3=O)n2)ccc1O] |
| 106 | 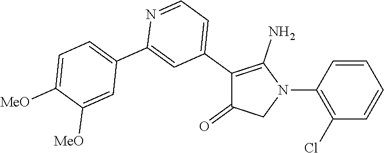 [SMILES: COc1ccc(-c2cc(C3=C(N)N(c4ccccc4Cl)CC3=O)ccn2)cc1OC] |
| 107 | 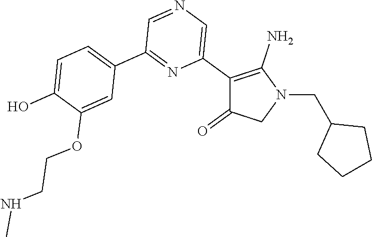 [SMILES: CNCCOc1cc(-c2cncc(C3=C(N)N(CC4CCCC4)CC3=O)n2)ccc1O] |
| 108 | 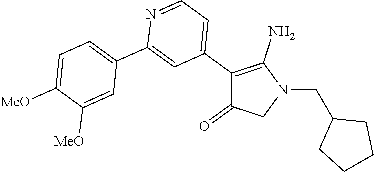 [SMILES: COc1ccc(-c2cc(C3=C(N)N(CC4CCCC4)CC3=O)ccn2)cc1OC] |
| 109 | 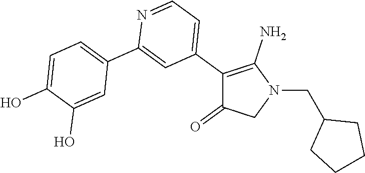 [SMILES: NC1=C(c2ccnc(-c3ccc(O)c(O)c3)c2)C(=O)CN1CC1CCCC1] |
| 110 | 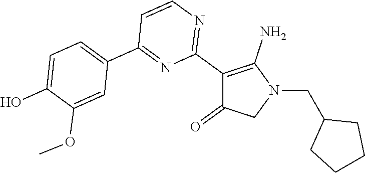 [SMILES: COc1cc(-c2ccnc(C3=C(N)N(CC4CCCC4)CC3=O)n2)ccc1O] |
| 111 | 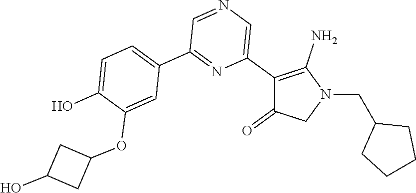 [SMILES: NC1=C(c2cncc(-c3ccc(O)c(OC4CC(O)C4)c3)n2)C(=O)CN1CC1CCCC1] |
| 112 | 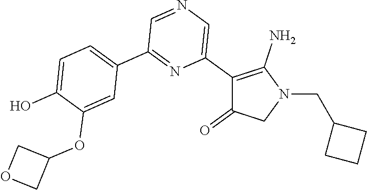 [SMILES: NC1=C(c2cncc(-c3ccc(O)c(OC4COC4)c3)n2)C(=O)CN1CC1CCC1] |
| 113 | 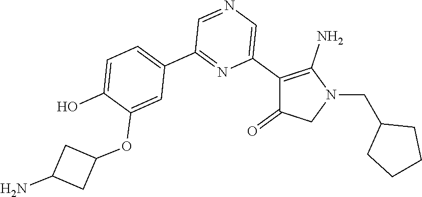 [SMILES: NC1=C(c2cncc(-c3ccc(O)c(OC4CC(N)C4)c3)n2)C(=O)CN1CC1CCCC1] |
| 114 | 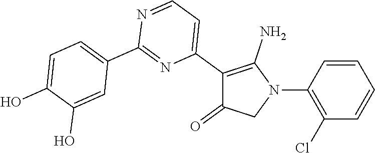 [SMILES: NC1=C(c2ccnc(-c3ccc(O)c(O)c3)n2)C(=O)CN1c1ccccc1Cl] |
| 115 | 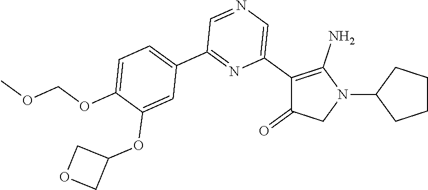 [SMILES: COCOc1ccc(-c2cncc(C3=C(N)N(C4CCCC4)CC3=O)n2)cc1OC1COC1] |
| 116 | 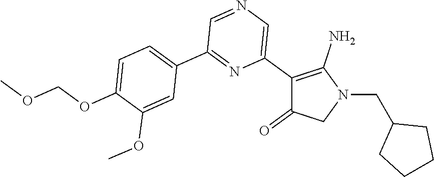 [SMILES: COCOc1ccc(-c2cncc(C3=C(N)N(CC4CCCC4)CC3=O)n2)cc1OC] |
| 117 | 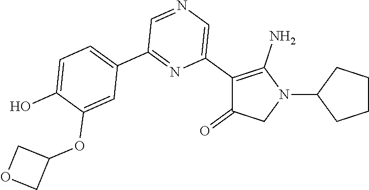 [SMILES: NC1=C(c2cncc(-c3ccc(O)c(OC4COC4)c3)n2)C(=O)CN1C1CCCC1] |
| 118 | 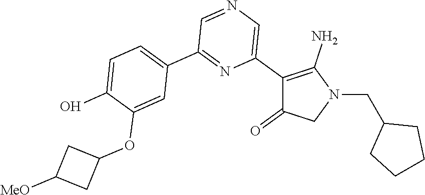 [SMILES: COC1CC(Oc2cc(-c3cncc(C4=C(N)N(CC5CCCC5)CC4=O)n3)ccc2O)C1] |
| 119 | 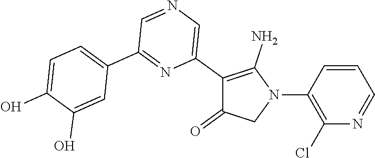 [SMILES: NC1=C(c2cncc(-c3ccc(O)c(O)c3)n2)C(=O)CN1c1cccnc1Cl] |
| 120 | 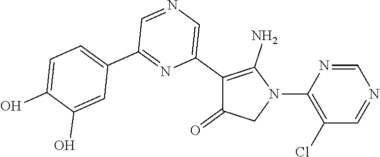 [SMILES: NC1=C(c2cncc(-c3ccc(O)c(O)c3)n2)C(=O)CN1c1ncncc1Cl] |

9. A composition comprising at least one compound, optical isomer thereof, a mixture of two isomers thereof, precursor thereof, pharmaceutically acceptable salt thereof or solvate thereof according to claim 1, as an active ingredient, together with at least one pharmaceutically acceptable carrier, excipient and/or diluent.

10. The compound according to claim 1, for use as a pharmaceutically active agent, preferably for use in a method of treating pendred syndrome or the related diseases thereof.

11. The compound according to claim 1 for use in a method of prevention and/or treatment of hearing loss, vesticular aqueduct dilation, goiter, hypertension, hypokalemia, hypothyroidism, hypochloremic alkalosis, renal tubular acidosis, volume depletion, hypovolemia, edema, cystic fibrosis, asthma, chronic obstructive pulmonary disease, rhinitis, sinusitis, cirrhosis, bone abnormality, malformation of the cochlea, chronic obstructive pulmonary disease, acute lung injury (ALI), acute respiratory distress syndrome (ARDS), bronchitis, speech impairment or rhinosinusitis.

12. A method for preventing, improving or treating pendred syndrom or the related diseases thereof comprising a step of administering the compound, optical isomer thereof, a mixture of two isomers thereof, precursor thereof, pharmaceutically acceptable salt thereof or solvate thereof according to claim 1.

13. A method for preventing, improving or treating hearing loss, vesticular aqueduct dilation, goiter, hypertension, hypokalemia, hypothyroidism, hypochloremic alkalosis, renal tubular acidosis, volume depletion, hypovolemia, edema, cystic fibrosis, asthma, chronic obstructive pulmonary disease, rhinitis, sinusitis, cirrhosis, bone abnormality, malformation of the cochlea, chronic obstructive pulmonary disease, acute lung injury (ALI), acute respiratory distress syndrome (ARDS), bronchitis, speech impairment or rhinosinusitis, comprising a step of administering the compound, optical isomer thereof, a mixture of two isomers thereof, precursor thereof, pharmaceutically acceptable salt thereof or solvate thereof according to claim 1.

14. (canceled)

15. (canceled)
